(19) United States
(12) Patent Application Publication
(10) Pub. No.: US 2025/0270549 A1
(43) Pub. Date: Aug. 28, 2025

(54) MSI2 AS A THERAPEUTIC TARGET FOR THE TREATMENT OF MYOTONIC DYSTROPHY

(71) Applicants: UNIVERSITAT DE VALÈNCIA, Valencia (ES); FUNDACIÓN PARA LA INVESTIGACIÔN DEL HOSPITAL CLINICO DE LA COMUNIDAD VALENCIANA (INCLIVA), Valencia (ES)

(72) Inventors: María SABATER ARCÍS, Burjassot (ES); Rubén Dario ARTERO ALLEPUZ, Burjassot (ES); Ariadna BARGIELA SCHONBRUNN, Burjassot (ES)

(21) Appl. No.: 18/277,744

(22) PCT Filed: Feb. 18, 2022
(86) PCT No.: PCT/EP2022/054129

§ 371 Date: Aug. 17, 2023

(30) Foreign Application Priority Data
Feb. 18, 2021 (EP) 21382133.3

(51) Int. Cl. C12N 15/113

(52) U.S. Cl. CPC C12N 15/113; C12N 2310/14; C12N 2310/313; C12N 2310/321; C12N 2310/341; C12N 2310/3515; C12N 2310/531

(57) ABSTRACT

The invention relates to inhibitors of MSI2 functions that are able to decrease MSI2 activity, MSI2 transcript levels or MSI2 protein levels in myotonic dystrophy cells or cells with MD phenotype, wherein said inhibitor is for use in the treatment of myotonic dystrophy in a mammal.

### BACKGROUND

### Technical Field

The present disclosure relates to the medical field. In particular, the present disclosure relates to an inhibitor of RNA-binding protein Musashi (MSI2) or an analogue thereof for use in the treatment of myotonic dystrophy.

### Description of the Related Art

Myotonic dystrophy (MD) is characterized by progressive skeletal muscle weakness. People with this disorder often have prolonged muscle contractions (myotonia) and are not able to relax certain muscles after use. Also, affected people may have slurred speech or temporary locking of their jaw. The Myotonic dystrophy foundation describes in their website (https://www.myotonic.org/what-dm/how-dm-affects-your-body) the symptoms of MD at different ranges of severity.

There are two major types of myotonic dystrophy: type 1 (MD1) and type 2 (MD2). Their signs and symptoms are similar, although type 2 tends to be milder than type 1. The two types of myotonic dystrophy are caused by mutations in different genes. MD1 is a life-threatening and chronically debilitating disease caused by an expansion of the CTG triplet repeat in the 3′ untranslated region of the DM1 protein kinase (DMPK) gene. MD1 is autosomal dominant and can affect from newborns to elderly people. Adult-onset patients may become physically disabled and may have a shortened life-span. Most symptoms are neuromuscular, including muscle weakness (myopathy), muscle stiffness and trouble relaxing muscles (myotonia), and progressive muscle wasting (atrophy), but MD1 is characteristically multisystemic and also affects the heart, the brain, and the smooth musculature. Pneumonia and respiratory distress due to muscle weakness and atrophy, and heart failure, are the most frequent causes of death. MD2 is also characterized by progressive muscle wasting and weakness. Symptoms typically begin in a person's twenties, and patients with this condition often have prolonged muscle contractions and are not able to relax certain muscles after use. Further, affected people may have slurred speech, temporary locking of their jaw and muscle pain and weakness that mainly affects the neck, shoulders, elbows, and hips. Less common symptoms in MD2 include abnormalities of the electrical signals that control the heartbeat (cardiac conduction defects), clouding of the lens in the eyes (cataracts), and diabetes.

In MD1, mutant DMPK transcripts in skeletal muscle, heart, and brain tissue get retained in the cell nucleus into microscopically visible ribonuclear foci. CTG trinucleotide expansions fold into stable double-stranded structures that sequester the splicing factors Muscleblind-like (MBNL) proteins. CUGBP Elav-like family member 1 (CELF1) regulates alternative splicing antagonistically to MBNL1. In contrast to MBNL1, CELF1 is not sequestered into ribonuclear foci, but is hyper-activated and stabilized in the cell nucleus in MD1.

As mentioned, muscle wasting is a key symptom in MD1 patients. Multiple alterations have been described to explain muscle dysfunction in MD1, including an increased autophagy. Muscle wasting and histological defects were observed in an inducible mouse model that express 960 CUG repeats that showed an imbalance between anabolic and catabolic pathways that normally regulate muscle mass. Specifically, the upregulation of proteins involved in the autophagic pathway has been described. Similarly, in a Drosophila MD1 model and in MD1 myotubes it was demonstrated that autophagy flux was overactivated and genetic or chemical blockade of this pathway, by mTOR upregulation or chloroquine treatment, resulted in an improvement of muscle function and molecular markers of muscle atrophy. Similarly, involvement of Muscleblind proteins with autophagy was demonstrated. In a Drosophila inducible model, overexpression of mbl isoform C (the ortholog in Drosophila of MBNL) was enough to recover muscle area and expression of autophagy-related genes. Moreover, in MD1 myotubes and MD1 model mice, treatment with chloroquine, an autophagy blocker, resulted in increased levels of MBNL1 and 2.

Additional studies demonstrated that miRNAs levels are also altered in skeletal muscle, heart, and blood samples from MD1 patients and MD1 muscle cells and mouse models. Specifically, it was reported that miR-7 levels are impaired in human biopsies from skeletal muscle and MD1 muscle cells. Independent studies demonstrated that this miRNA represses autophagy through up-regulation of mTOR signalling and direct inhibition of some autophagy genes (ATG7, ULK2 and ATG4A). miR-7 biogenesis is regulated by Musashi homolog 2 (MSI2) and Hu antigen R (HuR). However, MSI2 and HuR are proteins involved in complex and numerous cellular pathways, and their direct implications in miR-7 levels as well as their contribution to MD1 pathology in different tissues remains unknown.

In order to shed some light into the molecular pathways that regulate miR-7 biogenesis, we study herein the impact of MSI2 levels and its overexpression in MD cells and animal model, demonstrating that the intracellular concentration of MSI2 protein is an important factor directly related to MD1 pathology and thus we propose the use of inhibitors directed against MSI2 protein to increase miR-7 intracellular levels for the treatment of myotonic dystrophy.

### BRIEF SUMMARY

In one aspect, the present disclosure relates to an inhibitor of RNA-binding protein Musashi homolog 2 (MSI2), for use in the treatment of myotonic dystrophy in a mammal, wherein the inhibitor:
i) is an oligonucleotide that comprises at least 7 consecutive nucleotides in length, wherein said 7 consecutive nucleotides have at least 90% identity over the complementary sequence of the full length sequence of SEQ ID NO: 1, or
ii) is the molecule 2,3,4,10-Tetrahydro-7,10-dimethyl-2,4-dioxobenzo[g]pteridine-8-carboxaldehyde, any analogue or derivatives thereof.

Preferably, the inhibitor is an oligonucleotide according to i), and wherein the oligonucleotide is selected from the group consisting of small interfering RNAs, antisense oligonucleotides, gapmers, morpholino oligomers, FANA oligonucleotides, agomiRs, miRNA mimics, antagomiRs, blockmiRs, PNAs, LNAs (locked nucleic acid antisense oligonucleotides), splice-switching oligonucleotides (SSOs), LNA-based splice-switching oligonucleotides (LNA SSOs), LNA/DNA mixmers or miRNA sponges or variations thereof.

Preferably, the oligonucleotide comprises at least 10-25 nucleotides in length, from which at least 7 consecutive nucleotides in length have at least 90% identity over the complementary sequence of the full length sequence of any of SEQ ID: 2, or 109 to 137. Preferably, the oligonucleotide comprises at least 10-25 nucleotides in length, from which at least 7 consecutive nucleotides in length have at least 90% identity over the complementary sequence of the full sequence of any of the oligonucleotides of SEQ ID NO: 3 to SEQ ID NO: 37.

Preferably, the oligonucleotide comprises at least 10-25 nucleotides in length, from which at least 7 consecutive nucleotides in length have at least 95% identity over the full sequence of any of the oligonucleotides SEQ ID NO: 38 to 43 or 76, 77, 78.

Preferably, the inhibitor is for use in the treatment of myotonic dystrophy type 1. Preferably, the mammal is a human being.

Preferably, the inhibitor is comprised in a pharmaceutical composition, optionally further comprising a carrier and/or one or more pharmaceutically acceptable excipients.

In a further aspect, the present disclosure relates to an oligonucleotide that is between 10-50 nucleotides, preferably between 15-25, nucleotides in length, of which at least 5 consecutive nucleotides have at least 90% identity over the complementary full length sequence of any of SEQ ID NO: 1 to SEQ ID NO: 37. Preferably, the oligonucleotide consists of a sequence that is identical to the complementary sequence of any of SEQ ID NO: 3 to SEQ ID NO: 37.

In a further aspect, the present disclosure relates to an oligonucleotide that is between 10-50, preferably between 15-25, nucleotides in length, of which at least 5 consecutive nucleotides have at least 90% identity over the full length sequence of any of SEQ ID NO: 38 to 43 or 76, 77, 78. Preferably, the oligonucleotide is selected from the group consisting of any of SEQ ID NO: 38 to 43 or 76, 77, 78.

In a further aspect, the present disclosure relates an expression vector comprising a sequence encoding a short hairpin RNA molecule (shRNA), wherein the shRNA comprises:
(i) a first sequence comprising an oligonucleotide according to claims 9 to 12,
(ii) a second sequence that is adjacent to the first sequence, wherein said second sequence is at least 95% complementary to the first sequence, wherein the first and the second sequences form a hairpin by hybridization, and
wherein said sequence encoding an shRNA molecule is operably linked to an RNA polymerase promoter. Preferably, the vector is an adeno-associated vector.

In a further aspect, the present disclosure relates to a pharmaceutical composition comprising the oligonucleotide according to the previous aspects, or the expression vector according to the previous aspect, or a mixture of two or more of them, optionally further comprising a carrier and/or one or more pharmaceutically acceptable excipients.

In a further aspect, the present disclosure relates to a pharmaceutical composition according to the previous aspect for use in the treatment of myotonic dystrophy in a mammal. Preferably, said pharmaceutical composition is for use in the treatment of myotonic dystrophy type 1. Preferably, the mammal is a human being.

### BRIEF DESCRIPTION OF THE SEVERAL VIEWS OF THE DRAWINGS

FIG. 1. The processing machinery of pri-miR-7 is altered in MD1 myotubes. (A) Schematic representation of the secondary structure of pri-miR-7 with the conserved terminal loop of miR-7 showing the complex formed by pri-miR-7-1/HuR/MSI2 (obtained from Kumar et al., 2017). (B, C) Relative expression levels of HuR and MSI2 transcripts in healthy control and MD1 myotubes. GAPDH expression was used as internal reference. (D) Western blot quantification of total levels of MSI2 in control and MD1 cells. β-ACTIN was used as an endogenous control to normalize protein levels (n=3).

FIG. 2. MSI2 is upregulated in MD1 samples. Quantification of relative expression of miR-7 (left), MSI2 transcripts (middle), and MSI2 protein levels (right), and representative blots from patient-derived biopsies (A), primary myoblasts (B) and myotubes (C). In quantifications by RT-qPCR, U1 and U6 were used as endogenous controls of miR-7 while MSI2 levels are relative to GADPH and GPI expression. Data were obtained using the 2-ΔΔCt method (n=3). In western blots, β-ACTIN or GAPDH were used as endogenous controls to normalize protein levels (n=3). The graphs show the median with interquartile range (A) and mean±s.e.m (B and C). (D) Pearson's correlation between miR-7 relative expression and MSI2 protein levels in biopsies and transdifferentiated myotubes (TDMs). Representative confocal images of MSI2 immunostained healthy controls (CNT) (E) and MD1 myotubes (F). Nuclei were counterstained with DAPI. Scale bar=20 μm. Statistical analyses are Student's t-tests. * P<0.05, ** P<0.01, *** P<0.001, P<0.0001. (G) The graph represents MSI2 transcripts per million in RNA-seq experiments from biopsies from 40 MD1 patients and 10 controls according to Wang et al. 2019 [Wang, E. T. et al. Transcriptome alterations in myotonic dystrophy skeletal muscle and heart. Hum Mol Genet 28, 1312-1321, doi: 10.1093/hmg/ddy432 (2019)]. The graph shows the median with interquartile range (H) Pearson's correlations between MSI2 transcripts per million in MD1 biopsies and ankle dorsiflexion strength (ADS) measured in biopsies donors. Data according to Wang et al. 2019.

FIG. 3. Atrophic muscle markers ameliorate after MSI2 reduction using a gapmer in MD1 TDMs. (A) Cell growth inhibition assay by MTS method. Human CNT myotubes were transfected with increasing concentrations of ASO1, 2 and 3 (n=4). TC10 (31.2 nM, 110 nM and 33.45 nM for ASO1, 2 and 3, respectively) were obtained using the least-squares non-lineal regression model. Vertical dashed lines indicate used concentrations in the following experiments: 30 and 150 nM. (B) MSI2 quantification by RT-qPCR relative to GAPDH and GPI and (C) MSI2 quantification by Western blot. Graph and representative blot image of MSI2 immunodetection are shown. β-ACTIN expression was used as an endogenous control. (D) Quantification of miR-7 relative to U1 and U6 in MD1 TDMs transfected with ASOs. Data were obtained using the 2^{-ΔΔCt} method (n=3). (E) Quantification by RT-qPCR relative to GAPDH and GPI of the expression levels of P21 and TGFBR1 after treatment of the muscle cells with the indicated concentrations of gapmers targeting MSI2. The statistical analysis was performed comparing values from treated cells with their respective scramble (black dashed line). Data were obtained using the 2^{-ΔΔCt} method (n=3). (F) Analysis of myogenic fusion index and (G) myotube diameter of MD1 myoblasts transfected with the indicated concentrations of ASOs or scramble (n=10-15). (H-J) Representative confocal images of Desmin-immunostained human fibroblasts transdifferentiated for 7 days after ASOs transfection into MD1 cells and their respective scramble control at 150 nM. Nuclei were counterstained with DAPI. Scale bar, 40 μm. The statistical analysis was performed comparing values from treated cells with their respective scramble. The bar graphs show mean±s.e.m. * P<0.05, ** P<0.01, P<0.001, according to Student's t-test. (K) The scrambled (SC) control ASO does not affect MSI2 levels: Quantification by qRT-PCR of the MSI2 relative expression levels in healthy TDMs differentiated for 7 days treated with 150 nM of SC. The bar graph shows the mean #s.e.m. The mean of GAPDH and GPI expression was used as a reference for normalization (n=3).

FIG. 4. Degradation of MSI2 transcripts enhances MBNL1 levels in 7-days differentiated TDMs. (A) Quantification and representative blots of MBNL1 levels in protein extracts obtained from MD1 TDMs treated with the indicated concentrations of MSI2 or scramble ASOs. (B, C) Representative blots used for quantitative analyses of MBNL2 and CELF1 levels. β-ACTIN expression was used as an endogenous control (n=3). Representative confocal images of (D-I) MBNL1 or (J-O) MBNL2 immunodetection in MD1 TDMs treated for 48 h with scramble (sc) (D, J, G, M), ASO1 (E,K,H,N) or ASO3 (F,L,I,O) at the indicated concentration. Nuclei were counterstained with DAPI (scale bar, 50 μm). (P-R) Semiquantitative RT-PCR analyses and representative gels of splicing events altered in (P) PKM isoforms represented as the % of PKM2 and (Q) SERCA1 (exon 13), and (R) NFIX (exon 7) represented as the % of exon inclusion in MD1 cells. GAPDH was used as internal control (n=3). In all cases, the statistical analyses were performed comparing values from treated cells with those obtained for cells treated with the scramble ASO at the same concentration. The bar graphs show mean±s.e.m. * P<0.05, ** P<0.01, *** P<0.001, according to Student's t-test. (S-V) MBNL1 transcripts, MBNL2 and CELF1 protein levels do not significantly change upon treatment with MSI2 targeting ASOs: (S) MBNL1 transcripts quantification by RT-qPCR in DM1 myotubes transdifferentiated for 7 days and treated with the indicated molecules targeting MSI2 mRNA. GAPDH expression levels were used as endogenous controls (n=3). Western blot quantification of MBNL2 (T) and CELF1 (U) protein expression levels in 7-days transdifferentiated DM1 TDMs after treatment with the indicated ASOs. β-ACTIN expression was used as an endogenous control (n=3). None of the statistical comparisons of experimental to scrambled-treated TDMs reached the significance threshold. Green dashed lines indicate values detected in control myotubes. The bar graphs show the mean±s.e.m.

FIG. 5. Silencing MSI2 with a combination of two siRNAs is effective in MD1 muscle cells. (A) MSI2 relative expression analyzed by RT-qPCR. GAPDH and GPI were used as reference genes (n=3). (B) Relative expression levels of miR-7 measured by RT-qPCR (calculated using the 2^{-ΔΔCt} method). Data were normalized to U1 and U6 levels. (C) Quantification of the fusion index of MD1 myotubes treated with the indicated siRNA. (D, E) Representative confocal images of Desmin-immunostained human fibroblasts transdifferentiated for 7 days after siRNA transfection into MD1 cells. Nuclei were counterstained with DAPI. Error bars indicate s.e.m. * P<0.05, *** P<0.001, **** P<0.0001 according to Student's t-test. (F) Quantification of myotube diameter of DM1 myoblasts transfected with the indicated siRNA (between 10 and 15 images analyzed, totalling some 400-500 nuclei) (G) miR-7, P21, and TGFBR1 in DM1 TDMs treated with 100 nM scrambled (sc) siRNA or a combination of two siRNAs targeting MSI2 at a final concentration of 100 nM. Levels of MSI2, P21, and TGFBR1 are relative to the mean of GAPDH and GPI, whereas miR-7 was normalized to U1 and U6. Data come from three biological replicates.

FIG. 6. Downregulation of MSI2 by treatment with a small molecule improves MD1-related phenotypes. (A) MSI2 relative expression analyzed by RT-qPCR. GAPDH, HPRT1 and GPI were used as reference genes (n=3). (B) Relative expression levels of miR-7 measured by RT-qPCR (calculated using the 2^{-ΔΔCt} method). Data were normalized to U1 and U6 levels. Relative expression of (C) ATG4A, (D) ATG7, (E) TGFBR1 and (F) P21 analyzed by RT-qPCR. GAPDH and GPI were used as reference genes (n=3). Quantification by western blot of (G) MSI2, (H) ATG4A, (I) ATG7 and (J) P62. Representative blots used for quantification are shown below the bar graphs. β-ACTIN was used as endogenous control to normalize protein levels (n=3). (K) Representative confocal images of LysoTracker staining of control and MD1 cells treated with the indicated concentrations of the small molecules. Nuclei were counterstained with DAPI. (L) Representative confocal images of Desmin-immunostained human fibroblasts transdifferentiated for 7 days into myotubes. Nuclei were counterstained with DAPI. (M) Quantification of the fusion index of MD1 TDMs treated with the indicated compounds and concentrations. (N) myotube diameter of DM1 TDMs treated with the compound (n=10-15 images). Error bars indicate s.e.m. * P<0.05, ** P<0.01 *** P<0.001, **** P<0.0001 according to Student's t-test. Legend: CNT DMSO: control healthy cells (CNT) treated with DMSO. DMSO: MD1 cells treated with DMSO. Ro-08-2750 10 μM: MD1 cells treated with Ro-08-2750 at 10 μM.

FIG. 7. Inhibition of MSI2 expression by agomiR-107 addition is beneficial for MD1 cells. (A) MSI2 relative expression analyzed by RT-qPCR. GAPDH, HPRT1 and GPI were used as reference genes (n=3). (B) Relative expression levels of miR-7 measured by RT-qPCR (calculated using the 2^{-ΔΔCt} method). Data were normalized to U1 and U6 levels. Relative expression of (C) ATG4A, (D) ATG7, (E) TGFBR1 (F) and P21 analyzed by RT-qPCR. GAPDH, HPRT1 and GPI were used as reference genes (n=3). Quantification by western blot of (G) MSI2, (H) ATG4A, (I) ATG7 and (J) P62. Representative blots used for quantification are shown below the bar graphs. β-ACTIN was used as endogenous control to normalize protein levels (n=3). (K) Representative confocal images of LysoTracker staining of control and MD1 cells treated with the indicated concentrations of the small molecules. Nuclei were counterstained with DAPI. (L) Representative confocal images of Desmin-immunostained human fibroblasts transdifferentiated for 7 days. Nuclei were counterstained with DAPI. (M) Quantification of the fusion index of MD1 TDMs treated with the indicated compounds and concentrations. Error bars indicate s.e.m. * P<0.05, ** P<0.01 P<0.001, **** P<0.0001 according to Student's t-test. Legend: CNT: control healthy cells. MD1: untreated MD1 cells. AgomiR-107 100 nM: MD1 cells treated with agomiR-107 at 100 nM. AgomiR-107 300 nM: MD1 cells treated with agomiR-107 at 300 nM.

FIG. 8. MSI2 overexpression has a deleterious effect in MD1 mouse muscles. (A) MSI2 relative expression analyzed by RT-qPCR (B) and western blot, at the indicated post-injection times. Protein levels are relative to Gapdh, which was used as an internal control, and correspond to the signals obtained with the anti-MSI2 antibody (bar graph). (B) Representative blots used for quantification are shown below the bar graph using an anti-MSI2 (upper gels) or anti-V5-tag (lower gels). (C) Relative expression levels of miR-7 measured by RT-qPCR (calculated using the 2^{-ΔΔCt} method). Data were normalized to U1 and U6 levels. (D) Forelimb grip strength in 2-3 months-old mice (before injection), 3.5-4 months-old mice (intermediate time point) and in 4.5 months-old mice (before euthanasia). Data were normalized to body weight. (E) Representative confocal images of muscle fibers of the indicated mouse groups stained with WGA and (F) cross-sectional muscle area quantification. Nuclei were counterstained with DAPI. (G-H) Relative expression of the indicated genes analyzed by RT-qPCR. The average of Gapdh, Gtfb2, and Hprt1 expression levels was used as reference in A, G, and H (n=3). Error bars indicate s.e.m. * P<0.05, ** P<0.01 according to Student's t-test. Legend: FVB: control mice treated with PBS; PBS: HSA^{LR} mice treated with PBS; 1×10¹² 6 weeks: HSA^{LR} mice treated with AAV at a dose of 1×10¹² vg/mice and sacrificed 6 weeks after the injection. 1.75×10¹² 6 weeks: HSA^{LR} mice treated with a dose of AAV 1.75×10¹² vg/mice and sacrificed 6 weeks after the injection; 1×10¹² 10 weeks: HSA^{LR} mice treated with AAV at a concentration of 1×10¹² vg/mice and sacrificed 10 weeks after the injection. 1.75×10¹² 10 weeks: HSA^{LR} mice treated with AAV at a dose of 1.75×10¹² vg/mice and sacrificed 10 weeks after the injection.

FIG. 9. MSI2, miR-7 and autophagy are not altered in HSA^{LR} mice in comparison to control mice (FVB). (A) Quantification of relative expression of MSI2 transcripts, (B) MSI2 protein levels, (C) miR-7 levels and (D) autophagy-related protein levels from diaphragm, gastrocnemius, and quadriceps muscles of 4.5-months HSA^{LR} and control (FVB) mice. U1 and U6 were used as endogenous controls of miR-7 while MSI2 levels are relative to GAPDH, Gtf2b and Hprt1 expression. Data were obtained using the 2^{-ΔΔCt} method (n=4-8). In western blots GAPDH was used as endogenous control to normalize protein levels (n=4-8). The graphs show the median with interquartile range and mean±s.e.m.

FIG. 10. Inhibition of MSI2 expression in DM1 TDMs restores impaired autophagic markers. (A) qRT-PCR quantification of the relative expression of the indicated genes involved in different autophagy pathway steps in DM1 and control (green dashed lines) myotubes. Statistical analyses were performed comparing DM1 versus controls. (B) Analyses of the expression of genes quantified in (A) in DM1 myotubes treated with ASOs targeting MSI2 transcripts. All comparisons for the statistical analyses were performed using their corresponding scrambled control (dashed lines). The mean of GAPDH and GPI expression was used as reference (n=3). Quantification and representative western blots of (C) ATG4A, (D) ATG7, (E) AKT-P/AKT total protein levels, and (F) LC3-II/LC3-I ratio in DM1 TDMs treated with MSI2 targeting or scrambled ASOs at the indicated concentrations. Green dashed lines indicate protein expression levels in control myotubes. β-actin was used as an endogenous control to normalize protein levels (n=3). The bar graphs show mean #SEM. * p<0.05, ** p<0.01, *** p<0.001, and **** p<0.0001 according to Student's t test.

FIG. 11. P62 levels of DM1 TDMs remain unchanged upon treatment with MSI2-targeting ASOs. Quantification and representative western blots of the P62 levels in protein extracts from TDMs treated with the indicated ASOs against the MSI2 transcripts. β-ACTIN expression was used as an endogenous control (n=3). Dashed line indicates relative levels of P62 detected in control myotubes. Statistical analyses compared experimentally treated-TDMs with the scrambled gapmer at the same concentration and found no significant differences by Student's t-tests. The bar graphs show the mean±s.e.m.

FIG. 12. Blocking MSI2 expression improves muscle wasting markers in DM1 TDMs. (A-F) Fluorescence confocal images of LC3 immunostaining (red) or (H-M) LysoTracker staining (red) in 7-day TDMs treated for 48 h with (A, D, H, and K) scramble, (B, E, I, and L) ASO1, or (C, F, J, and M) ASO3 at the indicated concentrations. Nuclei were counterstained with DAPI. Scale bar, 20 μm. (G) Quantification of LC3 puncta per square micrometer. Each condition was compared to cells treated with its corresponding scrambled oligonucleotides. The green dashed line shows the mean value obtained for control muscle cells. Quantification by qRT-PCR of the relative expression of genes involved in catabolic pathways leading to muscle degradation in DM1 and control myotubes (N) or DM1 cells treated with the indicated concentration of ASOs (O). Statistical analyses were carried out comparing DM1 with the control condition (N) or comparing ASOs with their corresponding scramble (O). GAPDH and GPI were used as endogenous controls (n=3). The bar graphs show mean±SEM. * p<0.05, ** p<0.01, *** p<0.001, and **** p<0.0001 according to Student's t test.

FIG. 13. MSI2 inhibition in DM1 myoblasts improves pathological phenotypes. (A) MSI2 quantification by western blot and representative blots in control (orange) and DM1 myoblasts treated with vehicle 0.8% DMSO (gray) or 10 μM Ro-08-2750 (red) during the last 48 h of 7-day differentiated myotubes; GAPDH expression was used as an endogenous control. (B) Quantification by qRT-PCR of MSI2 and its direct targets miR-7, P21, and TGFBR1 in the same conditions as those described in (A). (C) Quantification by qRT-PCR of the relative expression of genes involved in catabolic pathways leading to muscle degradation in DM1 treated with vehicle (gray dashed line) and control myoblasts (orange) or DM1 cells treated with compound (red). Statistical analyses were carried out comparing control and DM1 treated with Ro-08-2750 with DM1 myoblasts treated with vehicle. mRNA levels shown in (A) and (B) are relative to the mean of GAPDH, GPI, and HPRT1; miR-7 was normalized to the mean of U1 and U6 (n=3). Representative confocal images of Desmin immunostaining (green; D-F) and LC3 staining (red; H-J) of 7-day control and DM1 myoblasts treated with DMSO (D, H, and E-I) as control or with 10 μM Ro 08-2750. (F and J) Scale bars correspond to 40 μm, and the nuclei were counterstained with DAPI. (G) Quantification of myogenic fusion index of the conditions represented in (D)-(F) at 7, 10, and 14 days of differentiation (n=10-15 images). (K) Quantification of the number of LC3 puncta per square micrometer. (G and K) Each condition was compared to DM1 cells treated with DMSO. Quantification by western blot of (L) ATG4A and (M) ATG7. Representative blots used for quantification are shown on the side of the bar graphs. GAPDH was used as an endogenous control to normalize protein levels (n=3). The bar graphs show mean±SEM. * p<0.05, ** p<0.01, *** p<0.001, and **** p<0.0001 according to Student's t test.

FIG. 14. Ro 08-2750 treatment of immortalized DM1 myoblasts differentiated for 7 days validates MSI2 as a new therapeutic target. (A and B) Quantification by qRT-PCR of the indicated genes in control primary myoblasts (line CNT-10, orange dashed line) and two DM1 lines, DM1-14 and DM1-16, treated with vehicle (0.8% DMSO; gray) or 10 μM Ro-08-2750 (red) for 48 h in 7-day differentiation cultures. Asterisks above gray bars indicate statistical differences between CNT-10 and the DM1 lines. Asterisks over the black lines indicate the significant differences between both treatments (DMSO or Ro 08-2750). Gene expression is relative to the mean of GAPDH and GPI, and miR-7 was normalized to the mean of U1 and U6 (n=3). (C) Quantification of the myogenic fusion index and (D-H) representative confocal images of Desmin immunostaining (green) of control primary myoblasts (D) and DM1-14 and DM1-16 lines treated with vehicle (E and G) or with 10 μM Ro-08-2750 (F and H). (I-M) Representative confocal micrographs of LC3 immunostaining (red) and (N) quantification of the number of LC3 puncta per square micrometer in cell cultures identical to (D)-(H). Scale bar corresponds to 40 μm, and the nuclei were counterstained with DAPI (blue). The bar graphs show mean±SEM. * p<0.05, ** p<0.01, *** p<0.001, and **** p<0.0001 according to Student's t-test.

FIG. 15. Effect of Ro-08-2750 treatment in DM1 myotubes. (A) Cell growth inhibition assay by the MTS method. Human normal TDMs were transfected with a range of Ro 08-2750 concentrations (n=4). TC10 was obtained using the least-squares non-linear regression model. Quantification of mRNA relative expression by qRT-PCR of (B) P21, (C) MSI2, (E) miR-7, and (F) TGFBR1 in DM1 TDMs treated with the vehicle (DMSO), or the indicated concentration of Ro-08-2750. GAPDH, GPI, and HPRT1 or U1 and U6 expression levels were used as endogenous controls in b, c, f and e, respectively (n=3). (D) MSI2 relative protein level was quantified by western blot relative to β-ACTIN in myotubes treated with vehicle or with the indicated concentrations of the compound (n=3). Representative blots from each condition are also shown. (J) Quantification of the percentage of myogenic fusion index of DM1 TDMs with the indicated concentrations of the compound (n=10-15 images). Representative confocal images of Desmin-immunostained (green) human DM1 myotubes transdifferentiated for 7 days after treatment with (G) DMSO as control or with 1 or 3 μM Ro 08-2750 (H,I). Scale bar 40 μm. Nuclei were counterstained with DAPI. The bar graphs show mean±s.e.m. * P<0.05 according to Student t-test

FIG. 16: MSI2 inhibition by Ro 08-2750 improves fusion capacity of 10 and 14 days differentiated DM1 myoblasts. Representative confocal images of Desmin-immunostained (green) immortalized myoblasts from control (A, D), DM1 treated with 0.8% DMSO (B, E) or DM1 treated with 10 μM Ro 08-2750 (C, F) differentiated for 10 (A-C) or 14 (D-F) days. Scale bar 40 μm. Nuclei were counterstained with DAPI.

FIG. 17. MSI2 overexpression enhances DM1-like phenotypes in HSALR mice (A) Body weight measured at the indicated time points after the injection of control and DM1 model mice P2-P3 treated with PBS as vehicle (FVB and PBS, respectively), with AAV9 with the human DESMIN promoter without transgene AAV9-hDES-Nanoluc (hDES) or with the murine MSI2 AAV9-hDES-MSI2 (V4)-V5-T2A-Nanoluc (MSI2). (B) Force of the forelegs was analyzed at the indicated intermediate time point, and before the sacrifice in the same biological groups as described in A. Plots represent mean±SEM according to one-way ANOVA test. (C) Analysis of the percentage of muscle fibers with central nuclei in quadriceps and gastronemius muscles of control FVB (green bar), and PBS (gray bar), AAV9-hDESMIN-Nanoluc or AAV9-hDESMIN-MSI2-V5-T2A-Nanoluc treated (blue and orange bars) HSALR mice. (D,E) Representative haematoxylin and eosin staining of quadriceps (D) and gastrocnemius (E) muscles from all four experimental groups. Arrows point to examples of centrally located nuclei in muscle fibers. Scale bar=100 μm. The scatterplots show the median with the interquartile range with the minimum and maximum values.* p<0.05, ** p<0.01, *** p<0.001, and **** p<0.0001 according to one-way ANOVA test with Tukey's HSD post hoc test; FVB (n=8), HSALRPBS (n=8), HSALR AAV9-hDES (n=10) and HSALR AAV9-hDES-MSI2 (n=15).

FIG. 18. MSI2 is overexpressed in skeletal muscle upon AAV9 infection. (A) Quantification of the HSA transgene by qRT-PCR in quadriceps and gastrocnemius from control and DM1 model mice P2-P3 injected (i.v) with PBS (FVB and PBS, respectively), AAV9-hDES-Nanoluc (hDES) or AAV9-hDES-MSI2 (V4)-V5-T2A-Nanoluc (MSI2). The expression of the AAV9 in hDES and MSI2 mice was assessed by quantifying Nanoluc transgene expression by qRT-PCR (2-ΔCt) (B) and by immunoblotting the V5 tag of the exogenous MSI2 using automated western blot analyzer (Jess). V5 was normalized to total protein (C). (D) Representative images of V5 Jess western in quadriceps and gastrocnemius muscles are also shown. Blue dots represent de percentage of total protein in each sample relative to hDES in gastrocnemius. V5 band appears around 46 kDa. The percentages in blue indicate the percentage of total protein. (E,F) Western Blotting analyses and representative blots of MSI2 levels in each condition. Protein levels are relative to Gapdh, which was used as an internal control. Quantification by qRT-PCR of the relative expression of (G) MSI2 and (H) miR-7 in the indicated biological groups. miR-7 quantification is relative to endogenous U1 and U6 levels and MSI2 is referenced to Gapdh, Gtf2b and Hprt1 expression. In all cases, relative levels were normalized to hDES samples. P values were calculated using one-way ANOVA test with Tukey's HSD post hoc test. Plots represent mean±SEM. * p<0.05, ** p<0.01, *** p<0.001, and **** p<0.0001. FVB (n=8), HSALRPBS (n=8), HSALR AAV9-hDES (n=6-10) and HSALR AAV9-hDES-MSI2 (n=13-15).

FIG. 19. MSI2 overexpression promotes reduced area of muscular fibers. (A) Representative confocal images of WGA-stained quadriceps and gastrocnemius sections from control and DM1 model mice treated with PBS (FVB, PBS, respectively) or DM1 mice injected with AAV9-hDES-Nanoluc (hDES) or AAV9-hDES-MSI2 (V4)-V5-T2A-Nanoluc (MSI2). Nuclei were counterstained with DAPI. Scale bar=50 μm. Quantification of the cross-sectional area of muscle fibers from quadriceps (B) and gastrocnemius (C) muscles in the indicated biological groups. Tables indicate the minimum, maximum, and median values (in μm2) of the area of the fibers in the indicated conditions. Plots represent mean±SEM. ** p<0.01 according to one-way ANOVA test with Tukey's HSD post hoc test. FVB (n=6), HSALRPBS (n=8), HSALR AAV9-hDES (n=9) and HSALR AAV9-hDES-MSI2 (n=13-14).

FIG. 20. The distribution of the area of myofibers is impaired upon MSI2 overexpression. Distribution of myofibers size in quadriceps (A) and gastrocnemius (B) muscles in wild type and HSALR mice treated with PBS (FVB, PBS, respectively) or DM1 mice injected with AAV9-hDES-Nanoluc (hDES) or AAV9-hDES-MSI2 (V4)-V5-T2A-Nanoluc (MSI2). Values above the vertical dashed lines indicate the median area of each biological condition. Data are expressed as mean±SEM. * p<0.05, ** p<0.01, *** p<0.001, and **** p<0.0001 according to two-way ANOVA with Tukey's HSD post hoc test. FVB (n=6), HSALRPBS (n=4-5), HSALR AAV9-hDES (n=9) and HSALR AAV9-hDES-MSI2 (n=13-14). In total, 3000 or more fibers were analyzed in each sample.

FIG. 21. Alterations in muscle homeostasis related genes are detected in MSI2-overexpressing mice (A-L) Quantification by qRT-PCR of the indicated genes in samples obtained from quadriceps or gastrocnemius from control and model mice treated with PBS (FVB and PBS, respectively), with AAV9 with the human DESMIN promoter without transgene AAV9-hDES-Nanoluc (hDES) or with the murine MSI2 AAV9-hDES-MSI2 (V4)-V5-T2A-Nanoluc (MSI2). All values are relative to hDES in each muscle (dashed line). Gene's expression is referenced to Gapdh, Gtf2b and, Hprt1 expression. Plots represent mean±SEM. * p<0.05, ** p<0.01, *** p<0.001, and **** p<0.0001 according to one-way ANOVA test with Tukey's HSD post hoc test. FVB (n=8), HSALRPBS (n=8), HSALR AAV9-hDES (n=6-10) and HSALR AAV9-hDES-MSI2 (n=13-15).

### GENERAL DEFINITIONS

It must be noted that, as used herein, the singular forms “a”, “an”, and “the”, include plural references unless the context clearly indicates otherwise. Further, unless otherwise indicated, the term “at least” preceding a series of elements is to be understood to refer to every element in the series. Those skilled in the art will recognize or be able to ascertain using no more than routine experimentation, many equivalents to the specific embodiments of the disclosure described herein. Such equivalents are intended to be encompassed by the present disclosure.

The term “about” when referred to a given amount or quantity is meant to include deviations of plus or minus five percent.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. Any of the aforementioned terms (comprising, containing, including, having), whenever used herein in the context of an aspect or embodiment of the present disclosure may be substituted with the term “consisting of”, though less preferred.

When used herein “consisting of” excludes any element, step, or ingredient not specified in the claim element. When used herein, “consisting essentially of does not exclude materials or steps that do not materially affect the basic and novel characteristics of the claim.

The term “oligonucleotide” refers to short DNA or RNA molecules that are preferably composed of a maximum of 200 nucleotides, preferably a maximum of 100 nucleotides, most preferably a maximum of 50 nucleotides in length.

The term “complementary” and “complementarity” are interchangeable and refer to the ability of polynucleotides to form base pairs with one another. Base pairs are typically formed by hydrogen bonds between nucleotide units in antiparallel polynucleotide strands or regions. Complementary polynucleotide strands or regions can base pair, for instance, as in the Watson-Crick manner (e.g., A to T, A to U, C to G). 100% (or total) complementary refers to the situation in which each nucleotide unit of one polynucleotide strand or region can hydrogen bond with each nucleotide unit of a second polynucleotide strand or region. Less than perfect (or partial) complementarity refers to the situation in which some, but not all, nucleotide units of two strands or two regions can hydrogen bond with each other and can be expressed as a percentage. The terms “sequence identity” or “percent identity” in the context of two or more nucleotides or polypeptides or proteins refers to two or more sequences or subsequences that are the same (“identical”) or have a specified percentage of nucleobases or amino acid residues that are identical (“percent identity”) when compared and aligned for maximum correspondence with a second molecule, as measured using a local sequence comparison algorithm (e.g., by a BLAST alignment, or any other local algorithm known to persons of skill), or alternatively, by visual inspection. It should be noted that the “percentage of identity” as used herein is measured in the context of a local alignment, i.e., it is based on the alignment of regions of local similarity between sequences, contrary to a global alignment, which aims to align two sequences across their entire span. Thus, in the context of the present disclosure, percentage identity is calculated only based on local alignment comparison algorithm.

Another indication that polynucleotide sequences are complementary is if two molecules hybridize to each other under stringent conditions. Stringent conditions are sequence-dependent and will be different in different circumstances. Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions for a Southern blot protocol involve washing at room temperature with a 5.times.SSC, 0.1% SDS wash.

The term “hybridization” is used to refer to the structure formed by 2 or more independent strands of RNA or DNA that form a double- or triplex-stranded structure via base pairings from one strand to the other. These base pairs are considered to be G-C, A-U/T and G-U/t. (A-Adenine, C-Cytosine, G-Guanine, U-Uracil, T-Thymine). As in the case of the complementarity, the hybridization can be total or partial.

“Myotonic dystrophy” (MD) is part of a group of inherited disorders called muscular dystrophies. There are two major types of myotonic dystrophy: type 1 (MD1) and type 2 (MD2). Myotonic dystrophy type 1 (MD1), as defined herein, is a multisystem disorder that affects skeletal and smooth muscle as well as other tissues such as the eye, heart, endocrine system, and central nervous system. The clinical manifestations of MD span a continuum from mild to severe and have been broadly categorized into three phenotypes: mild, classic, and congenital. Mild MD1 is characterized by cataracts and mild myotonia (sustained muscle contraction); life span is normal. Classic MD1 is characterized by muscle weakness and wasting, myotonia, cataracts, and often cardiac conduction abnormalities; adults may become physically disabled and may have a shortened life span. Congenital MD1 is characterized by hypotonia and severe generalized weakness at birth, often with respiratory insufficiency and early death; intellectual disability is common.

“Treatment of myotonic dystrophy” involves treatment of symptomatic or asymptomatic myotonic dystrophy, restoring at least partially the phenotype of a patient with said disease to a non-diseased state. “Prevention of myotonic dystrophy” involves measures taken to avoid the development of myotonic dystrophy (i.e., prevents the onset of the histopathology and/or symptomatology associated with myotonic dystrophy).

“RNA-binding proteins” (RBPs) are proteins that bind to the double or single-stranded RNA in cells and participate in forming ribonucleoprotein complexes. “Musashi” proteins are a family of RNA binding proteins comprised of Musashi homolog 1 (MSI1) and Musashi homolog 2 (MSI2) proteins and their critical roles in multiple biological processes, include those relevant to cancer initiation and progression. “RNA-binding protein Musashi (MSI2) or an analogue thereof” refers to MSI2 protein, variants, isoforms, or a structural analogue, such as a chemical or peptide analogue, that, despite not having an identical structure as MSI2 protein, exerts a similar function. The term “RNA-binding protein Musashi homolog 2” (MSI2) is thus to be understood as to comprise all isoforms of MSI2 protein, including the larger canonical isoform (MSI2 isoform 1) and the shorter, splice-variant isoform (MSI2 isoform 2), or structural analogues thereof.

“MicroRNA” (miRNA, miR) are a class of small non-coding RNA molecules that function in RNA silencing and post-transcriptional regulation of gene expression, playing a critical role in mRNA expression. miR-7 is a miRNA that plays an important role in the occurrence and development of some pathologies, but it is also essential in a variety of normal tissues and it is involved in the development of multiple organs and biological functions of cells. The primary transcript of miR-7 (pri-mir-7) is cleaved into precursor pre-mir-7 and further processed to mature miR-7. The products of three different DNA sequences (termed as pri-mir-7-1, pri-mir-7-2, and pri-mir-7-3) can be processed into the same mature miR-7 sequence, which comprises more or less 23 nucleotides. Human antigen R (HuR) protein mediates the binding of MSI2 to the conserved terminal loop of pri-miR-7.

“Small interfering RNAs (siRNA)”, also referred to as short interfering RNA or silencing RNA, are a class of double-stranded non-coding RNA molecules, typically 20-27 base pairs in length, that operate within the RNA interference (RNAi) pathway by interfering with the expression of specific genes with complementary nucleotide sequences, causing the degradation of the target mRNA after transcription and therefore preventing its translation. By “small interfering RNAs” (siRNAs) is thus meant oligonucleotide molecules used for transient silencing of gene of interest by eliciting RNA interference response upon binding to their target transcript based on the sequence complementarity. “Asymmetric siRNAs” (asiRNAs) are a class of siRNA characterized by long guide and short passenger strands, and they represent a novel and efficient scaffold structure for RNAi duplexes that do not have symmetry in their structure but present a similar mechanism of action as siRNAs.

“Antisense oligonucleotides” (ASOs) consist of short single-stranded short fragments of RNA or DNA that are used to alter the function of target RNAs or DNAs to which they hybridize without necessarily producing target mRNA degradation but sterically blocking the binding of another biomolecule and/or preventing a given secondary structure formation. The goal of the antisense oligonucleotide is often the downregulation of a molecular target, usually achieved by induction of RNase H endonuclease activity that cleaves the RNA-DNA heteroduplex with a significant reduction of the target gene translation. Antisense oligonucleotides might present nucleotide modifications in order to improve their characteristics.

“Gapmers” are a class of an antisense oligonucleotide that contains a central block of deoxynucleotide monomers sufficiently long to induce RNAse H cleavage protected by flanking highly modified ribonucleotide blocks.

“2-deoxy-2-fluoro-Darabinonucleic acid analog” (FANA) are a fully 2′ nucleotide that has both high-affinity RNA binding and retains RNAse H-compatible properties, being a class of antisense oligonucleotide highly useful for gene targeting applications.

“Morpholino oligomers” or “Phosphorodiamidate morpholino oligomers” (PMO) are short single-stranded DNA analogs that are built upon a backbone of morpholine rings connected by phosphorodiamidate linkages. PMOs bind to complementary sequences of target mRNA by Watson-Crick base pairing to block protein translation through steric blockade, which may be independent of RNase H, or modulate splicing events, among other potential mechanisms of action.

“AgomiR” or “miRNA mimetics” are a type of microRNA designed based on the mature microRNA sequence of interest, which inhibits the expression of endogenous RNA by mimicking it.

“AntagomiRs”, on the other hand, are generally used to silence endogenous miRNAs. Therefore, antagomiRs refer to small synthetic RNAs, chemically modified with respect to the corresponding RNA oligomer composed only of ribonucleotide units, and that are complementary to a target miRNA. Therefore, they can be considered oligonucleotide analogues that bind specifically to particular miRNAs and therefore act as miRNA inhibitors/blockers.

“BlockmiRs” are small RNAs with a special chemistry designed against the sequence that a particular miRNA detects in a particular messenger RNA (mRNA), so that, in principle, each one of them should only derepress the effect of that miRNA on that transcript, a very specific effect being expected. Therefore, they are designed so that they have a sequence that is complementary to that of a fragment of the mRNA sequence that serves as a binding site for a microRNA, in such a way that they usually bind at the 3′ end of the untranslated region (UTR) of an mRNA, in other words, in the area where the endogenous microRNAs usually bind.

“Anti-miRNA oligonucleotides” (AMOS) are molecules that neutralize microRNAs (miRNAs or miRs) function to which they hybridize. “AntimiR” or “antagomiR” refer to a group of anti-miRNAs that are specific and complementary to a miRNA and interfere with its function in the cell, that can be repressive or activatory on certain mRNAs, by for instance sequestering the miRNAs in competition with their cellular target mRNA or degrading them (the miRNA), inducing miRNA repression.

“Splice-switching oligonucleotides” (SSOs) are molecules that bind to target sequences in pre-mRNA and prevent the interaction of said target with various splicing modulators. Thus, SSOs are able to modulate pre-mRNA splicing and repair defective RNA without inducing the RNase H-mediated cleavage of mRNA.

“Locked nucleic acids” (LNA), also known as 2′-O,4′-C-methylene-bridged nucleic acid (2′,4′-BNA), are artificial nucleic acid derivatives that contain a methylene bridge connecting the 2′-O with the 4′-C position in the furanose rings, which enables it to form a strictly N-type conformation that offers high binding affinity against complementary RNA. Given these features, LNAs can be used for various gene silencing techniques, such as antisense, short interfering RNA, blocking of microRNA and triplex-forming oligonucleotides. Previous studies also showed that LNA could be used in SSOs and LNA-based SSOs (LNA SSOs) have been shown to be functional in vivo in mice models.

“LNA/DNA mixmers” are antisense oligonucleotides composed of alternating LNA and DNA nucleotides that are able to induce exon skipping in target mRNAs (LNA-based splice-switching oligonucleotide) or inhibit miRNAs. One of the advantages of using LNA/DNA mixmers for treatment is that they have negatively charged backbones, making delivery into cells more efficient than PMOs, which are neutrally charged.

“Peptide nucleic acid” (PNA) are artificially synthesized polymers that mimic DNA or RNA and hybridize to either single-stranded DNA or RNA, or to double-stranded DNA, with high affinity and specificity, acting as antisense molecules and interfering with the post transcriptional regulation of the target molecule.

“MicroRNA sponges” or miRNA sponges are constructs either transiently or stably transfected or infected or delivered by gymnosis into mammalian cells containing multiple miR-binding sites for a chosen miRNA gene, interfering with their function.

A “derivative” as used herein may be a compound, molecule, oligonucleotide, protein, or analogue thereof that arises from a parent compound by replacement of a portion of said compound, without significantly altering its function.

By “substantially identical” is meant a nucleic acid or amino acid sequence that, when optimally aligned, share at least 60%, 65%, 70%, 75%, 80%, 85%, 90%, 95%, 96%, 97%, 98%, 99%, or 100% sequence identity with a second nucleic acid or amino acid sequence.

By “fragment” is meant a portion of a polypeptide or nucleic acid molecule that contains, preferably, at least 10%, 20%, 30%, 40%, 50%, 60%, 70%, 80%, 90%, 95%, or more of the entire length of the reference nucleic acid molecule or polypeptide. A fragment may contain at least 10, 20, 30, 40, 50, 60, 70, 80, 90, or at least 100, 200, 300, 400, 500, 600, 700, 800, 900, 1000, 1200, 1500, 1750, 1800 or more nucleotides or at least 10, 20, 30, 40, 50, 60, 70, 80, 90, 100, 150, 200, 250, 300, 350, 400, 450, 500, 550, 600, 640 amino acids or more.

An “effective dose” or “therapeutically effective dose” is a sufficient amount to achieve a beneficial or desired clinical outcome.

The expressions “pharmaceutically acceptable” or “pharmacologically acceptable” refer to molecular entities and compositions that do not produce any adverse, allergic or other reactions when administered to an animal or human being. As used herein, “pharmaceutically acceptable vehicle” includes solvents, cells, buffers, solutions, dispersion media, coatings, antibacterial and antifungal agents, isotonic and absorption retarding agents and similar acceptable for use in formulation pharmaceuticals, such as pharmaceutical products suitable for administration to human beings. The use of such media and agents for pharmaceutical-active substances is well known in the art. Except where any conventional medium or agent is incompatible with the active ingredients of this disclosure, their use in the pharmaceutical compositions of the disclosure is contemplated. Additional active ingredients may also be incorporated into the compositions, provided that they do not inactivate the molecules of this disclosure or their expression vectors. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present disclosure, its use in therapeutic compositions is contemplated. Supplementary active ingredients also can be incorporated into the compositions, provided they do not inactivate the inhibitors of the compositions.

“Expression vector” is a nucleic acid molecule, usually a plasmid, a virus, DNA molecule, minichromosomes, or cell, designed to endogenously express a specific biomolecule, natural or artificially designed, such as RNAs or proteins, including the CRISPR-associated systems such as Cas9, Cas12a, and Cas13 and their corresponding guide RNAs, in a target cell. The expression vector is introduced into a host cell using well-known techniques such as infection or transfection, including calcium phosphate transfection, liposome-mediated transfection, electroporation and sonoporation. Expression constructs and methods for their generation and use to express a desired protein are known in the art.

Other features and advantages of the disclosure will be apparent from the following description, the drawings, and the claims.

### DETAILED DESCRIPTION

MSI2 and HuR proteins form a complex with pri-mir-7 and regulate the tissue-specific control of miR-7 biogenesis. Further, MSI2 is a transcriptional regulator that targets genes involved in development and cell cycle regulation, having an important role in the development of the nervous system, regulation of hematopoietic stem cells, cell division, cell cycle regulation and it has even been associated with poor prognosis in certain types of cancer. Thus, the function of MSI2 varies depending on the tissue where it is located. The authors of the present disclosure have surprisingly found that the function of MSI2 does not only depend on where and when the protein is located (which type of cells, tissue, and in which stage of development) but also depends on the concentration at which it is present in said cell. That is, the levels of MSI2 protein found in a cell also influence its regulation function and this is pivotal when it comes to the use of MSI2 as a therapeutic target to treat myotonic dystrophy (MD). It is shown in the present disclosure that MD can be treated by reducing the activity or levels of MSI2 protein or transcript in a myotonic dystrophy cell when said cells have reached a certain threshold of MSI2 protein concentration.

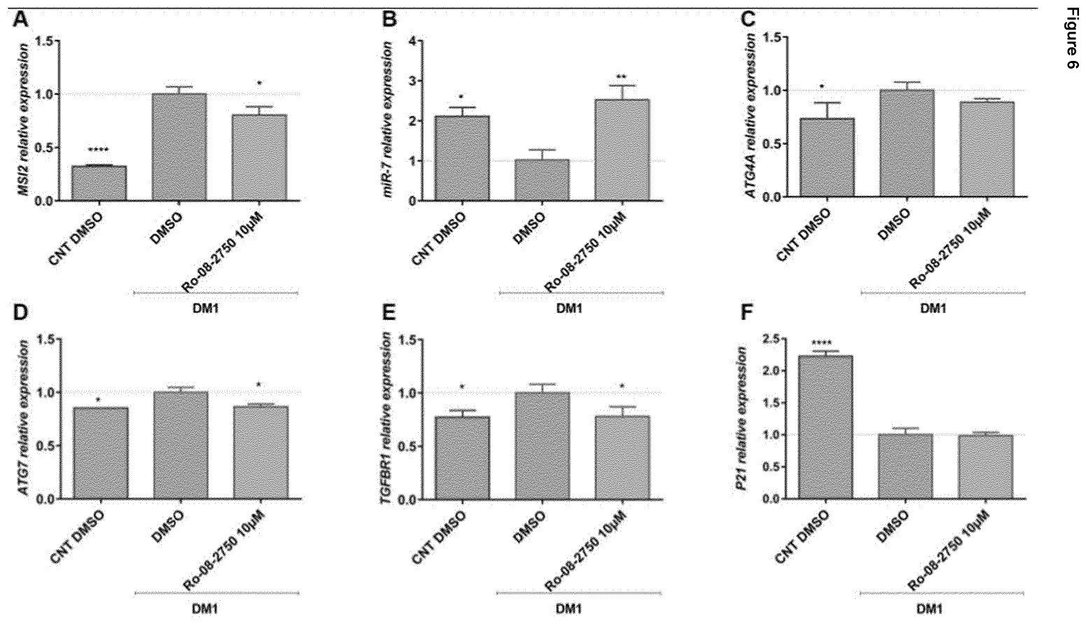

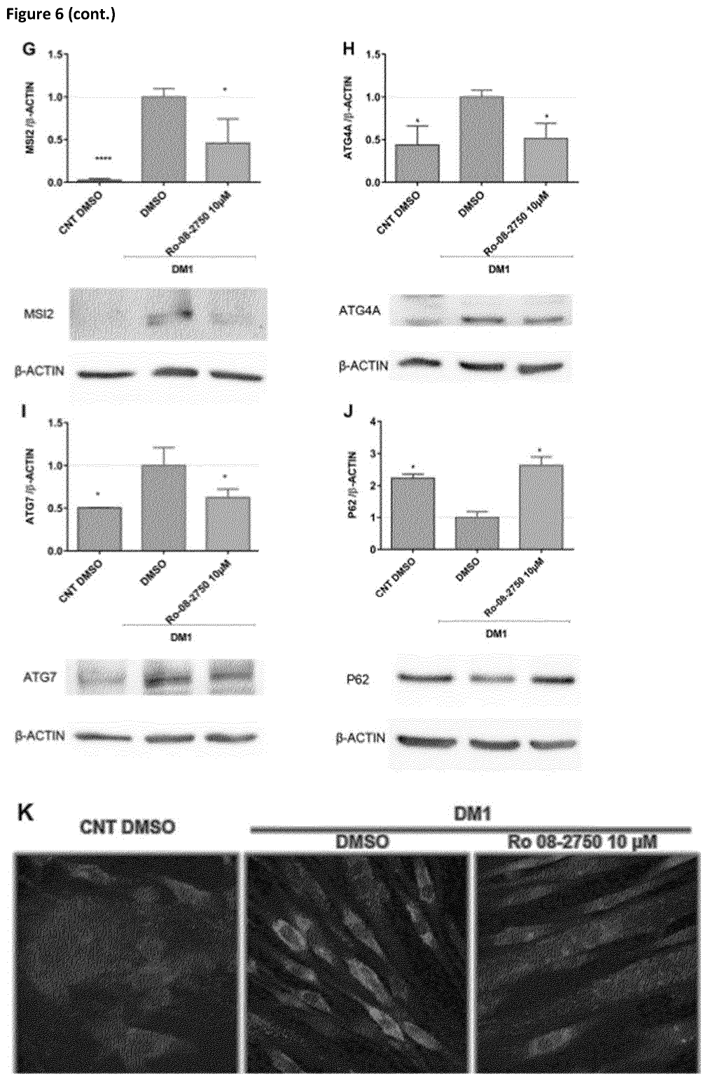

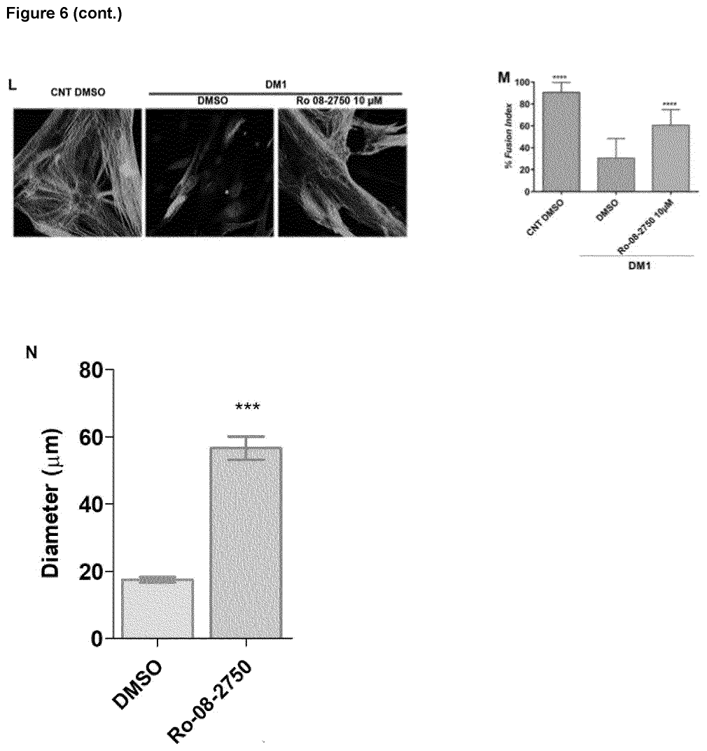

On the one hand, the present disclosure relates to inhibitors of MSI2 function by reducing MSI2 protein activity. Such inhibitors may be small molecules that are able to bind to or interact with MSI2 protein and avoid or compete for its RNA binding function, decreasing its activity in the cell. For instance, as shown in Example 5, a small molecule that binds directly to MSI2 protein was tested. This molecule, named Ro-08-2750 (2,3,4,10-Tetrahydro-7,10-dimethyl-2,4-dioxobenzo[g]pteridine-8-carboxaldehyde), is a compound that competes for RNA binding function of MSI2, causing a reduction in MSI2 activity and an increase in miR-7 (FIG. 6). Thus, the present disclosure encompasses inhibitors that exert their function at a protein level, and even if they do not decrease the levels of MSI2 protein or transcript, they are able to reduce MSI2 activity, causing an increase in miR-7 levels.

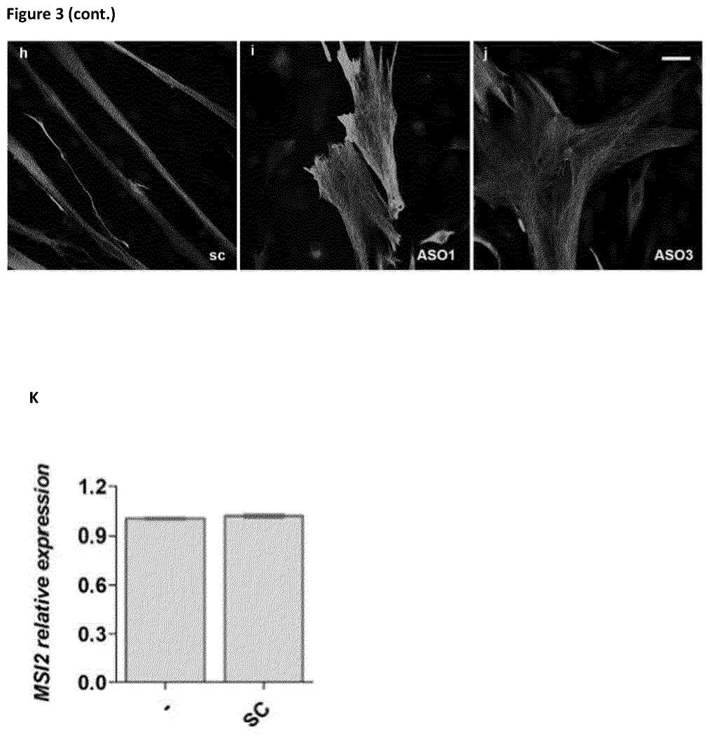

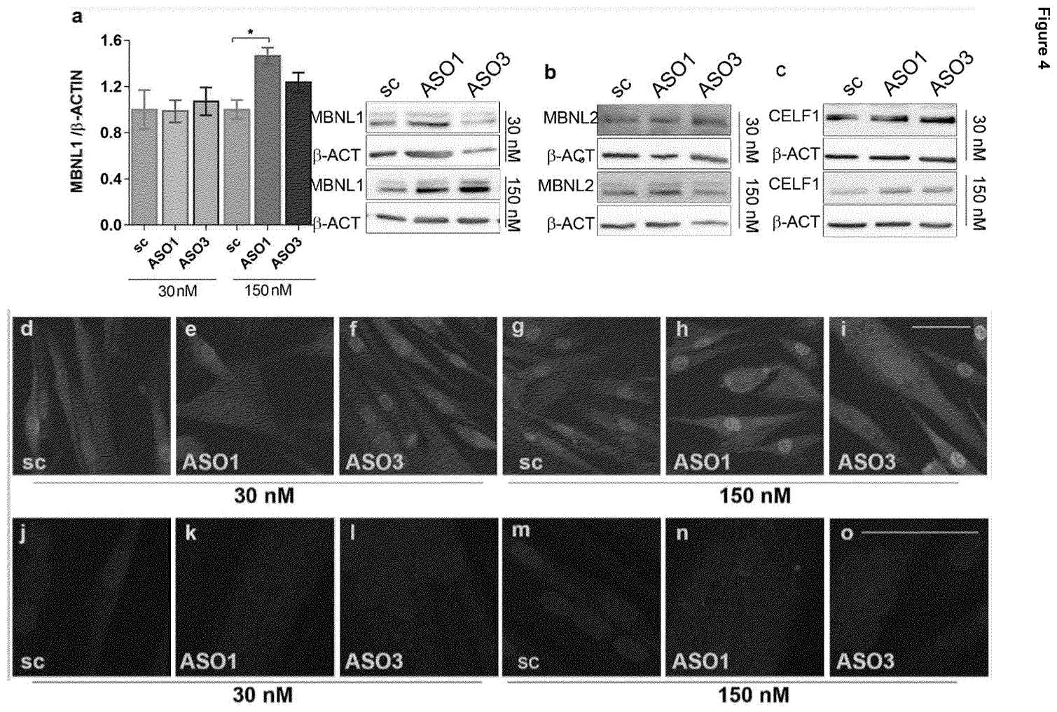

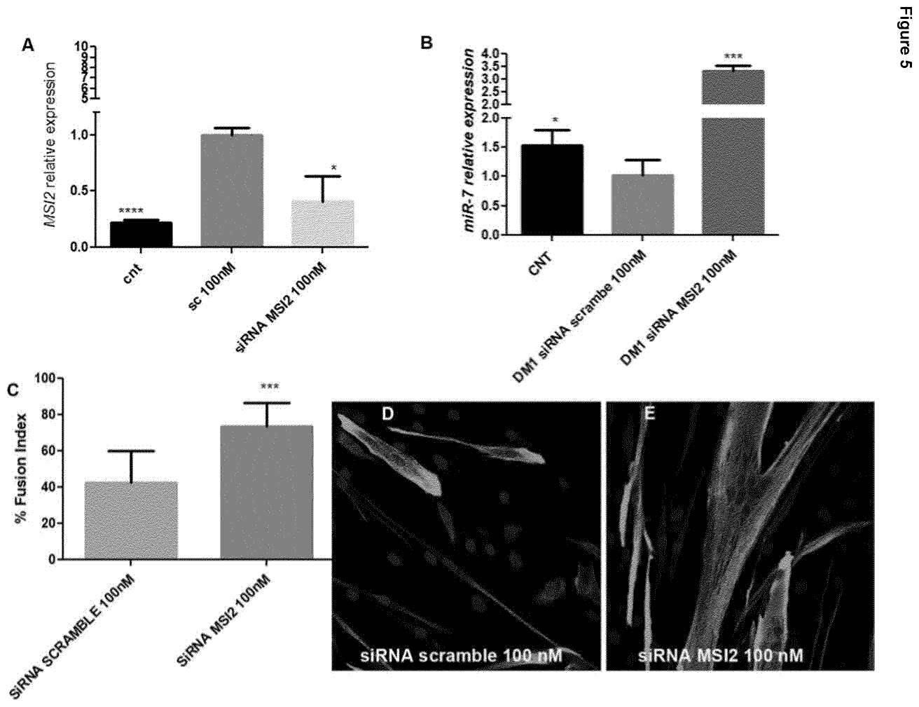

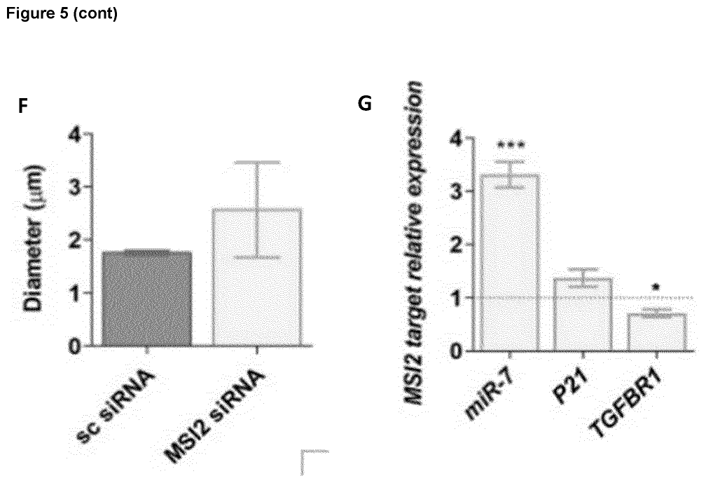

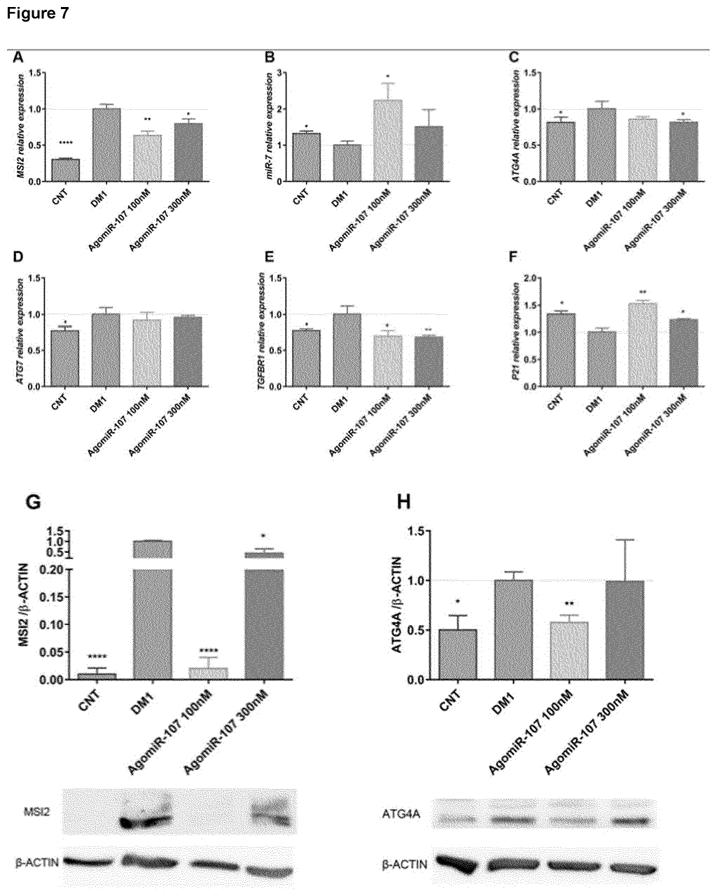

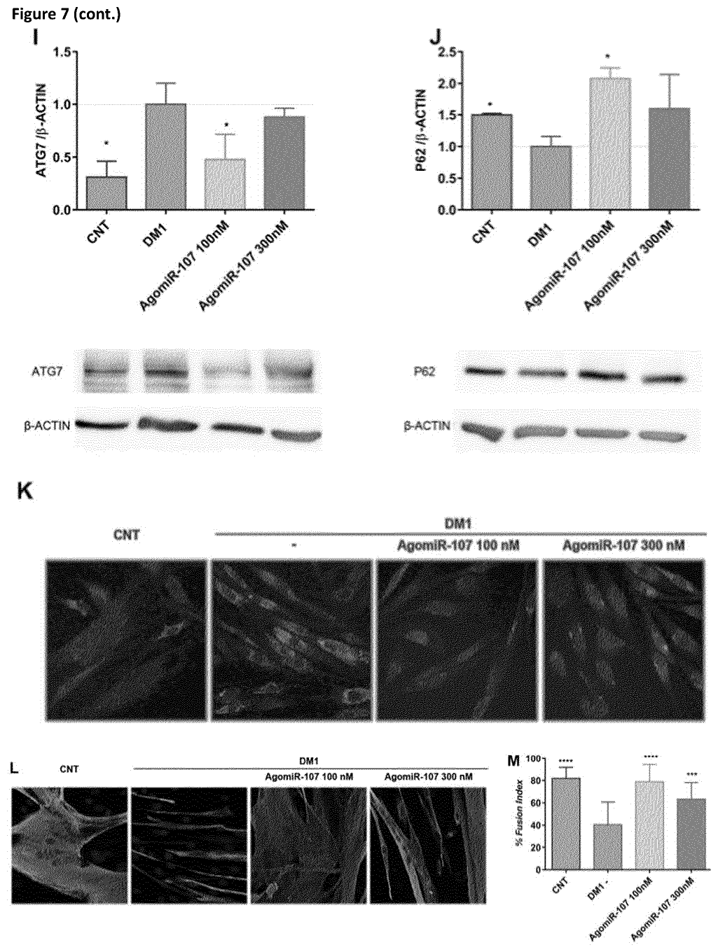

On the other hand, the present disclosure also relates to inhibitors of MSI2 function by reducing the MSI2 intracellular transcript levels or MSI2 intracellular protein levels. Such inhibitors may be oligonucleotides, oligoribonucleotides, or other compounds that reduce the MSI2 intracellular transcript or protein levels. For instance, as shown in Example 4, antisense oligonucleotides ASO1 and ASO3 directed against MSI2 transcript significantly lowered the level of MSI2 transcripts in MD1-derived myotubes at two different concentrations, causing a strong rescue in two atrophy-related myotube parameters: fusion index and myotube diameter (see FIG. 3). Other approaches to inhibit intracellular levels of MSI2 transcripts or protein were tested, such as small interfering RNA or miRNA mimetics: FIG. 5 shows the co-transfection of two siRNAs designed to target MSI2 transcript, causing an increase of miR-7 levels, and eventually rescuing the fusion index in patient cells. In Example 5, miR-107 analogues (miR-107 is a microRNA that negatively regulates MSI2 transcript by directly binding to its 3′ UTR region) were designed, and their use caused a downregulation of MSI2 that correlated with significant derepression of miR-7 (FIG. 7).

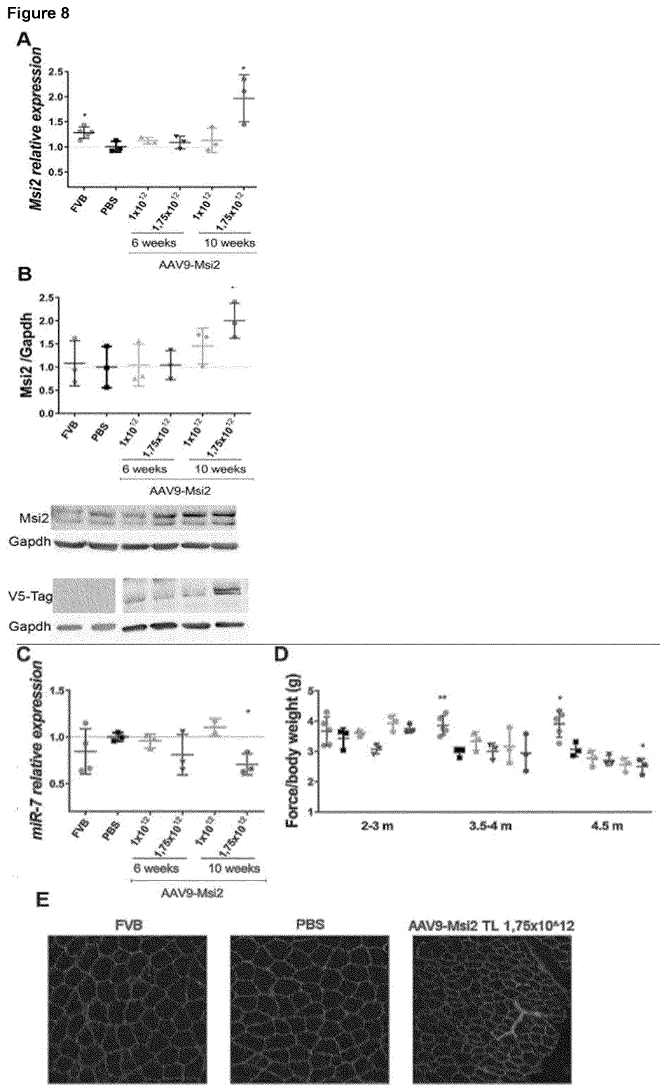

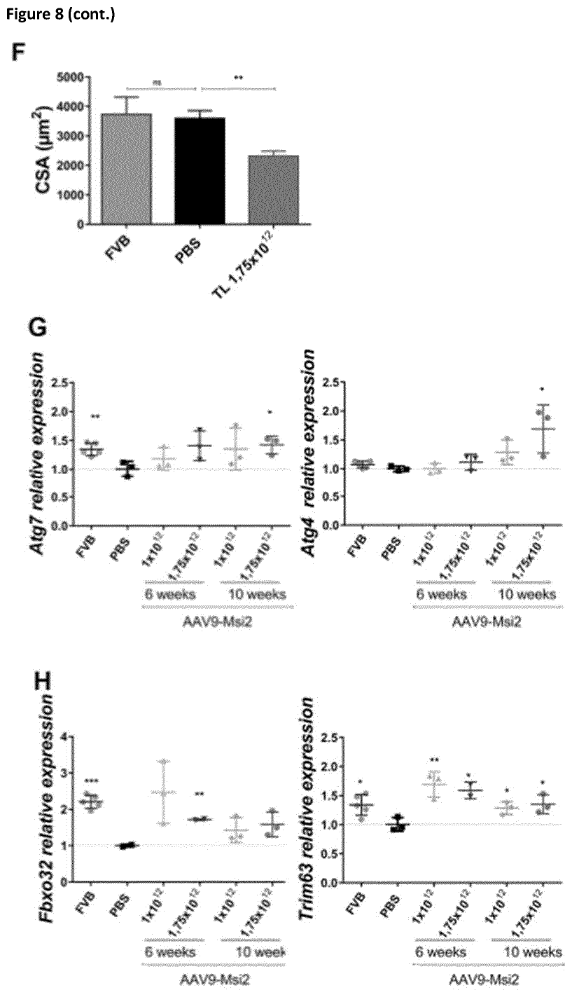

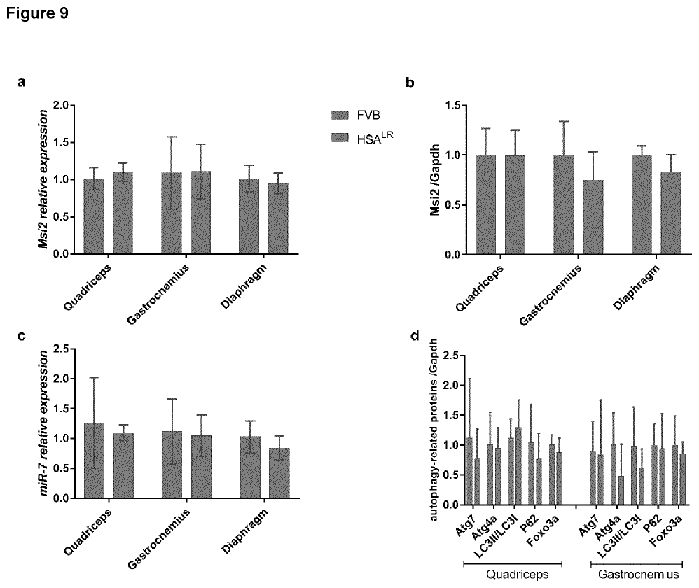

All of the above indicates that MSI2 levels can be related to miR-7 levels, and therefore, the regulation of MSI2 has a direct impact in the phenotype of MD1 cells. Further, it has also been found that the relationship between MSI2 and MD1 phenotype is also influenced by the levels of MSI2 protein present in the cell. Example 6 shows that miR-7 and autophagy-related proteins were quantified in different tissues in the mouse model HSA^{LR}, but no differences were observed between the mouse model and control mice (FVB), see FIG. 9. The authors of the present disclosure found that the reason why this mouse model did not present MD1 phenotype is because its cells did not have enough levels of overexpression of MSI2 protein. Therefore, when murine Msi2 protein was overexpressed in mice's tissues by administrating adeno-associated virus (AAV), it was observed that only in the group with the highest Msi2 levels (group administrated with the highest dose of AAV (1.75×10¹²)), miR-7 levels were significantly reduced, and the mice also showed decreased forelimb grip strength in comparison with control mice or mice administrated with a lower dose of AAV overexpressing murine Msi2 (FIGS. 8 and 9). These results demonstrate that when certain level of overexpression of MSI2 protein is achieved, the MD1 phenotype appears. In particular, as shown in Table 3 in the examples, the level of overexpression of MSI2 transcript and protein in MD1 patients are higher in at least 1.358 and 1.59-fold change, respectively, than those found in healthy human biopsies. Thus, if it is desired that a patient suffering from MD reaches the physiological levels of MSI2 present in healthy persons, the levels of MSI2 transcript and protein should preferably be reduced by about 35% and 60%, respectively.

Further, as also shown in Table 3, it was also found that MD1 patients have reduced levels of miR-7 in comparison to healthy controls. In particular, the miR-7 intracellular values of MD patients' biopsies were 0.58-fold reduced compared to healthy controls. Thus, for a MD patient to reach physiological or normal levels of miR-7, these levels should be preferably increased by about 70%.

Importantly, it was also found that, as shown in Table 5 in the examples, an increase of at least 1.68-fold (68%) in the intracellular miR-7 levels in treated cells compared to MD1 reference cells, particularly compared to immortalized skin fibroblasts expressing conditional MyoD, is enough to observe phenotypical changes in the cells such as fusion index and cell's diameters, indicating a partially rescued phenotype. These observations support the notion that the levels of miR-7 in a MD cell need to be increased at least 1.68 times in order to treat MD in a mammal.

As can be noted, the adjustment between MSI2 levels and miR-7 levels can be compared to those of a healthy individual (Table 3), or to those in a reference cell (Table 5). For the sake of simplicity, we herein define the changes in a MD1 cell treated with the inhibitor in terms of changes in miR-7 levels of a reference cell, particularly of immortalized skin fibroblasts expressing conditional MyoD. The reference cell will be defined below.

In view of the above, a first aspect of the present disclosure relates to an inhibitor of RNA-binding protein Musashi (MSI2) function or an analogue thereof for use in the treatment of myotonic dystrophy in a mammal, wherein the inhibitor is capable of reducing in a myotonic dystrophy (MD) cell or in a cell with the phenotype of a MD cell, at least one of
i) MSI2 protein activity,
ii) intracellular transcript levels of MSI2, and/or
iii) intracellular protein levels of MSI2,
thereby causing in the same MD cell an increase in the intracellular miR-7 levels of at least 1.5-fold of the intracellular miR-7 levels of a reference cell, as measured by a gene expression quantification technique such as quantitative reverse transcription PCR (RT-qPCR), Northern blotting, or RNA-sequencing (RNA-Seq) techniques.

By “inhibitor” is meant any substance or group of substances that reduce or suppress the activity and/or the amount of another substance. Particularly, in the present disclosure, the substance whose amount or activity is reduced or suppressed by the effect of the inhibitor is the MSI2 protein or transcript. At the time of defining the inhibitor of the first aspect, its function is defined in terms that it causes an increase in the intracellular levels of miR-7, since it has been shown in the present disclosure that when MSI2 function was reduced, the intracellular levels of miR-7 were increased. However, it is to be understood that the definition of the inhibitor of the present disclosure is not limited to only an effect over the levels of miR-7, but it also includes the inhibitors of MSI2 function that exert their effect by other mechanisms of action not directly related to miR-7, for example on TGFBR1 levels, as shown in Table 3.

Further, as referred herein, an “inhibitor” is not necessarily limited to only one compound, but it can be formed by the interaction of one or more than one compound (referred from now on as a “system of one or more compounds”). The term “inhibitor” may thus also include those technical approaches requiring the combination of two or more elements for its repressive activity such as, for example, a Cas family protein requiring the presence of a given guide RNA to recognize a specific DNA or RNA complementary sequence to exert their inhibitory function. That is, the inhibitor according to the first aspect of the present disclosure may consist of only one molecule type, such as an oligonucleotide, or it may be a complex comprising several components, such as a CRISPR/Cas and gRNA system. In general terms, inhibitors as referred herein can be divided into A) Oligonucleotides for RNA therapies, B) Proteins and small molecules, and C) Compounds or systems of one or more compounds for gene therapies. Each of these groups are explained in detail below.

“A myotonic dystrophy (MD) cell” is a cell suffering from MD or a cell with the phenotype of a MD cell. This cell might come from a subject suffering from MD or from a cell that has been genetically engineered so that MD is artificially induced. “A myotonic dystrophy cell or a cell with the phenotype of a MD cell” may also be defined as a cell having more than 50 repetitions of the CTG triplet in its genome, which are expressed as an isolated non-coding RNA or in the context of a coding messenger. The MD cell treated with the inhibitor may be a cell from any organ or tissue of a mammal. In a preferred embodiment, the MD cell or a cell with the phenotype of a MD cell is selected from the group of muscular cells, nervous cell, or cardiac cells. Preferably, the cell with the phenotype of a MD cell is selected from the group consisting of muscle satellite cells, myoblast, myocyte, or myotubes. More preferably, the cell with the phenotype of a MD cell is a myocyte or one of its precursor cell types. The MD cell may be a cell obtained from human muscle biopsies from a person suffering from MD disease. The MD cell may be one or more cells from a primary culture or an established cell line derived from muscle tissue (including induced pluripotent stem cells, known as iPSCs) or stem cells of said tissue. The MD cell may also be a transdifferentiated or genetically engineered cell from a human being or an animal that has been modified to be phenotypically or genotypically similar to a different cell, such as skin fibroblasts transdifferentiated into myoblasts (TDM cells).

It is noted that the function inhibited by the inhibitor of the present disclosure may be the natural function of MSI2, or the function derived due to its overexpression in the MD cells.

By “reference cell” is meant a cell affected by MD and that has not been treated with the inhibitor of the present disclosure. The reference cell can be any cell in any organ or tissue in a mammal suffering from MD. A reference cell is a cell whose levels of MSI2 activity, and/or MSI2 intracellular levels of protein and transcripts are taken as reference in the present disclosure because they are affected by MD disease. As also explained below, the reference cell can serve as a reference cell for the study of the increase of intracellular levels of miR-7 caused by the inhibitor according to the present disclosure.

In a particular embodiment, the reference cell is a standardized MD myoblast cell line. In a preferred embodiment, the reference cell is immortalized skin fibroblast cell line expressing conditional MyoD, as described by Arandel et al. 2017 [Arandel et al. (2017). Dis Model Mech. April 1; 10(4):487-497. doi: 10.1242/dmm.027367. Epub 2017 Feb. 10]. Immortalized skin fibroblasts expressing conditional MyoD, are a renewable and reliable source of converted human muscle cells that allow the assessment of therapeutic strategies against Muscular dystrophies. To obtain the reference cells, human primary fibroblasts isolated from skin biopsies obtained from a MD1 patient were used to establish immortalized myo-converted or transdifferentiated cell lines (also referred to as transdifferentiated myotubes) following the methodology described in Chaouch et al. [Chaouch, S., et al. (2009). Immortalized Skin Fibroblasts Expressing Conditional MyoD as a Renewable and Reliable Source of Converted Human Muscle Cells to Assess Therapeutic Strategies for Muscular Dystrophies: Validation of an Exon-Skipping Approach to Restore Dystrophin in Duchenne Muscular Dystrophy Cells. Human Gene Therapy, 20(7), 784-790. doi: 10.1089/hum.2008.163]. Briefly, primary fibroblasts isolated from a skin biopsy are immortalized by the sole re-expression of TERT before a subsequent transduction with lentiviral vectors containing an inducible Tet-on MYOD1 construct. Under non-permissive conditions, immortalized cells can proliferate indefinitely, but when supplemented with doxycycline, cells express MYOD1 that activates the myogenic program and their fusion into myotubes. The forced expression of MYOD1 following doxycycline supplementation leads to the formation of large multinucleated MD1 transdifferentiated myotubes comparable to healthy-derived transdifferentiated myotubes. FISH and immunofluorescence experiments revealed that MD1 myotubes contain nuclear CUGexp-RNA aggregates that colocalized with MBNL1 splicing factor. MD1 transdifferentiated myotubes displayed splicing changes of many transcripts, including ATP2A1, BIN1, INSR, LDB3, MBNL1 and TNNT2 that are also misregulated in skeletal muscles of DM1 patients. Immortalized MD1 cell lines, thereafter referred to as reference cells, exhibit the characteristic features of MD1 pathology, making them excellent cell lines to assess dystrophic rescue and molecular analyses to assess the effectiveness of therapeutic approaches to cure and/or treat MD.

By “healthy cell” is meant a cell not affected by MD, i.e having less than 37 CTG repeats in the DMPK locus. Healthy cells can be cells from any organ or tissue having physiological, i.e., normal, intracellular levels of MSI2. In an embodiment, the healthy cell is a muscular, cardiac, or nervous cell.

“MSI2 activity” is meant the activity or function of the MSI2 protein, which may be the regulation of target gene transcripts involved in cellular pathways, such as cell development and cell cycle regulation, including but not limited to those directed to regulate the biogenesis of miR-7.

By “MSI2 transcript” is meant the single-stranded ribonucleic acid product synthesized by transcription of MSI2 gene. The term MSI2 transcript includes all kinds of transcripts produced after transcription, including mature and immature transcripts (such as pre-mRNAs or mature mRNAs). By “transcript levels” is meant the number, amount or quantity of total transcript molecules measured by a gene expression quantification technique. By “intracellular transcript levels” is meant the number, amount or quantity of total transcripts molecules located inside one or more cells. Preferably, the MSI2 transcripts are those defined in SEQ IDs No: 1, 2, and 109 to 137.

By “protein levels” is meant the number, amount or quantity of the proteins measured by a protein quantification technique. “Intracellular protein levels” refers to the number, amount or quantity of the proteins located inside one or more cells.

By “reducing in a myotonic dystrophy cell at least one of i) MSI2 protein activity, ii) intracellular transcript levels of MSI2, or iii) intracellular protein levels of MSI2” is meant that the protein activity of MS2, its intracellular transcript levels or protein levels are decreased, i.e., lowered.

In the context of the first aspect of the present disclosure, “causing in the same MD cell an increase in the intracellular miR-7 levels” is meant that the intracellular levels of miR-7 of the MD cell or the cell with the phenotype of a MD cell treated with the inhibitor are increased in comparison to those present in a reference cell, wherein the reference cell is preferably a skin fibroblast cell line expressing conditional MyoD. As explained above, Table 5 in the examples shows that the cells treated with Ro 08-2750, ASO3, ASO1 and siRNA inhibitors reverted to normal phenotype when the miR-7 levels were increased up to 2.52, 1.68, 1.49 or 3.3-fold, respectively. This demonstrates that it is plausible to revert MD1 phenotype with the treatment of the inhibitor according to the present disclosure.

In an embodiment, the intracellular increase in miR-7 in the MD cell or the cell with the phenotype of a MD cell after treatment with the inhibitor may be of at least 1.2, 1.25, 1.3, 1.35, 1.4, 1.5, 1.6, 1.65, 1.7, 1.75, 1.8, 1.85, 1.9, or 1.95-fold increase of the intracellular miR-7 levels of the reference cell, wherein the reference cell is preferably a skin fibroblast cell line expressing conditional MyoD, as measured by a gene expression quantification technique. In a preferred embodiment, the increase is of at least 1.6-fold, more preferably at least 1.7-fold.

In a preferred embodiment, the increase in the intracellular miR-7 level is of at least 1.7-fold of the intracellular miR-7 levels of a reference cell and the MD cell or a cell with the phenotype of a MD cell is a myocyte or one of its precursor cell types.

By a “gene expression quantification technique” is meant any analytical method used to detect and quantify RNA expression levels or differential RNA expression within one or more cells. Northern blotting is a technique used to detect specific RNA molecules present within an RNA mixture, and it is employed in the analysis of an RNA sample from a cell type or tissue so as to determine the RNA expression of certain genes. Quantitative reverse transcription polymerase chain reaction (RT-qPCR) is one of the most sensitive techniques available for detecting and quantifying RNA. Using RT-PCR, extremely small sample sizes can be used in the quantification of RNA and the technique can in fact do this using just a single cell and in real-time. RNA-sequencing (RNA-Seq) refers to methods used to measure the amount of RNA molecules. Examples include shotgun sequencing of cDNA molecules acquired from RNA through reverse transcription and technologies used to sequence RNA molecules from a biological sample so that the primary sequence and abundance of each RNA molecule can be determined.

The intracellular levels of miR-7 as measured by a gene expression quantification technique may be expressed or measured by an “absolute” quantification or by a “relative” or comparative quantification.

The inhibitor exerts its effects by causing a reduction in the MSI2 activity, by reducing intracellular transcript levels of MSI2, or by reducing intracellular protein levels of MSI2, as defined above. Preferably, the MSI2 inhibitor reduces the intracellular transcript levels of MSI2.

In an embodiment, the inhibitors according to the first aspect of the present disclosure are selected from the group comprising:
A) Oligonucleotides for RNA therapies
B) Proteins and small molecules
C) Compounds or systems for gene therapies

### A) Oligonucleotides for RNA Therapies

Some examples of possible inhibitors may be oligonucleotide molecules, oligoribonucleotide molecules, hybrid DNA-RNA molecules, proteins and small molecules, and analogues or derivatives thereof. Further, they may be formed of naturally occurring nucleic acids or synthetic or modified nucleic acids. In a preferred embodiment, the inhibitors may be oligonucleotides molecules. The MSI2 inhibitor may be an oligoribonucleotide molecule or an analogue thereof. The oligoribonucleotides (RNA) that are composed of a phosphate group, the nitrogenous bases adenine (A), cytosine (C), guanine (G) or uracil (U), and pentose known as ribose. Based on their widespread use, molecules whose units include nucleotide inosine are also considered within that definition.

The oligonucleotide molecules, according to the first aspect of the present disclosure, may be oligonucleotides or oligoribonucleotides molecules derived therefrom that include, among others, some of the usual chemical modifications that modify the oligonucleotide molecules to make them more resistant to degradation or bioavailable. Thus, as used in this disclosure, the term “oligonucleotide molecules” includes both oligonucleotides as such, as well as the “oligonucleotide analogues”.

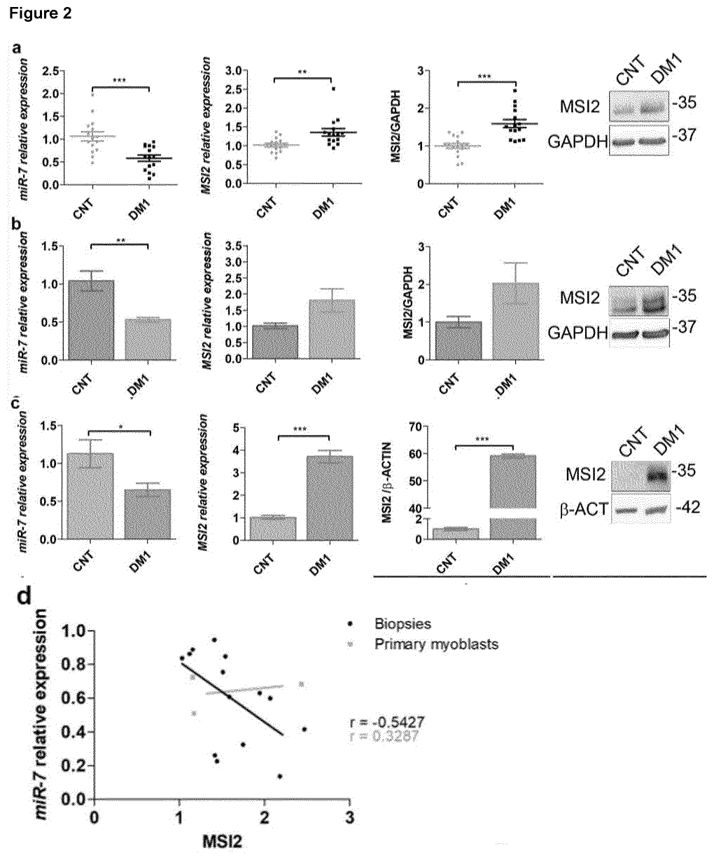

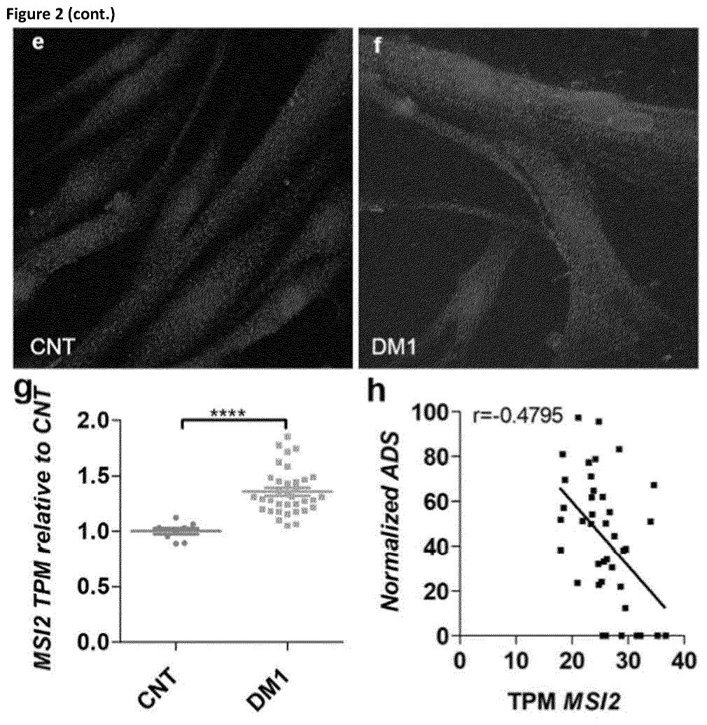

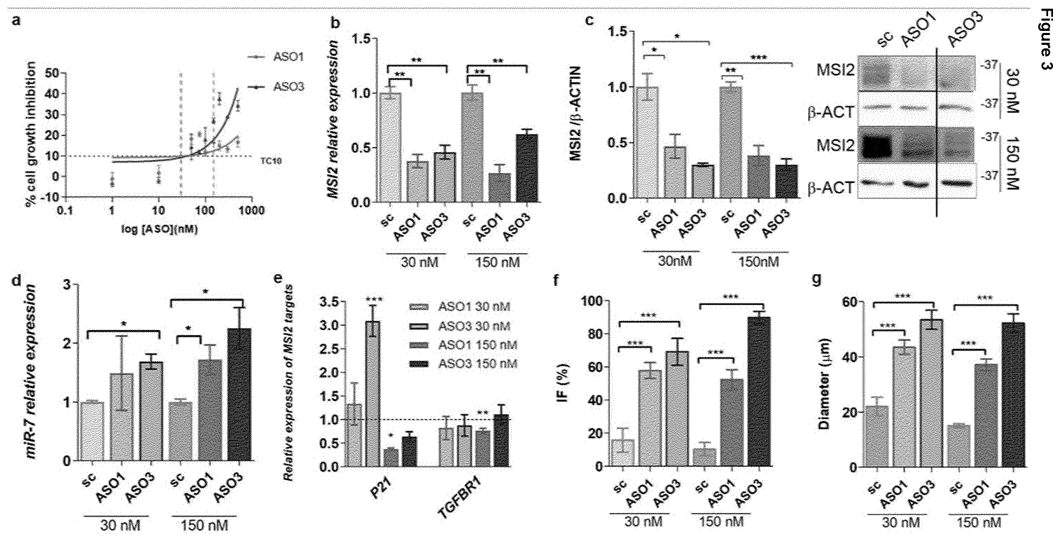

“Oligoribonucleotide analogues” are the molecules derived therefrom that incorporate some chemical modification in at least one of the ribonucleotide units that form them, either in the phosphate group, the pentose or one of the nitrogenous bases, the modifications consisting in the addition of non-nucleotide groups at the 5′ and/or 3′ ends are also included. A skilled person would know which type of chemical modifications are suitable for the purposes of the present disclosure. As reference, FIG. 2 in Crooke et al. 2018 [Crooke, S. T., Witztum, J. L., Bennett, C. F., & Baker, B. F. (2018). RNA-Targeted Therapeutics. Cell Metabolism, 27 (4), 714-739. doi: 10.1016/j.cmet.2018.03.004], shows the different modifications that can be performed in RNA and DNA analogues.

These modifications can be made in the pentose, in the internucleotide linkage, or in the nucleobase, or in a combination thereof. In an embodiment, the oligonucleotide comprises at least six, seven, eight, nine, ten, eleven, twelve, thirteen, fourteen, fifteen or sixteen chemical modifications along the whole molecule. In an embodiment, the oligonucleotide comprises all its nucleotides chemically modified. Such modifications include 2′-Fluoro, 2′-O-methyl, 5-methylcytosine, 2′-MOE (2′-O-methoxyethyl), cEt ((S)-constrained ethyl), LNA (locked nucleic acid), PMO (Phosphorodiamidate morpholino), thiophosphoramidate, PS (phosphorothioate), among others. The oligoribonucleotide analogues may also be conjugated with other molecules, such as antibodies, lipids, lipid nanoparticles or GalNac (N-acetyl galactosamine) clusters, among others. By extension, for the purposes of this disclosure and as used herein, the terms “oligoribonucleotide molecule” and “oligoribonucleotide analogue” or “oligonucleotide analogue molecule” also include miRNA sponges, as it can be considered that the main constituent of the same are tandem repeats of oligonucleotides, characterized in that each of these oligonucleotides are in themselves or contain a binding site of a micro-RNA of interest.

Other chemical modifications in the oligonucleotides or oligoribonucleotides of the present disclosure are possible and known, which are also comprised within the possible modifications that give rise to oligonucleotide analogues. As can be deduced from the definition of “oligonucleotide molecules” and that of “oligonucleotide analogues”, also included within these definitions are hybrid molecules, in which some units present modifications and others do not, as well as hybrids between analogues of nucleic acids and peptides or, even, hybrid molecules in which some of the nucleotide units are ribonucleotides (or analogues thereof) and others are deoxyribonucleotides (nucleotides in which the sugar is deoxyribose), as well as analogues of the latter, i.e., RNA-DNA hybrids and analogues thereof.

Lastly, further modifications that can be added to the oligonucleotides include the conjugation at their 3′ and/or 5′ ends with a non-nucleotide molecule, such as a fatty acid, preferably cholesterol.

In a preferred embodiment, the MSI2 inhibitor for use in the treatment of myotonic dystrophy is an oligonucleotide molecule that comprises a fragment composed of one or more nucleotide unit sequence or a succession of nucleotide units. In an embodiment, the oligonucleotide molecule has an effect in the RNA interference pathway. By “RNA interference pathway” or “RNAi” is meant the biological process in which RNA molecules inhibit or alter gene expression or translation by neutralizing targeted mRNA molecules. Particularly, the three types of oligonucleotide molecules that are central in the RNAi pathway are microRNAs (miRNA), small interfering RNAs (siRNA) and piwiRNAs.

In an embodiment, the oligonucleotide molecules or oligonucleotide analogues according to the first aspect of the present disclosure is selected from the group consisting of small interfering RNAs, antisense oligonucleotides, gapmers, morpholino oligomers, FANA oligonucleotides, agomiRs, miRNA mimics, antagomiRs, blockmiRs, PNAs, LNAs (locked nucleic acid antisense oligonucleotides), splice-switching oligonucleotides (SSOs), LNA-based splice-switching oligonucleotides (LNA SSOs), LNA/DNA mixmers or miRNA sponges. Also comprised within the concept of oligonucleotide molecules or oligonucleotide analogues useful for the purpose of the present disclosure and comprised within its scope are those mRNA inhibitors, blockers or antagonists that act on pre-mRNAs, usually altering mRNAs biogenesis or maturation and having a negative effect on mRNAs activity, mainly due to a decrease of the available active mRNA.

The design of oligonucleotides molecules as inhibitors/antagonists is usually based on a short basic sequence of nucleotides, which may be the sequence complementary to the RNA to be inhibited or an RNA-binding protein recognized. As used in this specification, it is understood that two chains of nucleotide molecules are 100% complementary (or, as is expressed in a more abbreviated way herein, that their sequences are complementary) when the nucleotide or nucleotide analogue sequence of one of them, read in the 5′-3′ sense, is the sequence of nucleotides or nucleotide analogues that present the nitrogenous bases which pair with the nitrogenous bases of nucleotides or nucleotide analogues of the other sequence, read in the 3′-5′ sense. That is to say, the sequence 5′-UAGC-3′ would be complementary to the sequences 3′-AUCG-5′ (in the case of being the ribonucleotide or ribonucleotide analogue units) and 3′-ATCG-5′ (in the case of being the deoxyribonucleotide or deoxyribonucleotide analogue units), which would be, respectively, sequences 5′-GCUA-3′ and 5′-GCTA-3′ read in the 5′-3′ sense.

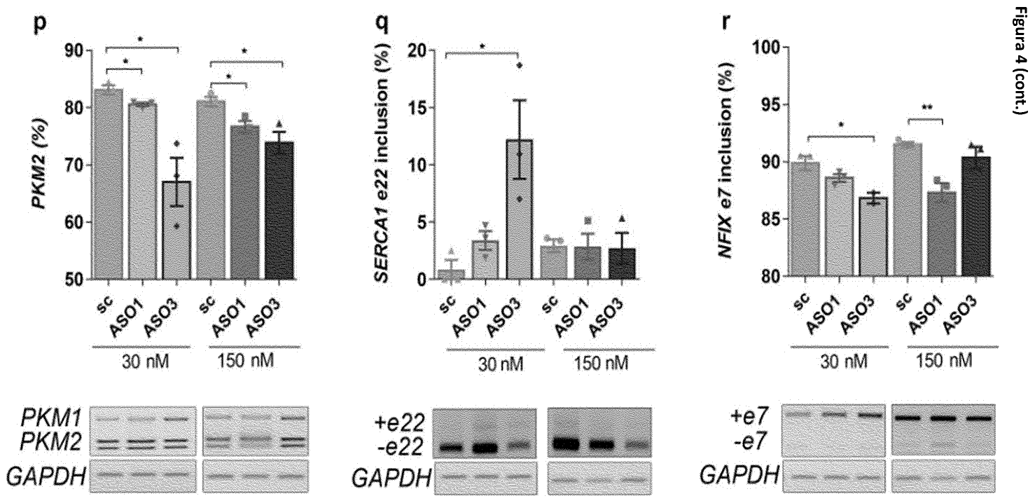

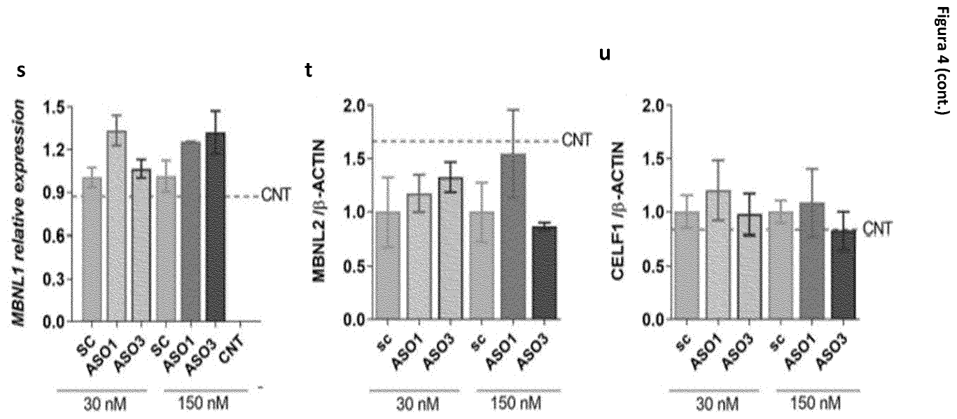

To design the antagonist or inhibitor according to the first aspect, it is important to note that there should be sufficient complementarity with the endogenous molecules to which they must bind so that the desired effect of inhibition/antagonism/silencing is actually produced. In this sense, examples can be taken into account where the “typical” complementarity between a miRNA and its target may be 50% (see, for example, the reference http://mirtarbase.mbc.nctu.edu.tw/php/detail.php′?mirtid=MIRT000125 #target or FIG. 4a in Dong, P., et al. Musashi-2, a novel oncoprotein promoting cervical cancer cell growth and invasion, is negatively regulated by p53-induced miR-143 and miR-107 activation. J Exp Clin Cancer Res 36, 150 (2017).), therefore it is advisable that the oligonucleotide molecule of the disclosure comprises a fragment of nucleotide or of nucleotide analogue unit sequence, in which the sequence of the nitrogenous bases of the nucleotide or nucleotide analogue units is at least 50% (or at least 55%, 60%, 70%, 80%, 85%, 90%, 91%, 92%, 93%, 94%, 95%, 96%, 97%, 98%, 99% or 99.5%, or 100%) identical to the sequence complementary to that of the fragment of the endogenous molecule with which it should pair, that is to say, to the sequence of endogenous RNA transcript to which it should bind. Overall, this is the criterion that has been followed for the design of the oligonucleotides of the present disclosure against human MSI2 transcript (SEQ ID NO: 1 or 2).

In the case of some oligonucleotides molecules, for instance agomiRs, the most important thing is that they comprise a fragment in which the sequence of nitrogenous bases of nucleotides or nucleotide analogues is complementary (preferably, 85%, 90%, 95% or 100% complementary) to the sequence of nitrogen bases of the nucleotides of the seed region of the molecule, for instance the mRNA of MSI2 protein, which is their target, so the complementarity in the rest of 25 nucleotides or nucleotide analogues that are present in the nucleotide molecule, if any, is less important. This is because some oligonucleotides are usually complementary to only a part, the so-called seed region, of their target mRNA, which spans around 7-10 nucleotides, but they bind to it with great affinity. In this case, the whole complementary between both molecules may not be more than 50%, but it in should be considered that the seed region, comprised within the sequence of the oligonucleotides, comprises a sequence with high complementary of at least 80%, 90% or even 100% to the target mRNA where they hybridize.

In an embodiment, MSI2 transcription levels may be reduced according to the present disclosure by using agomiRs oligonucleotides. As defined above, agomiRs are micro-RNA that mimic endogenous RNAs, for instance microRNAs. An agomiR according to the present disclosure may be a molecule that mimics the endogenous micro-RNA-107, which negatively regulates MSI2 by directly binding to its 3′ UTR. In a further embodiment, the inhibitor may be an oligonucleotide sequence that, alone or in combination with a protein activity, edits or modifies MSI2 transcripts, causing a decrease in their stability and therefore reducing the levels of intracellular MSI2 transcripts and/or intracellular MSI2 protein.

The oligonucleotide molecule, which comprises a fragment composed of a succession of nucleotide units, may comprise at least 5, 6, 7, 8, 9, 10 consecutive nucleotides in length that have at least 70%, 75%, 80%, 85%, 90%, 95%, 96%, 97%, 98%, 99% or 100% identity with the sequence target where they bind or to the complementary sequence thereof. In an embodiment, the target sequence where they bind is SED ID NO: 1 or, preferably, SEQ ID NO: 2, or any of SEQ ID NOs: 109 to 137, or a complementary thereof. Any combination of consecutive nucleotides in length with the percentage of identity with the sequence target where they bind or to the complementary sequence thereof is included in the present disclosure. For instance, in an embodiment, the oligonucleotide molecule of the present disclosure may comprise at least 5 consecutive nucleotides in length that have at least 80% identity over the full length sequence of SEQ ID NO: 1 or, preferably SEQ ID NO: 2, or any of SEQ ID NOs: 109 to 137, or to the complementary sequence thereof. In a preferred embodiment, the oligonucleotide comprises at least 7 consecutive nucleotides in length that have at least 90% identity over the full length sequence of SEQ ID NO: 1 or, preferably SEQ ID NO: 2, or any of SEQ ID NOs: 109 to 137, or to the complementary sequence thereof. In the context of the present disclosure, it is to be understood as a preferred embodiment that the inhibitor oligonucleotides molecules according to the present disclosure have at least 70%, 75%, 80%, 85%, 90%, 95%, 96%, 97%, 98%, 99% or 100% identity over the complementary sequence of the full sequence of SEQ ID NO: 1 or, preferably SEQ ID NO: 2, or any of SEQ ID NOs: 109 to 137.

The length of the oligonucleotide can be adjusted and may vary according to various criteria known by the skilled person in the art. The oligonucleotide molecule according to the present disclosure, which comprises a fragment composed of a succession of nucleotide units, may be at least 5-25, 10-25, 15-25, 20-50, or 25-50, etc., nucleotides in length. In an embodiment, the oligonucleotide molecule is at least 5, 6, 7, 8, 9, 10, 11, 12, 13, 14, 15, 16, 17, 18, 19, 20, 21, 22, 23, 24, 25, 26, 27, 28, 29, 30 nucleotides in length. In another embodiment, the oligonucleotide molecule which comprises a fragment composed of a succession of nucleotide units may be more than 30 nucleotides in length. In another embodiment according to the first aspect, the oligonucleotide molecule is at least 5, 6, 7, 8, 9, 10, 11, 12, 13, 14, 15, 16, 17, 18, 19, 20, 21, 22, 23, 24, 25, 26, 27, 28, 29, 30 nucleotides in length, of which at least 5, 6, 7, 8, 9, or 10 nucleotides are consecutive and have at least 70%, 75%, 80%, 85%, 90%, 95%, 96%, 97%, 98%, 99% or 100% identity with the sequence target where they bind or to the complementary sequence thereof. The sequence identity of the consecutive nucleotides comprised in the oligonucleotide molecule of the present disclosure may be at least 70%, 75%, 80%, 85%, 90%, 95%, 96%, 97%, 98%, 99% or 100% identical over the full-length sequence of the target sequence where they bind, which may be SEQ ID NO: 1 or, preferably, SEQ ID NO: 2, or any of SEQ ID NOs: 109 to 137, or the complementary sequence thereof. Any combination between nucleotide length, consecutive nucleotides length and percentage of identity is included in the present disclosure. For instance, in a preferred embodiment, the oligonucleotide molecule comprises at least 10-25 nucleotides in length, from which at least 5 consecutive nucleotides in length have at least 80% identity over the full-length sequence of SEQ ID: 1 or, preferably, SEQ ID NO: 2, or any of SEQ ID NOs: 109 to 137, or to the complementary sequence thereof. In a further preferred embodiment, the oligonucleotide molecule comprises at least 10-25 nucleotides in length, from which at least 7 consecutive nucleotides in length have at least 90% identity over the full-length sequence of SEQ ID NO: 1 or, preferably SEQ ID: 2, or any of SEQ ID NOs: 109 to 137, or the complementary sequences thereof.

In a preferred embodiment, the inhibitor oligonucleotides molecules according to the present disclosure are capable of hybridizing under stringent conditions to a region of at least 5, 6, 7, 8, 9, or 10 consecutive nucleotides comprised in SEQ ID NO: 1 or, preferably SEQ ID NO: 2, or any of SEQ ID NOs: 109 to 137. In a further embodiment, the oligonucleotide sequence comprises a first fragment and a second fragment, wherein the first fragment is a seed region composed of a succession of at least 5-8 nucleotide wherein the sequence of the nitrogenous bases of said first fragment is identical in at least 90%, 91%, 92%, 93%, 94%, 95%, 96%, 97%, 98%, 99% or 100% to the complementary sequence SEQ ID NO: 1 or 2 or 109 to 137, and wherein the second fragment is adjacent to the first fragment (i.e., it is located upstream and/or downstream of the first fragment) and it is composed of a succession of at least 7, 8, 9, 10, 11, 12, 13, 14, 15, or 16 consecutive nucleotides that are identical in at least 30%, 40%, 50%, 60%, 70%, 80%, 85%, 90%, 91%, 92%, 93%, 94%, 95%, 96%, 97%, 98%, 99% or 99.5%, or 100% to the complementary sequence of SEQ ID NOs: 1 or 2 or any of SEQ ID NOs: 109 to 137.

In an embodiment, the nucleotide molecule according to the first aspect of the present disclosure has at least 65%, 70%, 75%, 80%, 85%, 90%, 95%, 96%, 97%, 98%, 99%, or 100% identify over the full sequence of any of the nucleotides of SEQ ID NO: 3 to SEQ ID NO: 37. In another embodiment according to the first aspect, the oligonucleotide molecule is at least 5, 6, 7, 8, 9, 10, 11, 12, 13, 14, 15, 16, 17, 18, 19, 20, 21, 22, 23, 24, 25, 26, 27, 28, 29, 30 nucleotides in length, of which at least 5, 6, 7, 8, 9, or 10 nucleotides are consecutive and have at least 70%, 75%, 80%, 85%, 90%, 95%, 96%, 97%, 98%, 99% or 100% identity with the SEQ ID: 3 to 37. In a preferred embodiment, the oligonucleotide molecule has at least 90% identity over the full sequence of any of the oligonucleotides SEQ ID NO:3 to SEQ ID NO:37, or the complementary sequences thereof. In the context of the present disclosure, it is to be understood as a preferred embodiment that the inhibitor oligonucleotides molecules according to the present disclosure have at least 70%, 75%, 80%, 85%, 90%, 95%, 96%, 97%, 98%, 99% or 100% identity over the complementary sequence of the full sequence of SEQ ID NO: 3 to SEQ ID NO: 37.

In a preferred embodiment, the inhibitor oligonucleotides molecules according to the present disclosure are capable of hybridizing under stringent conditions to a region of at least 5, 6, 7, 8, 9, or 10 consecutive nucleotides comprised in any of SEQ ID NO: 3 to SEQ ID NO: 37. In a further embodiment, the oligonucleotide sequence comprises a first fragment and a second fragment, wherein the first fragment is a seed region composed of a succession of at least 5-8 nucleotides wherein the sequence of the nitrogenous bases of said first fragment is identical in at least 90%, 91%, 92%, 93%, 94%, 95%, 96%, 97%, 98%, 99% or 100% to the complementary sequence of any of SEQ ID NO: 3 to SEQ ID NO: 37, and wherein the second fragment is adjacent to the first fragment (i.e., it is located upstream and/or downstream of the first fragment) and it is composed of a succession of at least 7, 8, 9, 10, 11, 12, 13, 14, 15, or 16 consecutive nucleotides that are identical in at least 30%, 40%, 50%, 60%, 70%, 80%, 85%, 90%, 91%, 92%, 93%, 94%, 95%, 96%, 97%, 98%, 99% or 99.5%, or 100% to the complementary sequence of any of SEQ ID NO: 3 to SEQ ID NO: 37.

It is further preferred that the sequence of the nitrogenous bases of the nucleotide units of the fragment comprised in the oligonucleotide molecule has at least 80%, 85%, 90%, 91%, 92%, 93%, 94%, 95%, 96%, 97%, 98%, 99%, 99.5%, 100% identity over the full sequence of the nitrogenous bases of the oligonucleotide of the ASO1 (SEQ ID NO: 38 or 76) or of the oligonucleotide sequence of ASO3 (SEQ ID NO: 39 or 77), or of the siRNAs (SEQ ID NOs: 40 and 43), or the miR-107 analogue (agomiR-107) (SEQs ID NO: 41 and 42 or 78). Preferably, the oligonucleotide sequence has at least 80% identity over the full sequence of any the oligonucleotides of SEQ ID NO: 38 to 43 or 76, 77, 78, preferably SEQ ID: 38 and 39. Also, preferably, the oligonucleotide sequence has at least 90% identity over the full sequence of any the oligonucleotides of SEQ ID NO:38 to 43 or 76, 77, 78, preferably SEQ ID: 38 and 39. Also preferably, the oligonucleotide sequence has at least 95%, 96%, 97%, 98%, 99%, or 100% identity over the full sequence of any the oligonucleotides of SEQ ID NO: 38 to 43 or 76, 77, 78, preferably SEQ ID: 38 and 39, or the complementary sequences thereof.

In a further embodiment, the oligonucleotide sequence comprises a first fragment and a second fragment, wherein the first fragment is a seed region composed of a succession of at least 5-8 nucleotides wherein the sequence of the nitrogenous bases of said first fragment is identical in at least 90%, 91%, 92%, 93%, 94%, 95%, 96%, 97%, 98%, 99% or 100% to the sequence of any of SEQ ID NO: 38 to 43 or 76, 77, 78, and wherein the second fragment is adjacent to the first fragment (i.e., it is located upstream and/or downstream of the first fragment) and it is composed of a succession of at least 7, 8, 9, 10, 11, 12, 13, 14, 15, or 16 consecutive nucleotides that are identical in at least 30%, 40%, 50%, 60%, 70%, 80%, 85%, 90%, 91%, 92%, 93%, 94%, 95%, 96%, 97%, 98%, 99% or 99.5%, or 100% to the sequence of any of SEQ ID NO: 38 to 43 or 76, 77, 78.

In another embodiment according to the first aspect, the oligonucleotide molecule is at least 5, 6, 7, 8, 9, 10, 11, 12, 13, 14, 15, 16, 17, 18, 19, 20, 21, 22, 23, 24, 25, 26, 27, 28, 29, 30 nucleotides in length, of which at least 5, 6, 7, 8, 9, or 10 nucleotides are consecutive and have at least 70%, 75%, 80%, 85%, 90%, 95%, 96%, 97%, 98%, 99% or 100% identity over the full sequence of any of nucleotides of SEQ ID: 38 to 43 or 76, 77, 78.

Preferably, the oligonucleotide molecule comprises at least 1, at least 2, or at least 3, locked nucleic acid nucleotides in their 5′ and 3′ ends. Preferably, the oligonucleotide molecule comprises 3 locked nucleic acid nucleotides at its 5′ and 3′ ends. Preferably, the oligonucleotide comprises at least 1, 2, 3, 4, 5, 6, 7, 8, 9, 10 or 11 nucleotides that are bound by phosphorothioate linkages. Preferably, the phosphorothioate internucleotide linkages are placed where there are not locked nucleic acid nucleotides.

Public and private tools can be used to sequence alignment for comparison between two nucleotide sequences. Some of them are the BLAST family programs, which are of public access (for example, through the webpage of the U.S. National Center for Biotechnological Information: http://blast.ncbi.nlm.nih.gov/Blast.cgi) and can be used to carry out searches and calculations of identity, especially for the purposes of the disclosure, BLAST TWO SEQ, dedicated to nucleotides. As stated above, the percentage of identity is calculated based on any suitable local alignment algorithm.

Importantly, in all the SEQ IDs and sequences disclosed herein, “T” nucleotide is to be understood as interchangeable for “U” nucleotide if the oligonucleotides according any aspect of the present disclosure are produced as RNA instead of DNA, and the other way around (“U” can be “T” if an RNA oligonucleotide is produced as DNA). Similar applies to the complementary sequences thereof.

In a further embodiment, the inhibitor according to the first aspect of the present disclosure may be a molecule that acts as a decoy and hijacks MSI2 protein or transcripts interfering with their normal function. In an embodiment, the inhibitor hijacks MSI2 protein or transcript in the cytoplasm of the cell, reducing the normal activity of MSI2 protein within this cell. For instance, the inhibitor may be an RNA sequence with the consensus binding sites for the MSI2 protein so that the protein is sequestered by the decoy and depleted for its normal functions.

### B) Proteins and Small Molecules

In another preferred embodiment, the MSI2 inhibitor for use in the treatment of myotonic dystrophy according to the first aspect of the present disclosure reduces the activity of MSI2 protein. Proteins and small molecule inhibitors or modulators of MSI2 able to interact with MSI2 protein and inhibit its function are also included in the present disclosure. In a preferred embodiment, the inhibitor is a small molecule

It is known that there are key residues in MSI2 protein that are involved in its activity and therefore any inhibitor able to interact with them will potentially be an inhibitor of the activity of MSI2 protein. These residues were described in Minuesa et al. [Minuesa, G., Albanese, S. K., Xie, W. et al. Small-molecule targeting of MUSASHI RNA-binding activity in acute myeloid leukemia. Nat Commun 10, 2691 (2019). https://doi.org/10.1038/s41467-019-10523-3] and are K22, F66, F97 and R100. In an embodiment of the first aspect, the inhibitor is a protein or a small molecule that is able to interact with the residues K22, F66, F97 and R100 thereby reducing the activity of MSI2 protein.

In a further preferred embodiment, the MSI2 inhibitor that reduces the activity of the MSI2 protein is a small molecule such as 2,3,4,10-Tetrahydro-7,10-dimethyl-2,4-dioxobenzo[g]pteridine-8-carboxaldehyde (also called Ro-08-2750), analogues and derivatives thereof. Ro-08-2750 is an inhibitor of NGF that also binds directly and selectively to MSI2 and competes for its RNA binding in biochemical assays.

In another embodiment, the inhibitor according to the first aspect of the present disclosure may be directed to modify or target the DNA sequence of MSI2 gene, thereby reducing the expression of MSI2 gene and thus reducing the levels of MSI2 transcript and/or proteins. In a further embodiment, the inhibitor may modify the methylation status of the MSI2 gene, thereby reducing the expression of the MSI2 gene and thus reducing the levels of MSI2 transcript and proteins. In a further embodiment, the inhibitor may be an oligonucleotide sequence or a small molecule that edits or modifies MSI2 transcripts, alone or in combination with a protein activity, causing a decrease in their stability and therefore reducing the levels of intracellular MSI2 transcripts and/or intracellular MSI2 protein. Alternatively, in a further embodiment, inhibitors according to the present disclosure include molecules or proteins that target DNA regulatory elements, such as promoters or enhancers, wherein this targeting by the inhibitors induce a reduction of the expression of MSI2 gene.

### C) Compounds or Systems of Compounds for Gene Therapies

The inhibitor according to the first aspect may be a system comprising one or more compounds. Thus, also included in the present disclosure are compounds, systems, or complexes of proteins and/or oligonucleotides that are commonly used for gene therapies and that have an effect as inhibitors according to this aspect of the present disclosure.

In an embodiment, and as defined above, the inhibitor might consist of only one type of compound (i.e., oligonucleotides) or it may be comprised of a system of one or more than one compounds that work together and to modulate the expression of MSI2 gene, transcripts, protein or its activity. For instance, the inhibitor of the present disclosure may be accompanied or fused or administered together with a CRISPR/cas system. In an embodiment, the CRISPR/cas system comprises at least a gRNA, a Cas protein or the nucleotide sequence comprising the Cas protein, and, optionally, a donor DNA. In this case, the CRISPR/Cas system will provide the tools to modify the gene sequence of MSI2, or its regulatory sequences, such as its promoter, in order to eventually lead to a reduction in MSI2 protein or MSI2 transcripts levels, or a reduction in MSI2 activity.

In another embodiment, the CRISPR/Cas9 genetic system is designed to be used in therapeutic approaches consisting of targeting the removal of gene parts at the MSI2 DNA level, preventing transcription of the gene (e.g., by sterically hindering RNA polII transcription), or targeting degradation of its RNA.

In another embodiment, the complex comprises at least a cas13 protein and a guide RNA that form a small complex (crRNA) that is able to bind to MSI2 RNA rather to the DNA and modify it, promoting or inhibiting the endogenous expression of target genes, for instance, MSI2 gene.

In an embodiment, a complex formed by Cas13 that specifically binds to RNA is used to effectively target the RNA of MSI2, thereby the overexpression of MSI2 in MD1 cells. This system is explained in detail in Cov et al. 2017 [Cox, D., Gootenberg, J. S., Abudayyeh, O. O., Franklin, B., Kellner, M. J., Joung, J., & Zhang, F. (2017). RNA editing with CRISPR-Cas13. Science (New York, N.Y.), 358 (6366), 1019-1027. https://doi.org/10.1126/science.aaq0180].

In a further embodiment, engineered nuclease-deficient versions of Cas9 or Cas13, termed dCas9 or dCas13, when fused to effector domains with distinct regulatory functions, enables the repurposing of the CRISPR-Cas9 system to a general platform for RNA-guided DNA or RNA targeting for selective degradation or editing, among other possibilities.

### Treatment, Uses, Administration

In an embodiment according to the first aspect of the present disclosure, the inhibitor is for use in mammal. In a preferred embodiment, the mammal is a human being.

In a preferred embodiment, the MSI2 inhibitor is for use in the treatment of myotonic dystrophy type 1 or type 2, preferably type 1. As defined in the first aspect, treatment of MD1 is to be preferably understood as a palliative treatment of one or more symptoms of myotonic dystrophy type 1, preferably a palliative treatment of one or more of the muscular disorders that are part of the symptoms of myotonic dystrophy type 1. It is noted that the myotonic dystrophy type 1 which may be prevented or treated in a patient by the present disclosure is selected from asymptomatic, mild, severe or terminal MD1 or severe congenital myotonic dystrophy type 1, adult-onset MD1, infantile and juvenile MD1 and/or late-onset MD1.

Given the stability of the inhibitors of the present disclosure, direct administration to human beings and animals can be considered, for example via subcutaneous or systemic routes, preferably intravenously, for example dissolved or suspended in a pharmaceutically acceptable carrier, such as water or an aqueous solution such as saline or phosphate buffer. The composition in which they are administered may contain pharmaceutically acceptable excipients. As to the route of administration, the inhibitors of the present disclosure can be administered via different routes: intravenous (IV), intraperitoneal (IP), intradermal (ID), intramuscular (IM), intranasal (IN), intratracheal, hydrodynamic tail injection, inhalation, or local organ delivery.

In another embodiment, the inhibitor is in the form of a prodrug such that, after administration, it is metabolized or transformed inside the body or cell where it is administrated and is converted into a pharmacologically active drug. The prodrug can be designed to improve the bioavailability, or to improve how the inhibitor according to the first aspect of the present disclosure is absorbed, distributed, metabolized and/or excreted.

In an embodiment, the present disclosure includes a method of integrating the inhibitor into the genome of a cell. In certain embodiments, the cell is a eukaryotic cell, e.g., a mammalian or human cell. In certain embodiments, the cell is a genetically engineered cell.

In a second aspect, this disclosure relates to a pharmaceutical composition comprising an inhibitor (or a system of one or more compounds) as defined in the first aspect of the present disclosure, or a mixture of two or more of them, optionally further comprising a carrier and/or one or more pharmaceutically acceptable excipients. In another embodiment of the second aspect, the pharmaceutical composition comprises an expression vector comprising the nucleotide sequence of at least one of the oligonucleotide molecules as defined in the first aspect of the present disclosure or a designed protein or peptide that recognizes the same sequences that the oligonucleotide sequences defined in the first aspect binds to. In a preferred embodiment, compatible with the preceding one, the composition comprises at least one of the oligonucleotide molecules described in the present disclosure, such oligonucleotides targeting the sequences represented by SEQ ID: 3 to SEQ ID: 37, preferably the nucleotides as set forth in sequences SEQ ID NO: 38 to 43 or 76, 77, 78, preferably SEQ ID: 38 and 39. In another possible embodiment, the composition comprises small molecules such as Ro-08-2750 (2,3,4,10-Tetrahydro-7,10-dimethyl-2,4-dioxobenzo[g]pteridine-8-carboxaldehyde), analogues or derivatives thereof. In an embodiment, the inhibitor for use according to the first aspect is comprised in a composition, preferably a pharmaceutical composition, optionally further comprises a carrier and/or one or more pharmaceutically acceptable excipients.

The expressions “pharmaceutically acceptable” or “pharmacologically acceptable” refer to molecular entities and compositions that do not produce any adverse, allergic or other reactions when administered to an animal or human being. As used herein, “pharmaceutically acceptable vehicle” includes solvents, buffers, solutions, dispersion media, cells, coatings, antibacterial and antifungal agents, isotonic and absorption retarding agents and similar acceptable for use in pharmaceutical formulations, such as pharmaceutical products suitable for administration to human beings. The use of such media and agents for pharmaceutical-active substances is well known in the art. Except where any conventional medium or agent is incompatible with the active ingredients of this disclosure, their use in the pharmaceutical compositions of the disclosure is contemplated. Additional active ingredients may also be incorporated into the compositions, provided that they do not inactivate the molecules of this disclosure or their expression vectors. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present disclosure, its use in therapeutic compositions is contemplated. Supplementary active ingredients or other compounds, such as those directed to increase the uptake or the solubility of the inhibitor, also can be incorporated into the compositions, provided they do not inactivate the inhibitor of the compositions.

The pharmaceutical compositions provided herein in the second aspect may generally include one or more pharmaceutically acceptable and/or approved carriers, additives, antibiotics, preservatives, adjuvants, diluents and/or stabilizers. Such auxiliary substances can be water, saline, glycerol, ethanol, wetting or emulsifying agents, pH buffering substances, or the like. Suitable carriers are typically large, slowly metabolized molecules such as proteins, polysaccharides, polylactic acids, polyglycolic acids, polymeric amino acids, amino acid copolymers, lipid aggregates, or the like.

The composition is administrated in an effective dose. An effective dose of an inhibitor/antagonist of an oligonucleotide can be from approximately 1 mg/kg to approximately 100 mg/kg, from approximately 2.5 mg/kg to approximately 50 mg/kg, or from approximately 5 mg/kg to approximately 25 mg/kg. The precise determination of what would be considered an effective dose can be based on individual factors for each patient, including size, age, and the nature of the inhibitor or antagonist (for example, if it is an expression construct, an oligoribonucleotide analogue . . . ). Therefore, the dosages can be easily determined by ordinary experts skilled in the art based on this description and the knowledge in the art. It may be necessary or convenient to administer multiple doses to the subject during a particular treatment period, administering doses daily, weekly, monthly, every two months, every three months or every six months. In certain embodiments, the subject receives an initial dose at the beginning which is larger than one or more subsequent doses or maintenance doses.

The composition of this aspect may be administered parenterally, subcutaneously, intravenously, intramuscularly, or intranasally, in particular subcutaneously or intramuscularly. Preferably, the pharmaceutical composition is administrated intramuscularly. In other embodiments, the said pharmaceutical composition is administered by any other path of administration known to the skilled practitioner. In a further preferred embodiment, the said pharmaceutical composition is administered intramuscularly, preferably the pharmaceutical composition is administered intramuscularly in a volume ranging between about 0.1 and 1.0 ml. Preferably, the pharmaceutical composition is administered in a volume ranging between 0.25 and 1.0 ml. More preferably, the said pharmaceutical composition is administered in a volume of about 0.5 ml.

In a third aspect, this disclosure relates to an inhibitor or to a pharmaceutical composition as defined in the first and the second aspect of the present disclosure, respectively, for use in the treatment of myotonic dystrophy, particularly myotonic dystrophy type 1 or myotonic dystrophy type 2, preferably myotonic dystrophy type 1. Preferably, the pharmaceutical composition is for use in the palliative treatment of one or more symptoms of myotonic dystrophy, more preferably type 1. In a preferred embodiment, the pharmaceutical composition is for use in the palliative treatment of one or more of the muscular disorders that are part of the symptoms of MD1.

In a fourth aspect, the present disclosure relates to an inhibitor of the RNA-Binding protein Musashi homolog 2 (MSI2) function. Preferably, the inhibitor of the RNA-Binding protein Musashi homolog 2 (MSI2) function is capable of reducing in a myotonic dystrophy (MD) cell or in a cell with the phenotype of a MD cell, at least one of
i) MSI2 protein activity,
ii) intracellular transcript levels of MSI2, and/or
iii) intracellular protein levels of MSI2,
thereby causing in the same MD cell an increase in the intracellular miR-7 levels of at least 1.5-fold of the intracellular miR-7 levels of a reference cell, as measured by a gene expression quantification technique such as quantitative reverse transcription PCR (qRT-PCR), Northern blotting, or RNA-sequencing (RNA-Seq) techniques. The definitions of the terms “myotonic dystrophy (MD) cell”, “inhibitor”, “reference cell”, “healthy cell”, “MSI2 activity”, “MSI2 transcript”, “protein levels”, “reducing in a myotonic dystrophy cell at least one of i) MSI2 protein activity, ii) intracellular transcript levels of MSI2, or iii) intracellular protein levels of MSI2”, “causing in the same MD cell an increase in the intracellular miR-7 levels”, and “gene expression quantification technique” and the embodiments related to A) Oligonucleotides for RNA therapies, B) Proteins and small molecules, and C) Compounds or systems of compounds for gene therapies, explained and defined under the first aspect of the present disclosure also apply herein and thus they should also be considered as embodiments of the fourth aspect.

Preferably, the MSI2 inhibitor according to the fourth aspect that reduces the activity of the MSI2 protein is a small molecule such as 2,3,4,10-Tetrahydro-7,10-dimethyl-2,4-dioxobenzo[g]pteridine-8-carboxaldehyde (also called Ro-08-2750), or any analogues or derivatives thereof.

Preferably, the MSI2 inhibitor according to the fourth aspect that is capable of reducing the intracellular transcript levels of MSI2 is an oligonucleotide molecule as defined under the section a) Oligonucleotides for RNA therapies, in the first aspect of the disclosure. Preferably, the MSI2 inhibitor is an oligonucleotide that is sufficiently complementary to a region comprised in any of the MSI2 transcripts, so that the inhibitor is able to hybridize under stringent conditions to the MSI2 transcript thereby inhibiting the expression of said transcript.

Preferably, the oligonucleotide molecule according to the first aspect is between 10-50, 10-40, 10-30, 15-30, preferably 15-25, nucleotides in length and comprises a sequence that is at least 60%, 65%, 70%, 75%, 80%, 85%, 90%, 95%, 96%, 97%, 98%, 99% or 100% identical over the full-length sequence of the target sequence where they bind, which may be SEQ ID NO: 1 or, preferably, SEQ ID NO: 2, or any of SEQ ID NOs: 109 to 137, or the complementary sequence thereof. In another embodiment according to the fourth aspect, the oligonucleotide molecule is between 10-50, 10-40, 10-30, 15-30, preferably 15-25, nucleotides in length, of which at least 5, 6, 7, 8, 9, or 10 consecutive nucleotides have at least 60%, 65%, 70%, 75%, 80%, 85%, 90%, 91%, 92%, 93%, 94%, 95%, 96%, 97%, 98%, 99%, 99.5%, 100% identity with the SEQ ID NO: 1 or 2 or any of SEQ ID NOs: 109 to 137, or their complementary sequence thereof. In a preferred embodiment, the inhibitor oligonucleotides molecules according to the present disclosure are between 10-50, 10-40, 10-30, 15-30, preferably 15-25, nucleotides in length and are capable of hybridizing under stringent conditions to a region of at least 5, 6, 7, 8, 9, or 10 consecutive nucleotides comprised in SEQ ID NO: 1 or preferably SEQ ID NO: 2, or any of SEQ ID NOs: 109 to 137.

In a further embodiment, the oligonucleotide sequence is between 10-50, 10-40, 10-30, 15-30, preferably 15-25, nucleotides in length, and it comprises a first fragment and a second fragment, wherein the first fragment is a seed region composed of a succession of at least 5-8 nucleotides wherein the sequence of the nitrogenous bases of said first fragment is identical in at least 90%, 91%, 92%, 93%, 94%, 95%, 96%, 97%, 98%, 99% or 100% to the complementary sequence SEQ ID NO: 1 or 2 or any of SEQ ID NOs: 109 to 137, and wherein the second fragment is adjacent to the first fragment (i.e., it is located upstream and/or downstream of the first fragment) and it is composed of a succession of at least 7, 8, 9, 10, 11, 12, 13, 14, 15, or 16 consecutive nucleotides that are identical in at least 30%, 40%, 50%, 60%, 70%, 80%, 85%, 90%, 91%, 92%, 93%, 94%, 95%, 96%, 97%, 98%, 99% or 99.5%, or 100% to the complementary sequence of SEQ ID NOs: 1 or 2 or any of SEQ ID NOs: 109 to 137.

In an embodiment, the oligonucleotide molecule according to the fourth aspect of the present disclosure is between 10-50, 10-40, 10-30, 15-30, preferably 15-25, nucleotides in length and comprises a sequence with at least 60%, 65%, 70%, 75%, 80%, 85%, 90%, 91%, 92%, 93%, 94%, 95%, 96%, 97%, 98%, 99%, 99.5%, or 100% identity over the full sequence of any of the nucleotides of SEQ ID NO: 3 to SEQ ID NO: 37 or their complementary sequence thereof. In another embodiment according to the fourth aspect, the oligonucleotide molecule is between 10-50, 10-40, 10-30, 15-30, preferably 15-25, nucleotides in length, of which at least 5, 6, 7, 8, 9, or 10 consecutive nucleotides have at least 60%, 65%, 70%, 75%, 80%, 85%, 90%, 91%, 92%, 93%, 94%, 95%, 96%, 97%, 98%, 99%, 99.5%, 100% identity with any of SEQ ID: 3 to 37 or their complementary sequence thereof. In a preferred embodiment, the oligonucleotide molecule is between 15-25 nucleotides in length and has at least 90%, preferably 100%, identity over the full sequence of any of the oligonucleotides SEQ ID NO: 3 to SEQ ID NO: 37, or the complementary sequences thereof. In a preferred embodiment, the inhibitor oligonucleotides molecules according to the present disclosure are between 10-50, 10-40, 10-30, 15-30, preferably 15-25 nucleotides in length and are capable of hybridizing under stringent conditions to a region of at least 5, 6, 7, 8, 9, or 10 consecutive nucleotides comprised in any of SEQ ID NO: 3 to SEQ ID NO: 37.

In a further embodiment, the oligonucleotide molecule is between 10-50, 10-40, 10-30, 15-30, preferably 15-25, nucleotides in length and comprises a first fragment and a second fragment, wherein the first fragment is a seed region composed of a succession of at least 5-8 nucleotides wherein the sequence of the nitrogenous bases of said first fragment is identical in at least 90%, 91%, 92%, 93%, 94%, 95%, 96%, 97%, 98%, 99% or 100% to the complementary sequence of any of SEQ ID NO: 3 to SEQ ID NO: 37, and wherein the second fragment is adjacent to the first fragment (i.e., it is located upstream and/or downstream of the first fragment) and it is composed of a succession of at least 7, 8, 9, 10, 11, 12, 13, 14, 15, or 16 consecutive nucleotides that are identical in at least 30%, 40%, 50%, 60%, 70%, 80%, 85%, 90%, 91%, 92%, 93%, 94%, 95%, 96%, 97%, 98%, 99% or 99.5%, or 100% to the complementary sequence of any of SEQ ID NO: 3 to SEQ ID NO: 37. Most preferably, the oligonucleotide consists of a sequence that is identical to the complementary sequence of any of SEQ ID NO: 3 to SEQ ID NO: 37.

It is further preferred that the oligonucleotide molecule is between 10-50, 10-40, 10-30, 15-30, preferably 15-25, nucleotides in length, and comprises a sequence with at least 60%, 65%, 70%, 75%, 80%, 85%, 90%, 91%, 92%, 93%, 94%, 95%, 96%, 97%, 98%, 99%, 99.5%, 100% identity over the full sequence of the ASO1 (SEQ ID NO: 38 or 76) or of the oligonucleotide sequence of ASO3 (SEQ ID NO: 39 or 77), or of the siRNAs (SEQ ID NOs: 40 and 43), or the miR-107 analogue (agomiR-107) (SEQs ID NO: 41 and 42 or 78) or the complementary sequences thereof. Preferably, the oligonucleotide is between 10-50, 10-40, 10-30, 15-30, preferably 15-25, nucleotides in length and comprises a sequence with at least 80% identity over the full sequence of any the oligonucleotides of SEQ ID NO: 38 to 43 or 76, 77, 78, preferably SEQ ID: 38 and 39. Also, preferably, the oligonucleotide sequence is between 10-50, 10-40, 10-30, 15-30, preferably 15-25, nucleotides in length and comprises a sequence with at least 90% identity over the full sequence of any the oligonucleotides of SEQ ID NO: 38 to 43 or 76, 77, 78, preferably SEQ ID: 38 and 39. Also preferably, the oligonucleotide is between 10-50, 10-40, 10-30, 15-30, preferably 15-25 nucleotides in length and comprises a sequence with at least 95%, 96%, 97%, 98%, 99%, or 100% identity over the full sequence of any the oligonucleotides of SEQ ID NO: 38 to 43 or 76, 77, 78, preferably SEQ ID: 38 and 39, or the complementary sequences thereof. In another embodiment according to the fourth aspect, the oligonucleotide molecule is between 10-50, 10-40, 10-30, 15-30, preferably 15-2,5 nucleotides in length, of which at least 5, 6, 7, 8, 9, or 10 consecutive nucleotides have at least 60%, 65%, 70%, 75%, 80%, 85%, 90%, 91%, 92%, 93%, 94%, 95%, 96%, 97%, 98%, 99%, 99.5%, 100% identity over the full sequence of any the oligonucleotides of SEQ ID NO: 38 to 43 or 76, 77, 78, preferably SEQ ID: 38 and 39, or the complementary sequences thereof.

In a further embodiment, the oligonucleotide sequence is between 10-50, 10-40, 10-30, 15-30, preferably 15-25, nucleotides in length, and it comprises a first fragment and a second fragment, wherein the first fragment is a seed region composed of a succession of at least 5-8 nucleotides wherein the sequence of the nitrogenous bases of said first fragment is identical in at least 90%, 91%, 92%, 93%, 94%, 95%, 96%, 97%, 98%, 99% or 100% to the sequence of any of SEQ ID NO: 38 to 43 or 76, 77, 78, and wherein the second fragment is adjacent to the first fragment (i.e., it is located upstream and/or downstream of the first fragment) and it is composed of a succession of at least 7, 8, 9, 10, 11, 12, 13, 14, 15, or 16 consecutive nucleotides that are identical in at least 30%, 40%, 50%, 60%, 70%, 80%, 85%, 90%, 91%, 92%, 93%, 94%, 95%, 96%, 97%, 98%, 99% or 99.5%, or 100% to the sequence of any of SEQ ID NO: 38 to 43 or 76, 77, 78. Most preferably, the oligonucleotide is selected from the group consisting of any of SEQ ID NO: 38 to 43 or 76, 77, 78.

Preferably, the oligonucleotide molecule comprises at least 1, at least 2, or at least 3, locked nucleic acid nucleotides in their 5′ and 3′ ends. Preferably, the oligonucleotide molecule comprises 3 locked nucleic acid nucleotides at its 5′ and 3′ ends. Preferably, the oligonucleotide comprises at least 1, 2, 3, 4, 5, 6, 7, 8, 9, 10 or 11 nucleotides that are bound by phosphorothioate linkages. Preferably, the phosphorothioate internucleotide linkages are placed where there are not locked nucleic acid nucleotides. The oligonucleotide may be conjugated to a fatty acid molecule, preferably cholesterol.

In a fifth aspect, the present disclosure provides a vector comprising at least one of the inhibitors of the fourth aspect or any of its embodiments. In a preferred embodiment, the vector is an expression vector and it comprises at least one of the oligonucleotide inhibitors defined in the fourth aspect or any of its embodiments. Preferably, the expression vector is selected from the group consisting of a cell, a viral vector, a DNA vector, a RNA vector, or a liposome vector. Preferably, the viral vector is a non-integrative vector, such as an adenoviral or adeno-associated vector. Preferably, the viral vector is an integrative vector such as a retroviral vector. Preferably, the DNA vector is a plasmid DNA.

Preferably, the expression vector comprises a short hairpin RNA molecule (shRNA), or a sequence encoding a shRNA. A short hairpin RNA or small hairpin RNA (shRNA/Hairpin Vector) is an artificial RNA molecule with a tight hairpin turn that can be used to silence target gene expression via RNA interference (RNAi). shRNA is an advantageous mediator of RNAi in that it has a relatively low rate of degradation and turnover.

In an embodiment, the shRNA comprises:
(i) a first sequence comprising at least one of the oligonucleotide inhibitors defined in the present disclosure, preferably those defined under the fourth aspect or any of its embodiments, and
(ii) a second sequence adjacent to the first sequence, wherein said second sequence has at least 80%, 85%, 90%, 91%, 92%, 93%, 94%, 95%, 96%, 97%, 98%, 99%, or 100% complementarity to the first sequence.

Preferably, the complementarity between the first and the second sequences is sufficient to form a hairpin by hybridization between them. In some embodiments, the second sequence is directly following, i.e., adjacent to, the first sequence. Alternatively, in other embodiments, the second sequence is separated from the first sequence in at least 1, 2, 3, 4, 5, 6, 7, 8, 9, 10, or more than 10 nucleotides. The distance between the first and the second sequence is not limiting as long as the second sequence is able to fold back and hybridize with the first sequence, thereby forming a hairpin. Preferably, the nucleotide complementarity between the first and the second sequence is of at least 80%, 85%, 90%, 91%, 92%, 93%, 94%, 95%, 96%, 97%, 98%, 99%, or 100%.

Preferably, the first sequence is between 10-50, 10-40, 10-30, 15-30, preferably 15-25, nucleotides in length and comprises a sequence that is complementary to any of the sequences of SEQ ID NO: 1 to 37 or any of SEQ ID NOs: 109 to 137, or to a sequence that is at least 60%, 70%, 80%, 85%, 90%, 91%, 92%, 93%, 94%, 95%, 96%, 97%, 98%, 99% or 99.5%, or 100% identical to the complementary sequence of SEQ ID NO: 1 to SEQ ID NO: 37 or any of SEQ ID NOs: 109 to 137. Preferably, the first sequence is 100% identical to the complementary sequence of any of SEQ ID NO: 3 to SEQ ID NO: 37 or to a region of any of SEQ ID NOs: 1, 2, 109 to 137. Preferably, the first sequence is between 10-50, 10-40, 10-30, 15-30, preferably 15-25, nucleotides in length and comprises any of the sequences of SEQ ID NO: 38 to 43 or 76, 77, 78, or a sequence that is at least 60%, 70%, 80%, 85%, 90%, 91%, 92%, 93%, 94%, 95%, 96%, 97%, 98%, 99% or 99.5%, or 100% identical to any of the sequences of SEQ ID NO: 38 to 43 or 76, 77, 78. Preferably, the first sequence is selected from the group consisting of any of SEQ ID NO: 38 to 43 or 76, 77, 78.

Preferably, the second sequence is between 10-25, 15-25, preferably between 16-23 nucleotides in length. Preferably, the first sequence is of at least 15, preferably between 16-23 nucleotides in length, and the second sequence is complementary, preferably fully complementary, to a sequence of the first nucleotides counted from the 5′ end of the first sequence, so that the shRNA molecule forms a hairpin. Preferably, the second sequence is complementary to the sequence of the first 15, 16, 17 or 18 nucleotides counted from the 5′ end of the first sequence.

Preferably, the shRNA molecule is expressed in a mammalian cell in an amount sufficient to attenuate expression of the target gene (MSI2) in a sequence specific manner, whereby expression of the target gene is reduced.

In certain embodiments, the expression vector comprises a sequence encoding the shRNA as defined herein operably linked to an RNA polymerase promoter. The shRNA can be introduced into the cell directly, or can be complexed with cationic lipids, packaged within liposomes, or otherwise delivered to the cell. In certain embodiments the shRNA can be a synthetic shRNA, including shRNAs incorporating modified nucleotides, such as those with chemical modifications to the 2′-OH group in the ribose sugar backbone, such as 2′-O-methyl (2′OMe), 2′-fluoro (2′F) substitutions, and those containing 2′OMe, or 2′F, or 2′-deoxy, or “locked nucleic acid” (LNA) modifications. In some embodiments, an shRNA contains modified nucleotides that increase the stability or half-life of the shRNA molecule in vivo and/or in vitro. Further, the shRNA can comprise one or more aptamers, which interact(s) with a target of interest to form an aptamer: target complex. The aptamer can be at the 5′ or the 3′ end of the shRNA. Aptamers can be developed through the SELEX screening process and chemically synthesized. Suitable targets include small organic molecules, polynucleotides, polypeptides, and proteins. Proteins can be cell surface proteins, extracellular proteins, membrane proteins, or serum proteins, such as albumin. Such target molecules may be internalized by a cell, thus effecting cellular uptake of the shRNA. Other potential targets include organelles, viruses, and cells.

Alternatively, delivery and expression of shRNA in cells can be accomplished by delivery of plasmids (plasmid transfection) or through viral or bacterial vectors. Thus, also included in the present disclosure is an expression vector comprising the shRNA as defined herein or comprising a DNA sequence encoding for the shRNA. Examples of suitable expression vectors comprising the shRNA or encoding for the shRNA are viral vectors or plasmid vectors. Preferably, a non-replicating viral vector is used to deliver the shRNA, preferably an adeno-associated viral vector.

In an embodiment, the shRNA may be introduced into a mammalian cell in an amount sufficient to attenuate target gene expression (MSI2) in a sequence specific manner. In some embodiments, the present disclosure also relates to a cell comprising the shRNA described herein. Said cell may be a mammalian cell, preferably a human cell. Alternatively, the cell may also be a non-human mammal cell comprising the shRNA described herein. In certain embodiments, the non-human mammal may be a chimeric mammal, some of whose somatic or germ cells comprising the shRNAs described herein. Alternatively, the non-human mammal may be a transgenic mammal, all of whose somatic or germ cells comprise the shRNAs described herein. Thus, transgenic mammals whose genomes comprise a sequence encoding the shRNAs of the disclosure are also provided. In one embodiment, the transgenic mammal is a mouse.

In a sixth aspect, the present disclosure further provides a composition, preferably a pharmaceutical composition, comprising an inhibitor as defined in the fourth aspect or the vector as defined in the fifth aspect or any of their embodiments, or a mixture of two or more of them, optionally further comprising a carrier and/or one or more pharmaceutically acceptable excipients. The embodiments defined in the third aspect relating to a composition also apply herein.

In a seventh aspect, the present disclosure relates to an inhibitor, a vector, or to a composition according to the fourth, fifth, or sixth aspects, for use in the treatment of myotonic dystrophy, particularly myotonic dystrophy type 1 or myotonic dystrophy type 2, preferably myotonic dystrophy type 1. Preferably, the inhibitor, vector, or composition according to the fourth, fifth, or sixth aspects is for use in the palliative treatment of one or more symptoms of myotonic dystrophy, more preferably type 1. In a preferred embodiment, the inhibitor, vector, or composition is for use in the palliative treatment of one or more of the muscular disorders that are part of the symptoms of MD1.

In one more aspect, this disclosure relates to an inhibitor or to a pharmaceutical composition as defined in the first and the second aspect of the present disclosure, respectively, or as defined in any of the other aspects of the present disclosure, a mixture of two or more thereof, for the manufacture of a medicinal product for the treatment of myotonic dystrophy, preferably myotonic dystrophy type 1. In this aspect of the disclosure referred to the use for the manufacture of a medicinal product and, therefore, related to the use in the treatment of myotonic dystrophy type 1, a possible embodiment is that the inhibitor is, or the pharmaceutical composition comprises, a molecule that reduces the intracellular levels of MSI2 transcript, an inhibitor of MSI2 protein, or a molecule that reduces the intracellular protein levels of MSI2, a mixture thereof as defined in the first and second aspects of the present disclosure, or as defined in any of the other aspects of the present disclosure.

In a further aspect, the disclosure relates to a method of treating a subject for myotonic dystrophy, preferably myotonic dystrophy type 1, comprising administering to a mammal at least one inhibitor of RNA-binding protein Musashi (MSI2) function or an analogue thereof, capable of reducing in a myotonic dystrophy (MD) cell or a cell with the phenotype of a MD cell, as defined in the first, second, and third aspects of the present disclosure, or as defined in any of the other aspects of the present disclosure.

According to this aspect of the present disclosure, the inhibitor used in the method of treating a subject for myotonic dystrophy, preferably myotonic dystrophy type 1, may be an oligonucleotide, a protein, a small molecule, a derivative or analogue thereof, or a system of one or more compounds, as defined in the first, fourth or fifth aspects of the present disclosure. In another embodiment, the disclosure relates to a method of treating a subject for myotonic dystrophy, preferably myotonic dystrophy type 1, comprising administering the composition of the second or sixth aspects of the present disclosure. Preferably, this aspect relates to the palliative treatment of one or more symptoms of myotonic dystrophy, more preferably type 1. In a preferred embodiment, this method is for the palliative treatment of one or more of the muscular disorders that are part of the symptoms of MD1. In a preferred embodiment, the subject is a mammal. In a further preferred embodiment, the subject is a human being.

Each embodiment disclosed herein is contemplated as being applicable to each of the other disclosed embodiments. Thus, all combinations of the various elements described herein are within the scope of the disclosure.

SEQUENCE LISTING

SEQ ID NO 1: MSI2 transcript (NCBI Reference Sequence: NM_138962.4)
Homo sapiens musashi RNA binding protein 2 (MSI2), transcript variant 1, mRNA
GTGCGAGGCAGCGGGGCTGAGCTAAGCCGAGCCCACGTGTGACGGCTCTCGCCGCTGC
CCCGGCTCCGCCGCTCGCAGAGAGATTCGGAGGAGCCCGGGCGGGGGGGAGGAGGAG
GGGGAGGAGGGAGCGGAGATCTCGGGGCTCGGAGCCGGCCGCCGCTCCGCTCCGATCG
CTGTGGGGCTTGGTTTTTTGGGGGTGGGGGGGCGGGGGGGCTCAGATATGGAGGCAAA
TGGGAGCCAAGGCACCTCGGGCAGCGCCAACGACTCCCAGCACGACCCCGGTAAAATG
TTTATCGGTGGACTGAGCTGGCAGACCTCACCAGATAGCCTTAGAGACTATTTTAGCAA
ATTTGGAGAAATTAGAGAATGTATGGTCATGAGAGATCCCACTACGAAACGCTCCAGA
GGCTTCGGTTTCGTCACGTTCGCAGACCCAGCAAGTGTAGATAAAGTATTAGGTCAGCC
CCACCATGAGTTAGATTCCAAGACGATTGACCCCAAAGTTGCATTTCCTCGTCGAGCGC
AACCCAAGATGGTCACAAGAACAAAGAAAATATTTGTAGGCGGGTTATCTGCGAACAC
AGTAGTGGAAGATGTAAAGCAATATTTCGAGCAGTTTGGCAAGGTGGAAGATGCAATG
CTGATGTTTGATAAAACTACCAACAGGCACAGAGGGTTTGGCTTTGTCACTTTTGAGAA
TGAAGATGTTGTGGAGAAAGTCTGTGAGATTCATTTCCATGAAATCAATAATAAAATGG
TAGAATGTAAGAAAGCTCAGCCGAAAGAAGTCATGTTCCCACCTGGGACAAGAGGCCG
GGCCCGGGGACTGCCTTACACCATGGACGCGTTCATGCTTGGCATGGGGATGCTGGGAT
ATCCCAACTTCGTGGCGACCTATGGCCGTGGCTACCCCGGATTTGCTCCAAGCTATGGC
TATCAGTTCCCAGGCTTCCCAGCAGCGGCTTATGGACCAGTGGCAGCAGCGGCGGTGGC
GGCAGCAAGAGGATCAGGCTCCAACCCGGCGCGGCCCGGAGGCTTCCCGGGGGCCAAC
AGCCCAGGACCTGTCGCCGATCTCTACGGCCCTGCCAGCCAGGACTCCGGAGTGGGGA
ATTACATAAGTGCGGCCAGCCCACAGCCGGGCTCGGGCTTCGGCCACGGCATAGCTGG
ACCTTTGATTGCAACGGCCTTTACAAATGGATACCATTGAGCAGGTGCTTTCGTTGCCAT
CTCACTCTGAGAGCATACCTGGATGTCCAGGCAAGACTGGGCGAAGTTTCTGAGTGGCC
CTTTGTTTAGGTGATGTCCTCAGACCTGGACCCCCACCAGCCTCACTCCCCATCCCAACC
AGAGATGGCTCACTTCGGATCGAGGGTTGACTACATCTCATCATCTCACGAATCTGCTG
TAATATAAGACAACAGCTTTTAAATGTGTATATAACCCATGATTTCGGTTTTGTTTTGTT
TTGTTTTTCTTGATGGTTTCCCTCTCCCTCCCTCTCTTCCCATTCTCCTTTTAAATCTCTTT
GAATCACATTTGGTAGTGATTTTGACTTAGTCCAGTAGTCACATAGCTTTAATATCTAGT
TCAAAGCTAACCATAGTATAATTGTTATATTAAGGAGTTATTTTTTCTTAAAACATTTTT
TTTTGCTTGTTTTGGTTCTGTTCTCACTTTTAAAGGATGCTGAGATGGTAATATGACTCTC
CATATTTTGGTACCAATTCTGAGACTGTATGAATTTTCAGGTGGAACTTTAGCACACACT
GAAGCAAAGTTGTGAAGTGCAGGGCGGGAGGTGGGCGTGAGCTTTCTATTTTGCGTTGT
AGAAGAAGTGAGATGTAGTAAGCTAATTAACAGACTTTCTAGCAGTTCTTTTTGTGATG
TCTCTTTGTTAATCTGAGTCTATCTATTTTCGGCAATAAGGTAAGGACGACAGTGTTTTG
AGTGTCCTCCTTTTCTATAAGTGCTTTTTTTCTGTTGAAAGAGGTGATATTATAAGGTTTT
TTGAAATTGTGAATTCTAAAAAAGAAATGTTGTAAATACAATTCCATTAACTACATAGA
AACTATTAAGAAAGAGAGAATCAAAAATATTTTTGTGAGGGAGTCGGTCCCAGGCAGT
TTGATGCTCTGTGGAAGGAGGCGGGAAGGGAACGTTGGCCAAGTCAGTTACTGAGATG
AAGATCGCCCAGCTGCCAGGACCACCCCAGGACAAGTTAGAGCACTGTTTAGCTCCTTT
GTCTGTGTGATAGACCTAAGAACTGTATTAGTGTTGTACCAGCCTATTAACCTCTTGTCT
GTGCACAGCTTCAAATGTTACCGTCTAGTTAGATTTTTATTTAAAATATGAAAAACTGCT
TTTCCCAAGATGTTTTTTAAAAACAAAGCTACAATTTTAATATTTAACATATTTAAAGTT
TCAAAGCACACCTGTTTGGCTTGGGTGGGGGTGGGGTGGGGGGGACATTCTTTTTCAGT
CTTAATTTTTAAATATTTGATCATTTTCTATTGTCCAATCATTTCAGCACCTCCAAAGGTC
CCTAGGACACTTTGCCTCTCTTCTCCCCCTGCCCCCCACCCTGCTCCCACATCTGGGGGC
CCATGGGCCAGGAGTGGATAAGCCTGCATTAATACAACCTTTCTCCATTCACTTTCTATT
TACAAATTAGGAAAGCAACCTTTTGGTTTATATATATTTTTTTTAATACCTCAGTGCTGC
AAGTATCACCAGAGAGGCTATGGAAGAATTTTTTTTTAATTTATTGTAGATGTAAACAG
AATTTTAAAAATAAAAAGTATAAACATCACTGCACTGTGACTGGTGGGAAAAACTGAC
AGTTTCCTCTTTGCACATGTTTAACATTTGGCTGTTATAATATATGGTCCTCGGTTGGGG
AAAGATACTTATGATGAAGGATATTTTTTAATTTAACTTTTTTTTAAATATTGGTAATAG
GTCGGCAACAGCAACTATAGAAGTACAACTCAATAGATGGCATTAAAACATATTGTAGT
GTGGATATATATTTTTTCTTTTTTAAAATGTGATATTGACGTTTTATTAATATTTTTTAAA
TTGTTACGTTTATAAATTTGGTACTTAAGGCACAGCCAGTATGAGACACTGAATGCGAC
ATTTATTATAAAGAGCTGCTGCACTCCTATTTTTATAAATTTTACTAACAAAGTAGACTA
ATGTAGACATTCACAGACATGGTAGGGCAAAAGCATCTTCAAACTAAAGACTCCAAAA
TGCTAACTCAGAAAGAAAGAAAAAACCCGTTTTCAATTCTAATGAAACAGCAACAACA
TTTTTTTTAATTAAAAAAAAAATCATGTTCTTTGTTTTTCTAATAAAATGTTAGGTTGTTT
GGTGAGGTTTTTTTGTTGTTTTTTTCCTTTCTTTGTTTCTTTTCTTTTCCCCCAAACAACCA
GATTAAATGCTGAGCGCTTTTAAAATATCAAAACTTGTTCAAGTCATAAAACAACAAAA
CATAAGTTTTATTTAAAAAAAAAAAAAGAAAGAAAAAATAGTAAATTCCCAGAAATTC
AGTGTTTAGACGAGGGAATTTAATTCCTATTTTGTCCATGTTGGTGATGTACTGTACTTC
CCTTCCTTTTCTCTGCATCCCCCATCACCTCATAGAAGACTCTTTGTTGATCATTGTATGT
TAATAATGTATAAAATGGCTATCTTGTAAGCGTGCTGTCCTGGTACTAGTGTAGCGACTT
TTTTTCTCCTCTTTCTTCTAGTACATATTGATAGGTATAACATAATTAAGGTTTAAAAAA
AATTAGACATAGTTATTCAGATTTAGGACCAGTAAGGATAGAACTTTCTCTTATTTATGA
AAAAAAATGCTAATAATTTTGGGGCAGTTTTTTCCTTTAATTATTTTTTTCAATTTCAAGT
TTAATTTTATTTTAGCTGATCTGATGTGGTTTCAACTAACCCAAGGTCTCACCATGTTAA
AATGCCGGCGGACTCTACGGCGTTTTGTAGATCCCCCCCCCCCCACCCACTGTGAAGGG
GTGCCATACTACCTTAAATGCTAATGCTAGATATGCAAAACTGGATTTTTTTAATTTATT
TTTTAAAAGAGGGAGGCATGGTATATTAAAATGATTTTACTAAGAGAAAAAATATTTTT
TTAAGAATGCTCAGAAGAAATTGATAATCTGTGTGAATATGTTTTAGATGTTTATATACC
TTTTGAAGAGACCCAGTAGCCCATAGCACAAATCTTGTGGAAATCCGATATGTTTTAAT
GTGGCTACCTAGGTTTAAGGTTCACGTTAGTCCCCCCATTCCATCTAGAAGTCCATTTTG
AAAGATTTTTGTAAATTCTTTTAACACTGATGTTTCAGCCTCGTCTTTGTTTCAGTTAAGC
TCAATGGCGAACATGGGAACCACCTTTCGCCTTCCCTGGGGGAGAAACCCTCTTGGCTG
ATGGCTTTTCCCCGGAATTATCAAACAGCCACCGGGTGACTTTCTGGCTTCCAGATCCAT
CTGCCTGAGACCCCCGAACTCCTCTCCTCCCAAGCAGAGGCGAGTGAGTGGCATTAGCT
CCCGGACCCATTCCCGGTCCTAGCTGGGCATGGGGCTGACGGAGATGACCAAGCCTTGG
TCTGCTCTCTAGCAGCTTCCACAGACTTGGCTCGTGGCCTTCCTTATATCCACTGGGAAC
AAACAGCCTCGCGCTCTATACCAAACAGCCTCGCGCTCTATCCCAGTCGCCCATTAGCT
TGATTCAAACAAAGCCCCAGCAGGCCTTTGCGTTTTTATCCTTCATAACCTTCATCTTAA
TTTGAACTTGTAGCTTGGACTTTAAGGTAGCATGGCTCTATTGCTGTCAATTTACTGTTT
CACTGCACAGCAATCACAGCCAGTGAATGTTACACACATCTTGCTAGACTAGTATAAAA
ATCATTGGGTAATTGTTGGTTCTAATGACCTGAAAGGTGTTCAGTTTTTGTTCTTGGTTTT
GTTTTGTTTTTGATTTCTTGGGGGTGGGTTTTGCTTGTTGTTCCTTTTCATTTGGGGGTTTT
TGGGGAAAAAATTTATTTTTGGTTCCAAATAGAAAAACAAAACCTATTTTGATCTTTAG
TGCAAACGAGGGCTAGGGACTTAGCCTCCTCCACCACCTCCACACTGCTTCATTCTGCC
ATTCACTCACTGCAGCATATTCAAGAATAAAGCAATATCGTTTACTACATTTTTTATTGA
AGGTCAGCCATGCTTTCTGTATTATATTGCATATGAAATTGTTTACAAAAGAAACACTA
ACTCATACTTCTCTTTATTGGTTGCAAGTGGCACGCAGGAACAGAGGGAGAGTGGGGGG
CTGGTGGGGGAGGGGAGATTTTTTTTTTTTTTTCTTGAATGTGCTTCGCAAGCCAGGCTA
TCTTCCAAGGAAGGCAGACAGTGGGAGAGCAGAGGGACTGACTGCAGGCAAGCACATT
GAAGAAAGACACTGGCGGGTTTCCCCACCCTCACCCCAAAGCAGAAAACTAGCAGACG
TCAGCTCAGCCCCGTCCTGGGCACAGACACTACACAAGGAGACGCTGGAAGTTAAGCA
ATACTTTAATACTGTAATATGTTTGTTTTCTTTTTCTTTCTTTTTTTTCTACCAAAAAAAA
GTAAGTAAACTAAAACACAAAAACATATAAATAAAATCCATCCCTCTTGTCGGGGACCT
GCAGGGGGGCAACCTTAATCCAAACACCTGGCTATCAAATAATCAGAATGTATTGTCTC
AGACAGGATTTCAGTTCCGGGAGGCAGGGGCATGATGGGGGAGGGGGCCGGAGGCTGA
GAGACAAAAGTTCCAGAGCCTCCCTCGAAGGTTCTCTACTACTGTATTCTGTACATAAT
GTACCATCCCATGTGGAATCTGTGAGTGTCCTCTTAAGTAGCGTGGGCTAGCCAATCTG
CCGTTCATGGTGTATTGTAAACTCCGAATTCCATATGTAATAGGATGCAAGTCTAAGCG
TTTCATGTGGACATAAATGTATCTAAATAAAACTTTCCCTAGCACTGTGGCTGACCTCAC
CCTTACTTTTATACTTTAGTATGAAACTGATGAGAACTTTGGTAGTGAGTATTTTTTTTAT
ATATATACATATATATGTACTATCTATATATATATCTCAAGCATCTTTCAGGTCTTTGTG
TGTGGCTTTCTTAAAGCCCTGTTGTAAAAAATTACTATGTGGATGGCAGTCTCTCACATC
ACAGATGTGGAAAGTATAATTTTATATTTGTATTTTCAAATAAATAAGTTTGTGAAAGG
TTTCCATCCTCTACTGTGGTCCAGAAATCAATGTGTTTGTCTGACAAAAAAAAAAATAA
AATAAAATAAACTGTTTTGAACAGA
SEQ ID NO 2: Partial MSI2 transcript (nucleotides 327-1618):
GCCTTAGAGACTATTTTAGCAAATTTGGAGAAATTAGAGAATGTATGGTCATGAGAGAT
CCCACTACGAAACGCTCCAGAGGCTTCGGTTTCGTCACGTTCGCAGACCCAGCAAGTGT
AGATAAAGTATTAGGTCAGCCCCACCATGAGTTAGATTCCAAGACGATTGACCCCAAAG
TTGCATTTCCTCGTCGAGCGCAACCCAAGATGGTCACAAGAACAAAGAAAATATTTGTA
GGCGGGTTATCTGCGAACACAGTAGTGGAAGATGTAAAGCAATATTTCGAGCAGTTTGG
CAAGGTGGAAGATGCAATGCTGATGTTTGATAAAACTACCAACAGGCACAGAGGGTTT
GGCTTTGTCACTTTTGAGAATGAAGATGTTGTGGAGAAAGTCTGTGAGATTCATTTCCAT
GAAATCAATAATAAAATGGTAGAATGTAAGAAAGCTCAGCCGAAAGAAGTCATGTTCC
CACCTGGGACAAGAGGCCGGGCCCGGGGACTGCCTTACACCATGGACGCGTTCATGCTT
GGCATGGGGATGCTGGGATATCCCAACTTCGTGGCGACCTATGGCCGTGGCTACCCCGG
ATTTGCTCCAAGCTATGGCTATCAGTTCCCAGGCTTCCCAGCAGCGGCTTATGGACCAG
TGGCAGCAGCGGCGGTGGCGGCAGCAAGAGGATCAGGCTCCAACCCGGCGCGGCCCGG
AGGCTTCCCGGGGGCCAACAGCCCAGGACCTGTCGCCGATCTCTACGGCCCTGCCAGCC
AGGACTCCGGAGTGGGGAATTACATAAGTGCGGCCAGCCCACAGCCGGGCTCGGGCTT
CGGCCACGGCATAGCTGGACCTTTGATTGCAACGGCCTTTACAAATGGATACCATTGAG
CAGGTGCTTTCGTTGCCATCTCACTCTGAGAGCATACCTGGATGTCCAGGCAAGACTGG
GCGAAGTTTCTGAGTGGCCCTTTGTTTAGGTGATGTCCTCAGACCTGGACCCCCACCAG
CCTCACTCCCCATCCCAACCAGAGATGGCTCACTTCGGATCGAGGGTTGACTACATCTC
ATCATCTCACGAATCTGCTGTAATATAAGACAACAGCTTTTAAATGTGTATATAACCCA
TGATTTCGGTTTTGTTTTGTTTTGTTTTTCTTGATGGTTTCCCTCTCCCTCCCTCTCTTCCC
ATTCTCCTTTTAAATCTCTTTGAATCACATTTGGTAGTGATTTTGACTTAGTCCAGTAGTC
ACATAGCTTTAATATCTAGTTCAAAGCTAACCATAGTATAATTGTTATATT
SEQ ID NO 3:
GCCTTAGAGACTATTTTAGCAAA
SEQ ID NO 4:
TAGAGACTATTTTAGCAAATTTG
SEQ ID NO 5:
GACTATTTTAGCAAATTTGGAGA
SEQ ID NO 6:
AGCAAATTTGGAGAAATTAGAGA
SEQ ID NO 7:
AGCAAGTGTAGATAAAGTATTAG
SEQ ID NO 8:
CACAAGAACAAAGAAAATATTTG
SEQ ID NO 9:
AAGAACAAAGAAAATATTTGTAG
SEQ ID NO 10:
GTGGAAGATGTAAAGCAATATTT
SEQ ID NO 11:
TGGAAGATGTAAAGCAATATTTC
SEQ ID NO 12:
TGCTGATGTTTGATAAAACTACC
SEQ ID NO 13:
TGGCTTTGTCACTTTTGAGAATG
SEQ ID NO 14:
GGCTTTGTCACTTTTGAGAATGA
SEQ ID NO 15:
CACTTTTGAGAATGAAGATGTTG
SEQ ID NO 16:
AAGATGTTGTGGAGAAAGTCTGT
SEQ ID NO 17:
GAGAAAGTCTGTGAGATTCATTT
SEQ ID NO 18:
TTCATTTCCATGAAATCAATAAT
SEQ ID NO 19:
TTCCATGAAATCAATAATAAAAT
SEQ ID NO 20:
TCCAAGCTATGGCTATCAGTTCC
SEQ ID NO 21:
TTGATTGCAACGGCCTTTACAAA
SEQ ID NO 22:
CACGAATCTGCTGTAATATAAGA
SEQ ID NO 23:
AACAGCTTTTAAATGTGTATATA
SEQ ID NO 24:
CAGCTTTTAAATGTGTATATAAC
SEQ ID NO 25
AGCTTTTAAATGTGTATATAACC
SEQ ID NO 26:
TCCCATTCTCCTTTTAAATCTCT
SEQ ID NO 27:
CTCCTTTTAAATCTCTTTGAATC
SEQ ID NO 28:
TCCTTTTAAATCTCTTTGAATCA
SEQ ID NO 29:
CTCTTTGAATCACATTTGGTAGT
SEQ ID NO 30:
ATCACATTTGGTAGTGATTTTGA
SEQ ID NO 31:
TGGTAGTGATTTTGACTTAGTCC
SEQ ID NO 32:
CACATAGCTTTAATATCTAGTTC
SEQ ID NO 33:
TAGCTTTAATATCTAGTTCAAAG
SEQ ID NO: 34:
AGCTTTAATATCTAGTTCAAAGC
SEQ ID NO: 35:
TTCAAAGCTAACCATAGTATAAT
SEQ ID NO: 36:
AGCTAACCATAGTATAATTGTTA
SEQ ID NO: 37:
AACCATAGTATAATTGTTATATT
SEQ ID NO 38: ASO 1
TGACTTCTTTCGGCTG
SEQ ID NO 39: ASO 3
TTGGATTAAGGTTGCC
SEQ ID NO 40: siRNA 1
TGACTTCTTTCGGCTGAGCTT
SEQ ID NO 41: Sense AgomiR-7 (mimic of miR-107)
AGCAGCAUUGUACAGGGCUAUCA
SEQ ID NO 42: Antisense AgomiR-7 (mimic of miR-107)
UGAUAGCCCUGUACAAUGCUGCU
SEQ ID NO 43: siRNA 2
AGTTTTATCAAACATCAGCAT
SEQ ID NO 44: ASOS SCRAMBLE (control)
TTCCCTGAAGGTTCCT
SEQ ID NO 45-68: See Table 2.
SEQ ID NO 69: AAV9-hDES-mMSI2(V4)-V5-T2A-Nanoluc
GCTAGCGCGGCCGCACCCATGCCTCCTCAGGTACCCCCTGCCCCCCACAGCTCCTCTCCT
GTGCCTTGTTTCCCAGCCATGCGTTCTCCTCTATAAATACCCGCTCTGGTATTTGGGGTT
GGCAGCTGTTGCTGCCAGGGAGATGGTTGGGTTGACATGCGGCTCCTGACAAAACACA
AACCCCTGGTGTGTGTGGGCGTGGGTGGTGTGAGTAGGGGGATGAATCAGGGAGGGGG
CGGGGGACCCAGGGGGCAGGAGCCACACAAAGTCTGTGCGGGGGTGGGAGCGCACAT
AGCAATTGGAAACTGAAAGCTTATCAGACCCTTTCTGGAAATCAGCCCACTGTTTATAA
ACTTGAGGCCCCACCCTCGAGATAACCAGGGCTGAAAGAGGCCCGCCTGGGGGCTGCA
GACATGCTTGCTGCCTGCCCTGGCGAAGGATTGGCAGGCTTGCCCGTCACAGGACCCCC
GCTGGCTGACTCAGGGGCGCAGGCCTCTTGCGGGGGAGCTGGCCTCCCCGCCCCCACGG
CCACGGGCCGCCCTTTCCTGGCAGGACAGCGGGATCTTGCAGCTGTCAGGGGAGGGGA
GGCGGGGGCTGATGTCAGGAGGGATACAAATAGTGCCGACGGCTGGGGGCCCTGTCTC
CCCTCGCCGCATCCACTCTCCGGCCGGCCGCCTGCCCGCCGCCTCCTCCGTGCGCCCGCC
AGCCTCGCCCGCGCCGTCACCGGTTCGAACAGGTAAGCGCCCCTAAAATCCCTTTGGCA
CAATGTGTCCTGAGGGGAGAGGCAGCGACCTGTAGATGGGACGGGGGCACTAACCCTC
AGGGTTTGGGGTTCTGAATGTGAGTATCGCCATCTAAGCCCAGTATTTGGCCAATCTCA
GAAAGCTCCTGGCTCCCTGGAGGATGGAGAGAGAAAAACAAACAGCTCCTGGAGCAGG
GAGAGTGTTGGCCTCTTGCTCTCCGGCTCCCTCTGTTGCCCTCTGGTTTCTCCCCAGGTT
CGAAGGATCCATGGAGGCAAATGGGAGCCCAGGCACCTCGGGCAGCGCCAACGACTCC
CAGCACGACCCCGGTAAAATGTTTATCGGTGGACTGAGCTGGCAGACCTCACCAGATA
GCCTTAGAGACTATTTTAGCAAATTTGGAGAAATTAGAGAATGTATGGTCATGAGAGAT
CCCACAACGAAACGCTCCAGAGGCTTCGGTTTCGTCACCTTCGCAGACCCAGCAAGTGT
AGATAAAGTATTAGGTCAGCCCCACCATGAGTTAGATTCCAAGACGATTGACCCAAAA
GTTGCATTTCCTCGTCGAGCGCAACCTAAGATGGTCACAAGAACAAAGAAAATCTTCGT
AGGAGGATTGTCTGCAAACACAGTAGTGGAAGATGTAAAGCAGTATTTCGAGCAGTTT
GGCAAGGTAGAGGATGCGATGCTGATGTTCGACAAAACCACCAACAGGCACAGAGGGT
TTGGCTTTGTCACCTTTGAGAATGAAGACGTTGTGGAGAAAGTCTGTGAGATTCATTTCC
ATGAAATCAATAATAAAATGGTAGAATGTAAGAAAGCTCAGCCGAAAGAAGTCATGTT
CCCACCTGGGACAAGAGGCCGGGCCCGGGGGCTGCCATACACCATGGATGCGTTCATG
CTTGGCATGGGGATGCTGGGCTACCCCAACTTTGTGGCAACCTATGGCAGAGGCTACCC
CGGATTTGCTCCTAGCTATGGCTACCAGTTCCCAGGCTTCCCGGCGGCAGCTTATGGAC
CAGTGGCAGCGGCAGCTGTGGCAGCGGCTCGAGGATCAGGCTCCAACCCGGCGCGGCC
CGGAGGCTTCCCGGGGGCCAACAGCCCAGGACCTGTCGCCGATCTCTACGGCCCTGCCA
GCCAGGACTCCGGAGTGGGGAATTACATAAGCGCGGCCAGCCCACAGCCGGGCTCCGG
CTTCGGCCACGGCATAGCTGGACCTTTGATTGCAACGGCCTTTACAAATGGATACCACT
GAGGTAAGCCTATCCCTAACCCTCTCCTCGGTCTCGATTCTACGGAGGGCAGAGGAAGT
CTGCTAACATGCGGTGACGTCGAGGAGAATCCTGGCCCAGGATCCATGGTCTTCACACT
CGAAGATTTCGTTGGGGACTGGCGACAGACAGCCGGCTACAACCTGGACCAAGTCCTT
GAACAGGGAGGTGTGTCCAGTTTGTTTCAGAATCTCGGGGTGTCCGTAACTCCGATCCA
AAGGATTGTCCTGAGCGGTGAAAATGGGCTGAAGATCGACATCCATGTCATCATCCCGT
ATGAAGGTCTGAGCGGCGACCAAATGGGCCAGATCGAAAAAATTTTTAAGGTGGTGTA
CCCTGTGGATGATCATCACTTTAAGGTGATCCTGCACTATGGCACACTGGTAATCGACG
GGGTTACGCCGAACATGATCGACTATTTCGGACGGCCGTATGAAGGCATCGCCGTGTTC
GACGGCAAAAAGATCACTGTAACAGGGACCCTGTGGAACGGCAACAAAATTATCGACG
AGCGCCTGATCAACCCCGACGGCTCCCTGCTGTTCCGAGTAACCATCAACGGAGTGACC
GGCTGGCGGCTGTGCGAACGCATTCTGGCGTAAGTATAC

### SEQ ID NO 70-75 and 79-108: See Table 2.

ASO1 (SEQ ID NO: 76, comprised in SEQ ID: 38 but specifying the chemical modifications):

5′ +T+G+A*C*T*T*C*T*T*T*C*G*G+C+T+G 3′

Wherein + denotes locked nucleic acid and * denotes phosphorothioate linkages.

ASO3 (SEQ ID NO: 77, comprised in SEQ ID: 39 but specifying the chemical modifications):

5′ +T+T+G*G*A*T*T*A*A*G*G*T*T+G+C+C 3′

Wherein + denotes locked nucleic acid and * denotes phosphorothioate linkages.

AgomiR-107 antisense strand (SEQ ID NO: 78, comprised in SEQ ID: 42 but specifying the chemical modifications and the conjugation to cholesterol):

5′mU*mG*mAmUmAmGmCmCmCmUmGmUAmCmAmAmUmGmCmU*mG*mC*mU*3′chol where m denotes 2′-O-methyl-modified phosphoramidites, * denotes phosphorothioate linkages, and “chol” denotes cholesterol groups conjugated to the 3′ end of the molecule.

SEQ ID NO: 109
>ENST00000579180|ENSE00002692932; ENSE00003688116; ENSE00002719516;
ENSE00003508406; ENSE00003652249; ENSE00003627192; ENSE00003758332
CAAGTCAGTCGATCCTGTGTTTGTGCTCCCGAAGGACTTGGCGACTTGGCTAACAAGGAG
ACATTTGTTATTTCATTTCCTCATCCATTATTCATGCAGTGAGAGATGGTCACAAGAACA
AAGAAAATATTTGTAGGCGGGTTATCTGCGAACACAGTAGTGGAAGATGTAAAGCAATAT
TTCGAGCAGTTTGGCAAGGTGGAAGATGCAATGCTGATGTTTGATAAAACTACCAACAGG
CACAGAGGGTTTGGCTTTGTCACTTTTGAGAATGAAGATGTTGTGGAGAAAGTCTGTGAG
ATTCATTTCCATGAAATCAATAATAAAATGGTAGAATGTAAGAAAGCTCAGCCGAAAGAA
GTCATGTTCCCACCTGGGACAAGAGGCCGGGCCCGGGGACTGCCTTACACCATGGACGCG
TTCATGCTTGGCATGGGGATGCTGGGATATCCCAACTTCGTGGCGACCTATGGCCGTGGC
TACCCCGGATTTGCTCCAAGCTATGGCTATCAGTTCCCAGACTATTTGCCGGTTTCACAA
GACATAATTTTTATCAACTAGCTCTTAAGAGAGGCATAGCAAAGTGGGGGTTGCTACCAT
TTCTAGAGAGAGAGGACACAGTCCCGGCCTGGGCTGCCCCCGCTCCAGTCAATGCTCACT
GAAAGTCTGTCTTAGCTGCCTGTTTGAATGACTGTTCTTTTTCTCATTTTTAATTCTTGG
ACTCATGTCCTCATTGCTTCACTCAATTAAAAAAAAATTATTCTCCAGTCCCCTCCCACT
TTGCTTCTTGTATGCATTGTGACCGACCCCACTTCCTCAGAATGTAACGGGGCCAGAGGG
AAACTTCTCACAAACTTCGTAGAGCCTCCTCAGGGGAAGCTAGGAAGAAGACATCAAATG
TTTTTAAGTCATGACCAAACAGGCTTGTTGGGGACATATCATGGGGTGAGCTTTGAAGTG
CTGGTGGTCCAGAGGGGTTGCAGATACGTGACTTGGAGCACCCGTGTCTTACGATGGACA
GTGATAAAGGTGAACACACAGAGACAGACTATTCTCTAAGAATGTGAGAAACCTGATCTG
GAAGAGGAGCTATATAAACACTATCTGACTATCTTTGTCTTTTGGGGCCAGTGGCTGTTG
GCATAATCACAAGCCTGTCTGTCTTCGAGAAGGGACAGTGGAGTCATCCAGGTGCTGCCA
CATGACAGGCACGGTGGGCACCGATCCACAGTGGGCCCCGCCTTCCCCAGCTCGCCTCCC
TGCCTGTGCTGGCCTGGCCTTGCCTGCTGGCACCATTGGAGTAGGAGGGGGTGGAACACA
GGGGGCCCATCCTGATCAGGCCCCATCTCAAGGTTGGCACTCCTGCCCATCACCCTTAGA
AGGATCTTTTCCCATGGCTTGACTTCCTTCATTTCCCTAACTGAAATACACCCACTCTCT
TGGAATAATGACGTACCACTCAGTTGGACCCTCAAGAGTCACTGCTTTGTCTGTGCTGGT
AGTTTGTGAGAAGTGACCCGCACGCTTCCATTTGATGCATTTGATGTGAGTGAATCCATA
CATTTGAATGTCATTGTCCTTGAGACCCTACATGTGCAGTTTGGCTCATCTCATTAAAGA
TGCTTGATGTAATAA
SEQ ID NO: 110
>ENST00000674961|ENSE00003532300; ENSE00003615026; ENSE00003694227; ENSE00003661268;
ENSE00003602774; ENSE00003900362; ENSE00003903343; ENSE00003901826; ENSE00003649314;
ENSE00003525930; ENSE00003641220; ENSE00003606151
GCCAAGGCACCTCGGGCAGCGCCAACGACTCCCAGCACGACCCCGGTAAAATGTTTATCG
GTGGACTGAGCTGGCAGACCTCACCAGATAGCCTTAGAGACTATTTTAGCAAATTTGGAG
AAATTAGAGAATGTATGGTCATGAGAGATCCCACTACGAAACGCTCCAGAGGCTTCGGTT
TCGTCACGTTCGCAGACCCAGCAAGTGTAGATAAAGTATTAGGTCAGCCCCACCATGAGT
TAGATTCCAAGACGATTGACCCCAAAGTTGCATTTCCTCGTCGAGCGCAACCCAAGAGTT
CCTTACAAGACAATCTGATTTGATCCGCAGGAGAATCCTATGGTCACAAGAACAAAGAAA
ATATTTGTAGGCGGGTTATCTGCGAACACAGTAGTGGAAGATGTAAAGCAATATTTCGAG
CAGTTTGGCAAGGTGGAAGATGCAATGCTGATGTTTGATAAAACTACCAACAGGCACAGA
GGGTTTGGCTTTGTCACTTTTGAGAATGAAGATGTTGTGGAGAAAGTCTGTGAGATTCAT
TTCCATGAAATCAATAATAAAATGGTAGAATGTAAGAAAGCTCAGCCGAAAGAAGTCATG
TTCCCACCTGGGACAAGAGGCCGGGCCCGGGGACTGCCTTACACCATGGACGCGTTCATG
CTTGGCATGGGGATGCTGGGATATCCCAACTTCGTGGCGACCTATGGCCGTGGCTACCCC
GGATTTGCTCCAAGCTATGGCTATCAGTTCCCAGACTATTTGCCGGTTTCACAAGACATA
ATTTTTATCAACTAGCTCTTAAGAGAGGCATAGCAAAGTGGGGGTTGCTACCATTTCTAG
AGAGAGAGGACACAGTCCCGGCCTGGGCTGCCCCCGCTCCAGTCAATGCTCACTGAAAGT
CTGTCTTAGCTGCCTGTTTGAATGACTGTTCTTTTTCTCATTTTTAATTCTTGGACTCAT
GTCCTCATTGCTTCACTCAATTAAAAAAAAATTATTCTCCAGTCCCCTCCCACTTTGCTT
CTTGTATGCATTGTGACCGACCCCACTTCCTCAGAATGTAACGGGGCCAGAGGGAAACTT
CTCACAAACTTCGTAGAGCCTCCTCAGGGGAAGCTAGGAAGAAGACATCAAATGTTTTTA
AGTCATGACCAAACAGGCTTGTTGGGGACATATCATGGGGTGAGCTTTGAAGTGCTGGTG
GTCCAGAGGGGTTGCAGATACGTGACTTGGAGCACCCGTGTCTTACGATGGACAGTGATA
AAGGTGAACACACAGAGACAGACTATTCTCTAAGAATGTGAGAAACCTGATCTGGAAGAG
GAGCTATATAAACACTATCTGACTATCTTTGTCTTTTGGGGCCAGTGGCTGTTGGCATAA
TCACAAGCCTGTCTGTCTTCGAGAAGGGACAGTGGAGTCATCCAGGTGCTGCCACATGAC
AGGCACGGTGGGCACCGATCCACAGTGGGCCCCGCCTTCCCCAGCTCGCCTCCCTGCCTG
TGCTGGCCTGGCCTTGCCTGCTGGCACCATTGGAGTAGGAGGGGGTGGAACACAGGGGGC
CCATCCTGATCAGGCCCCATCTCAAGGTTGGCACTCCTGCCCATCACCCTTAGAAGGATC
TTTTCCCATGGCTTGACTTCCTTCATTTCCCTAACTGAAATACACCCACTCTCTTGGAAT
AATGACGTACCACTCAGTTGGACCCTCAAGAGTCACTGCTTTGTCTGTGCTGGTAGTTTG
TGAGAAGTGACCCGCACGCTTCCATTTGATGCATTTGATGTGAGTGAATCCATACATTTG
AATGTCATTGTCCTTGAGACCCTACATGTGCAGTTTGGCTCATCTCATTAAAGATGCTTG
ATGTAATAATTGGTTAGTTTCCTTTTATTTTCCTGCAGGCTTTTCCATGAGTATTATTTT
TTTCAAAGAACAAATCTGTATGGCTTTTCCCCATCTCCATATTTTGTTTTGCTATGAATT
GCTTTGCTTTGGTGAACTTGTCCTAGTATGCTTGCCTCACAAACGTTTTAGCCATTGTGA
ATTTTCTTCATCTCTGTAAATAGTTCATCTGTGCTTCTCCCTGATGACGTTTTATTTTTT
TTCCCCTGTAAGCAACCGAGGTAG
SEQ ID NO: 111
>ENST00000579205|ENSE00003649314; ENSE00003615026; ENSE00002697052; ENSE00002692484
TTTCCAATTCAGTGGGCTGCTACAATCCGGCCTTTATCACGGGCTGCTCTGGTTCCCATG
TCCCTTCTGGGCCAGTGCCTGAGTTTGTGGTTTGACATGAGGGCTTTAACCCCTTCCTGT
GCCAGCCCCTCACTGTGGTGGCCGGGAAGAAAAGGCCTTCCAGGAACCGGCGTGGAAAGT
TACAGCTCTTGAGGTCTCGTCAAAGTGGGAGATCGCTGTGGAAACCAGGGTTTGGCTTTG
TCACTTTTGAGAATGAAGATGTTGTGGAGAAAGTCTGTGAGATTCATTTCCATGAAATCA
ATAATAAAATGGTAGAATGTAAGAAAGCTCAGCCGAAAGAAGTCATGTTCCCACCTGGGA
CAAGAGGCCGGGCCCGGGGACTGCCTTACACCATGGACGCGTTCATGCTTGGCATGGGGA
TGCTGGGATATCCCAACTTCGTGGCGACCTATGGCCGTGGCTACCCCGGATTTGCTCCAA
GCTATGGCTATCAGTTCCCA
SEQ ID NO: 112
>ENST00000674522|ENSE00001157143; ENSE00003903810
GCTCCAACCCGGCGCGGCCCGGAGGCTTCCCGGGGGCCAACAGCCCAGGACCTGTCGCCG
ATCTCTACGGCCCTGCCAGCCAGGACTCCGGAGTGGGGAATTACATAAGTGCGGCCAGCC
CACAGCCGGGCTCGGGCTTCGGCCACGGCATAGCTAGCATACCTGGATGTCCAGGCAAGA
CTGGGCGAAGTTTCTGAGTGGCCCTTTGTTTAGGTGATGTCCTCAGACCTGGACCCCCAC
CAGCCTCACTCCCCATCCCAACCAGAGATGGCTCACTTCGGATCGAGGGTTGACTACATC
TCATCATCTCACGAATCTGCTGTAATATAAGACAACAGCTTTTAAATGTGTATATAACCC
ATGATTTCGGTTTTGTTTTGTTTTGTTTTTCTTGATGGTTTCCCTCTCCCTCCCTCTCTT
CCCATTCTCCTTTTAAATCTCTTTGAATCACATTTGGTAGTGATTTTGACTTAGTCCAGT
AGTCACATAGCTTTAATATCTAGTTCAAAGCTAACCATAGTATAATTGTTATATTAAGGA
GTTATTTTTTCTTAAAACATTTTTTTTTGCTTGTTTTGGTTCTGTTCTCACTTTTAAAGG
ATGCTGAGATGGTAATATGACTCTCCATATTTTGGTACCAATTCTGAGACTGTATGAATT
TTCAGGTGGAACTTTAGCACACACTGAAGCAAAGTTGTGAAGTGCAGGGCGGGAGGTGGG
CGTGAGCTTTCTATTTTGCGTTGTAGAAGAAGTGAGATGTAGTAAGCTAATTAACAGACT
TTCTAGCAGTTCTTTTTGTGATGTCTCTTTGTTAATCTGAGTCTATCTATTTTCGGCAAT
AAGGTAAGGACGACAGTGTTTTGAGTGTCCTCCTTTTCTATAAGTGCTTTTTTTCTGTTG
AAAGAGGTGATATTATAAGGTTTTTTGAAATTGTGAATTCTAAAAAAGAAATGTTGTAAA
TACAATTCCATTAACTACATAGAAACTATTAAGAAAGAGAGAATCAAAAATATTTTTGTG
AGGGAGTCGGTCCCAGGCAGTTTGATGCTCTGTGGAAGGAGGCGGGAAGGGAACGTTGGC
CAAGTCAGTTACTGAGATGAAGATCGCCCAGCTGCCAGGACCACCCCAGGACAAGTTAGA
GCACTGTTTAGCTCCTTTGTCTGTGTGATAGACCTAAGAACTGTATTAGTGTTGTACCAG
CCTATTAACCTCTTGTCTGTGCACAGCTTCAAATGTTACCGTCTAGTTAGATTTTTATTT
AAAATATGAAAAACTGCTTTTCCCAAGATGTTTTTTAAAAACAAAGCTACAATTTTAATA
TTTAACATATTTAAAGTTTCAAAGCACACCTGTTTGGCTTGGGTGGGGGTGGGGTGGGGG
GGACATTCTTTTTCAGTCTTAATTTTTAAATATTTGATCATTTTCTATTGTCCAATCATT
TCAGCACCTCCAAAGGTCCCTAGGACACTTTGCCTCTCTTCTCCCCCTGCCCCCCACCCT
GCTCCCACATCTGGGGGCCCATGGGCCAGGAGTGGATAAGCCTGCATTAATACAACCTTT
CTCCATTCACTTTCTATTTACAAATTAGGAAAGCAACCTTTTGGTTTATATATATTTTTT
TTAATACCTCAGTGCTGCAAGTATCACCAGAGAGGCTATGGAAGAATTTTTTTTTAATTT
ATTGTAGATGTAAACAGAATTTTAAAAATAAAAAGTATAAACATCACTGCACTGTGACTG
GTGGGAAAAACTGACAGTTTCCTCTTTGCACATGTTTAACATTTGGCTGTTATAATATAT
GGTCCTCGGTTGGGGAAAGATACTTATGATGAAGGATATTTTTTAATTTAACTTTTTTTT
AAATATTGGTAATAGGTCGGCAACAGCAACTATAGAAGTACAACTCAATAGATGGCATTA
AAACATATTGTAGTGTGGATATATATTTTTTCTTTTTTAAAATGTGATATTGACGTTTTA
TTAATATTTTTTAAATTGTTACGTTTATAAATTTGGTACTTAAGGCACAGCCAGTATGAG
ACACTGAATGCGACATTTATTATAAAGAGCTGCTGCACTCCTATTTTTATAAATTTTACT
AACAAAGTAGACTAATGTAGACATTCACAGACATGGTAGGGCAAAAGCATCTTCAAACTA
AAGACTCCAAAATGCTAACTCAGAAAGAAAGAAAAAACCCGTTTTCAATTCTAATGAAAC
AGCAACAACATTTTTTTTAATTAAAAAAAAAATCATGTTCTTTGTTTTTCTAATAAAATG
TTAGGTTGTTTGGTGAGGTTTTTTTGTTGTTTTTTTCCTTTCTTTGTTTCTTTTCTTTTC
CCCCAAACAACCAGATTAAATGCTGAGCGCTTTTAAAATATCAAAACTTGTTCAAGTCAT
AAAACAACAAAACATAAGTTTTATTTAAAAAAAAAAAAAGAAAGAAAAAATAGTAAATTC
CCAGAAATTCAGTGTTTAGACGAGGGAATTTAATTCCTATTTTGTCCATGTTGGTGATGT
ACTGTACTTCCCTTCCTTTTCTCTGCATCCCCCATCACCTCATAGAAGACTCTTTGTTGA
TCATTGTATGTTAATAATGTATAAAATGGCTATCTTGTAAGCGTGCTGTCCTGGTACTAG
TGTAGCGACTTTTTTTCTCCTCTTTCTTCTAGTACATATTGATAGGTATAACATAATTAA
GGTTTAAAAAAAATTAGACATAGTTATTCAGATTTAGGACCAGTAAGGATAGAACTTTCT
CTTATTTATGAAAAAAAATGCTAATAATTTTGGGGCAGTTTTTTCCTTTAATTATTTTTT
TCAATTTCAAGTTTAATTTTATTTTAGCTGATCTGATGTGGTTTCAACTAACCCAAGGTC
TCACCATGTTAAAATGCCGGCGGACTCTACGGCGTTTTGTAGATCCCCCCCCCCCCACCC
ACTGTGAAGGGGTGCCATACTACCTTAAATGCTAATGCTAGATATGCAAAACTGGATTTT
TTTAATTTATTTTTTAAAAGAGGGAGGCATGGTATATTAAAATGATTTTACTAAGAGAAA
AAATATTTTTTTAAGAATGCTCAGAAGAAATTGATAATCTGTGTGAATATGTTTTAGATG
TTTATATACCTTTTGAAGAGACCCAGTAGCCCATAGCACAAATCTTGTGGAAATCCGATA
TGTTTTAATGTGGCTACCTAGGTTTAAGGTTCACGTTAGTCCCCCCATTCCATCTAGAAG
TCCATTTTGAAAGATTTTTGTAAATTCTTTTAACACTGATGTTTCAGCCTCGTCTTTGTT
TCAGTTAAGCTCAATGGCGAACATGGGAACCACCTTTCGCCTTCCCTGGGGGAGAAACCC
TCTTGGCTGATGGCTTTTCCCCGGAATTATCAAACAGCCACCGGGTGACTTTCTGGCTTC
CAGATCCATCTGCCTGAGACCCCCGAACTCCTCTCCTCCCAAGCAGAGGCGAGTGAGTGG
CATTAGCTCCCGGACCCATTCCCGGTCCTAGCTGGGCATGGGGCTGACGGAGATGACCAA
GCCTTGGTCTGCTCTCTAGCAGCTTCCACAGACTTGGCTCGTGGCCTTCCTTATATCCAC
TGGGAACAAACAGCCTCGCGCTCTATACCAAACAGCCTCGCGCTCTATCCCAGTCGCCCA
TTAGCTTGATTCAAACAAAGCCCCAGCAGGCCTTTGCGTTTTTATCCTTCATAACCTTCA
TCTTAATTTGAACTTGTAGCTTGGACTTTAAGGTAGCATGGCTCTATTGCTGTCAATTTA
CTGTTTCACTGCACAGCAATCACAGCCAGTGAATGTTACACACATCTTGCTAGACTAGTA
TAAAAATCATTGGGTAATTGTTGGTTCTAATGACCTGAAAGGTGTTCAGTTTTTGTTCTT
GGTTTTGTTTTGTTTTTGATTTCTTGGGGGTGGGTTTTGCTTGTTGTTCCTTTTCATTTG
GGGGTTTTTGGGGAAAAAATTTATTTTTGGTTCCAAATAGAAAAACAAAACCTATTTTGA
TCTTTAGTGCAAACGAGGGCTAGGGACTTAGCCTCCTCCACCACCTCCACACTGCTTCAT
TCTGCCATTCACTCACTGCAGCATATTCAAGAATAAAGCAATATCGTTTACTACATTTTT
TATTGAAGGTCAGCCATGCTTTCTGTATTATATTGCATATGAAATTGTTTACAAAAGAAA
CACTAACTCATACTTCTCTTTATTGGTTGCAAGTGGCACGCAGGAACAGAGGGAGAGTGG
GGGGCTGGTGGGGGAGGGGAGATTTTTTTTTTTTTTTCTTGAATGTGCTTCGCAAGCCAG
GCTATCTTCCAAGGAAGGCAGACAGTGGGAGAGCAGAGGGACTGACTGCAGGCAAGCACA
TTGAAGAAAGACACTGGCGGGTTTCCCCACCCTCACCCCAAAGCAGAAAACTAGCAGACG
TCAGCTCAGCCCCGTCCTGGGCACAGACACTACACAAGGAGACGCTGGAAGTTAAGCAAT
ACTTTAATACTGTAATATGTTTGTTTTCTTTTTCTTTCTTTTTTTTCTACCAAAAAAAAG
TAAGTAAACTAAAACACAAAAACATATAAATAAAATCCATCCCTCTTGTCGGGGACCTGC
AGGGGGGCAACCTTAATCCAAACACCTGGCTATCAAATAATCAGAATGTATTGTCTCAGA
CAGGATTTCAGTTCCGGGAGGCAGGGGCATGATGGGGGAGGGGGCCGGAGGCTGAGAGAC
AAAAGTTCCAGAGCCTCCCTCGAAGGTTCTCTACTACTGTATTCTGTACATAATGTACCA
TCCCATGTGGAATCTGTGAGTGTCCTCTTAAGTAGCGTGGGCTAGCCAATCTGCCGTTCA
TGGTGTATTGTAAACTCCGAATTCCATATGTAATAGGATGCAAGTCTAAGCGTTTCATGT
GGACATAAATGTATCTAAATAAAACTTTCCCTAGCACTGTGGCTGACCTCACCCTTACTT
TTATACTTTAGTATGAAACTGATGAGAACTTTGGTAGTGAGTATTTTTTTTATATATATA
CATATATATGTACTATCTATATATATATCTCAAGCATCTTTCAGGTCTTTGTGTGTGGCT
TTCTTAAAGCCCTGTTGTAAAAAATTACTATGTGGATGGCAGTCTCTCACATCACAGATG
TGGAAAGTATAATTTTATATTTGTATTTTCAAATAAATAAGTTTGTGAAAGGTTTCCATC
CTCTACTGTGGTCCAGAAATCAATGTGTTTGTCTGACAAAAAAAAAAATAAAATAAAATA
AACTGTTTTGAACAGA
SEQ ID NO: 113
>ENST00000582453|ENSE00003654833; ENSE00003618729; ENSE00003575978; ENSE00003661268;
ENSE00003641220; ENSE00003543198; ENSE00002705951; ENSE00002730750
GCAGCGCCAACGACTCCCAGCACGACCCCGGTAAAATGTTTATCGGTGGACTGAGCTGGC
AGACCTCACCAGATAGCCTTAGAGACTATTTTAGCAAATTTGGAGAAATTAGAGAATGTA
TGGTCATGAGAGATCCCACTACGAAACGCTCCAGAGGCTTCGGTTTCGTCACGTTCGCAG
ACCCAGCAAGTGTAGATAAAGTATTAGGTCAGCCCCACCATGAGTTAGATTCCAAGACGA
TTGACCCCAAAGTTGCATTTCCTCGTCGAGCGCAACCCAAGATGGTCACAAGAACAAAGA
AAATATTTGTAGGCGGGTTATCTGCGAACACAGTAGTGGAAGATGTAAAGCAATATTTCG
AGCAGTTTGGCAAGGTGGAAGATGCAATGCTGATGTTTGATAAAACTACCAACAGGCACA
GAGGGAGAGTAGCGATTTACGAAGAGCAAATGGAAGCGAAAACCCCTTTTCTTCTTTGGG
CCGGCTGTGTATTGCT
SEQ ID NO: 114
>ENST00000675656|ENSE00003508406; ENSE00003902086; ENSE00003902030; ENSE00003652249;
ENSE00003627192; ENSE00003606151; ENSE00003899600; ENSE00003602774; ENSE00003758332;
ENSE00001102120; ENSE00003580062; ENSE00003694227; ENSE00003532300; ENSE00001662724;
ENSE00003688116
AACGACTCCCAGCACGACCCCGGTAAAATGTTTATCGGTGGACTGAGCTGGCAGACCTCA
CCAGATAGCCTTAGAGACTATTTTAGCAAATTTGGAGAAATTAGAGAATGTATGGTCATG
AGAGATCCCACTACGAAACGCTCCAGAGGCTTCGGTTTCGTCACGTTCGCAGACCCAGCA
AGTGTAGATAAAGTATTAGGTCAGCCCCACCATGAGTTAGATTCCAAGACGATTGACCCC
AAAGTTGCATTTCCTCGTCGAGCGCAACCCAAGATGGTCACAAGAACAAAGAAAATATTT
GTAGGCGGGTTATCTGCGAACACAGTAGTGGAAGATGTAAAGCAATATTTCGAGCAGTTT
GGCAAGGTGGAAGATGCAATGCTGATGTTTGATAAAACTACCAACAGGCACAGAGGGTTT
GGCTTTGTCACTTTTGAGAATGAAGATGTTGTGGAGAAAGTCTGTGAGATTCATTTCCAT
GAAATCAATAATAAAATGGTAGAATGTAAGAAAGCTCAGCCGAAAGAAGTCATGTTCCCA
CCTGGGACAAGAGGCCGGGCCCGGGGACTGCCTTACACCATGGACGCGTTCATGCTTGGC
ATGGGGATGCTGGGATATCCCAACTTCGTGGCGACCTATGGCCGTGGCTACCCCGGATTT
GCTCCAAGCTATGGCTATCAGTTCCCAGCCCTATTACCATATTTAAATGCAAGCTTCCCA
GCAGCGGCTTATGGACCAGTGGCAGCAGCGGCGGTGGCGGCAGCAAGAGGATCAGTCCTG
AATAGCTACAGTGCTCAACCGAATTTTGGCGCGCCCGCTTCCCCGGCAGGCTCCAACCCG
GCGCGGCCCGGAGGCTTCCCGGGGGCCAACAGCCCAGGACCTGTCGCCGATCTCTACGGC
CCTGCCAGCCAGGACTCCGGAGTGGGGAATTACATAAGTGCGGCCAGCCCACAGCCGGGC
TCGGGCTTCGGCCACGGCATAGCTGGACCTTTGATTGCAACGGCCTTTACAAATGGATAC
CATTGAGCAGGTGCTTTCGTTGCCATCTCACTCTGAGAGCATACCTGGATGTCCAGGCAA
GACTGGGCGAAGTTTCTGAGTGGCCCTTTGTTTAGGTGATGTCCTCAGACCTGGACCCCC
ACCAGCCTCACTCCCCATCCCAACCAGAGATGGCTCACTTCGGATCGAGGGTTGACTACA
TCTCATCATCTCACGAATCTGCTGTAATATAAGACAACAGCTTTTAAATGTGTATATAAC
CCATGATTTCGGTTTTGTTTTGTTTTGTTTTTCTTGATGGTTTCCCTCTCCCTCCCTCTC
TTCCCATTCTCCTTTTAAATCTCTTTGAATCACATTTGGTAGTGATTTTGACTTAGTCCA
GTAGTCACATAGCTTTAATATCTAGTTCAAAGCTAACCATAGTATAATTGTTATATTAAG
GAGTTATTTTTTCTTAAAACATTTTTTTTTGCTTGTTTTGGTTCTGTTCTCACTTTTAAA
GGATGCTGAGATGGTAATATGACTCTCCATATTTTGGTACCAATTCTGAGACTGTATGAA
TTTTCAGGTGGAACTTTAGCACACACTGAAGCAAAGTTGTGAAGTGCAGGGCGGGAGGTG
GGCGTGAGCTTTCTATTTTGCGTTGTAGAAGAAGTGAGATGTAGTAAGCTAATTAACAGA
CTTTCTAGCAGTTCTTTTTGTGATGTCTCTTTGTTAATCTGAGTCTATCTATTTTCGGCA
ATAAGGTAAGGACGACAGTGTTTTGAGTGTCCTCCTTTTCTATAAGTGCTTTTTTTCTGT
TGAAAGAGGTGATATTATAAGGTTTTTTGAAATTGTGAATTCTAAAAAAGAAATGTTGTA
AATACAATTCCATTAACTACATAGAAACTATTAAGAAAGAGAGAATCAAAAATATTTTTG
TGAGGGAGTCGGTCCCAGGCAGTTTGATGCTCTGTGGAAGGAGGCGGGAAGGGAACGTTG
GCCAAGTCAGTTACTGAGATGAAGATCGCCCAGCTGCCAGGACCACCCCAGGACAAGTTA
GAGCACTGTTTAGCTCCTTTGTCTGTGTGATAGACCTAAGAACTGTATTAGTGTTGTACC
AGCCTATTAACCTCTTGTCTGTGCACAGCTTCAAATGTTACCGTCTAGTTAGATTTTTAT
TTAAAATATGAAAAACTGCTTTTCCCAAGATGTTTTTTAAAAACAAAGCTACAATTTTAA
TATTTAACATATTTAAAGTTTCAAAGC
SEQ ID NO: 115
>ENST00000675822|ENSE00003641220; ENSE00003602774; ENSE00003901904
ATTGACCCCAAAGTTGCATTTCCTCGTCGAGCGCAACCCAAGGCCTGCCTGGACCTGTGT
TCTGCTTCTGTATCCTGGACTGAGAATCACTGTGCTGCCTCCTGAGGAGCTGCTGCTGGA
GTGGACCTTGGGGCAGCTAGCACATTGCCTGTCTTCCTAAAGGCATCGGTGCACAGGGCA
CCTGGGAATGACAGCGGGCCTGAGAGCAGAGCTCAGTGGAGATGCTGGACCCCTGAAGAT
GGTCACAAGAACAAAGAAAATATTTGTAGGCGGGTTATCTGCGAACACAGTAGTGGAAGA
TGTAAAGCAATATTTCGAGCAGTTTGGCAAG
SEQ ID NO: 116
>ENST00000675379|ENSE00003580062; ENSE00003899915
GCTTCCCAGCAGCGGCTTATGGACCAGTGGCAGCAGCGGCGGTGGCGGCAGCAAGAGGAT
CAGAGCATACCTGGATGTCCAGGCAAGACTGGGCGAAGTTTCTGAGTGGCCCTTTGTTTA
GGTGATGTCCTCAGACCTGGACCCCCACCAGCCTCACTCCCCATCCCAACCAGAGATGGC
TCACTTCGGATCGAGGGTTGACTACATCTCATCATCTCACGAATCTGCTGTAATATAAGA
CAACAGCTTTTAAATGTGTATATAACCCATGATTTCGGTTTTGTTTTGTTTTGTTTTTCT
TGATGGTTTCCCTCTCCCTCCCTCTCTTCCCATTCTCCTTTTAAATCTCTTTGAATCACA
TTTGGTAGTGATTTTGACTTAGTCCAGTAGTCACATAGCTTTAATATCTAGTTCAAAGCT
AACCATAGTATAATTGTTATATTAAGGAGTTATTTTTTCTTAAAACATTTTTTTTTGCTT
GTTTTGGTTCTGTTCTCACTTTTAAAGGATGCTGAGATGGTAATATGACTCTCCATATTT
TGGTACCAATTCTGAGACTGTATGAATTTTCAGGTGGAACTTTAGCACACACTGAAGCAA
AGTTGTGAAGTGCAGGGCGGGAGGTGGGCGTGAGCTTTCTATTTTGCGTTGTAGAAGAAG
TGAGATGTAGTAAGCTAATTAACAGACTTTCTAGCAGTTCTTTTTGTGATGTCTCTTTGT
TAATCTGAGTCTATCTATTTTCGGCAATAAGGTAAGGACGACAGTGTTTTGAGTGTCCTC
CTTTTCTATAAGTGCTTTTTTTCTGTTGAAAGAGGTGATATTATAAGGTTTTTTGAAATT
GTGAATTCTAAAAAAGAAATGTTGTAAATACAATTCCATTAACTACATAGAAACTATTAA
GAAAGAGAGAATCAAAAATATTTTTGTGAGGGAGTCGGTCCCAGGCAGTTTGATGCTCTG
TGGAAGGAGGCGGGAAGGGAACGTTGGCCAAGTCAGTTACTGAGATGAAGATCGCCCAGC
TGCCAGGACCACCCCAGGACAAGTTAGAGCACTGTTTAGCTCCTTTGTCTGTGTGATAGA
CCTAAGAACTGTATTAGTGTTGTACCAGCCTATTAACCTCTTGTCTGTGCACAGCTTCAA
ATGTTACCGTCTAGTTAGATTTTTATTTAAAATATGAAAAACTGCTTTTCCCAAGATGTT
TTTTAAAAACAAAGCTACAATTTTAATATTTAACATATTTAAAGTTTCAAAGCACACCTG
TTTGGCTTGGGTGGGGGTGGGGTGGGGGGGACATTCTTTTTCAGTCTTAATTTTTAAATA
TTTGATCATTTTCTATTGTCCAATCATTTCAGCACCTCCAAAGGTCCCTAGGACACTTTG
CCTCTCTTCTCCCCCTGCCCCCCACCCTGCTCCCACATCTGGGGGCCCATGGGCCAGGAG
TGGATAAGCCTGCATTAATACAACCTTTCTCCATTCACTTTCTATTTACAAATTAGGAAA
GCAACCTTTTGGTTTATATATATTTTTTTTAATACCTCAGTGCTGCAAGTATCACCAGAG
AGGCTATGGAAGAATTTTTTTTTAATTTATTGTAGATGTAAACAGAATTTTAAAAATAAA
AAGTATAAACATCACTGCACTGTGACTGGTGGGAAAAACTGACAGTTTCCTCTTTGCACA
TGTTTAACATTTGGCTGTTATAATATATGGTCCTCGGTTGGGGAAAGATACTTATGATGA
AGGATATTTTTTAATTTAACTTTTTTTTAAATATTGGTAATAGGTCGGCAACAGCAACTA
TAGAAGTACAACTCAATAGATGGCATTAAAACATATTGTAGTGTGGATATATATTTTTTC
TTTTTTAAAATGTGATATTGACGTTTTATTAATATTTTTTAAATTGTTACGTTTATAAAT
TTGGTACTTAAGGCACAGCCAGTATGAGACACTGAATGCGACATTTATTATAAAGAGCTG
CTGCACTCCTATTTTTATAAATTTTACTAACAAAGTAGACTAATGTAGACATTCACAGAC
ATGGTAGGGCAAAAGCATCTTCAAACTAAAGACTCCAAAATGCTAACTCAGAAAGAAAGA
AAAAACCCGTTTTCAATTCTAATGAAACAGCAACAACATTTTTTTTAATTAAAAAAAAAA
TCATGTTCTTTGTTTTTCTAATAAAATGTTAGGTTGTTTGGTGAGGTTTTTTTGTTGTTT
TTTTCCTTTCTTTGTTTCTTTTCTTTTCCCCCAAACAACCAGATTAAATGCTGAGCGCTT
TTAAAATATCAAAACTTGTTCAAGTCATAAAACAACAAAACATAAGTTTTATTTAAAAAA
AAAAAAAGAAAGAAAAAATAGTAAATTCCCAGAAATTCAGTGTTTAGACGAGGGAATTTA
ATTCCTATTTTGTCCATGTTGGTGATGTACTGTACTTCCCTTCCTTTTCTCTGCATCCCC
CATCACCTCATAGAAGACTCTTTGTTGATCATTGTATGTTAATAATGTATAAAATGGCTA
TCTTGTAAGCGTGCTGTCCTGGTACTAGTGTAGCGACTTTTTTTCTCCTCTTTCTTCTAG
TACATATTGATAGGTATAACATAATTAAGGTTTAAAAAAAATTAGACATAGTTATTCAGA
TTTAGGACCAGTAAGGATAGAACTTTCTCTTATTTATGAAAAAAAATGCTAATAATTTTG
GGGCAGTTTTTTCCTTTAATTATTTTTTTCAATTTCAAGTTTAATTTTATTTTAGCTGAT
CTGATGTGGTTTCAACTAACCCAAGGTCTCACCATGTTAAAATGCCGGCGGACTCTACGG
CGTTTTGTAGATCCCCCCCCCCCCACCCACTGTGAAGGGGTGCCATACTACCTTAAATGC
TAATGCTAGATATGCAAAACTGGATTTTTTTAATTTATTTTTTAAAAGAGGGAGGCATGG
TATATTAAAATGATTTTACTAAGAGAAAAAATATTTTTTTAAGAATGCTCAGAAGAAATT
GATAATCTGTGTGAATATGTTTTAGATGTTTATATACCTTTTGAAGAGACCCAGTAGCCC
ATAGCACAAATCTTGTGGAAATCCGATATGTTTTAATGTGGCTACCTAGGTTTAAGGTTC
ACGTTAGTCCCCCCATTCCATCTAGAAGTCCATTTTGAAAGATTTTTGTAAATTCTTTTA
ACACTGATGTTTCAGCCTCGTCTTTGTTTCAGTTAAGCTCAATGGCGAACATGGGAACCA
CCTTTCGCCTTCCCTGGGGGAGAAACCCTCTTGGCTGATGGCTTTTCCCCGGAATTATCA
AACAGCCACCGGGTGACTTTCTGGCTTCCAGATCCATCTGCCTGAGACCCCCGAACTCCT
CTCCTCCCAAGCAGAGGCGAGTGAGTGGCATTAGCTCCCGGACCCATTCCCGGTCCTAGC
TGGGCATGGGGCTGACGGAGATGACCAAGCCTTGGTCTGCTCTCTAGCAGCTTCCACAGA
CTTGGCTCGTGGCCTTCCTTATATCCACTGGGAACAAACAGCCTCGCGCTCTATACCAAA
CAGCCTCGCGCTCTATCCCAGTCGCCCATTAGCTTGATTCAAACAAAGCCCCAGCAGGCC
TTTGCGTTTTTATCCTTCATAACCTTCATCTTAATTTGAACTTGTAGCTTGGACTTTAAG
GTAGCATGGCTCTATTGCTGTCAATTTACTGTTTCACTGCACAGCAATCACAGCCAGTGA
ATGTTACACACATCTTGCTAGACTAGTATAAAAATCATTGGGTAATTGTTGGTTCTAATG
ACCTGAAAGGTGTTCAGTTTTTGTTCTTGGTTTTGTTTTGTTTTTGATTTCTTGGGGGTG
GGTTTTGCTTGTTGTTCCTTTTCATTTGGGGGTTTTTGGGGAAAAAATTTATTTTTGGTT
CCAAATAGAAAAACAAAACCTATTTTGATCTTTAGTGCAAACGAGGGCTAGGGACTTAGC
CTCCTCCACCACCTCCACACTGCTTCATTCTGCCATTCACTCACTGCAGCATATTCAAGA
ATAAAGCAATATCGTTTACTACATTTTTTATTGAAGGTCAGCCATGCTTTCTGTATTATA
TTGCATATGAAATTGTTTACAAAAGAAACACTAACTCATACTTCTCTTTATTGGTTGCAA
GTGGCACGCAGGAACAGAGGGAGAGTGGGGGGCTGGTGGGGGAGGGGAGATTTTTTTTTT
TTTTTCTTGAATGTGCTTCGCAAGCCAGGCTATCTTCCAAGGAAGGCAGACAGTGGGAGA
GCAGAGGGACTGACTGCAGGCAAGCACATTGAAGAAAGACACTGGCGGGTTTCCCCACCC
TCACCCCAAAGCAGAAAACTAGCAGACGTCAGCTCAGCCCCGTCCTGGGCACAGACACTA
CACAAGGAGACGCTGGAAGTTAAGCAATACTTTAATACTGTAATATGTTTGTTTTCTTTT
TCTTTCTTTTTTTTCTACCAAAAAAAAGTAAGTAAACTAAAACACAAAAACATATAAATA
AAATCCATCCCTCTTGTCGGGGACCTGCAGGGGGGCAACCTTAATCCAAACACCTGGCTA
TCAAATAATCAGAATGTATTGTCTCAGACAGGATTTCAGTTCCGGGAGGCAGGGGCATGA
TGGGGGAGGGGGCCGGAGGCTGAGAGACAAAAGTTCCAGAGCCTCCCTCGAAGGTTCTCT
ACTACTGTATTCTGTACATAATGTACCATCCCATGTGGAATCTGTGAGTGTCCTCTTAAG
TAGCGTGGGCTAGCCAATCTGCCGTTCATGGTGTATTGTAAACTCCGAATTCCATATGTA
ATAGGATGCAAGTCTAAGCGTTTCATGTGGACATAAATGTATCTAAATAAAACTTTCCCT
AGCACTGTGGCTGACCTCACCCTTACTTTTATACTTTAGTATGAAACTGATGAGAACTTT
GGTAGTGAGTATTTTTTTTATATATATACATATATATGTACTATCTATATATATATCTCA
AGCATCTTTCAGGTCTTTGTGTGTGGCTTTCTTAAAGCCCTGTTGTAAAAAATTACTATG
TGGATGGCAGTCTCTCACATCACAGATGTGGAAAGTATAATTTTATATTTGTATTTTCAA
ATAAATAAGTTTGTGAAAGGTTTCCATCCTCTACTGTGGTCCAGAAATCAATGTGTTTGT
CTGACAAAAAAAAAAATAAAATAAAATAAACTGTTTTGAACAGA
SEQ ID NO: 117
>ENST00000579483|ENSE00002718590; ENSE00002728538
CTTTATTGACTCCTGCTTTTCTATTTTTTTTCCCAGATTGACCCCAAAGTTGCATTTCCT
CGTCGAGCGCAACCCAAGAGGGCTTCCCAGAGTTAGCAGTATTTCCTGGGTTTGGAGGAA
TTGCTGTTTCTTCCATAAGTGTGAATCTTCAAATTTCCAGGTTAGTCATTTGATCACTCA
GAGCTTGGATCAGATTACCCAAGAGGGAAGCTGGAAAAATCCCTTACTTGGTAAAACCAT
GGGTTTGACCTGTTTTTCTCTCCCCTTTGTTGTCCCCCTTCCTTAACTTTGTTTTTAAAT
GAGAAAAGCAATATTTCACTTTAATACACTCTGCAGTGATGCATTCAACTTGAAAACGAG
ACAGAATGTTTCTCAAACTTGTCAAAGTGTGCATTCCTTTCCCAACCCTCAACTCCACAT
CTGGAATGTTTGTAGGTGAATTAACATGAAACCACTTGGGGGAGAACATCTTTCCATGAA
TTCTAGGGATTTAATAATAACACCTTACTGTGTATGACATACTTCACTTTACGGAGGACT
TGGCTGTTCATTATCTCCATCTGAATTCCATCTCCTGGGGCATTTGGCTGTTGTCCCTAA
CCTTCTCTCAGTCTCAGTGTGCTCATCTGTAAAGTAGGAGTGATGACATAGCTCTCCCAG
AAGGTTTTTGTGTAGAGTAAAAGAGCATGGTCTGCGACAAAGGTCTTCGTCATTGTCATT
ATTTTTCTTGTCGGTTTTCTCATCATTGTTCCGGTTGTTGTTTGCAAGTGGGACAGCCTG
GGTTCGGATGCTGGTGGTGCCACTCCTTAGCTGTGTGAATTTGAACAGGTCGTTTAACTT
CCTTGAACCTTCGTTTTTCCATCTCATAGAGTTTTTCTGCAGATTACGTAAATTAATAAA
GGTAAAGGGACTGAAAAAAAAAAGACGTTGCATGGTAATGCCATAGAAGCGTTAACCTGC
TATCTTATTTATTTATTTATTTATTTTTGAGATGGGATCTCACTGTGTTGCCCAGGCTGG
TCTTAAACTCCTGGGCCAAGCTATCCTCCCACCTCTGCCCCCCAGAGTGCTGGGATTATA
GGCGTGAGCCACTGTGCCTGGCCAACCTGCTATTTTTTTCTGCCATTTTTATTAAGTCCT
CACATCAATCCTGTGAGAGGTTGAGTAATAATAATGATACTAGTTGAATTTATATGCCAG
GCACTATTGCAGAGACTTTATTAATCCATTTAATTCTCATGCAGCCCTATGAGGAGAGTA
CTAGTATGAGCTCATTTTGTAAATGAGGAAACGGAGGCACAGATGGATGTCCAGGAGCCA
TACCCAGGCAACCTGGCTGTAGAACTGGGCCCTTCACCACTGTGCCATGCTGTGGGGGTA
CCTCAGGGGATGGCCCCTCTGCTATGCGACAGGAGATGGGCTCGGAGAGGTCAGCGGGCT
GTGCTGAGGGAACCATAGCCAGAGGCCCAGTACTTTGAAGTTCTGCGTTCCTGGGCCTAG
GTGATTTGCCTTGCCCCATCATGTTATACCAGTGTGTGACACTGTCTTGTATCTTTTGAT
CTGACCCCTCAGGCCATCTTTTCAGATGGTTCTGGAGTTACTCAACCCCAGTGAGGCAAC
AGAGTAAATGCCAAGGTTGTATGGTGGTTGGGAATTGGGACAGGGTGGGGAGAAAGTGTC
TTCCCTGTCTACGGTTATTTTCCAATAGAATTTGAGCCTGGCCACCGGGATTTCAGACTG
GAGTTAGCGCTGGGGGCCAAAACCCAGGAGCATAGAACTTTAAGTCGACCCTGATTTATT
TATCGATTATCTCCCTTCAATAAAACTTCTGTGTTTTTGTGTTCAGGTCACAGTGGCAGA
GGGAACCCTGCCCCTCCTCGTGGGATGGGCGAGGAGCTTGTAAATGATATGTCAGTGTGT
GTTTCAGTTAAAATGAGTTTCTCTGCTTTCTGGCTGTCCCTCCTTGCTAACGCTCGCCAG
AGAAATATTCCGTGGAGGTGTTAAAACAAGACTAGTGGAAAGAAATGGTAACTTAAAAAG
AAAGAAAATCCATGGCAGTGAAGAACAGATTTCATATTAGTACTGTCATAATATTCTCTA
ATGTATCATATAAATAAAAATCTGTTTAGACCAGAATTTTAATAAGTCACCAAAGGAAGG
TTTTGAATCCTACAGTTTATGTATCCCCCTTTGCGATGTAAATGTTCACATCTCAAGTGA
AGTAGAGTGGGGAGACGCTTCGGAGACTAGAGGCTGTTAATTAGTGAGGTATATTTTGAT
CAGGGCAAGAAGACTGGTTCATTCTATATCTTCGTATTTCTCTGGATGATTATTTCATTT
TGTAAAATATGTAAGAGTTCCCCCCCAGCCCCTTATCTTTTTAAAAATTAAAATGGTCAT
GCTCTTCTAAAAAGATGACTTAATTTGAAGGCTTTGGAGACATGAGGATGATGGGGATCC
ATCACTCCCTGTTCTTTGCTCCCTTTGGACTGGAGTGTTGGCTGTCTGGAAGAGTTTTGC
TTGAGCTCGTGGGTTATTTTCTTCTATGTGGAGGAACCAAGAGACTCTTTAGCATCTTTC
AGCAAGAGCGAGGTCTGGGTGTACTCACCATCTGTCTTAATTATCCTTGTGTATAAGAAA
ACATTCCCATCTTTTCCCAATTGCCATTTCCTTCCTTAACTTTTAGAGGCCAGAACCTCA
TCTGTTGATGCAGGGAGATCTCACTTCTTTAACCTATTTCTAGTTGCCTTCAGTCACAGC
GGGGTGGCCAGGGGCGAGTCACTTAAGCCTCCCCAAGGCTCAGTTTCCTCCTTTGTAAAA
GAGGGAGAATTATAGTACTCACTGCCTGCAGTTAGTTGTTTGGAGGATAAATGCAAAATT
CTTCACTGGGAGTCTGGCATAAAACAGCACTCCGCAAATGTCTGTTGATCTTATTATTAT
AGAATTATTATTTTTATTATTATTATTTTTAAGACGGAGCCTCACTCTGTCGCCCAGGCT
GGAGTGCAGTGGTGAGATCTTGGCTCACTGCAACCTCCGCCTCCCGGGTTCAAGCAATTC
TCCCGCCTCAGCCTCCCGACTAGCTGGGATTACAGGCACCTGCCATCATGCCTGGCTAAT
TTTTGTATTTTTGTAGAAATGGGGTTTCACCATGTTGGCCAGGCTGGTCTTGAACTCCTG
ACCTCAGGTGATCCACCTGCCTTGGCCTCCCAAAGTGCTGGGATTACAGGCATGAGCCAC
CGTGCCCGACCAGATAATTATTTTAATAGAATAAAAAAAAGTACAATGGTGAAATGACTG
AAGGAAAATCTTAATTGGAATGTAAGCTCCCTGAGGGCAGGGAACTGTTGGTGGCTGCTG
TTTGGTAACTAAATGAACGAACTGAGTCCAGCCTGCTTTTTTCACCGTGGAGGTAGCTGC
TACCCAGTAAAGTTACGTTGGTTGCTTCAGATCCACACTTAGGTTGTAGCAGGGCTGAGC
TAGCAGTCAGGTTTTCCTGTTCCTGTCTTGTCCTCTGACGTGGCTTCAGGGCCCTGGAAG
GTTCTCTGCTAAGCACGCATCCAGATGGGTGCAATGGCAGCCCGAGCGTGTACACGCACA
CCTCCTGTTCTGGGGGAGTGGTTTCTTGGCAGCTTCTCAAGGGCGAAGGGTGAGTTTTCG
GCATCTGGCCTTCCCTTGCTGCTGTGGGTCGGGTCATTCTAGCATCTTGCCATCTTGGAT
GATCTGCAGCTGTCATCTCGGCAGCCACCATGAACTGGCCTGCCAGTGGGTTTTCTCGTT
CCCAGCGAGGATGTGGTGGTGTGTCTGCAGCCCTTTTCCACAGCAGCGAGGACCTGGGAG
GATTAGTGGCTTAGCTTCTTTCTTGTCGGTGAGCACCGCTCCTTCCTATGTTCCAAGTCA
GTAGCAGGTGTCAGCTTAAGGAGGAGGGGCACCTTGTCCTACAAATGTCCTTTCCCTGGA
TTTTGCCGTTCCTCACATGCTTTCGTTTCATTGTTGAGGTCAGTCTCCTAGGACTATGAA
GCTCCTGTGTTCTTTGTCAGATGATTTTTGGCAGACAGGTGAGCCTCCTCTGCTGCCCGG
GAAGGGAGGAGAAGGCAGTCTCGGAGGAACCAGCTGACCATCTCAGGTGCCGCTTGGGGC
AGCGTCCATTCTTTGTGTCCTATTTTGTATTTTTAAACCCGGACAATCTTCGGCCGAAAA
CCCCGAGAACACACAGGGGAGATGTAGTTCATCATCTCTCTCTTCCCGTTTGATCTAGTG
TGTTCATTTAACAAGAGCTGGGTTAATCCTTGTTCATTGCCAACTGTATGCGTGCACCCA
CTTTTGGAGGGATTCAGGAAATACTCGGTGCTTCCTGATGTGCATGGAACACAGTCTCGA
CGTCTTAGCTTGGTGCTTAATTAAGGTCCTTCAAAATCTGTACCAGGGTCTTTTCCACCT
TATCTCTCAATTCTTCTAGTTTTTGCTGCCTTGAAACTGAATGCTCACCCTTCTTTGAGA
AACCTGTCCTTTTGCAGATTTCCCTCTCTGCACTGCCCTTCCCTTCTCTGTACACAACAG
ACCCTCTTTGAAAGCCCAGCTTCATCATTATCTTTTCCACGAAGCTGTCCCTGCCCCTTT
GTGGAAACAGTCATACTTCTTTGGATTTCCACAGCACTTACCTTTTCTTTGGTTAAGCCC
CTTCCAACAATAATAAATTAGCAGAAACTTGTGGAGCACATACATCTGTTGCATGTTGTG
CTAGACATTCACTCATTCATTTATTCACCAAACAAGCACCGACCGTTTGCCAGATACTGT
GCAAAATTCGGGGGATAGTGTGGAGAATATGACAAAGTTCCTGCTGTCACCAAACTTTTG
CTCTAGTGGGGCAAATAAGCAGTGAGCAAACAGATAAGTTTATGACATGCTCTCAGGTGT
GATCACCAAAGCCAGGGAGGAGCGATGGCGGGAGTGGGGTGGACCATATCTATGGTACAG
TCAGTTGCCACTTTCAGTAGTTCAGAAATTATTTACCAAGTTTCTGCTCAGCACCGGGGA
TTGCGTTAGGTACTTTGTTCTGAGTTTTATGGTATTTAGGTTCATGCCTTATCTTCTCCA
TTGGACACAAGCATATTTTACTAATAATTAAAAAAAAAAAA
SEQ ID NO: 118
>ENST00000674574|ENSE00003652249; ENSE00003902711; ENSE00003649314
GTGGAAGATGCAATGCTGATGTTTGATAAAACTACCAACAGGCACAGAGGTGGATTGGCT
GAGGCCTGAAATGGAGAAGGGACTTGTCCAAGATGGTTCAGGGCATGGAGTATGGAGCCC
ACTGTTGGGATCGCCAAGATGCCCGATAATAGCGTTCCTCATCCCTGCCTGAGGAGTGAA
CAGACTGAGGGGGTTTGGCTTTGTCACTTTTGAGAATGAAGATGTTGTGGAGAAAGTCTG
TGAGATTCATTTCCATGAAATCAATAATAAAATG
SEQ ID NO: 119
>ENST00000579531|ENSE00003575978; ENSE00002710534; ENSE00002704432; ENSE00003543198;
ENSE00003654833
GTCAAAATGGCCGATCTGACATCGGTGCTCACTTCTGTTATGTTTTCTCCCTCTAGTAAA
ATGTTTATCGGTGGACTGAGCTGGCAGACCTCACCAGATAGCCTTAGAGACTATTTTAGC
AAATTTGGAGAAATTAGAGAATGTATGGTCATGAGAGATCCCACTACGAAACGCTCCAGA
GGCTTCGGTTTCGTCACGTTCGCAGACCCAGCAAGTGTAGATAAAGTATTAGGTCAGCCC
CACCATGAGTTAGATTCCAAGACGATTGACCCCAAAGTTGCATTTCCTCGTCGAGCGCAA
CCCAAGGTGTTCTAGCTGAGCGTTTAAGAGCATGGACTCTGGAACCAGACTTTGAATCCT
TGCTCTGCCACTGCAGCTGTGTGACCTTGAGCAAGCTATCTAAATATTCTGTGCCTTTGT
TTTGTCATCTGTACAATGGAGATGATGATGGTATCACCTTCATGGGGTTGTC
SEQ ID NO: 120
>ENST00000442934|ENSE00003758332; ENSE00001157143; ENSE00003652249; ENSE00002285149;
ENSE00003627192; ENSE00003508406; ENSE00003688116; ENSE00001102120; ENSE00003580062;
ENSE00001718933
TTTCTGTTGTGTAGTTCTGATTTTCCGAACAGGTGTTTAGATGATGAGGGGTTATATTAA
GAAAGTACTAAAGAATACGTCTTATCACGTGAATCAGTATCTTTTTATTCAACATGGATG
GAGTGGGAGTGGAGACCAATCTGTTTTATATTCCTGCAATCTGATGAGGATGGTCACAAG
AACAAAGAAAATATTTGTAGGCGGGTTATCTGCGAACACAGTAGTGGAAGATGTAAAGCA
ATATTTCGAGCAGTTTGGCAAGGTGGAAGATGCAATGCTGATGTTTGATAAAACTACCAA
CAGGCACAGAGGGTTTGGCTTTGTCACTTTTGAGAATGAAGATGTTGTGGAGAAAGTCTG
TGAGATTCATTTCCATGAAATCAATAATAAAATGGTAGAATGTAAGAAAGCTCAGCCGAA
AGAAGTCATGTTCCCACCTGGGACAAGAGGCCGGGCCCGGGGACTGCCTTACACCATGGA
CGCGTTCATGCTTGGCATGGGGATGCTGGGATATCCCAACTTCGTGGCGACCTATGGCCG
TGGCTACCCCGGATTTGCTCCAAGCTATGGCTATCAGTTCCCAGGCTTCCCAGCAGCGGC
TTATGGACCAGTGGCAGCAGCGGCGGTGGCGGCAGCAAGAGGATCAGGCTCCAACCCGGC
GCGGCCCGGAGGCTTCCCGGGGGCCAACAGCCCAGGACCTGTCGCCGATCTCTACGGCCC
TGCCAGCCAGGACTCCGGAGTGGGGAATTACATAAGTGCGGCCAGCCCACAGCCGGGCTC
GGGCTTCGGCCACGGCATAGCTGGACCTTTGATTGCAACGGCCTTTACAAATGGATACCA
TTGAGCAGGTGCTTTCGTTGCCATCTCACTCTGAGAGCATACCTGGATGTCCAGGCAAGA
CTGGGCGAAGTTTCTGAGTGGCCCTTTGTTTAGGTGATGTCCTCAGACCTGGACCCCCAC
CAGCCTCACTCCCCATCCCAACCAGAGATGGCTCACTTCGGATCGAGGGTTGACTACATC
TCATCATCTCACGAATCTGCTGTAATATAAGACAACAGCTTTTAAATGTGTATATAACCC
ATGATTTCGGTTTTGTTTTGTTTTGTTTTTCTTGATGGTTTCCCTCTCCCTCCCTCTCTT
CCCATTCTCCTTTTAAATCTCTTTGAATCACATTTGGTAGTGATTTTGACTTAGTCCAGT
AGTCACATAGCTTTAATATCTAGTTCAAAGCTAACCATAGTATAATTGTTATATTAAGGA
GTTATTTTTTCTTAAAACATTTTTTTTTGCTTGTTTTGGTTCTGTTCTCACTTTTAAAGG
ATGCTGAGATGGTAATATGACTCTCCATATTTTGGTACCAATTCTGAGACTGTATGAATT
TTCAGGTGGAACTTTAGCACACACTGAAGCAAAGTTGTGAAGTGCAGGGCGGGAGGTGGG
CGTGAGCTTTCTATTTTGCGTTGTAGAAGAAGTGAGATGTAGTAAGCTAATTAACAGACT
TTCTAGCAGTTCTTTTTGTGATGTCTCTTTGTTAATCTGAGTCTATCTATTTTCGGCAAT
AAGGTAAGGACGACAGTGTTTTGAGTGTCCTCCTTTTCTATAAGTGCTTTTTTTCTGTTG
AAAG
SEQ ID NO: 121
>ENST00000583705|ENSE00002715708
GGGATGTTTTTAAAGGGTTTGTGTAGAAGGGCAGAGACACCCGTGGAAAGTGTCACTGTT
GCCCTCAATTGCTGGAGAGCCTGGAGGCTGTCATAAGTCCACTCATCAAAGTAATTGTTT
GCCAGTGTTTTGATAACAAGCAACAGAAACAACCCTGGCTGCTGTAAGCAGCAAAGGAGA
TTATTAGAAGGAGCTTGGGCAGTGCTGGAGAATTGACAGGATGGCTGGAGAACAGGACTC
AGAAAACAGGCAGGAACTGGGGGAGCTGGGTGGTCAGAGCCCCAGCCCTAGCCAAGGGGA
CAACTCAGGAATGACCTGGTCAGGGGGCCACTGTTGCCAACACTGGATGCCAGATTCTGC
TACTGACACCAGAATAAATTCGGAGCCAGTCCCTCCTCCTCTGCCCTACCAGCTCCAGAG
GCAGATCCTAAGGAGGGCTTCCGTCTGTCGAGCCCAGGTTCACAGGCCTGCCTCCCAGCC
TCCAGCCCCAGGAAGAGCCCTGAGGCCTTCTCAGCTTCTGTGATGGCCGGGGGGGTGGGC
CTCTCTCCAAGACTTACACACAGGGAGAGGCTCAGGGGCTGGGCAGCCAAAAAATGACCA
CAGACATTCACCTCCAAAGTCAAAGCGAAAGTCACGTTCTCGGTGGTTGGGTGGCCGTGG
TGGCATCTTAAGAGCAAGAATGTACTGAGGTTACATATACACTGAGTGGAGGGAGGCAGA
GTGAGCCTGACCCATACGGCCTTCATTTAGCCTGAATCAGGCAGGGACTTGAAGAACGAA
GTGGCCTGAGCAGCCACAATGACTCCCTGAGAGCTGAATGACACCTCACATCACTGGGGG
TTAGTGACAGATCTGAGATGGTCACACCAGCCCCTGGACTTGCCAGCTGAGTTTCTTGTG
TGCCCAGTGGCCAGCCCAGTGGCAGTTTCTACAAGTGTCTTTCAGTGAACCACTCACCCC
CTAGCTTGCCTGCTGAGACAGGGGTCTAGAAACAATACGCTTGTGGTGAAACGCCCATCC
AGTCACACGGCACGGGGCTCCTGAGTCGGAGCTACACTTTGTGGTATGCATCTTAACACA
TCCAGCCTCTCACCATTCTCCAAAGATCTTGGCACCTCCGTGGAGTGTCAAAACTGCAGT
GTGGGCCATGGCTCATCAGGCCCAGCGTTTTCCAGAGGCCCAGGGTGAGCCCTGAGTTTG
GCCGCTAAACTGTGACTCCTGAGTTCGCATGCTCTGTCTGGTGCTGGCTCTGTTGTCCAC
TGCCTTGGGAGGATGCATCACGAGGACTTTAATCTCCTACTGTCAATAGCTTTAAGGTAA
AGTACATGCAGGGTAAGCTCTCCAAATGTCAGAGCCAGGCGCAGGCTTTGTGTTTGGTGC
TGATGCATGGCATTTACTCTGAAGGTTCAGGAAAGTACATGGGCTTTGCGGGGTCTGGCT
GCCCAAATATGGGTTAATTAGCAACAGCTGACAGCAGCGCCCCCGGCCACAGGAGAGAGG
TGACCCAGACCCTTAATAAAAAAACCCTCATGAGAGACAAATTATTCTGTGAAGGAAAAT
AACTCAGGCTTTCCTCATTGCCACCCTCCGTGAGATTTTACCCCAGACCTGAGGCGGCTG
TACTAACAGGACTCTGATCTTTCTCTTTGTGTTCAAGGATATCCCAACTTCGTGGCGACC
TATGGCCGTGGCTACCCCGGATTTGCTCCAAGCTATGGCTATCAGTTCCCAGGTGAGTGG
CTTGGTCTCCCAGGGCTTTGGAAGCACAAGAGGTGGGCTGCATTTGCGGGGAGGTGAGAG
GACCCCTAAAGAGAATGCATTTCTTACATGCATCCACTTGAAAATGACCTATACATGATA
AATTTCAAATCCACTGAAGTTCAAGGCCAGGATGCAGCTCAGAGTTTTTGATTAACTCAG
GTATAACTCACTGGTGCCGGGTATTTGAGAACGGCAGCTTTTAAAGGGAAAGCAGAACGG
AGGCAGGAGGCCTCCCTGTGCTCACCTCCTTTTTCTGAAGCCTCTTCCGGGTTTTTTCTC
ACTGGGGACTGAACTCTAGGCCCAGGGCTTTCTTTCACCCTCTACCACCCCTTGCCCGCC
TCCCCGCTCCCTGTGTCCACCTGCCCAATGTTTGATGTCTCCCCGCCCTTGCTTCCTTTC
CTCCACCCCTCCTCCTCCTGCTCCGTCCTGACTGCTGTGGCTTCGGCGTCCTTCTGGGCT
GTGTTGATGTTGAGCTGCTAGGGGTTGGGACTGCTGTCCATCCTGGGTGAGCTGTTTAGA
TTATCCTTTCCCTCTGCGTGGGTATCAAGACTTGAGGCAGAGCCCATGGTGATGCCCGCC
ACATGCCAGGGCTGCTCCAGCTCCAACTCCAGGTAGCTGGCAGGCCTGGGACCTCACCTG
GGAGAAGCAGCGAGGGGAAACATGGTCTTGTAGGGATGGGGTAGGGACAATGAAGGAAGC
GATGTTTTACAGGGTATGCTTTTCCCCAGCCTTTCTCAAATCTGGAGTACAGATAAGAAT
ATATTGGGGTGTTTGGGGGTCTCCATGTGCAAAGGACAGCCGAGATTCCAGAGGAGGTGT
AGGGACAGAGTCTTGTCCCCTGTGGGTTTTCTGCTGAGGGAGGAGGCTCCCTGGGGAGCT
GGGCTGCAGGCGGAGTTTCACCCCTGCCTTGTCGTCATGCCTAGACCTGGAGGTCTGGGA
GTGTCAGGGTCTGAGATGAATTAGGAAGCAAAGGGAGTGAAGACGCGTCGCTGGAAGATG
GCGCCCTGACACTGATGACAGGAGGGCAGAGTGAGGGCTTGGCCTGGTCTTTGCCTCAGA
CCAAGCATTGGGGCGGGAAGGGTGTAGTTGGAGAGATGAGGAGGCATTTGGGGGCACCTA
GAGCCGTGCTCTATGGCAGACAGATCCTTCAACCAGCGGTGGAAGCCCCAAGCTCTTGTC
CTGTCTCCGATACCACCTGGCCAAGTGGCCATGGGCGGGTCACTCAGCTTCTAGGGCCTT
ACTTCTCTCTTTTGGAAAAGGGGGTCTACGAGGAATGGTCTTTGGGGTCTGTGCCCCTCT
CCGATTGTGTGGGTCAGAGGGTGCAGGGAAGGCAGGCACAGAAGCAGAAGTCCAAGATGG
AGGAGTTTGAGTGCAGGTGGCTCCCCCACCAGTGTCCCCTGGGGCTGGGATGGTTCCCCA
TCTGGGAGGGGCCTCAACAAGACTCCTCGGCCCGCACTAAGGAAGCCGTCCCCAAAGATG
CCCCCCAGCAGCAGGCTGCTTCAGGATGGCCACAGGCTCCCAGACCTGAAGAAAAGAAGG
GCACATGCAAGAAGGACCACCACAAGAGAGGCCAGCACTCAACCAGCCACTGTCATTAGA
CTTTCCCATTTCCAGGCAGAGACCCCCACCAGGCACAGGCACAGAGGTAGAAACTCCTTC
TGAAGGGGTCACAGTACGCAAGGGGTAGGACCTGCATTTGGACCAGGCGGTCTGGGCCTT
AACTGCCACCCTCCACCATCTCCCTAGTGTCCATTTTTCTGGTACAATAACACTAGCCAC
AAAGAGCTACTAAAACTTAGGTTTAAATTAAAGTTGGCTGGGTGCGGTGGCTCACGCCTG
TAATCCCAACACTTTGGGAGGCCGAGGTGGGAGGATCACTTGAGGTCAGGAGTTCGAGAC
CCGCCTGGCCAACATGGTGAAACCCCGTCTCTACTAAAAATATAAAAATGAGCCGGGCGT
GGTGCCCGGCATCTATAATCCCAGCTACTCGGGAGGCTGAGGCAAGAGAATCACTTGAAC
CCAGGAGGCAGAGGTTGCAGTGAGCCGAGATCACACCACTATACTCCAGCCTGGATAACA
GAGTGAGACTCTGTCTCAAAAATAAGAAGAAGAATAAGAAGAAGTAAAAAGAAAATTTCA
GTTCCTTTAGAAACACTGGCCACATTTCAGTGACTCAGTAACCATGTGTGGCAAGTGGCT
ATTGCCAGCACAGAATGTAGAACAGTCACCGTGGAAATTTCTTACGGGCTGGTGCTGTTC
CAGTGCCTAAAGGAAGAGGGCTGTTACTGGGGGGTGCACGGTCCACACACTCACACATGC
GCACCACTCCTTCCTGGAGACACCCATGGGGTCAGTGTCCCTGTGCTTCATTCTCTGCCA
GTCTTGCTCAGCCCAGAAGCTGGTGGCTTTTCCTCTCACTTCCTCCCCTCGCTTCCCCTC
CAGGGGATGACAGGTGCAAAGGCTCACGCATCTCGGCCCTCCAGGTGCAGCACTGATCTG
TGCACTCAGCCTGCACAACCTGCTCCCCTCCGTAATCAGGCTGCCATTGCCCCTGAGTGT
GATGCTAGTCCCAGGAGGACAGAGTTGAGGGATCTTAACATGGAATAATTGGGAGAGGAC
ATCAATGTGGACCCTATGCTAAGGCCCCTGTCTCATCAGAGGGCCTGCCCCGGGGCCCCC
ATGAGCTGGCTCAGGAGACATCTAGTAGAACTCTGCCTCCATGGCCTGCATCCCTGGGCC
TGCCCCAGGCAGACGGACATGCAGCAGTGACCAGATTGAGGCTGGGCAGAGTGAGATAGG
GTCCCCTGGGTGCCCAGCCCTTCACCAAAGCCAAGACTTTGGGAACTGGGGGGGCGGGGG
ATGGACTGGGCTGCAGGAAGGAGGATAGAGTGGGGTTGGAGTGTTTTTGCCCAGGGCCCA
TCCGTTTGTCATGGCCACAGTCCCTGCAGCTTAATACAATCAGGTGGGTTTGAATGGGAC
AGAAATCAGGCATTACAATGTACAGACAGCAGCTTCCTGACCTTTGGGAGTTAGCTGCCC
TGTTCTCAACCATCAAACTTTTATTTTTCCATCCTTCCCACCTTCCCACTCACTATGGAA
GCCTCTTGATTCTCTTAACTAATGGCCAGAAAGGGGCCACCAGTGGCCACAAGAGTAAGG
TTGCCTGCTCTCAGGGAGGGCAAGAGTGGAGTGGCCCTGAACTGGGAAGGGTGGCCCCAT
GGCTGGGGCCGCCCACCCCACTCATCTGAGAAGCCAGGCTAGGGTCAGAGTCTATTCCCC
TCCTGACCCCCTCCCAAAGGATATGCTCCTTGTGGAGTGTTGGGTCTGTGCTTATCCCAG
ATGCCTCCCCAAGCAGCCAAATGTTTCCAGACACAAAGCCTAGGTAGGTCCATGTGTCAT
TCCAGTCAAGAGCGCAGGGAGTAGCTGAGCCCCTCTAGAACCAAACTTGATGACTCCTGG
AGCCACAGCCTCAGGGCCAGGGCTACAGAGAAGCTTCTGGATGCCCTGACTTCCATCTGT
CACCTCATGCCAATAAGGAAAGTTTCGGGTGGATCTGGCCGAGGCGGCTGGGGTACAGAA
GGGGGACCCAGAGATGGGGCAGCCCAGCCCTCTGCCCTCTCCTCATCCCCATCAGGTAGA
TTCTGCGAACCCCTGCCATGCCCCGGGGATCCTGTGACTTCGTGACATTACACAGCAGTC
ATGTCACCTCCCAACTATCCTGATGTTTTCTTTGTGGTCTTTCTTTTCAGTGTCCTCACT
GTATGCCCACTTTACCCACTTCCCACCTCCGGTCCCCCTCATGCTGTGCATGGATCCCGA
ACCTCTCCCAGCCCCTTTCTGAGTCCCCCTAGCAGGGCCATGCAAGACAGCTAGCTGGGA
GAAATGAAAGCACGCCCAGGTCCTAGAGAGCTCCGAGACTCTTCATCCTGGCTGTTCTCA
CTCATAAACCAGGTAGCCTGGGTCCCCTTTCAGGAAGATGCAATCTGATCCCCTTTATGA
AGGGTCCTCCTTTCCACCTGGACTTACCCTTTCTATGAACATCCATCTCATCTGGTTGGG
GGTGCAGATATCACTCTTAGTAGAGCCTCATTCTCACCATGTGACATGGGGCTGCACGGA
CCTTCCTGGGCCACACAGCTGGTGGCAAGTGGCACCTGGCTGTGCCCATCTTCAGAATTC
TGTCAGAATAAATGTTCCTAGAGAAGGCTAGACACTGGATAGTTTTTAGAGTGGGGAGTA
GTTCTTTTCTAGAAAGGCTGGGTGGTGACCAACCACTCTGGGAACAGCTCCTTCTTCCCA
CATCCAGGTGTCCACCCCAGCCTGGAATTTGGATCAGGATCCCGTCTTGAATTATTATGG
GTTGTGATCAGGGATCTCCCCTGCTTCCTCTCTTTTCCCCACTCTGGACTTCTCATTTCA
GTTTAATCTGTTAATTTCTGTTCTTATTTCACTTTGCAGACTATTTGCCGGTTTCACAAG
ACATAATTTTTATCAACTAGCTCTTAAGAGAGGCATAGCAAAGTGGGGGTTGCTACCATT
TCTAGAGAGAGAGGACACAGTCCCGGCCTGGGCTGCCCCCGCTCCAGTCAATGCTCACTG
AAAGTCTGTCTTAGCTGCCTGTTTGAATGACTGTTCTTTTTCTCATTTTTAATTCTTGGA
CTCATGTCCTCATTGCTTCACTCAATTAAAAAAAAATTATTCTCCAGTCCCCTCCCACTT
TGCTTCTTGTATGCATTGTGACCGACCCCACTTCCTCAGAATGTAACGGGGCCAGAGGGA
AACTTCTCACAAACTTCGTAGAGCCTCCTCAGGGGAAGCTAGGAAGAAGACATCAAATGT
TTTTAAGTCATGACCAAACAGGCTTGTTGGGGACATATCATGGGGTGAGCTTTGAAGTGC
TGGTGGTCCAGAGGGGTTGCAGATACGTGACTTGGAGCACCCGTGTCTTACGATGGACAG
TGATAAAGGTGAACACACAGAGACAGACTATTCTCTAAGAATGTGAGAAACCTGATCTGG
AAGAGGAGCTATATAAACACTATCTGACTATCTTTGTCTTTTGGGGCCAGTGGCTGTTGG
CATAATCACAAGCCTGTCTGTCTTCGAGAAGGGACAGTGGAGTCATCCAGGTGCTGCCAC
ATGACAGGCACGGTGGGCACCGATCCACAGTGGGCCCCGCCTTCCCCAGCTCGCCTCCCT
GCCTGTGCTGGCCTGGCCTTGCCTGCTGGCACCATTGGAGTAGGAGGGGGTGGAACACAG
GGGGCCCATCCTGATCAGGCCCCATCTCAAGGTTGGCACTCCTGCCCATCACCCTTAGAA
GGATCTTTTCCCATGGCTTGACTTCCTTCATTTCCCTAACTGAAATACACCCACTCTCTT
GGAATAATGACGTACCACTCAGTTGGACCCTCAAGAGTCACTGCTTTGTCTGTGCTGGTA
GTTTGTGAGAAGTGACCCGCACGCTTCCATTTGATGCATTTGATGTGAGTGAATCCATAC
ATTTGAATGTCATTGTCCTTGAGACCCTACATGTGCAGTTTGGCTCATCTCATTAAAGAT
GCTTGATGTAATAATTGGTTAGTTTCCTTTTATTTTCCTGCAGGCTTTTCCATGAGTATT
ATTTTTTTCAAAGAACAAATCTGTATGGCTTTTCCCCATCTCCATATTTTGTTTTGCTAT
GAATTGCTTTGCTTTGGTGAACTTGTCCTAGTATGCTTGCCTCACAAACGTTTTAGCCAT
TGTGAATTTTCTTCATCTCTGTAAATAGTTCATCTGTGCTTCTCCCTGATGACGTTTTAT
TTTTTTTCCCCTGTAAGCAACCGAGGTAGAAAAATAAATTGTTTACCATGGA
SEQ ID NO: 122
>ENST00000582772|ENSE00003649314; ENSE00002731414; ENSE00003661268
GGCAGAATTGCTTGAACCCGGGAGACAGATGTTGCAGTGAGCTGAGATCGTGCCACTGCA
CTCCAGACTGGGTGACAGAGCAAGACTCCGTCTCAAAAGAAAAAAAAAAAAGAACACAGC
ACGGAGAAGATGCCCAGAGCCCTTCACTGTGGGCCCATGAGATGACACAGATCTTTCCAC
TAGGCCATCCTGTTTTGTTGCTGGAGAACTTTGTCCCTGGAAGGGCAGAAATGATTATTC
TGGTTGCCCAAGGCCTGGTGGCCCAGGTGGAAGATGCAATGCTGATGTTTGATAAAACTA
CCAACAGGCACAGAGGGTTTGGCTTTGTCACTTTTGAGAATGAAGATGTTGTGGAGAAAG
TCTGTGAGATTCATTTCCATGAAATCAATAATAAAATG
SEQ ID NO: 123
>ENST00000674964|ENSE00003694227; ENSE00003532300; ENSE00003508406; ENSE00003758332;
ENSE00003652249; ENSE00003903464; ENSE00003899808; ENSE00001157143; ENSE00003606151;
ENSE00001102120; ENSE00003580062; ENSE00003688116; ENSE00003627192; ENSE00003602774
GCTAAGCCGAGCCCACGTGTGACGGCTCTCGCCGCTGCCCCGGCTCCGCCGCTCGCAGAG
AGATTCGGAGGAGCCCGGGCGGGGGGGAGGAGGAGGGGGAGGAGGGAGCGGAGATCTCG
G
GGCTCGGAGCCGGCCGCCGCTCCGCTCCGATCGCTGTGGGGCTTGGTTTTTTGGGGGTGG
GGGGGCGGGGGGGCTCAGATATGGAGGCAAATGGGAGCCAAGGCACCTCGGGCAGCGCCA
ACGACTCCCAGCACGACCCCGGTAAAATGTTTATCGGTGGACTGAGCTGGCAGACCTCAC
CAGATAGCCTTAGAGACTATTTTAGCAAATTTGGAGAAATTAGAGAATGTATGGTCATGA
GAGATCCCACTACGAAACGCTCCAGAGGCTTCGGTTTCGTCACGTTCGCAGACCCAGCAA
GTGTAGATAAAGTATTAGGTCAGCCCCACCATGAGTTAGATTCCAAGACGATTGACCCCA
AAGTTGCATTTCCTCGTCGAGCGCAACCCAAGATGGTCACAAGAACAAAGAAAATATTTG
TAGGCGGGTTATCTGCGAACACAGTAGTGGAAGATGTAAAGCAATATTTCGAGCAGTTTG
GCAAGGTGGAAGATGCAATGCTGATGTTTGATAAAACTACCAACAGGCACAGAGGGTTTG
GCTTTGTCACTTTTGAGAATGAAGATGTTGTGGAGAAAGTCTGTGAGATTCATTTCCATG
AAATCAATAATAAAATGGTAGAATGTAAGAAAGCTCAGCCGAAAGAAGTCATGTTCCCAC
CTGGGACAAGAGGCCGGGCCCGGGGACTGCCTTACACCATGGACGCGTTCATGCTTGGCA
TGGGGATGCTGGGATATCCCAACTTCGTGGCGACCTATGGCCGTGGCTACCCCGGATTTG
CTCCAAGCTATGGCTATCAGTTCCCAGGCTTCCCAGCAGCGGCTTATGGACCAGTGGCAG
CAGCGGCGGTGGCGGCAGCAAGAGGATCAGGCTCCAACCCGGCGCGGCCCGGAGGCTTCC
CGGGGGCCAACAGCCCAGGACCTGTCGCCGATCTCTACGGCCCTGCCAGCCAGGACTCCG
GAGTGGGGAATTACATAAGTGCGGCCAGCCCACAGCCGGGCTCGGGCTTCGGCCACGGCA
TAGCTGGACCTTTGATTGCAACGGCCTTTACAAATGGATACCATTGAGCAGGTGCTTTCG
TTGCCATCTCACTCTGAGGTGATGTCCTCAGACCTGGACCCCCACCAGCCTCACTCCCCA
TCCCAACCAGAGATGGCTCACTTCGGATCGAGGGTTGACTACATCTCATCATCTCACGAA
TCTGCTGTAATATAAGACAACAGCTTTTAAATGTGTATATAACCCATGATTTCGGTTTTG
TTTTGTTTTGTTTTTCTTGATGGTTTCCCTCTCCCTCCCTCTCTTCCCATTCTCCTTTTA
AATCTCTTTGAATCACATTTGGTAGTGATTTTGACTTAGTCCAGTAGTCACATAGCTTTA
ATATCTAGTTCAAAGCTAACCATAGTATAATTGTTATATTAAGGAGTTATTTTTTCTTAA
AACATTTTTTTTTGCTTGTTTTGGTTCTGTTCTCACTTTTAAAGGATGCTGAGATGGTAA
TATGACTCTCCATATTTTGGTACCAATTCTGAGACTGTATGAATTTTCAGGTGGAACTTT
AGCACACACTGAAGCAAAGTTGTGAAGTGCAGGGCGGGAGGTGGGCGTGAGCTTTCTATT
TTGCGTTGTAGAAGAAGTGAGATGTAGTAAGCTAATTAACAGACTTTCTAGCAGTTCTTT
TTGTGATGTCTCTTTGTTAATCTGAGTCTATCTATTTTCGGCAATAAGGTAAGGACGACA
GTGTTTTGAGTGTCCTCCTTTTCTATAAGTGCTTTTTTTCTGTTGAAAGAGGTGATATTA
TAAGGTTTTTTGAAATTGTGAATTCTAAAAAAGAAATGTTGTAAATACAATTCCATTAAC
TACATAGAAACTATTAAGAAAGAGAGAATCAAAAATATTTTTGTGAGGGAGTCGGTCCCA
GGCAGTTTGATGCTCTGTGGAAGGAGGCGGGAAGGGAACGTTGGCCAAGTCAGTTACTGA
GATGAAGATCGCCCAGCTGCCAGGACCACCCCAGGACAAGTTAGAGCACTGTTTAGCTCC
TTTGTCTGTGTGATAGACCTAAGAACTGTATTAGTGTTGTACCAGCCTATTAACCTCTTG
TCTGTGCACAGCTTCAAATGTTACCGTCTAGTTAGATTTTTATTTAAAATATGAAAAACT
GCTTTTCCCAAGATGTTTTTTAAAAACAAAGCTACAATTTTAATATTTAACATATTTAAA
GTTTCAAAGCACACCTGTTTGGCTTGGGTGGGGGTGGGGTGGGGGGGACATTCTTTTTCA
GTCTTAATTTTTAAATATTTGATCATTTTCTATTGTCCAATCATTTCAGCACCTCCAAAG
GTCCCTAGGACACTTTGCCTCTCTTCTCCCCCTGCCCCCCACCCTGCTCCCACATCTGGG
GGCCCATGGGCCAGGAGTGGATAAGCCTGCATTAATACAACCTTTCTCCATTCACTTTCT
ATTTACAAATTAGGAAAGCAACCTTTTGGTTTATATATATTTTTTTTAATACCTCAGTGC
TGCAAGTATCACCAGAGAGGCTATGGAAGAATTTTTTTTTAATTTATTGTAGATGTAAAC
AGAATTTTAAAAATAAAAAGTATAAACATCACTGCACTGTGACTGGTGGGAAAAACTGAC
AGTTTCCTCTTTGCACATGTTTAACATTTGGCTGTTATAATATATGGTCCTCGGTTGGGG
AAAGATACTTATGATGAAGGATATTTTTTAATTTAACTTTTTTTTAAATATTGGTAATAG
GTCGGCAACAGCAACTATAGAAGTACAACTCAATAGATGGCATTAAAACATATTGTAGTG
TGGATATATATTTTTTCTTTTTTAAAATGTGATATTGACGTTTTATTAATATTTTTTAAA
TTGTTACGTTTATAAATTTGGTACTTAAGGCACAGCCAGTATGAGACACTGAATGCGACA
TTTATTATAAAGAGCTGCTGCACTCCTATTTTTATAAATTTTACTAACAAAGTAGACTAA
TGTAGACATTCACAGACATGGTAGGGCAAAAGCATCTTCAAACTAAAGACTCCAAAATGC
TAACTCAGAAAGAAAGAAAAAACCCGTTTTCAATTCTAATGAAACAGCAACAACATTTTT
TTTAATTAAAAAAAAAATCATGTTCTTTGTTTTTCTAATAAAATGTTAGGTTGTTTGGTG
AGGTTTTTTTGTTGTTTTTTTCCTTTCTTTGTTTCTTTTCTTTTCCCCCAAACAACCAGA
TTAAATGCTGAGCGCTTTTAAAATATCAAAACTTGTTCAAGTCATAAAACAACAAAACAT
AAGTTTTATTTAAAAAAAAAAAAAGAAAGAAAAAATAGTAAATTCCCAGAAATTCAGTGT
TTAGACGAGGGAATTTAATTCCTATTTTGTCCATGTTGGTGATGTACTGTACTTCCCTTC
CTTTTCTCTGCATCCCCCATCACCTCATAGAAGACTCTTTGTTGATCATTGTATGTTAAT
AATGTATAAAATGGCTATCTTGTAAGCGTGCTGTCCTGGTACTAGTGTAGCGACTTTTTT
TCTCCTCTTTCTTCTAGTACATATTGATAGGTATAACATAATTAAGGTTTAAAAAAAATT
AGACATAGTTATTCAGATTTAGGACCAGTAAGGATAGAACTTTCTCTTATTTATGAAAAA
AAATGCTAATAATTTTGGGGCAGTTTTTTCCTTTAATTATTTTTTTCAATTTCAAGTTTA
ATTTTATTTTAGCTGATCTGATGTGGTTTCAACTAACCCAAGGTCTCACCATGTTAAAAT
GCCGGCGGACTCTACGGCGTTTTGTAGATCCCCCCCCCCCCACCCACTGTGAAGGGGTGC
CATACTACCTTAAATGCTAATGCTAGATATGCAAAACTGGATTTTTTTAATTTATTTTTT
AAAAGAGGGAGGCATGGTATATTAAAATGATTTTACTAAGAGAAAAAATATTTTTTTAAG
AATGCTCAGAAGAAATTGATAATCTGTGTGAATATGTTTTAGATGTTTATATACCTTTTG
AAGAGACCCAGTAGCCCATAGCACAAATCTTGTGGAAATCCGATATGTTTTAATGTGGCT
ACCTAGGTTTAAGGTTCACGTTAGTCCCCCCATTCCATCTAGAAGTCCATTTTGAAAGAT
TTTTGTAAATTCTTTTAACACTGATGTTTCAGCCTCGTCTTTGTTTCAGTTAAGCTCAAT
GGCGAACATGGGAACCACCTTTCGCCTTCCCTGGGGGAGAAACCCTCTTGGCTGATGGCT
TTTCCCCGGAATTATCAAACAGCCACCGGGTGACTTTCTGGCTTCCAGATCCATCTGCCT
GAGACCCCCGAACTCCTCTCCTCCCAAGCAGAGGCGAGTGAGTGGCATTAGCTCCCGGAC
CCATTCCCGGTCCTAGCTGGGCATGGGGCTGACGGAGATGACCAAGCCTTGGTCTGCTCT
CTAGCAGCTTCCACAGACTTGGCTCGTGGCCTTCCTTATATCCACTGGGAACAAACAGCC
TCGCGCTCTATACCAAACAGCCTCGCGCTCTATCCCAGTCGCCCATTAGCTTGATTCAAA
CAAAGCCCCAGCAGGCCTTTGCGTTTTTATCCTTCATAACCTTCATCTTAATTTGAACTT
GTAGCTTGGACTTTAAGGTAGCATGGCTCTATTGCTGTCAATTTACTGTTTCACTGCACA
GCAATCACAGCCAGTGAATGTTACACACATCTTGCTAGACTAGTATAAAAATCATTGGGT
AATTGTTGGTTCTAATGACCTGAAAGGTGTTCAGTTTTTGTTCTTGGTTTTGTTTTGTTT
TTGATTTCTTGGGGGTGGGTTTTGCTTGTTGTTCCTTTTCATTTGGGGGTTTTTGGGGAA
AAAATTTATTTTTGGTTCCAAATAGAAAAACAAAACCTATTTTGATCTTTAGTGCAAACG
AGGGCTAGGGACTTAGCCTCCTCCACCACCTCCACACTGCTTCATTCTGCCATTCACTCA
CTGCAGCATATTCAAGAATAAAGCAATATCGTTTACTACATTTTTTATTGAAGGTCAGCC
ATGCTTTCTGTATTATATTGCATATGAAATTGTTTACAAAAGAAACACTAACTCATACTT
CTCTTTATTGGTTGCAAGTGGCACGCAGGAACAGAGGGAGAGTGGGGGGCTGGTGGGGGA
GGGGAGATTTTTTTTTTTTTTTCTTGAATGTGCTTCGCAAGCCAGGCTATCTTCCAAGGA
AGGCAGACAGTGGGAGAGCAGAGGGACTGACTGCAGGCAAGCACATTGAAGAAAGACACT
GGCGGGTTTCCCCACCCTCACCCCAAAGCAGAAAACTAGCAGACGTCAGCTCAGCCCCGT
CCTGGGCACAGACACTACACAAGGAGACGCTGGAAGTTAAGCAATACTTTAATACTGTAA
TATGTTTGTTTTCTTTTTCTTTCTTTTTTTTCTACCAAAAAAAAGTAAGTAAACTAAAAC
ACAAAAACATATAAATAAAATCCATCCCTCTTGTCGGGGACCTGCAGGGGGGCAACCTTA
ATCCAAACACCTGGCTATCAAATAATCAGAATGTATTGTCTCAGACAGGATTTCAGTTCC
GGGAGGCAGGGGCATGATGGGGGAGGGGGCCGGAGGCTGAGAGACAAAAGTTCCAGAGCC
TCCCTCGAAGGTTCTCTACTACTGTATTCTGTACATAATGTACCATCCCATGTGGAATCT
GTGAGTGTCCTCTTAAGTAGCGTGGGCTAGCCAATCTGCCGTTCATGGTGTATTGTAAAC
TCCGAATTCCATATGTAATAGGATGCAAGTCTAAGCGTTTCATGTGGACATAAATGTATC
TAAATAAAACTTTCCCTAGCACTGTGGCTGACCTCACCCTTACTTTTATACTTTAGTATG
AAACTGATGAGAACTTTGGTAGTGAGTATTTTTTTTATATATATACATATATATGTACTA
TCTATATATATATCTCAAGCATCTTTCAGGTCTTTGTGTGTGGCTTTCTTAAAGCCCTGT
TGTAAAAAATTACTATGTGGATGGCAGTCTCTCACATCACAGATGTGGAAAGTATAATTT
TATATTTGTATTTTCAAATAAATAAGTTTGTGAAAGGTTTCCATCCTCTACTGTGGTCCA
GAAATCAATGTGTTTGTCTGACAAAAAAAAAAATAAAATAAAATAAACTGTTTTGAACAG
A
SEQ ID NO: 124
>ENST00000579466|ENSE00002711759; ENSE00002732163
GCCCCCCAAAATAAGTCTCCGTGCCTGGGGAAAGGCACCGTTCGCTTAGAAGGATCACTG
AGAATTGACTGGAGAATGACTTCCACAGAAAGCACCCTCAGGCTCCAACCCGGCGCGGCC
CGGAGGCTTCCCGGGGGCCAACAGCCCAGGACCTGTCGCCGATCTCTACGGCCCTGCCAG
CCAGGA
SEQ ID NO: 125
>ENST00000675127|ENSE00003580062; ENSE00003904035; ENSE00003900686; ENSE00003902640
GCTTCCCAGCAGCGGCTTATGGACCAGTGGCAGCAGCGGCGGTGGCGGCAGCAAGAGGAT
CAGGATCTGCACACCTCCTCTCAAACACTCTTCTGAGCACGCTCAGCGGGAAGGCTCCAA
CCCGGCGCGGCCCGGAGGCTTCCCGGGGGCCAACAGCCCAGGACCTGTCGCCGATCTCTA
CGGCCCTGCCAGCCAGGACTCCGGAGTGGGGAATTACATAAGTGCGGCCAGCCCACAGCC
GGGCTCGGGCTTCGGCCACGGCATAGCTGGACCTTTGATTGCAACGGCCTTTACAAATGG
ATACCATTGAGCAGGTGCTTTCGTTGCCATCTCACTCTGAG
SEQ ID NO: 126
>ENST00000676073|ENSE00003602774; ENSE00003532300; ENSE00003694227; ENSE00003606151;
ENSE00003901916; ENSE00003641220
TAAAATGTTTATCGGTGGACTGAGCTGGCAGACCTCACCAGATAGCCTTAGAGACTATTT
TAGCAAATTTGGAGAAATTAGAGAATGTATGGTCATGAGAGATCCCACTACGAAACGCTC
CAGAGGCTTCGGTTTCGTCACGTTCGCAGACCCAGCAAGTGTAGATAAAGTATTAGGTCA
GCCCCACCATGAGTTAGATTCCAAGACGATTGACCCCAAAGTTGCATTTCCTCGTCGAGC
GCAACCCAAGAGGGCTTCCCAGAGTTAGCAGTATTTCCTGGGTTTGGAGGAATTGCTGTT
TCTTCCATAAGTGTGAATCTTCAAATTTCCAGATGGTCACAAGAACAAAGAAAATATTTG
TAGGCGGGTTATCTGCGAACACAGTAGTGGAAGATGTAAAGCAATATTTCGAGCAGTTTG
GCAAG
SEQ ID NO: 127
>ENST00000322684|ENSE00003606151; ENSE00003602774; ENSE00003758332; ENSE00001264010;
ENSE00003627192; ENSE00003688116; ENSE00002728269; ENSE00003508406; ENSE00003694227;
ENSE00003652249
GCTTTCCTTTTAGCTTTTGTAAGTTACACGTCAAAATGGCCGATCTGACATCGGTGCTCA
CTTCTGTTATGTTTTCTCCCTCTAGTAAAATGTTTATCGGTGGACTGAGCTGGCAGACCT
CACCAGATAGCCTTAGAGACTATTTTAGCAAATTTGGAGAAATTAGAGAATGTATGGTCA
TGAGAGATCCCACTACGAAACGCTCCAGAGGCTTCGGTTTCGTCACGTTCGCAGACCCAG
CAAGTGTAGATAAAGTATTAGGTCAGCCCCACCATGAGTTAGATTCCAAGACGATTGACC
CCAAAGTTGCATTTCCTCGTCGAGCGCAACCCAAGATGGTCACAAGAACAAAGAAAATAT
TTGTAGGCGGGTTATCTGCGAACACAGTAGTGGAAGATGTAAAGCAATATTTCGAGCAGT
TTGGCAAGGTGGAAGATGCAATGCTGATGTTTGATAAAACTACCAACAGGCACAGAGGGT
TTGGCTTTGTCACTTTTGAGAATGAAGATGTTGTGGAGAAAGTCTGTGAGATTCATTTCC
ATGAAATCAATAATAAAATGGTAGAATGTAAGAAAGCTCAGCCGAAAGAAGTCATGTTCC
CACCTGGGACAAGAGGCCGGGCCCGGGGACTGCCTTACACCATGGACGCGTTCATGCTTG
GCATGGGGATGCTGGGATATCCCAACTTCGTGGCGACCTATGGCCGTGGCTACCCCGGAT
TTGCTCCAAGCTATGGCTATCAGTTCCCAGACTATTTGCCGGTTTCACAAGACATAATTT
TTATCAACTAGCTCTTAAGAGAGGCATAGCAAAGTGGGGGTTGCTACCATTTCTAGAGAG
AGAGGACACAGTCCCGGCCTGGGCTGCCCCCGCTCCAGTCAATGCTCACTGAAAGTCTGT
CTTAGCTGCCTGTTTGAATGACTGTTCTTTTTCTCATTTTTAATTCTTGGACTCATGTCC
TCATTGCTTCACTCAATTAAAAAAAAATTATTCTCCAGTCCCCTCCCACTTTGCTTCTTG
TATGCATTGTGACCGACCCCACTTCCTCAGAATGTAACGGGGCCAGAGGGAAACTTCTCA
CAAACTTCGTAGAGCCTCCTCAGGGGAAGCTAGGAAGAAGACATCAAATGTTTTTAAGTC
ATGACCAAACAGGCTTGTTGGGGACATATCATGGGGTGAGCTTTGAAGTGCTGGTGGTCC
AGAGGGGTTGCAGATACGTGACTTGGAGCACCCGTGTCTTACGATGGACAGTGATAAAGG
TGAACACACAGAGACAGACTATTCTCTAAGAATGTGAGAAACCTGATCTGGAAGAGGAGC
TATATAAACACTATCTGACTATCTTTGTCTTTTGGGGCCAGTGGCTGTTGGCATAATCAC
AAGCCTGTCTGTCTTCGAGAAGGGACAGTGGAGTCATCCAGGTGCTGCCACATGACAGGC
ACGGTGGGCACCGATCCACAGTGGGCCCCGCCTTCCCCAGCTCGCCTCCCTGCCTGTGCT
GGCCTGGCCTTGCCTGCTGGCACCATTGGAGTAGGAGGGGGTGGAACACAGGGGGCCCAT
CCTGATCAGGCCCCATCTCAAGGTTGGCACTCCTGCCCATCACCCTTAGAAGGATCTTTT
CCCATGGCTTGACTTCCTTCATTTCCCTAACTGAAATACACCCACTCTCTTGGAATAATG
ACGTACCACTCAGTTGGACCCTCAAGAGTCACTGCTTTGTCTGTGCTGGTAGTTTGTGAG
AAGTGACCCGCACGCTTCCATTTGATGCATTTGATGTGAGTGAATCCATACATTTGAATG
TCATTGTCCTTGAGACCCTACATGTGCAGTTTGGCTCATCTCATTAAAGATGCTTGATGT
SEQ ID NO: 128
>ENST00000584476|ENSE00003654833; ENSE00002734806; ENSE00002687828; ENSE00003543198
GGCAGACCTCACCAGATAGCCTTAGAGACTATTTTAGCAAATTTGGAGAAATTAGAGAAT
GTATGGTCATGAGAGATCCCACTACGAAACGCTCCAGAGGCTTCGGTTTCGTCACGTTCG
CAGACCCAGCAAGTGTAGATAAAGTATTAGGTCAGCCCCACCATGAGTTAGATTCCAAGA
CGATTGACCCCAAAGTTGCATTTCCTCGTCGAGCGCAACCCAAGGTAAGTAGGAGAATAA
ACAGTAGGATTTTAGCACTCAGAGATGATTGCCAAGAATTTCAAATTTCAAACAGCATTG
GCCATGAACTGTTGAAGCCTGGTATTCACTGTTCCTTCGGGGTATCAGGACAGGCTGGGC
AAGTAGTCCTGTGAGATAACCATGCGTCTCTAAGTTAGCCATCCCAAGGTGGCTTCTGAA
CATCCACCTGGGGGCAGGGACAAGTCTGATGCTTAGTGGGAGGAC
>ENST00000577241|ENSE00002709985; ENSE00002701177
GTGGAGAAAGTCTGTGAGATTCATTTCCATGAAATCAATAATAAAATGGTAGAATGTAAG
AAAGCTCAGCCGAAAGAAGTCATGTTCCCACCTGGGACAAGAGGCCGGGCCCGGGGACTG
CCTTACACCATGGACGCGTTCATGCTTGGCATGGGGATGCTGGGTGAGTCTGGACAGGAC
CGCAGGTCACCATGGACTGGGAGGGCTATGGAGGCCTCTACTCCCAACTGGGTCACCTAC
CAGTGGGGCAAACTGCTTCACCTTTCTAAGCCTCAGTTTCCTTGTCTGTAGATGAGGATG
ATAATTCCCCGTTCCAAGACAGTTGTGATGATTAAGTGTGGGTGTGTGTGTGTGCATGCA
TGTGTGTGTGTGTGTGTGTGTGTTTGTATTTATAATATTGCCCCATGCCTGGCTTATAGG
ATATGTTAGACTATTTTCTCTCTTTTCCATCTCCTTCCTCAAAAGAAGGAAAAGTCCCCC
TCTATCTGCCTCAGCCCTCTCATCTGAGTGGGAGTTCTTAAGATGTAAGGACTCCTGGCT
GACTTGACTTGTGTGGGCTAAGGCTACGTTTTCTAAAACTTGGGAGAGGAGGGAAGTGGT
AAGGGTGGGCGATAATCCTGTCTATTTAAATGATTAACATTTTTCTCTTGGGATATCAAA
ATTTGCATTTAAATGGATGTTTTAAATAGCCTGTTTTACTCTTTATTTGC
SEQ ID NO: 129
>ENST00000416426|ENSE00003627192; ENSE00001102120; ENSE00003652249; ENSE00002235435;
ENSE00003758332; ENSE00003688116; ENSE00002309580; ENSE00003694227; ENSE00003508406;
ENSE00003602774; ENSE00003531946; ENSE00001662724; ENSE00003606151; ENSE00003580062
AGAGCCAGAGAGAACTTCCAGCGCAAAAGGAAAATAAAACTTGTGGCTGGTGTTTGTGCA
GGAGGGTCTCCGCCATCCTGAAGCCCCCCGATCCTGGGGCGTCTCGGGGGCCGCCAAAGG
AGCGCCAGGGTTAAAATGTTTATCGGTGGACTGAGCTGGCAGACCTCACCAGATAGCCTT
AGAGACTATTTTAGCAAATTTGGAGAAATTAGAGAATGTATGGTCATGAGAGATCCCACT
ACGAAACGCTCCAGAGGCTTCGGTTTCGTCACGTTCGCAGACCCAGCAAGTGTAGATAAA
GTATTAGGTCAGCCCCACCATGAGTTAGATTCCAAGACGATTGACCCCAAAGTTGCATTT
CCTCGTCGAGCGCAACCCAAGATGGTCACAAGAACAAAGAAAATATTTGTAGGCGGGTTA
TCTGCGAACACAGTAGTGGAAGATGTAAAGCAATATTTCGAGCAGTTTGGCAAGGTGGAA
GATGCAATGCTGATGTTTGATAAAACTACCAACAGGCACAGAGGGTTTGGCTTTGTCACT
TTTGAGAATGAAGATGTTGTGGAGAAAGTCTGTGAGATTCATTTCCATGAAATCAATAAT
AAAATGGTAGAATGTAAGAAAGCTCAGCCGAAAGAAGTCATGTTCCCACCTGGGACAAGA
GGCCGGGCCCGGGGACTGCCTTACACCATGGACGCGTTCATGCTTGGCATGGGGATGCTG
GGATATCCCAACTTCGTGGCGACCTATGGCCGTGGCTACCCCGGATTTGCTCCAAGCTAT
GGCTATCAGTTCCCAGGCTTCCCAGCAGCGGCTTATGGACCAGTGGCAGCAGCGGCGGTG
GCGGCAGCAAGAGGATCAGTCCTGAATAGCTACAGTGCTCAACCGAATTTTGGCGCGCCC
GCTTCCCCGGCAGGCTCCAACCCGGCGCGGCCCGGAGGCTTCCCGGGGGCCAACAGCCCA
GGACCTGTCGCCGATCTCTACGGCCCTGCCAGCCAGGACTCCGGAGTGGGGAATTACATA
AGTGCGGCCAGCCCACAGCCGGGCTCGGGCTTCGGCCACGGCATAGCTGGACCTTTGATT
GCAACGGCCTTTACAAATGGATACCATTGAGCAGGTGCTTTCGTTGCCATCTCACTCTGA
GAGCATACCTGGATGTCCAGGCAAGACTGGGCGAAGTTTCTGAGTGGCCCTTTGTTTAGG
TGATGTCCTCAGACCTGGACCCCCACCAGCCTCACTCCCCATCCCAACCAGAGATGGCTC
ACTTCGGATCGAGGGTTGACTACATCTCATCATCTCACGAATCTGCTGTAATATAAGACA
ACAGCTTTTAAATGTGTATATAACCCATGATTTCGGTTTTGTTTTGTTTTGTTTTTCTTG
ATGGTTTCCCTCTCCCTCCCTCTCTTCCCATTCTCCTTTTAAATCTCTTTGAATCACATT
TGGTAGTGATTTTGACTTAGTCCAGTAGTCACATAGCTTTAATATCTAGTTCAAAGCTAA
CCATAGTATAATTGTTATATTAAGGAGTTATTTTTTCTTAAAACATTTTTTTTTGCTTGT
TTTGGTTCTGTTCTCACTTTTAAAGGATGCTGAGATGGTAATATGACTCTCCATATTTTG
GTACCAATTCTGAGACTGTATGAATTTTCAGGTGGAACTTTAGCACACACTGAAGCAAAG
TTGTGAAGTGCAGGGCGGGAGGTGGGCGTGAGCT
SEQ ID NO: 130
>ENST00000581523|ENSE00003575978; ENSE00003618729; ENSE00003654833; ENSE00003543198;
ENSE00002721534; ENSE00002714010
ATGGAGGCAAATGGGAGCCAAGGCACCTCGGGCAGCGCCAACGACTCCCAGCACGACCCC
GGTAAAATGTTTATCGGTGGACTGAGCTGGCAGACCTCACCAGATAGCCTTAGAGACTAT
TTTAGCAAATTTGGAGAAATTAGAGAATGTATGGTCATGAGAGATCCCACTACGAAACGC
TCCAGAGGCTTCGGTTTCGTCACGTTCGCAGACCCAGCAAGTGTAGATAAAGTATTAGGT
CAGCCCCACCATGAGTTAGATTCCAAGACGATTGACCCCAAAGTTGCATTTCCTCGTCGA
GCGCAACCCAAGGTGTTCTAGCTGAGCGTTTAAGAGCATGGACTCTGGAACCAGACTTTG
AATCCTTGCTCTGCCACTGCAGCTGTGTGACCTTGAGCAAGCTATCTAAATATTCTGTGC
CTTTGTTTTGTCATCTGTACAATGGAGATGATGATGGTATCACCTTCATGGGGTTGTCAT
AAGGATTATGGGACTTAACATAATAAAGTGTTTAGAATGGTGTCTGCAACATAA
SEQ ID NO: 131
>ENST00000579505|ENSE00003615026; ENSE00003525930; ENSE00002704420; ENSE00002703116;
ENSE00003649314; ENSE00003540766; ENSE00003661268
ATTTATGTGCCCCTGTGCATGGGTGTGGATGTGATTTCCTGGCGTGCAGGGTGGAAGATG
CAATGCTGATGTTTGATAAAACTACCAACAGGCACAGAGGGTTTGGCTTTGTCACTTTTG
AGAATGAAGATGTTGTGGAGAAAGTCTGTGAGATTCATTTCCATGAAATCAATAATAAAA
TGGTAGAATGTAAGAAAGCTCAGCCGAAAGAAGTCATGTTCCCACCTGGGACAAGAGGCC
GGGCCCGGGGACTGCCTTACACCATGGACGCGTTCATGCTTGGCATGGGGATGCTGGGAT
ATCCCAACTTCGTGGCGACCTATGGCCGTGGCTACCCCGGATTTGCTCCAAGCTATGGCT
ATCAGTTCCCAGGCTTCCCAGCAGCGGCTTATGGACCAGTGGCAGCAGCGGCGGTGGCGG
CAGCAAGAGGATCAGTCCTGAATAGCTACAGTGCTCAACCGAATTTTGGCGCGCCCGCTT
CCCCGGCAGGCTCCAACCCGGCGCGGCCCGGAGGCTTCCCGGGGGCCAACAGCCCAGGAC
CTGTCGCCGATCTCTACGGCCCTGCCAGCCAGGACTC
SEQ ID NO: 132
>ENST00000581776|ENSE00002721089; ENSE00003606151; ENSE00003532300; ENSE00003575978;
ENSE00003694227; ENSE00002724238; ENSE00002690135
GATATGGAGGCAAATGGGAGCCAAGGCACCTCGGGCAGCGCCAACGACTCCCAGCACGAC
CCCGGTAAAATGTTTATCGGTGGACTGAGCTGGCAGACCTCACCAGATAGCCTTAGAGAC
TATTTTAGCAAATTTGGAGAAATTAGAGAATGTATGGTCATGAGAGATCCCACTACGAAA
CGCTCCAGAGGCTTCGGTTTCGTCACGTTCGCAGACCCAGCAAGTGTAGATAAAGTATTA
GGTCAGCCCCACCATGAGTTAGATTCCAAGACGTGCAGGAGGCAGAAGCGGTTGGTTTAT
TGACATGGAAACGAGGATCTTGTTTACTGTTACCTTAAAAGCTTTGTTTAGATTGACCCC
AAAGTTGCATTTCCTCGTCGAGCGCAACCCAAGCATTGCTGACTTTCTTCCTCCATGGCA
CAGGATTTTTGCCCAAAACAAAAGTCTCAATTTATTTGTTCTTTAAAAAAATAAAAACCA
CACTGAACAAAAA
SEQ ID NO: 133
>ENST00000676105|ENSE00003532300; ENSE00003575978; ENSE00003902683; ENSE00003543198
TAAAATGTTTATCGGTGGACTGAGCTGGCAGACCTCACCAGCCTTAGAGACTATTTTAGC
AAATTTGGAGAAATTAGAGAATGTATGGTCATGAGAGATCCCACTACGAAACGCTCCAGA
GGCTTCGGTTTCGTCACGTTCGCAGACCCAGCAAGTGTAGATAAAGTATTAGGTCAGCCC
CACCATGAGTTAGATTCCAAGACGATTGACCCCAAAGTTGCATTTCCTCGTCGAGCGCAA
CCCAAG
SEQ ID NO: 134
>ENST00000674898|ENSE00003901695; ENSE00003688116; ENSE00003627192; ENSE00003508406;
ENSE00003904122; ENSE00003652249
ACATTTCAGCAGGCAGAGGCAGGCAATATGGCCCTGAGCAAGGGAAGTACAGAAGTGGGA
AGTGCAGGGGACTTCAGATCACGGGGTGAGGGAGGAGGCTCACTGAGAGAGAAGACCAGT
ACTGTGCTCTGTGGGGCCAGCTCATGAGACCGCAAAGATAGTAGCAGCTTTGATGCCAGG
CTGGGAATTCTGCCTGCGCTGTTGGGAGCCAGTCACGTGTTCCACACAGGTGGAAGATGC
AATGCTGATGTTTGATAAAACTACCAACAGGCACAGAGGGTTTGGCTTTGTCACTTTTGA
GAATGAAGATGTTGTGGAGAAAGTCTGTGAGATTCATTTCCATGAAATCAATAATAAAAT
GGTAGAATGTAAGAAAGCTCAGCCGAAAGAAGTCATGTTCCCACCTGGGACAAGAGGCCG
GGCCCGGGGACTGCCTTACACCATGGACGCGTTCATGCTTGGCATGGGGATGCTGGGATA
TCCCAACTTCGTGGCGACCTATGGCCGTGGCTACCCCGGATTTGCTCCAAGCTATGGCTA
TCAGTTCCCAGACTATTTGCCGGTTTCACAAGACATAATTTTTATCAACTAGCTCTTAAG
AGAGGCATAGCAAAGTGGGGGTTGCTACCATTTCTAGAGAGAGAGGACACAGTCCCGGCC
TGGGCTGCCCCCGCTCCAGTCAATGCTCACTGAAAGTCTGTCTTAGCTGCCTGTTTGAAT
GACTGTTCTTTTTCTCATTTTTAATTCTTGGACTCATGTCCTCATTGCTTCACTCAATTA
AAAAAAAATTATTCTCCAGTCCCCTCCCACTTTGCTTCTTGTATGCATTGTGACCGACCC
CACTTCCTCAGAATGTAACGGGGCCAGAGGGAAACTTCTCACAAACTTCGTAGAGCCTCC
TCAGGGGAAGCTAGGAAGAAGACATCAAATGTTTTTAAGTCATGACCAAACAGGCTTGTT
GGGGACATATCATGGGGTGAGCTTTGAAGTGCTGGTGGTCCAGAGGGGTTGCAGATACGT
GACTTGGAGCACCCGTGTCTTACGATGGACAGTGATAAAGGTGAACACACAGAGACAGAC
TATTCTCTAAGAATGTGAGAAACCTGATCTGGAAGAGGAGCTATATAAACACTATCTGAC
TATCTTTGTCTTTTGGGGCCAGTGGCTGTTGGCATAATCACAAGCCTGTCTGTCTTCGAG
AAGGGACAGTGGAGTCATCCAGGTGCTGCCACATGACAGGCACGGTGGGCACCGATCCAC
AGTGGGCCCCGCCTTCCCCAGCTCGCCTCCCTGCCTGTGCTGGCCTGGCCTTGCCTGCTG
GCACCATTGGAGTAGGAGGGGGTGGAACACAGGGGGCCCATCCTGATCAGGCCCCATCTC
AAGGTTGGCACTCCTGCCCATCACCCTTAGAAGGATCTTTTCCCATGGCTTGACTTCCTT
CATTTCCCTAACTGAAATACACCCACTCTCTTGGAATAATGACGTACCACTCAGTTGGAC
CCTCAAGAGTCACTGCTTTGTCTGTGCTGGTAGTTTGTGAGAAGTGACCCGCACGCTTCC
ATTTGATGCATTTGATGTGAGTGAATCCATACATTTGAATGTCATTGTCCTTGAGACCCT
ACATGTGCAGTTTGGCTCATCTCATTAAAGATGCTTGATGTAATAATTGGTTAGTTTCCT
TTTATTTTCCTGCAGGCTTTTCCATGAGTATTATTTTTTTCAAAGAACAAATCTGTATGG
CTTTTCCCCATCTCCATATTTTGTTTTGCTATGAATTGCTTTGCTTTGGTGAACTTGTCC
TAGTATGCTTGCCTCACAAACGTTTTAGCCATTGTGAATTTTCTTCATCTCTGTAAATAG
TTCATCTGTGCTTCTCCCTGATGACGTTTTATTTTTTTTCCCCTGTAAGCAACCGAGGTA
G
SEQ ID NO: 135
>ENST00000583821|ENSE00002695419; ENSE00002712215
AAATGTCAGAGCCAGGCGCAGGCTTTGTGTTTGGTGCTGATGCATGGCATTTACTCTGAA
GGTTCAGGAAAGTACATGGGCTTTGCGGGGTCTGGCTGCCCAAATATGGGTTAATTAGCA
ACAGCTGACAGCAGCGCCCCCGGCCACAGGAGAGAGGTGACCCAGACCCTTAATAAAAAA
ACCCTCATGAGAGACAAATTATTCTGTGAAGGAAAATAACTCAGGCTTTCCTCATTGCCA
CCCTCCGTGAGATTTTACCCCAGACCTGAGGCGGCTGTACTAACAGGACTCTGATCTTTC
TCTTTGTGTTCAAGGATATCCCAACTTCGTGGCGACCTATGGCCGTGGCTACCCCGGATT
TGCTCCAAGCTATGGCTATCAGTTCCCAGACTATTTGCCGGTTTCACAAGACATAATTTT
TATCAACTAGCTCTTAAGAGAGGCATAGCAAAGTGGGGGTTGCTACCATTTCTAGAGAGA
GAGGACACAGTCCCGGCCTGGGCTGCCCCCGCTCCAGTCAATGCTCACTGAAAGTCTGTC
TTAGCTGC
SEQ ID NO: 136
>ENST00000579590|ENSE00003694227; ENSE00003606151; ENSE00003758332; ENSE00003602774;
ENSE00002688031; ENSE00002709409
CCTTTTAGCTTTTGTAAGTTACACGTCAAAATGGCCGATCTGACATCGGTGCTCACTTCT
GTTATGTTTTCTCCCTCTAGTAAAATGTTTATCGGTGGACTGAGCTGGCAGACCTCACCA
GATAGCCTTAGAGACTATTTTAGCAAATTTGGAGAAATTAGAGAATGTATGGTCATGAGA
GATCCCACTACGAAACGCTCCAGAGGCTTCGGTTTCGTCACGTTCGCAGACCCAGCAAGT
GTAGATAAAGTATTAGGTCAGCCCCACCATGAGTTAGATTCCAAGACGATTGACCCCAAA
GTTGCATTTCCTCGTCGAGCGCAACCCAAGATGGTCACAAGAACAAAGAAAATATTTGTA
GGCGGGTTATCTGCGAACACAGTAGTGGAAGATGTAAAGCAATATTTCGAGCAGTTTGGC
AAGGTTTCCGCCGGCAGCCCAGGCCTCTCCCT
SEQ ID NO: 137
>ENST00000676345|ENSE00003902405; ENSE00003661268
GAGAAGGACCGCAAACTCACAGAGGAACGGGTGTGAGACCATTCATATCATCAGTCCACC
ATAGACCTGTGTGGAAGATGCAATGCTGATGTTTGATAAAACTACCAACAGGCACAGAG

It is noted herein that this disclosure is also directed to the following clauses or embodiments:
1. An inhibitor of RNA-binding protein Musashi (MSI2) function or an analogue thereof for use in the treatment of myotonic dystrophy in a mammal, wherein the inhibitor is capable of reducing in a myotonic dystrophy (MD) cell or in a cell with the phenotype of a MD cell, at least one of
   i) MSI2 protein activity,
   ii) intracellular transcript levels of MSI2, and/or
   iii) intracellular protein levels of MSI2,
thereby causing in the same MD cell an increase in the intracellular miR-7 levels of at least 1.5-fold of the intracellular miR-7 levels of a reference cell, as measured by a gene expression quantification technique such as quantitative reverse transcription PCR (RT-qPCR), Northern blotting, or RNA-sequencing (RNA-Seq) techniques,
wherein the cell with the phenotype of a MD cell is selected from the group of muscular cells, nervous cell, or cardiac cells, and
wherein the reference cell is immortalized skin fibroblasts expressing conditional MyoD.
2. The MSI2 inhibitor for use according to 1, wherein the increase in the intracellular miR-7 level is of at least 1.7-fold of the intracellular miR-7 levels of a reference cell.
3. The MSI2 inhibitor for use according to any of 1 and 2, wherein the MD cell or a cell with the phenotype of a MD cell is a myocyte.
4. The MSI2 inhibitor for use according to any of 2 and 3, wherein the increase in the intracellular miR-7 level is of at least 1.7-fold of the intracellular miR-7 levels of a reference cell and wherein the MD cell or a cell with the phenotype of a MD cell is a myocyte.
5. The MSI2 inhibitor for use according to any of the 1 to 4, wherein said inhibitor is for use in the treatment of myotonic dystrophy type 1.
6. The MSI2 inhibitor for use according to any of 1 to 5, wherein the mammal is a human being.
7. The MSI2 inhibitor for use according to 1 to 6, wherein the inhibitor reduces the intracellular transcript levels of MSI2, and wherein the inhibitor is an oligonucleotide molecule which comprises a fragment composed of a succession of nucleotide units, wherein the oligonucleotide is selected from the group consisting of small interfering RNAs, antisense oligonucleotides, gapmers, morpholino oligomers, FANA oligonucleotides, agomiRs, miRNA mimics, antagomiRs, blockmiRs, PNAs, LNAs (locked nucleic acid antisense oligonucleotides), splice-switching oligonucleotides (SSOs), LNA-based splice-switching oligonucleotides (LNA SSOs), LNA/DNA mixmers or miRNA sponges.
8. The oligonucleotide molecule for use according to 7, wherein said oligonucleotide comprises at least 7 consecutive nucleotides in length that have at least 90% identity over the complementary sequence of the full length sequence of SEQ ID NO: 1 or, preferably, SEQ ID: 2.
9. The oligonucleotide molecule for use according to 8, wherein said oligonucleotide comprises at least 10-25 nucleotides in length, from which at least 7 consecutive nucleotides in length have at least 90% identity over the complementary sequence of the full length sequence of SEQ ID NO: 1 or, preferably SEQ ID: 2.
10. The oligonucleotide molecule for use according to any of previous clauses, wherein said oligonucleotide comprises at least 10-25 nucleotides in length, from which at least 7 consecutive nucleotides in length have at least 90% identity over the complementary sequence of the full sequence of any of the oligonucleotides of SEQ ID NO:3 to SEQ ID NO:37.
11. The oligonucleotide molecule for use according to any of previous clauses, wherein said oligonucleotide comprises at least 10-25 nucleotides in length, from which at least 7 consecutive nucleotides in length have at least 95% identity over the full sequence of any of the oligonucleotides SEQ ID NO: 38 to 43.
12. The MSI2 inhibitor for use according to any of 1 to 6, wherein the inhibitor reduces the activity of MSI2 protein, and wherein the inhibitor is a molecule such as 2,3,4,10-Tetrahydro-7,10-dimethyl-2,4-dioxobenzo[g]pteridine-8-carboxaldehyde, analogues and derivatives thereof.
13. A pharmaceutical composition comprising an inhibitor as defined in any of 1 to 12, or a mixture of two or more of them, optionally further comprising a carrier and/or one or more pharmaceutically acceptable excipients.
14. A pharmaceutical composition according to 13, wherein the pharmaceutical composition comprises an expression vector comprising the oligonucleotide sequence of at least one of the oligonucleotide molecules as defined in any of 1 to 11.
15. A pharmaceutical composition according to 13 and 14, for use according to any of 1 to 12.

### EXAMPLES

### Example 1

### Materials and Methods

### Chemically-Modified Oligonucleotides

siRNAs targeting MSI2 transcripts and negative control were commercially available from Invitrogen (Madrid, Spain), Silencer® Select siRNA. Cat #4390843 for negative control and Cat #4392421 for siRNAs against MSI2 (ID S42755 and S42757). Gapmer oligonucleotides and scramble control were synthesized by biomers.net GmbH.

The ASOs sequences were as follows:

ASO1
(SEQ ID NO: 76)
5′ +T+G+A*C*T*T*C*T*T*T*C*G*G+C+T+G 3′
ASO3
(SEQ ID NO: 77)
5′ +T+T+G*G*A*T*T*A*A*G*G*T*T+G+C+C 3′
Scramble
(SEQ ID NO: 44)
5′ +T+T+C*C*C*T*G*A*A*G*G*T*T+C+C+T 3′

Where + denotes locked nucleic acid and * denotes phosphorothioate linkages.

Hsa-miR-107 agomiR (agomiR-107) (SEQ IDs 41 (sense) and 78 (antisense)) were synthesized by biomers.net GmbH according to the following sequences:

AgomiR-107 sense strand
5′ AGCAGCAUUGUACAGGGCUAUCA 3′
AgomiR-107 antisense strand (SEQ ID NO: 78):
5′mU*mG*mAmUmAmGmCmCmCmUmGmU-
mAmCmAmAmUmGmCmU*mG*mC*mU*3′chol

Where m denotes 2′-O-methyl-modified phosphoramidites, * denotes phosphorothioate linkages, and “chol” denotes cholesterol groups

### Cells

MD1 cells are cells derived from MD1 patients. In the present disclosure, these cells are obtained from patient-derived biopsies (FIG. 2 A), from primary cultures thereof (primary myoblasts, as shown in FIG. 2 B) or from immortalized skin fibroblast cell line expressing conditional MyoD (also referred to as “reference cells” as described in Arandel et al. 2017 and as explained above). These MD1 immortalized skin fibroblast cells expressing conditional MyoD are used in all figures except in FIGS. 2 A and B. The MD1 cells are the reference cells used to study the effects of the inhibitors described herein.

Skin fibroblasts transdifferentiated into multinucleated myotubes for 7 days are called herein TDM cells and are obtained as previously described¹

Non-DM control (Ctrl) cells (CNT), also referred to in the present disclosure as healthy cells, are cells obtained as above but from healthy or non-MD1 biopsies or from primary cultures thereof.

Cell Culture and Treatment with Oligonucleotides and Small Molecule

TDMs were cultured as previously described¹. After 4 days in differentiation medium (MDM), cells were transfected with a combination of two siRNAs (SEQ ID NO: 40 and 43) targeting MSI2 transcripts (final concentration 100 nM) using Lipofectamine RNAiMAX transfection reagent (Invitrogen, Madrid, Spain)) following the manufacturer's recommendations. After 48 h, the medium containing the siRNAs was replaced by fresh MDM. 24 h later, cells were collected. As a control, the same protocol was performed using 100 nM of a siRNA with a scrambled sequence (Cat #4390843 for negative control). Gapmers against MSI2 and agomiR-107 were transfected following the same protocol using two different concentrations, 30 and 150 nM in the case of Gapmers and 100 and 300 nM in the case of agomiR-107. To treat cells with the small molecule Ro-08-2750 (Tocris Cat. No. 2272, Bristol, UK)), cells were cultured as before. Briefly, after 96 h in MDM, the medium was supplemented with Ro-08-2750 at a final concentration of 10 μM (0.8% DMSO) or with the vehicle, DMSO 0.8%, for 48 h. Then cells were collected for further analyses.

Immortalized myoblasts were cultured in a growth medium consisting of a mix of 199:DMEM (1:4 ratio; Life Technologies) supplemented with 20% FBS (Life Technologies), 50 μg/ml gentamycin (Life technologies), 25 μg/ml fetuin, 0.5 ng/ml bFGF, 5 ng/ml EGF and 0.2 μg/ml dexamethasone (Sigma-Aldrich). Myogenic differentiation is induced by switching confluent cell cultures to DMEM supplemented with 5 μg/ml insulin for myoblasts. Immortalized myoblasts cultured for 5 days in MDM were treated with Ro 08-2750 to a final concentration of 10 mM for 48 h. In the case of fusion index determinations at 7, 10, and 14 days, the compound was added for the last 48 h.

### Primary Human Skeletal Muscle Cultures

Primary human myoblasts from muscle biopsies of 3 MD1 and 3 control patients were obtained as previously described² by clinician collaborators from the La Fe Hospital (Valencia, Spain). In order to obtain highly purified myoblast cell cultures, positive selection for the CD56 surface marker was performed using CD56-coated microbeads (Milteny Biotec, Bergisch Gladbach, Germany) following manufacturer instructions². Purified myoblasts were seeded in 6 well plates (2.5×10⁵ cells/well) and the following day the medium was substituted with differentiation medium (DMEM+2% horse serum). Medium was changed every 2 days, and pellets were collected at day 7 of differentiation.

To treat primary myoblasts, CNT-10, DM1-14, and DM1-16 with the small molecule Ro 08-2750 cells were cultured as before. After 5 days in differentiation conditions, the medium was supplemented with Ro 08-2750 to a final concentration of 10 mM (0.8% DMSO) or with 0.8% DMSO (vehicle) for 48 h. Information about biopsy donors is compiled in Table 1.

### MD1 Patients and Skeletal Muscle Biopsies

All muscle biopsies were taken by clinician collaborators after informed consent from patients with approval by the institutional review boards (IRBs) of the University Hospital La Fe (Valencia, Spain experimentation ethics committee authorization 2014/0799). Muscle biopsies used for miR-7 and MSI2 determinations were obtained at La Fe University Hospital. A detailed description of muscle type and repeats length is provided in Table 1

TABLE 1
Information of Biopsies from Skeletal Muscle

| | Sample | Muscle | Repeats length | |
|---|---|---|---|---|
| Controls | CNT-1 | Deltoid | nd | |
| | CNT-2 | Deltoid | nd | |
| | CNT-3 | Deltoid | nd | |
| | CNT-4 | Biceps | nd | |
| | CNT-5 | Deltoid | nd | |
| | CNT-6 | Deltoid | nd | |
| | CNT-7 | Deltoid | nd | |
| | CNT-8 | Deltoid | nd | |
| | CNT-9 | Deltoid | nd | |
| | CNT-10 | Medial gastrocnemius | nd | |
| | CNT-11 | Deltoid | nd | |
| | CNT-12 | Deltoid | nd | |
| | CNT-13 | Deltoid | nd | |
| | CNT-14 | Deltoid | nd | |
| | CNT-15 | Deltoid | nd | |
| | CNT-16 | Deltoid | nd | |
| | CNT-17 | Deltoid | nd | |
| Patients | MD1-1 | Deltoid | nd | |
| | MD1-2 | Deltoid | 0.3 | kb |
| | MD1-3 | Deltoid | 3.6 | kb |
| | MD1-4 | Deltoid | 1.1 | kb |
| | MD1-5 | Deltoid | nd | |
| | MD1-6 | Deltoid | nd | |
| | MD1-7 | Deltoid | 0.9 | kb |
| | MD1-8 | Deltoid | 0.4 | kb |
| | MD1-9 | Deltoid | 0.2 | kb |
| | MD1-10 | Deltoid | 0.75 | kb |
| | MD1-11 | Deltoid | nd | |
| | MD1-12 | Deltoid | nd | |
| | MD1-13 | Deltoid | 3 | kb |
| | MD 1-14 | Deltoid | 150 | |
| | MD1-15 | Deltoid | nd | |
| | MD1-16 | Deltoid | 0.8 | kb |

nd: not determined
MD1: cells affected with MD1 from human biopsies
CNT: Control healthy cells from human biopsies

### Immunofluorescence Methods

Desmin immunostaining and fusion index and diameter determination were performed as previously described³. For MSI2 immunodetection, 3.5×10⁴ fibroblasts/well were seeded in 24 well plates. After 7 days of differentiation into myotubes, cells were fixed with 4% paraformaldehyde (PFA) for 15 min. After 3 washes with PBS-T (0.1%×-TritonX-100 in PBS 1×) cells were blocked (PBS-T, 1% BSA, 5% normal goat serum) for 1 h and incubated with rabbit anti-MSI2 antibody (1:100, Abcam, EP1305Y, Cambridge, UK) in blocking buffer for 48 h at 4° C. After three washes with PBS-T, cells were incubated for 1 h with goat anti-rabbit-FITC IgG (1:200, Sigma-Aldrich, St. Louis, Missouri) in blocking buffer. Finally, cells were washed thrice with PBS 1× and were counterstained and mounted with VECTASHIELD® mounting medium containing DAPI (Vector Laboratories, London, UK) to detect the nuclei. Images were taken in a confocal microscope LSM800 (Zeiss) at 400× magnification.

For LC3B immunodetection, cells were differentiated in MDM for 7 days (3×105 cells/well in 24-well plates). After 48 h of gapmer treatment, cells were fixed with 4% PFA in PBS for 15 min at room temperature. After fixation, cells were permeabilized with methanol 100% for 15 min at −20° C. and blocked with blocking buffer (PBS 1×, 0.5% BSA, 1% goat serum) for 1 h at room temperature. Cells were incubated overnight (O/N) with rabbit anti-LC3B (1:200, ab51520; Abcam, Cambridge, UK) diluted in blocking buffer. Then, samples were incubated with goat anti-rabbit IgG (H+L) Alexa Fluor Plus 594 (1:200; Invitrogen, Carlsbad, CA, USA) for 2 h. Between antibodies, cells were washed three times with PBS 1×. Finally, cells were washed three times with PBS and mounted in Vectashield (Vector Laboratories, London, UK) with 2 mg/mL DAPI.

### LC3 Puncta Quantification

Image analyses of LC3-stained myotubes were performed using the Ifdotmeter software following the authors' recommendations (see Rodríguez-Arribas, M., et al. (2016). IFDOTMETER: A New Software Application for Automated Immunofluorescence Analysis. J. Lab. Autom. 21, 246-259). Briefly, the quantification of the number of LC3 dots or LC3 puncta was performed with 15-20 images per condition. Total LC3 dots per image were normalized relative to the total area corresponding to all myotubes observed in each image. Myotube area was measured by using ImageJ software. Data were expressed as the number of LC3 dots/mm2.

### LysoTracker Staining

2.5×10⁴ cells were seeded in a 24-well plate. After treatment with ASOs for 48 h, samples were incubated 30 min at 37° C. with 100 nM LysoTracker RED-DND99 (Invitrogen, Madrid, Spain) and 5 μg/ml Hoechst 33258 (Sigma-Aldrich, St. Louis, Missouri). After two washes with warmed PBS, cells were fixed with 4% PFA for 15 min followed by washes in 1×PBS. Then, cells were mounted using fluorescence mounting medium Dako (Agilent Technologies, Santa Clara, California). Images were acquired in an LSM800 confocal microscope (Zeiss) at 400× magnification.

### Toxicity Assay

Non-DM control (Ctrl) cells (CNT) were aliquoted in 96-well plates with 1.0×10⁵ cells per well. After 24 h, cells were transfected as described with different ASOs (concentrations ranging from 1 nM to 1 μM) or treated with Ro-08-2750 (Tocris Bioscience, Bristol, UK) at concentrations ranging from 0.0195 up to 80 μM (in 1:2 serial dilutions). Cell viability was measured as previously described³.

### RNA Extraction, Semiquantitative PCR and Real Time PCR.

All the procedures were performed as previously described¹. Specific primers not previously reported are listed in Table 2.

TABLE 2
Table 2a Sequences of Oligonucleotides Used for qRT-PCR and Semiquantitative RT-PCR

| | | | | SEQ ID |
|---|---|---|---|---|
| Primer name | Sequence (5′ → 3′) | technique | Species | NOs: |
| GAPDH fwd | CATCTTCCAGGAGCGAGATC | RT-qPCR/ | H. sapiens | 45 |
| | | RT-PCR | | |
| GAPDH rev | GTTCACACCCATGACGAACAT | RT-qPCR | H. sapiens | 46 |
| GPI fwd | CAGGGCATCATCTGGGACAT | RT-qPCR | H. sapiens | 47 |
| GPI rev | TCTTAGCCAGCTGCTTTCCC | RT-qPCR | H. sapiens | 48 |
| HPRTI fwd | TGACACTGGCAAAACAATGCA | RT-qPCR | H. sapiens | 49 |
| HPRTI REV | GGTCCTTTTCACCAGCAAGCT | RT-qPCR | H. sapiens | 50 |
| IGF1 fwd | CTCTTCAGTTCGTGTGTGGAGAC | RT-qPCR | H. sapiens | 51 |
| IGFI rev | CAGCCTCCTTAGATCACAGCTC | RT-qPCR | H. sapiens | 52 |
| MSI2 fwd | GCAGACCTCACCAGATAGCCTT | RT-qPCR | H. sapiens | 53 |
| MSI2 rev | AAGCCTCTGGAGCGTTTCGTAG | RT-qPCR | H. sapiens | 54 |
| MSTN fwd | TGAGAATGGTCATGATCTTGCTGT | RT-qPCR | H. sapiens | 55 |
| MSTN rev | TCATCACAGTCAAGACCAAAATCC | RT-qPCR | H. sapiens | 56 |
| mTOR fwd | AGCATCGGATGCTTAGGAGTGG | RT-qPCR | H. sapiens | 57 |
| mTOR rev | CAGCCAGTCATCTTTGGAGACC | RT-qPCR | H. sapiens | 58 |
| NFIX fwd | GAGCCCTGTTGATGACGTGTTCTA | RT-PCR | H. sapiens | 59 |
| NFIX rev | CTGCACAAACTCCTTCAGTGAGTC | RT-PCR | H. sapiens | 60 |
| P21 fwd | AGGTGGACCTGGAGACTCTCAG | RT-qPCR | H. sapiens | 61 |
| P21 rev | TCCTCTTGGAGAAGATCAGCCG | RT-qPCR | H. sapiens | 62 |
| PKM fwd | CTGAAGGCAGTGATGTCGCC | RT-PCR | H. sapiens | 63 |
| PKM rev | ACCCGGAGGTCCACGTCTC | RT-PCR | H. sapiens | 64 |
| ATP2A fwd | GATGATCTTCAAGCTCCGGGC | RT-PCR | H. sapiens | 65 |
| ATP2A rev | CAGCTCTGCCTGAAGATGTG | RT-PCR | H. sapiens | 66 |
| TGFBR1 fwd | GACAACGTCAGGTTCTGGCTCA | RT-qPCR | H. sapiens | 67 |
| TGFBR1 rev | CCGCCACTTTCCTCTCCAAACT | RT-qPCR | H. sapiens | 68 |

Table 2.b Sequences of Oligonucleotides Used for Mouse qRT-PCR

| | | | | SEQ ID |
|---|---|---|---|---|
| Primer name | Sequence (5′ → 3′) | technique | Species | NOs: |
| Gapdh fwd | ATCAACGGGAAGCCCATCAC | RT-qPCR | M. musculus | 70 |
| Gapdh rev | CTTCCACAATGCCAAAGTTGT | RT-qPCR | M. musculus | 71 |
| Gtf2b fwd | ATGGCGGACAGAATCAACCTCC | RT-qPCR | M. musculus | 72 |
| Gtf2b rev | ACAAGCAGAGGCTATCGCGTCA | RT-qPCR | M. musculus | 73 |
| Hprt1 fwd | GTTGGATACAGGCCAGACTTTGT | RT-qPCR | M. musculus | 74 |
| Hprt1 Rev | CACAGGACTAGAACACCTGC | RT-qPCR | M. musculus | 75 |
| Msi2 fwd | CAGCCGAAAGAAGTCATGTTCCC | RT-qPCR | M. musculus | 79 |
| Msi2 rev | CCTCTGCCATAGGTTGCCACAA | RT-qPCR | M. musculus | 80 |
| Mrf4 fwd | ATCAGCTACATTGAGCGTCTACA | RT-qPCR | M. musculus | 81 |
| Mrf4 rev | CCTGGAATGATCCGAAACACTTG | RT-qPCR | M. musculus | 82 |
| Mstn fwd | CTCAGACCCGTCAAGACTCC | RT-qPCR | M. musculus | 83 |
| Mstn rev | CTCTGCCAAATCAATACCAGTGCC | RT-qPCR | M. musculus | 84 |
| Atg4a fwd | CAGTCTCCACAGCGGATGAGTA | RT-qPCR | M. musculus | 85 |
| Atg4a rev | GTGTGATGGGTGCTTCTGAACC | RT-qPCR | M. musculus | 86 |
| Atg7 fwd | GACCGGTCTTACCCTGCTC | RT-PCR | M. musculus | 87 |
| Atg7 rev | TGTGGTTGCTTGCTTCAGAC | RT-PCR | M. musculus | 88 |
| P21 fwd | TCGCTGTCTTGCACTCTGGTGT | RT-qPCR | M. musculus | 89 |
| P21 rev | CCAATCTGCGCTTGGAGTGATAG | RT-qPCR | M. musculus | 90 |
| Pax7 fwd | GAGCACTCGGCTAATCGAAC | RT-qPCR | M. musculus | 91 |
| Pax7 rev | CCGTGTTTCTCATGGTTGTG | RT-qPCR | M. musculus | 92 |
| Tgfbr1 fwd | TGCTCCAAACCACAGAGTAGGC | RT-qPCR | M. musculus | 93 |
| Tgfbrl rev | CCCAGAACACTAAGCCCATTGC | RT-qPCR | M. musculus | 94 |
| Atrogin1 fwd | CTCTGTACCATGCCGTTCCT | RT-qPCR | M. musculus | 95 |
| Atrogin1 rev | GGCTGCTGAACAGATTCTCC | RT-qPCR | M. musculus | 96 |
| Trim63 fwd | ACCTGCTGGTGGAAAACATC | RT-qPCR | M. musculus | 97 |
| Trim63 rev | AGGAGCAAGTAGGCACCTCA | RT-qPCR | M. musculus | 98 |
| Foxo1 fwd | GAGAGCTCAGCCGAGAAGAG | RT-qPCR | M. musculus | 99 |
| Foxo1 rev | CTCCCTCTGGATTGAGCATC | RT-qPCR | M. musculus | 100 |
| Foxo3a fwd | CAGGCTCCTCACTGTATTCAGCTA | RT-qPCR | M. musculus | 101 |
| Foxo3a rev | CATTGAACATGTCCAGGTCCAA | RT-qPCR | M. musculus | 102 |
| Myod1 fwd | AGCACGCACACTTCTCTACT | RT-qPCR | M. musculus | 103 |
| Myod1 rev | GGCCTCATTCACTTTGCTCA | RT-qPCR | M. musculus | 104 |
| Nanoluc Fwd | GGGAGGTGTGTCCAGTTTGT | RT-qPCR | M. musculus | 105 |
| Nanoluc rev | TTCACCGCTCAGGACAATCC | RT-qPCR | M. musculus | 106 |
| HSA Fwd | ACGGGTGCGTGGTGTCTC | RT-qPCR | M. musculus | 107 |
| HSA rev | GGTCAGGATACCTCTCTTGCT | RT-qPCR | M. musculus | 108 |

### Western Blotting

Protein extraction, quantification and immunodetection were performed as previously described^{1,3}. Specifically, MSI2 detections were performed by incubating membranes overnight with a rabbit anti-MSI2 antibody (1:1000, Abcam, EP1305Y, Cambridge, UK). Goat horseradish peroxidase (HRP)-conjugated anti-rabbit-IgG (1:3500, Sigma-Aldrich, St. Louis, Missouri) was used as secondary antibody. Images were acquired with an ImageQuant LAS 4000 or AMERSHAM ImageQuant 800 (GE Healthcare). Quantification was performed using ImageJ software (NIH).

### Jess Fully Automated System

The quantification of V5-tag was determined by Western-blot using the Jess fully automated system (ProteinSimple; Bio-Techne) following the manufacturer's recommendations. Samples were prepared as follows: First, 5× master mix was prepared using reagents provided by Bio-Techne (EZ Standard Pack 1, cat. no.: S-ST01EZ-8). ½ 10× Sample buffer was mixed with ½ 400 mM DTT solution; a final concentration of 0.4 mg/mL of gastrocnemius or quadriceps muscle protein was mixed with 5× master mix, heated at 95° C. for 5 min and stored on ice. Here 12-230 kDa Jess/Wes Separation Module (cat #SM-W004) was used and 4 μl of each sample was loaded for 9 s. The incubation time of the primary and the secondary antibodies was 30 min. As a primary antibody, rabbit anti-V5-Tag (Cell Signalling, D3H8Q, Danvers, Massachusetts) was used at 1:20 dilution. Ready to use HRP-conjugated anti-rabbit antibody module (cat. #DM-001) was used as secondary antibody. Antibodies were prepared in antibody diluent 2 solution. The RePlex Module was used to remove primary and secondary antibodies in a RePlex assay with immunoassay with total protein assay in a single run (Cat #RP-001). Total protein quantification was performed using the total protein detection module for chemiluminescence (cat #DM-TP01).

### Statistical Analyses

In all molecular studies, for comparison on mean data, all parameters were assumed to follow a normal distribution, and the samples were compared using two-tailed t-tests (α=0.05), applying Welch's correction when necessary. The statistical differences were estimated by the Student's t-tests (p<0.05) on normalized data. In all AAV mice neonates studies, for comparison on mean data, all parameters were assumed to follow a normal distribution. The statistical differences were estimated by using One-Way ANOVA or Two-Way ANOVA (α=0.05) test, applying Tukey's HSD post hoc test on normalized data. Sample size (n) is included in the figure legends.

### AAV Synthesis

Cloning and production of adeno-associated virus (AAVs) vectors was done by the Viral Vector Production Unit from Universitat Autonoma de Barcelona (UAB). The construct used for overexpressing isoform 4 of murine Msi2 was AAV9-hDES-mMsi2 (v4)-V5-T2A-Nanoluc. The specific construct used herein is disclosed above in SEQ ID NO: 69.

### Transgenic Mice, AAV Administration and Blood and Tissue Collection

Mouse handling and experimental procedures conformed to the European law regarding laboratory animal care and experimentation (2003/65/CE) and were approved by Conselleria de Agricultura, Generalitat Valenciana (reference number 2019/VSC/PEA/0150). Homozygous transgenic HSA^{LR} (line 20 b) mice were provided by Prof. C. Thornton (University of Rochester Medical Center) and mice with the same genetic background (FVB) were used as controls. Experiments were performed in 4.5-month-old males. Animals received one intraperitoneal shot of 1×10¹² vg or 1.75×10¹² vg. Experimental groups were treated with PBS (n=5 FVB, n=3 HSA^{LR}) or MSI2-AAV (n=3 HSA^{LR}) and at six or ten weeks after the injection, animals were sacrificed and quadriceps and gastrocnemius were harvested. Each muscle was divided into three parts, two were snap-frozen in liquid nitrogen for protein and RNA isolation and the third was frozen in isopentane for histological analyses.

For mice neonates experiment, mouse handling and experimental procedures conformed to the European law regarding laboratory animal care and experimentation (2003/65/CE) and were approved by Conselleria de Agricultura, Generalitat Valenciana (reference number 2021/VSC/PEA/0019). Homozygous transgenic HSA^{LR} (line 20 b) mice were provided by Prof. C. Thornton (University of Rochester Medical Center) and mice with the same genetic background (FVB) were used as controls. Viral injections were performed on transgenic HSA^{LR}. For systemic expression, P2-P3 male pups were injected once in the temporal vein with 35 μl in PBS using a 31G insulin needle with AAV9-hDES-mMSI2 (v4)-V5-T2A-Nanoluc (AAV9-hDES-MSI2 n=10) or AAV9-hDES-Nanoluc (AAV9-hDES, n=15). In both cases, 5×10¹¹ vg was administered per animal. FVB (n=8) and HSA^{LR} (n=8) mice were injected with PBS as control. Six weeks after injection, mice were sacrificed, and quadriceps and gastrocnemius were harvested. Each muscle was divided into three parts, two were snap-frozen in liquid nitrogen for protein and RNA isolation and the third was frozen in isopentane for histological analyses. Body weight was monitored weekly since they were weaned at day 21.

### Electromyography Studies and Forelimb Grip Strength Test.

Both studies were performed before AAV injection and before sacrifice. Analysis was done under general anaesthesia, as previously described³. Briefly, five needle insertions were performed in each quadriceps muscle of both hind limbs, and myotonic discharges were graded on a five-point scale: 0, no myotonia; 1, occasional myotonic discharge in ≤50% of the needle insertions; 2, myotonic discharge in >50% of the insertions; 3, myotonic discharge in nearly all of the insertions; and 4, myotonic discharge in all insertions. The forelimb grip strength was measured at day 21 and before sacrifice as previously described³.

### Fluorescent Histological Analysis of Mouse Muscle Samples

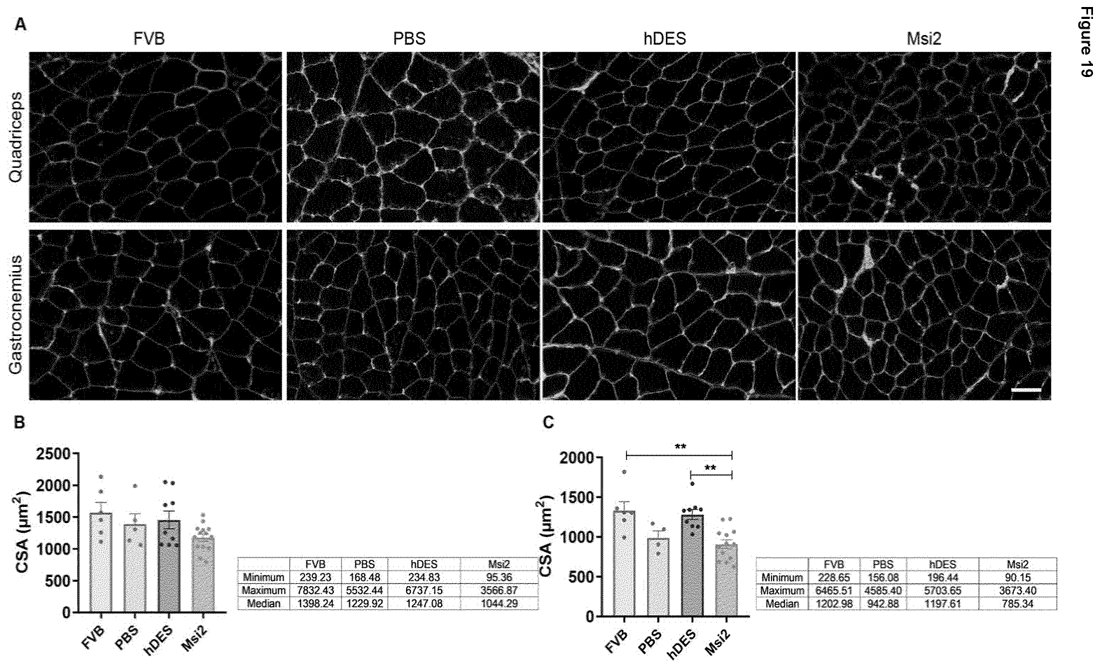

For quantification of muscle fiber cross-sectional area, gastrocnemius and quadriceps were frozen in isopentane and 15 μm-sections were obtained with a Leica CM 1510S cryostat. For muscle fiber cross-sectional area quantification, muscle sections were washed with PBS1× and immunostained with Wheat Germ Agglutinin, Alexa Fluor™ 488 Conjugate (1:200) for 45 min, washed three times with PBS1× and finally mounted with VECTASHIELD® mounting medium containing DAPI (Vector Laboratories, London, UK)) to detect the nuclei. Images were taken in a confocal microscope LSM800 (Zeiss) at 400× magnification. Around 5000 fibers were analyzed in each condition by using a plugin “Muscle morphometry” of Fiji-Image J software (NIH) for FIG. 8 and a Zeiss analysis software (NIH) for FIG. 19.

Sections were stained with hematoxylin-eosin (H&E) and mounted with DPX (Sigma-Aldrich) according to standard procedures. Images were taken at a 100× magnification with a Leica DM2500 microscope. All parameters were analyzed in a total of 500 fibers in each mouse by using ImageJ software (NIH).

### REFERENCES

1. Sabater-Arcis, M., Bargiela, A., Furling, D., and Artero, R. (2020). miR-7 Restores Phenotypes in Myotonic Dystrophy Muscle Cells by Repressing Hyperactivated Autophagy. Mol. Ther.-Nucleic Acids 19, 278-292.
2. de Luna N, Gallardo E, Soriano M, Dominguez-Perles R, de la Torre C, Rojas-García R, García-Verdugo J M, Illa I. (2006). Absence of dysferlin alters myogenin expression and delays human muscle differentiation “in vitro”. J Biol Chem. 28125:17092-8.
3. Bargiela, A., Sabater-Arcis, M., Espinosa-Espinosa, J., Zulaica, M., Lopez de Munain, A., and Artero, R. (2019). Increased Muscleblind levels by chloroquine treatment improve myotonic dystrophy type 1 phenotypes in in vitro and in vivo models. Proc. Natl. Acad. Sci. 116, 25203-25213.

### Example 2

The transcript and protein levels of MSI2 were measured and compared between human biopsies of healthy people and patients suffering from MD1. Table 3 below shows the fold change values of MD1 patients vs healthy (used herein as reference), where it can be observed that the MSI2 RNA levels are increased in patients suffering from MD in a fold change of 1.358, and MSI2 protein levels are increased in patients suffering from MD in a fold change of 1.594. Further, the miR-7 levels were also compared in healthy controls versus MD1 biopsies, showing a decrease in miR-7 intracellular levels in patients of 0.58 fold relative to controls. Lastly, the levels of RNA TGFBR1 were also measured, since TGFBR1 is a gene whose mRNA is directly increased by MSI2 activity. The results showed that the TGFBR1 levels were increased 1.4 fold with respect to healthy people.

TABLE 3
Fold Change Values MD1 versus Healthy Patients

| | | Human Biopsies |
|---|---|---|
| MSI2 | RNA | 1.358 |
| | Protein | 1.594 |
| Mir-7 | | 0.58 |
| TGFBR1 | RNA | 1.4 |

A similar comparison was performed in MD1 primary myoblasts in comparison with healthy control cells, where the fold change values are shown in Table 4 below:

TABLE 4
Fold Change Values MD1 Cells versus Control Cells

| | | MD1 primary |
|---|---|---|
| | | myoblasts |
| MSI2 | RNA | 1.8 |
| | Protein | 2 |
| Mir-7 | | 0.51 |

### Example 3

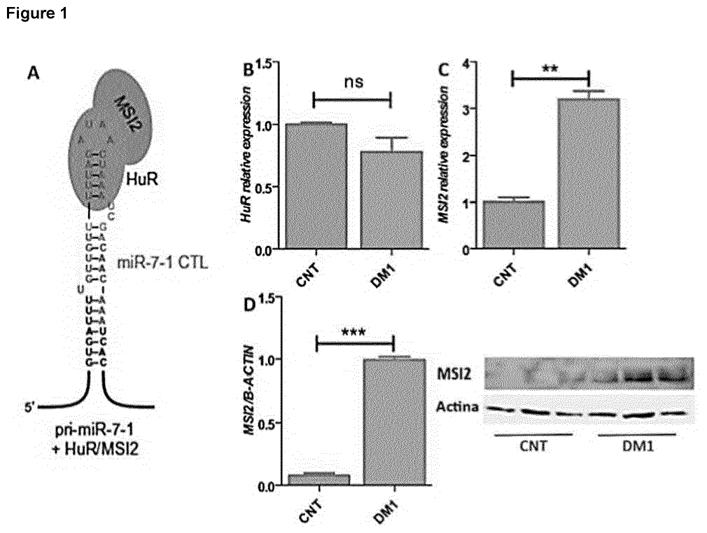

Relative expression levels of HuR and MSI2 transcripts in healthy control and MD1 cells were analysed, and the results showed that, while no significant differences were found in HuR at the transcript or protein levels, MSI2 was strongly upregulated in two cell models of disease, in muscle biopsies, and in RNA-seq data that were accessed through data mining. GAPDH expression was used as internal reference (FIGS. 1 B and C). Further, Western blot quantification of total levels of MSI2 in control and MD1 cells confirmed these results, as shown in FIG. 1 D.

The upregulation of MSI2 and downregulation of miR-7 was further confirmed in MD1 samples, as shown in FIG. 2, where the relative expression of miR-7, MSI2 transcripts and MSI2 protein levels were quantified in patient-derived biopsies (FIG. 2A), primary myoblasts (FIG. 2 B) and TDM (FIG. 2 C). In all cases, significantly lower levels of miR-7 in MD1 samples were detected. When the levels of MSI2 at both transcript and protein levels were studied, a MSI2 upregulation in muscle biopsies was found, but this upregulation was spectacularly increased in TDMs (near 60 times at protein levels compared MD1 TDM cells to control cells).

FIG. 2 G shows MSI2 transcripts per million in RNA-seq experiments from biopsies from 40 MD1 patients and 10 controls according to Wang et al. 2019, where it can be observed that MSI2 presents higher transcripts per million in comparison with control (CNT) biopsies.

Next, MSI2 was immunostained in healthy control (CNT) and MD1 myotubes (TDM), as shown in the confocal images of FIG. 2 E-F, where it can be observed that MSI2 is overexpressed in MD1 cells.

Lastly, FIG. 2 D shows the Pearson's correlation between miR-7 relative expression and MSI2 protein levels, which confirmed a negative correlation in biopsies and TDMs consistent with our findings that increased MSI2 in MD1 inhibits mature miR-7 and thus downregulate levels in MD1 (FIG. 1 D). This correlation was not observed when primary myoblasts were analysed. To further validate MSI2 overexpression in MD1 and that this alteration was relevant in the disease, publicly available RNA-Seq and ankle dorsiflexion strength (ADS) data from 40 MD1 patients and ten controls (obtained from Wang et al. 2019) were used, and it was found that MSI2 was significantly overexpressed (over 35%) in MD1 patients compared to controls in full agreement with results obtained in our own muscle samples. The analysis also supported that MSI2 levels negatively correlated to ankle dorsiflexion strength (FIG. 2H; r=−0.48, P=0.0017). Data according to Wang et al. 2019 [Wang, E. T. et al. Transcriptome alterations in myotonic dystrophy skeletal muscle and heart. Hum Mol Genet 28, 1312-1321, doi: 10.1093/hmg/ddy432 (2019)]

In view of these results, the inventors found a tight inverse correlation between miR-7 and MSI2: the more MSI2, the less miR-7.

### Example 4

Next, the inventors evaluated potential targets within the transcript sequence of MSI2 (SEQ ID NO: 1) that could be targeted by oligonucleotides or siRNA candidates, with the aim of impairing the function or activity or levels of the MSI2 transcript, and eventually reducing the activity or the protein levels of MSI2. First, to establish potential regions that could be targeted, the inventors performed a bioinformatics analysis that consisted in the selection of highly functional siRNA using the siRNA design software, siDirect 2.0 siDirect2.0 (http://siDirect2.RNAi.jp/). This software advocates the Ui-Tei rules, which satisfy the following four conditions simultaneously: (1) A or U at position 1 from 5′ terminus of siRNA guide strand, (2) G or C at position 19, (3) AU richness (AU≥4) in positions 1-7, and (4) no long GC stretch >10. To avoid seed-dependent off-target effects, selecting the siRNAs with low Tm of the seed-target duplex should minimize seed-dependent off-target silencing. The Tm of 21.5° C. may serve as the benchmark, which discriminates the almost off-target-free seed sequences from the off-target-positive ones. In the third step, siRNAs that have near-perfect matches to any other non-targeted transcripts were eliminated.

The analysis resulted in several target regions (SEQ ID NO: 3 to 37) within the MSI2 transcript that could be potentially targeted by oligonucleotide candidates, and it was found that these target regions are mainly concentrated between the nucleotides 327-1618 (SEQ ID NO: 2) of the MSI2 full transcript.

In view of this, the inventors designed several oligonucleotides comprised within this region (nucleotides 327-1618 (SEQ ID NO: 2)) but also one oligonucleotide that falls outside this region (from nucleotides 1618 to the end of the transcript), in order to test their effect in reducing MSI2 function. Thus, the oligonucleotides were designed and synthesized to be tested in vitro. Two gapmer antisense oligonucleotides (ASO1 and ASO3, SEQ IDS NO: 38 and 39, respectively) against MSI2 transcript were designed to trigger RNAse-H mediated degradation of the target where they bind. ASO1 falls within the region detected by the bioinformatics analysis, while ASO3 hybridized outside this region

Initially, ASOS' toxicity was assayed in control TDMs and it was observed that they present low toxicity as near 1000 nM the cell viability was still higher than 50%. Two different concentrations were used in subsequent experiments: 30 nM, at which around 90% of cells were viable, and 150 nM, at which between 70%-80% of the cells were viable when transfected with any of the two ASOs (FIG. 3A). To confirm the activity of the ASOs, MSI2 relative expression was measured upon transfection in DM1 TDMs. A significant reduction of MSI2 levels was observed with ASO1 and 3, but ASO1 was better at both tested concentrations. Similar results were observed quantifying MSI2 protein levels (FIGS. 3 B and C). miR-7 was derepressed in ASO-treated MD1 cells (FIG. 3 D). The effect of reduced MSI2 levels on P21 and TGFBR1 targets was also studied (FIG. 3 E). P21 was strongly derepressed in muscle cells treated with ASO3 at the lowest concentration, while TGFBR1 behaved the opposite at 150 nM of ASO1. After seven days of differentiation, TDMs were stained with an antibody against Desmin. Quantification of fusion capacity and myotube diameter showed that reducing MSI2 levels in DM1 TDMs restored the fusion index dramatically, in a dose-dependent manner in the case of ASO3, and the size of the myotubes (FIGS. 3 F and G). FIGS. 3 H, I and J show representative confocal images of Desmin-immunostained human fibroblasts transdifferentiated for 7 days after ASOs transfection into MD1 cells and their respective scramble control at 150 nM. The scrambled sequence ASO did not significantly reduce MSI2 transcripts at 150 nM in transfected control TDMs (Figure FIG. 3k).

Thus, as shown in FIG. 3, both ASO1 and 3 significantly lowered the amount of MSI2 transcripts in MD1-derived myoblasts at two concentrations (30 nM and 150 nM). The inventors quantified a reduction in 27% of MSI2 transcript after 150 nM ASO1 treatment, and in 46% when cells were transfected with 30 nM ASO3, which were the conditions at which the highest effect was observed.

Concomitant with reduced MSI2 levels, a strong rescue in two atrophy-related myotube parameters was found: the fusion index (percentage of nuclei within myotubes, indicating a normal or pathogenic ability to fuse of MD muscle cells) and the myotube diameter, which are very low in MD1 cells and the treatment brings back to close to normal levels.

### Reducing MSI2 Boosts MBNL1 Levels in DM1 TDMs.

Considering the key role of MBNL and CELF1 proteins in the pathogenesis of DM1 and previous results where inhibition of autophagy in DM1 promoted MBNL1 activity, we quantified MBNL1 and MBNL2, and CELF1 levels, after ASO treatments. Our data show that when DM1 muscle cells were treated with ASOs at 150 nM, MBNL1 expression was enhanced, achieving levels 50% higher than those obtained in cells treated with scrambled ASO (FIG. 4A). 2). In contrast, MBNL2 and CELF1 levels remained unchanged after ASOs treatment (FIG. 4 B-C and FIG. 4 S-V). These results were confirmed by immunofluorescence staining to detect MBNL1 and MBNL2 in ASO-treated DM1 TDMs. For MBNL1, a low concentration of ASOs did not change the intensity or the pattern of the signal compared to scrambled-treated controls but at the high ASO concentration, a boost in the green signal became evident both in the cytoplasm and in the cell nucleus (FIG. 4 D-I). An antibody against MBNL2 showed results consistent with the western blot quantification, except for a slight increase with 150 nM ASO1, although the signal was very weak given the low expression of MBNL2 (FIG. 4 J-O). To confirm that the additional protein detected was functional, we studied splicing defects regulated by MBNL proteins (FIG. 4 P-R). ASO-induced MBNL1 increase was sufficient to reduce the inclusion of pyruvate kinase (PK) exon 10 (fetal PKM2 isoform) in all the experimental conditions evaluated. The inclusion of SERCA1 exon 22 (ASO3) or NFIX exon 7 (both tested ASOs) further confirms the relevance of the extra MBNL1 detected.

Further, cells were transfected with a combination of two siRNAs (SEQ ID NO: 40 and 43) targeting MSI2 transcripts. The results showed that the siRNAs similarly reduce MSI2 relative expression (FIG. 5A), enhance miR-7 relative expression (FIG. 5 B), and rescue the fusion index in patient cells, as shown in FIG. 5 C-E. Myotube diameter trended higher than controls upon MSI2 silencing (FIG. 5F). Additionally, we quantified TGFBR1 levels, as it was demonstrated to be directly regulated by MSI224 and also P21, which, in contrast to TGFBR1, is inhibited by MSI2.25 TGFBR1 levels dropped (near 40%), and P21 levels, however, did not significantly change (FIG. 5G).

Thus, it is shown herein that oligonucleotides directed to target several regions within the SEQ ID NO: 1 (full transcript), but especially within the region of SEQ ID NO: 2 (partial transcript, nucleotides 327-1618) of MSI2, are able to reduce the levels of MSI2 mRNA, causing an increase in the intracellular levels of miR-7. Overall, the level of silencing achieved with ASOs was higher than that obtained with siRNAs, but both oligonucleotides led to significant changes in direct MSI2 targets.

### Example 5

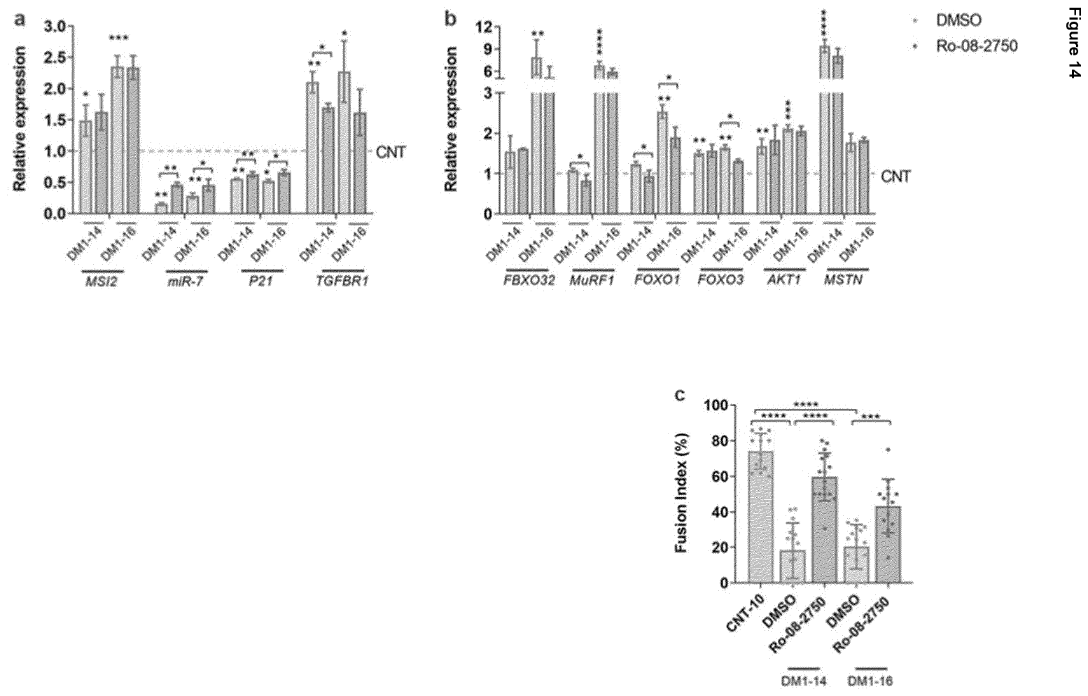

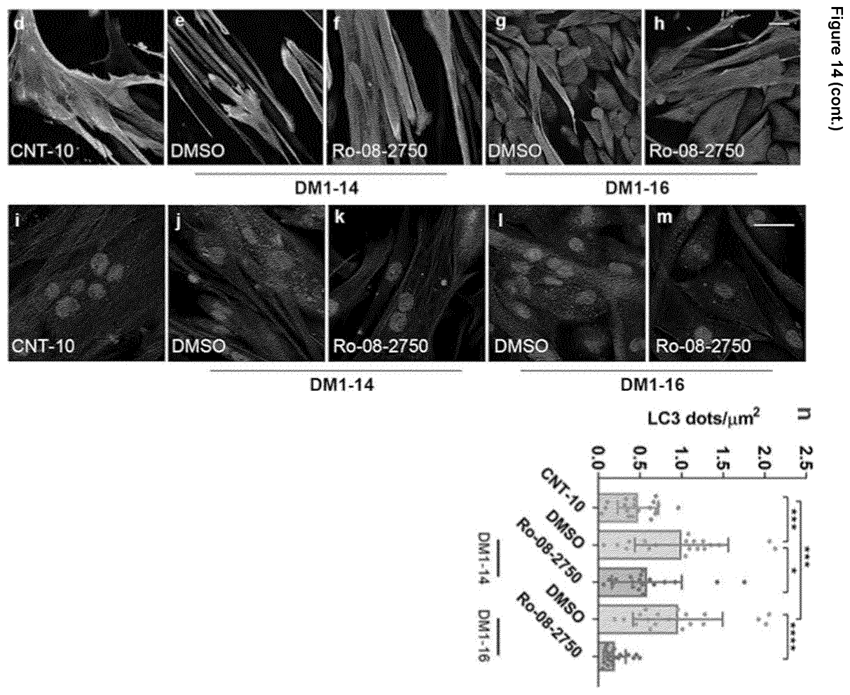

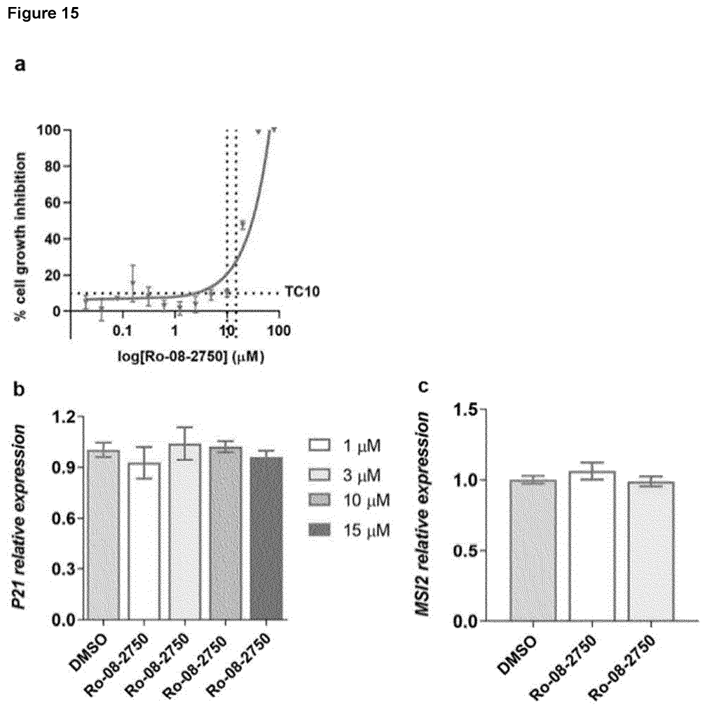

Additionally, other inhibitors and strategies were tested to reduce MSI2 levels with mechanisms very different from those presented in FIGS. 3-5. In this case, the small molecule Ro-08-2750 that has been shown to bind directly and selectively to MSI2 and competes for its RNA binding was used (FIG. 6, FIG. 15). Further, another mechanism was explored to achieve MSI2 reduction, which consisted in the treatment of muscle cells with an oligonucleotide that mimics miR-107, named AgomiR-107 (SEQ ID NO: 41). miR-107 negatively regulates MSI2 by directly binding to its 3′UTR region (FIG. 7). The inventors observed that MSI2 levels were significantly reduced at transcript and protein levels when cells were treated with the small molecule Ro-08-2750 at a concentration of 10 μM or the agomiR (FIGS. 6A and G and 7 A and G). Downregulation of MSI2 correlated with significant derepression of miR-7 (FIGS. 6B and 7B) and downregulation of autophagy markers (FIG. 6 C, D, H-J and FIG. 7 C, D, H-J) and autophagic flux (FIG. 6K and FIG. 7K). Next, expression levels of P21 and TGFBR1, two direct targets of MSI2 were analysed and it was found that treatment with the small molecule was sufficient to reduce TGFBR1 levels but not to produce a significant effect on P21 (FIGS. 6 E and F). However, the addition of agomiR-107 resulted in significant modulation of both targets (FIGS. 7 E and F). 3 μM was sufficient to significantly de-repress miR-7 biogenesis leading to levels similar to those obtained with the 10 μM treatment (FIG. 15).

Another parameter analysed was the fusion capacity by Desmin immunostaining of the cells after treatment with Ro-08-2750 or agomiR-107 (SEQ ID NO: 41). It was found that the fusion index was significantly improved after MSI2 reduction with any of the treatments (FIGS. 6 L and M and FIGS. 7 L and M). Lastly, the diameter of the cells improved significantly when cells were treated with Ro 08-2750 (FIG. 6 N).

### Example 6

The increase in intracellular miR-7 expression in the MD treated cell with the inhibitor at different concentrations was quantified. Table 5 below shows the fold-change increase of miR-7 expression in comparison to reference MD1 cells.

TABLE 5
Increase of miR-7 Levels in Treated Cells.

| | Low | High |
|---|---|---|
| FC miR-7 expression respect to MD1 | concentration | concentration |
| ASO 1 (30 and 150 nM) | 1.49 | 1.72* |
| ASO 3 (30 and 150 nM) | 1.68* | 2.25* |
| Ro 08-2750 (10 μM) | 2.52* | |
| siRNA (100 nM) | 3.3 | |

A significant miR-7 increase relative to MD1 cells is observed when treating MD1 cells with ASO3 at the lowest concentration (30 nM). Under these conditions, a significant 1.68-fold increase (68%) in miR-7 was obtained with respect to MD1 cells treated with the scramble sequence control at 30 nM. Importantly, this increase was sufficient to significantly rescue the fusion capacity of the MD1 myotubes, both fusion index and diameter, and some gene splicing events related to the disease (PKM2, SERCA and N-FIX), as shown in FIGS. 3 and 4.

Further, the reduction in MSI2 that leads to a lower increase in miR-7 but that does have a positive effect on MD1 cells is the treatment with ASO1 at a concentration of 30 nM. In this case, a 1.49-fold (49%) increase in miR-7 levels was obtained, which translated into a significant rescue in the fusion capacity (fusion index and diameter) (FIG. 3). Lastly, in the case of the small molecule Ro 08-2750 used at 10 μM, the increase in intracellular miR-7 levels achieved is of 2.52 times (252%). Phenotype rescues are observed in terms of the fusion index and diameter of the MD1 myoblasts after treatment with the inhibitors, as shown for ASOs in FIG. 3 or for small molecule Ro 08-2750 in FIG. 6.

Altogether, these results indicate that the use of inhibitors of MSI2 function of different nature (oligonucleotides, small compounds) are able to rescue the phenotype of treated cells. The effects of these inhibitors can be measured by the increase in miR-7 levels in comparison to MD1 untreated cells, where an increase of about 50% already produces a phenotype change in MD1 cells in terms of the fusion index and diameter.

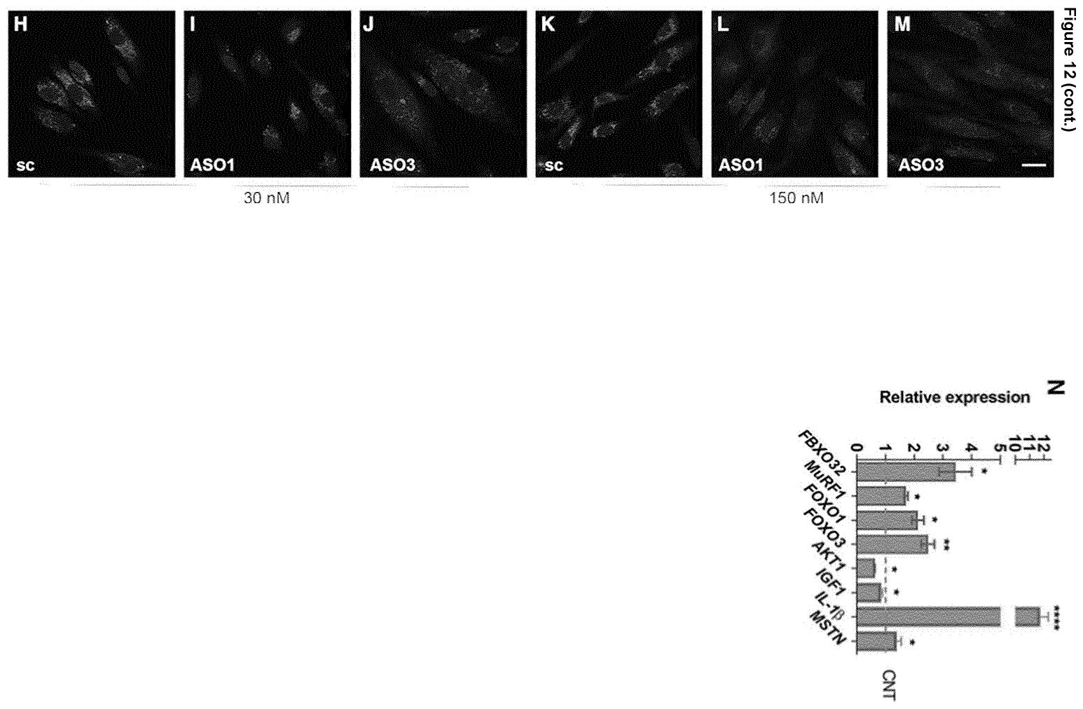

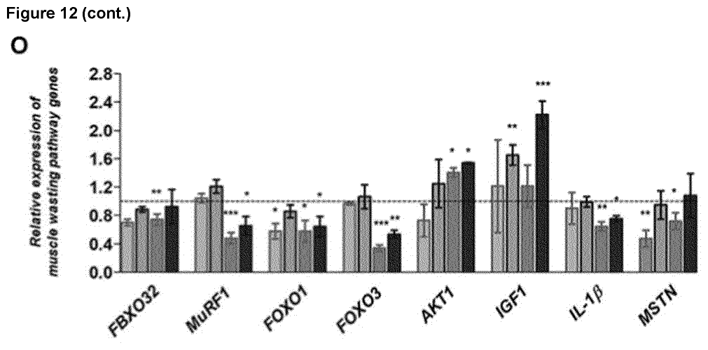

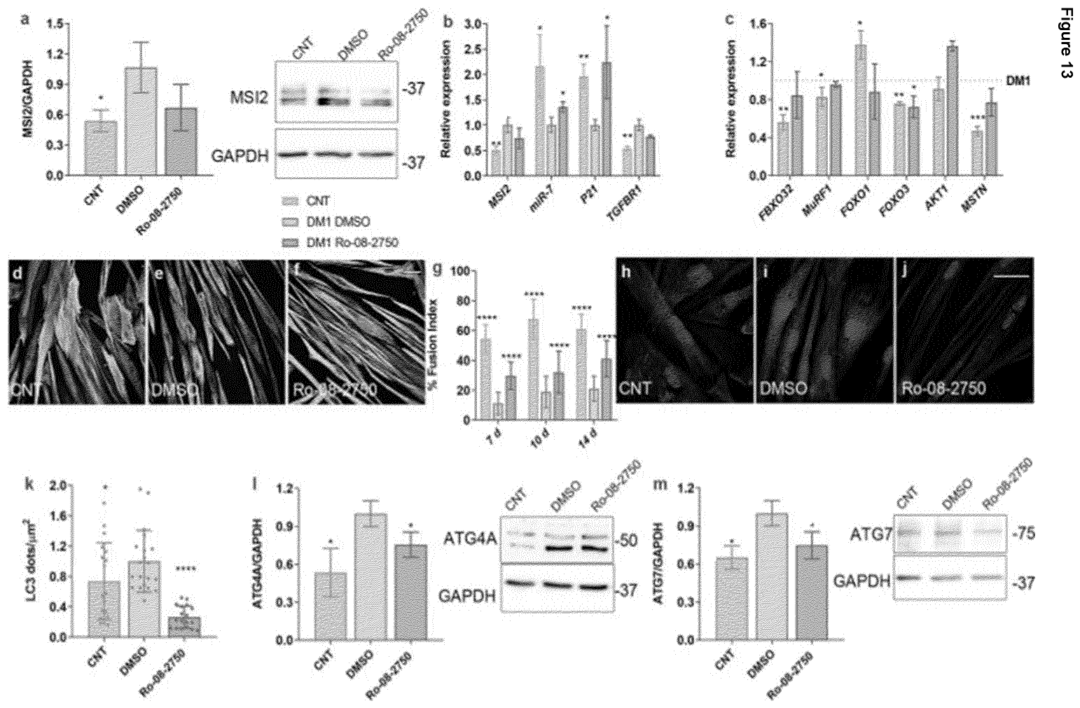

To validate results, we treated independent DM1 cell lines with the MSI2 inhibitor Ro 08-2750. Specifically, we treated immortalized myoblasts differentiated for 7 days from the same donor as for TDMs (FIG. 13) and two primary myoblast lines obtained from additional patient biopsies (Table 7; FIG. 14). We confirmed that MSI2 levels were significantly increased compared to controls, whereas miR-7 behaved inversely (FIGS. 13A and 13B; FIG. 14A).

TABLE 7
Information of Biopsies from Skeletal Muscle.

| | | | | | Repeats | |
|---|---|---|---|---|---|---|
| | Sample | Sex | Age | Muscle | length | |
| Controls | CNT-1 | Female | 61 | Deltoid | nd | |
| | CNT-2 | Male | 34 | Deltoid | nd | |
| | CNT-3 | Female | 35 | Deltoid | nd | |
| | CNT-4 | Male | 37 | Biceps | nd | |
| | CNT-5 | Male | 24 | Deltoid | nd | |
| | CNT-6 | Male | 25 | Deltoid | nd | |
| | CNT-7 | Female | 29 | Deltoid | nd | |
| | CNT-8 | Female | 46 | Deltoid | nd | |
| | CNT-9 | Male | 29 | Deltoid | nd | |
| | CNT-10(*) | Male | 18 | Medial | nd | |
| | | | | gastrocnemius | | |
| | CNT-11 | Male | 50 | Deltoid | nd | |
| | CNT-12 | Male | 63 | Deltoid | nd | |
| | CNT-13 | Male | 59 | Deltoid | nd | |
| | CNT-14 | Male | 47 | Deltoid | nd | |
| | CNT-15 | Female | 34 | Deltoid | nd | |
| | CNT-16 | Male | 55 | Deltoid | nd | |
| | CNT-17 | Female | 37 | Deltoid | nd | |
| Patients | DM1-1 | Male | 30 | Deltoid | nd | |
| | DM1-2 | Male | 61 | Deltoid | 0.3 | kb |
| | DM1-3 | Male | 45 | Deltoid | 3.6 | kb |
| | DM1-4 | Female | 69 | Deltoid | 1.1 | kb |
| | DM1-5 | Male | 28 | Deltoid | nd | |
| | DM1-6 | Female | 65 | Deltoid | nd | |
| | DM1-7 | Female | 33 | Deltoid | 0.9 | kb |
| | DM1-8 | Male | 32 | Deltoid | 0.4 | kb |
| | DM1-9 | Female | 68 | Deltoid | 0.2 | kb |
| | DM1-10 | Male | 27 | Deltoid | 0.75 | kb |
| | DM1-11 | Female | 44 | Deltoid | nd | |
| | DM1-12 | Male | 52 | Deltoid | nd | |
| | DM1-13 | Male | 36 | Deltoid | 3 | kb |
| | DM1-14(*) | Female | 50 | Deltoid | 150 | |
| | DM1-15 | Female | 47 | Deltoid | nd | |
| | DM1-16(*) | Male | 54 | Deltoid | 0.8 | kb |

nd: not determined
(*)biopsies used to isolate primary cells

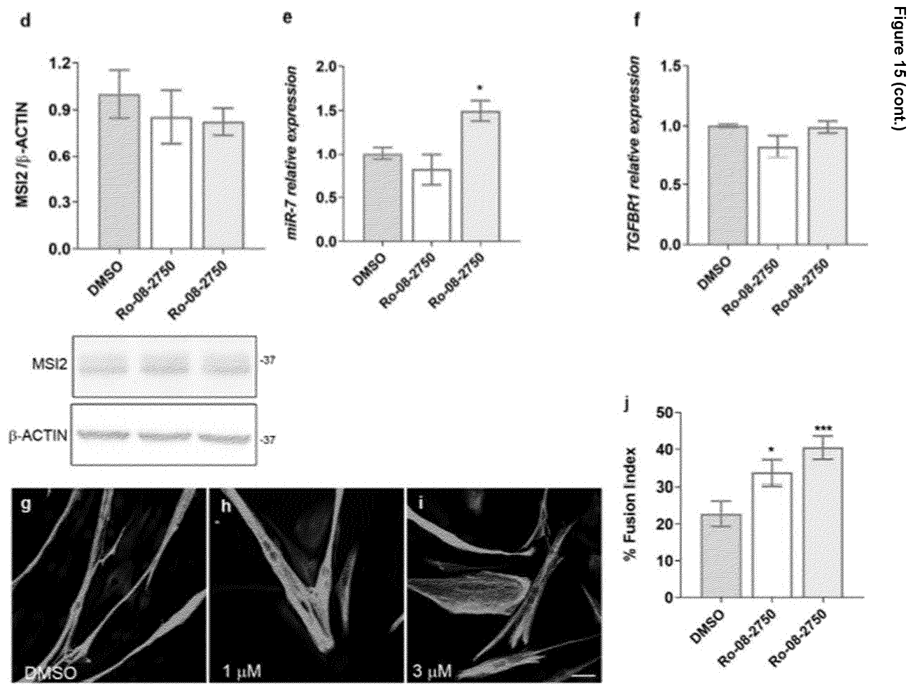

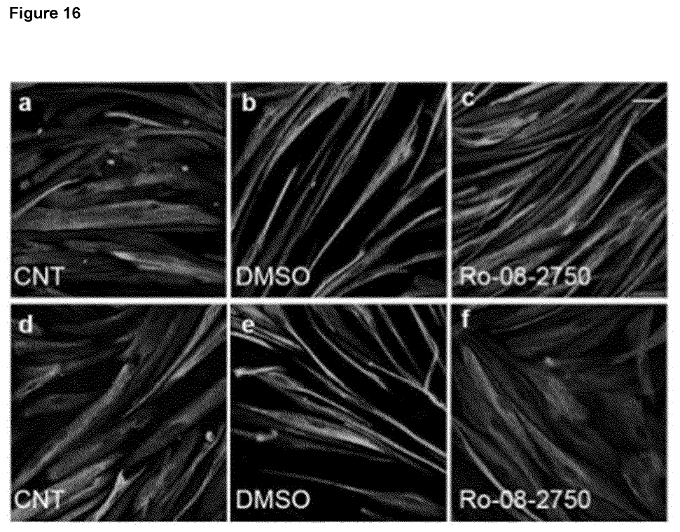

Notably, Ro 08-2750 at 10 μM rescued the expression of the direct MSI2 targets miR-7 and P21 in all cell lines and TGFBR1 in the DM1-14 (FIG. 13B; FIG. 14A). Similarly, UPS system and muscle degradation genes showing impaired expression in DM1 muscle cells were significantly rescued upon treatment (FIG. 13C; FIG. 14B). Additionally, we evaluated by anti-Desmin immunofluorescence the fusion capacity of immortalized myoblasts at 7, 10, and 14 days of differentiation (FIGS. 13D-13G; FIG. 16). In all cases, the capacity of DM1 muscle cells was much lower when compared to control counterparts. Notably, the fusion index was significantly improved after treatment with the compound, namely 160%, 77%, and 95% at days 7, 10, and 14, respectively, compared to DM1 cells treated with vehicle only. In the case of primary myoblasts cultured for 7 days, it was also confirmed that the fusion capacities of both DM1-14 and DM1-16 lines were significantly impaired compared to healthy controls (FIGS. 14C-14H) and that this phenotype was dramatically reversed after the addition of 10 μM Ro 08-2750. Finally, DM1 cells showed increased LC3 puncta formation (FIGS. 13H-13K; FIGS. 141-14N), confirming the hyperactivation of the autophagic pathway. In immortalized myotubes, this was reinforced by the detection of increased ATG4A and ATG7 protein levels (FIGS. 13L and 13M). Treatment with the small molecule was sufficient to reverse autophagic markers to a non-DM1 state dramatically.

### Example 7

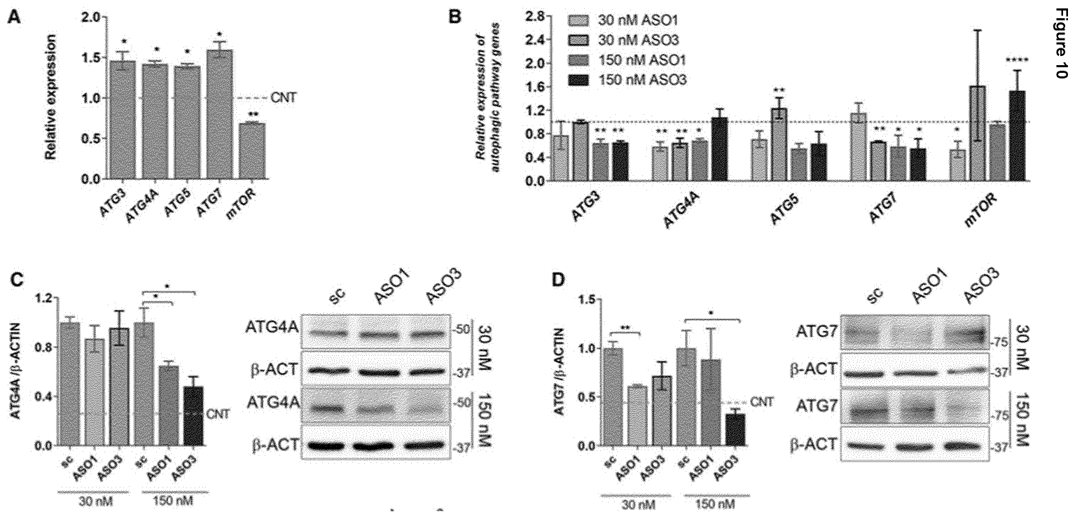

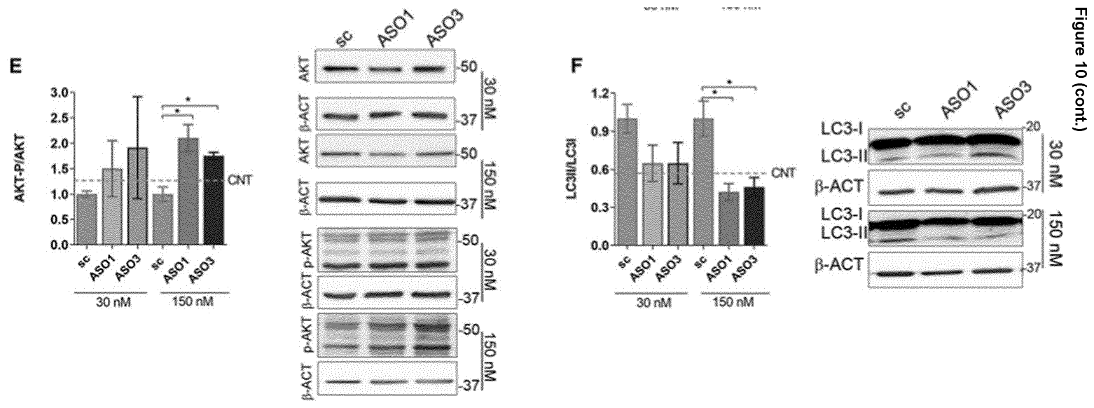

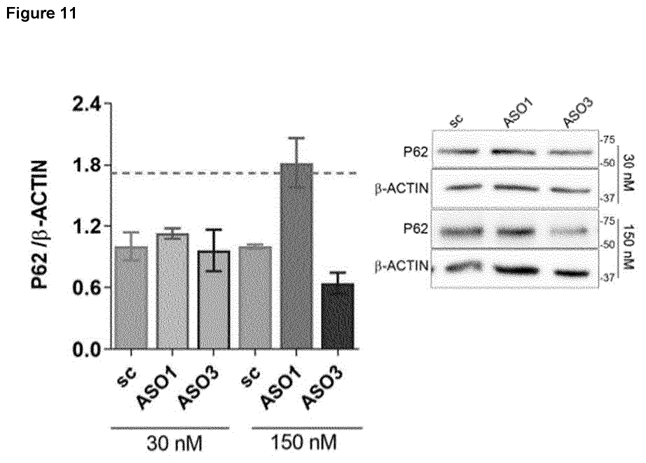

Given the involvement of MSI2 in miR-7 biogenesis, we investigated the expression of critical autophagy pathway genes in DM1 and control TDMs, namely ATG3, ATG4A, ATG5, ATG7, and mTOR (FIG. 10A). Their expression was rescued significantly, approaching normal levels in all cases, after treatment with ASOs at one or both concentrations tested, except ATG5, which only significantly reduced its expression at the highest concentration of ASO1 (FIG. 10B). Autophagy status in DM1 TDMs in response to MSI2 levels was also assessed at the protein level. We evaluated the expression of ATG4A and ATG7, direct miR-7 targets, and observed that ATG4A was dramatically reduced (around 50% compared to scrambled ASO) at the highest concentration of ASOs, whereas ATG7 was also repressed in response to low MSI2 upon ASO transfection (FIGS. 10C and 10D). Additionally, we tested the activation of the AKT pathway by calculating the phospho (Ser473)-AKT (AKT-P)-to-total-AKT ratio. This is a relevant parameter, as AKT controls both protein synthesis via mTOR and protein degradation (including autophagy) via the FoxO family of transcription factors.26,27 Our data show a robust increase in AKT-P/AKT ratio in cells treated with 150 nM ASO1 or ASO3 (FIG. 10E). The soluble-to-autophagosome-associated-LC3 (LC3-I and LC3-II, respectively) ratio was also quantified since the transformation of LC3-I into LC3-II is an indicator of increased autophagic flux in cells.28 Notably, autophagic flux dropped to about one-half when MSI2 was silenced with the highest concentration of ASOs (FIG. 10F). Finally, we quantified P62 scaffold protein levels that deliver proteins committed for lysosomal degradation to the autophagosome. Low levels of autophagic activity lead to the accumulation of P62, as autophagy itself degrades the protein. We did not detect any significant increase in P62 levels upon ASO treatment (FIG. 11).

To further evaluate the autophagy status in DM1 TDMs treated with ASOs, we performed immunofluorescence staining to detect LC3. In cells transfected with the scrambled ASO, a heavily punctated pattern was observed. These spots correspond to LC3 conjugated to membrane-bound phosphatidylethanolamine (LC3-II) in autophagosomes.

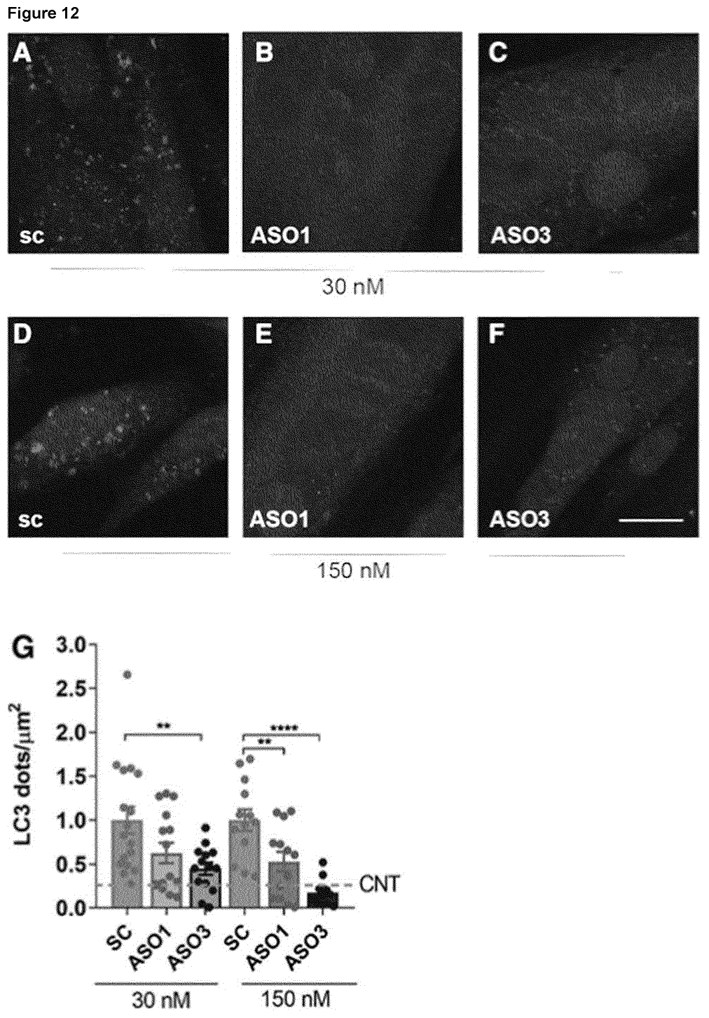

However, upon reduction in MSI2 levels, these spots disappear, and the signal becomes mainly cytoplasmic and diffuse, indicating an increase in soluble LC3-I and, therefore, lower autophagic flux (FIGS. 12A-5F). We analyzed LC3 immunofluorescence results by quantifying LC3 dots per unit of area (FIG. 12G). Analyzed dots correspond to the LC3-II isoform involved in the autophagosome formation, an essential step in the mechanism underlying autophagy. Our results confirmed increased puncta formation in DM1 TDMs (around four times) and also that this parameter can be modulated upon MSI2 targeting with ASOs. Both ASO1 and -3 caused a significant decrease in LC3 dots when added to DM1 TDMs compared to cells treated with the scrambled ASO at the same concentration. The acidotropic dye LysoTracker marks the acidic cellular compartments, including lysosomes and autophagolysosomes, which are excellent markers of autophagy flux levels. When the muscle cells were treated with the ASOs at the lowest concentration, no noticeable change was observed in the signal; however, treatment with high ASO concentration strongly lowered the signal, thus confirming the reduction in autophagic activity when MSI2 gets silenced (FIGS. 12H-12M).

### Example 8

Finally, miR-7 levels in different tissues (quadriceps, gastrocnemius, diaphragm) were measured in an MD1 mouse model, known as HSA^{LR} and, surprisingly, no significant differences were found in comparison to control mice (FVB) (FIG. 9 C) MSI2 levels were quantified (RNA and protein), and it also did not display significant alterations compared to normal controls (FIGS. 9 A and B). Also, consistent with the hypothesis that overactivation of autophagy causes muscle atrophy, it was observed that the activity of this pathway was normal in HSA^{LR} model mice, which was assessed by quantification at the protein level of the ratio LC3I/LC3II, Atg4a, Atg7, and P62 (see FIG. 9 D).

Thus, combining patient-derived cell data and in vivo observations, it was hypothesized that the ultimate reason why the HSA^{LR} model mice fail to show strong muscle degeneration is its failure to increase MSI2 protein levels. Therefore, if artificially overexpressed, MSI2 may interfere with miR-7 biogenesis, and reduced levels of mature miR-7 will release repression of several autophagy-related genes, which in turn will enhance muscle catabolismbeyond normal homeostasis and lead to muscle degradation. Considering this hypothesis, a pilot study was carried out with a reduced number of animals in which the inventors overexpressed murine Msi2 by using AAV. The tested doses were 1×10¹² and 1.75×10¹² vg/mouse and animals were sacrificed at 6 and 10 weeks after the infection. Significantly increased Msi2 levels were observed in mice treated at the highest dose and sacrificed 10 weeks after the injection (FIGS. 8A and B). Moreover, miR-7 was reduced in this experimental group (FIG. 8 C). At the functional level, a forelimb grip strength test was performed before starting the treatment, at an intermediate time point, and after euthanasia (FIG. 8 D). It was found that mice overexpressing Msi2 showed reduced force in their forelimbs in a dose and time-dependent manner. Additionally, stained cryosections of quadriceps with WGA (wheat germ agglutinin) were performed to visualize cell membranes and quantify the cross-sectional area of the muscle fibers (FIG. 8 E, F). Data showed that there was not a significant difference between control mice (FVB) and model (PBS). However, the area was strongly reduced in MD1 mice treated with the highest AAV dose. Similar results were observed when the analysis was performed on gastrocnemius. Finally, RT-qPCR in treated muscle samples revealed increased levels of autophagy-related genes Atg7 and Atg4 (FIG. 8 G), suggesting increased activity of this pathway. Moreover, treated mice showed overexpression of the atrogenes Fbxo32 (Atrogin-1) and Trim63 (MuRF1), thus indicating increased activity of the UPS system.

Table 6 below shows the fold change values in mice treated with AAV versus control mice caused by the overexpression of MSI2 by the AAV, showing that the overexpression of MSI2 induces a reduction in miR-7 and an increase in TGFBR1 levels.

TABLE 6
Fold Change Values in Mice Treated
with AAV versus Control Mice

| | | AAV | AAV |
|---|---|---|---|
| | | GASTROCNEMIUS | QUADRICEPS |
| MSI2 | RNA | 1.31 | 1.96 |
| | Protein | 1.51 | 1.56 |
| Mir-7 | | 0.78 | 0.7 |
| TGFBR1 | RNA | 1.22 | 1.43 |
| CSA (% reduction) | | 45.44% | 35.66% |

Taken together these results reveal that MSI2 overexpression is sufficient to trigger activation of molecular mechanisms that contribute to muscle atrophy in MD1.

### Example 9

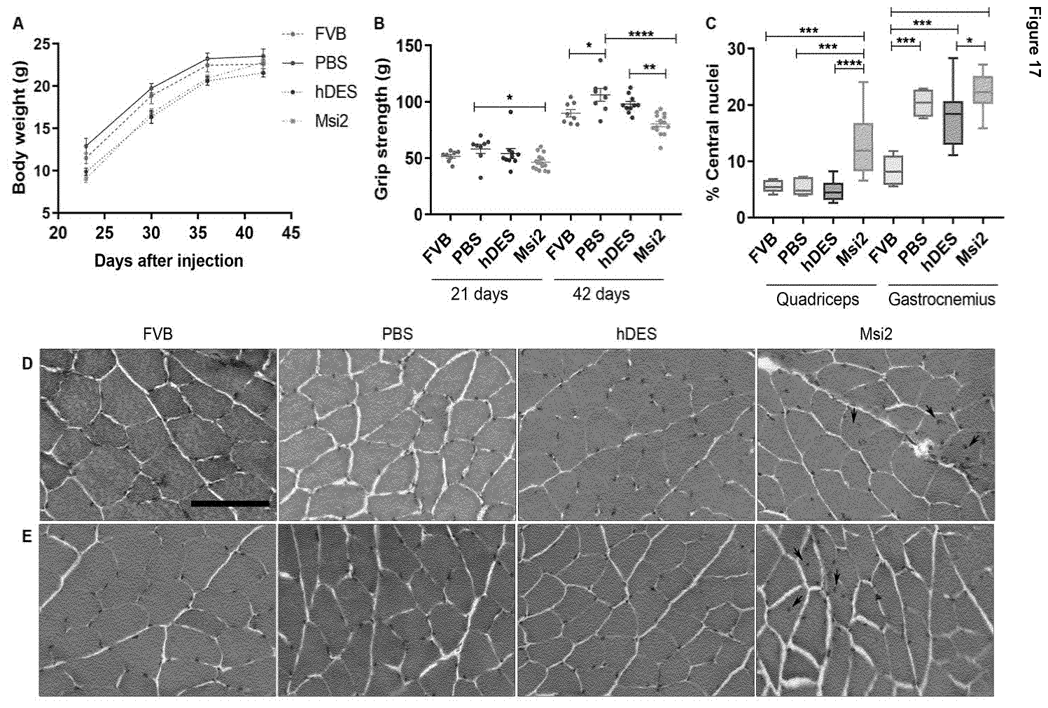

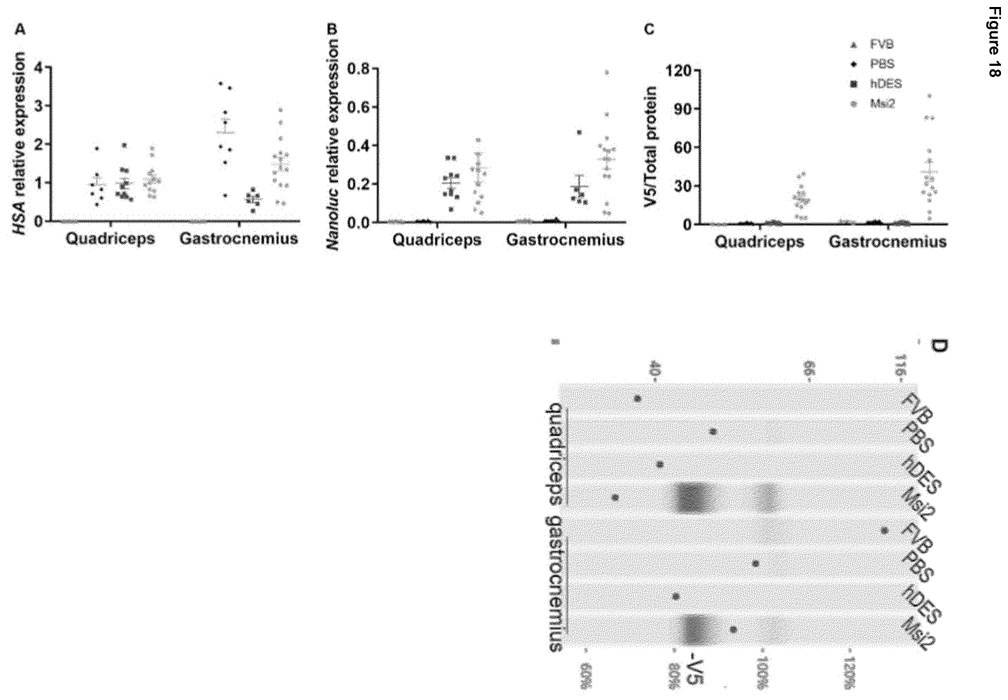

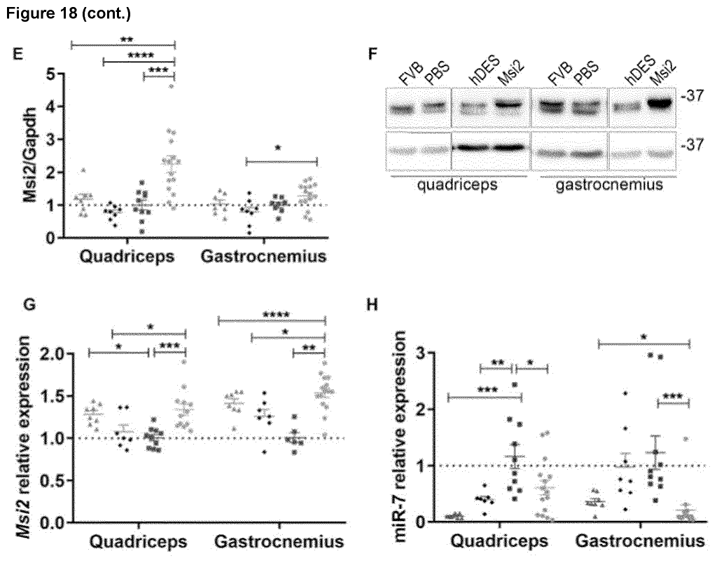

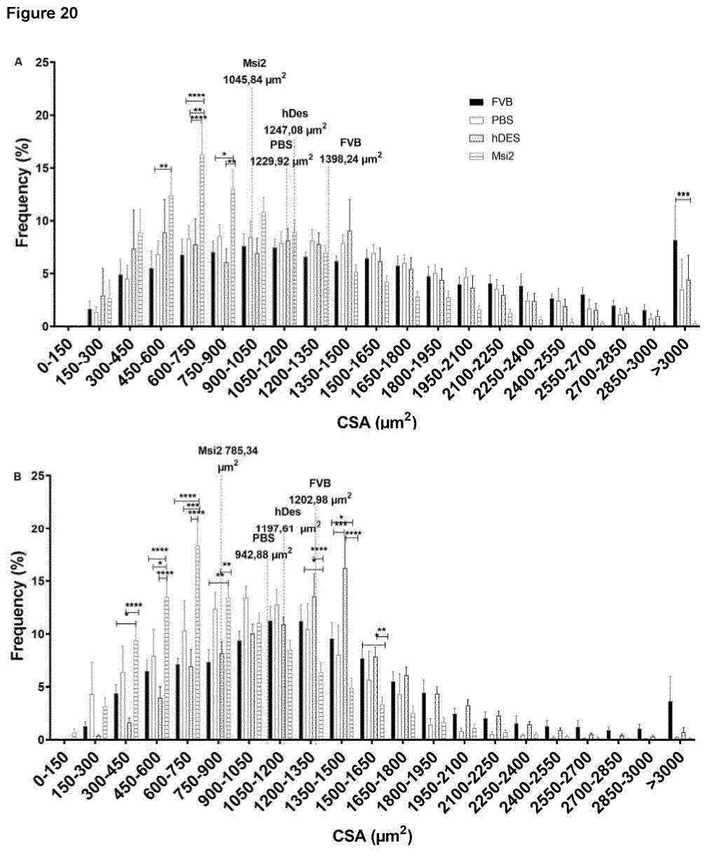

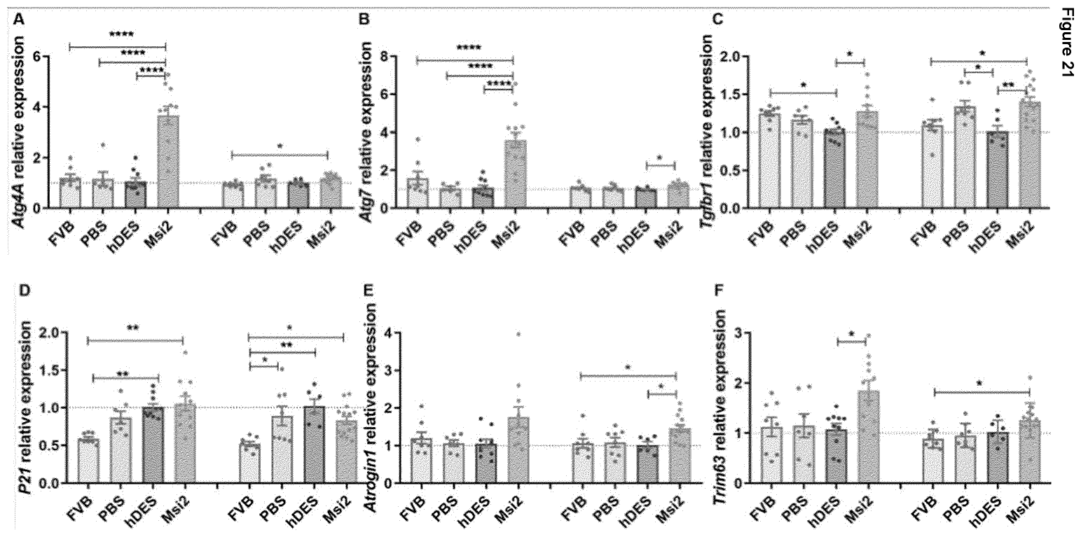

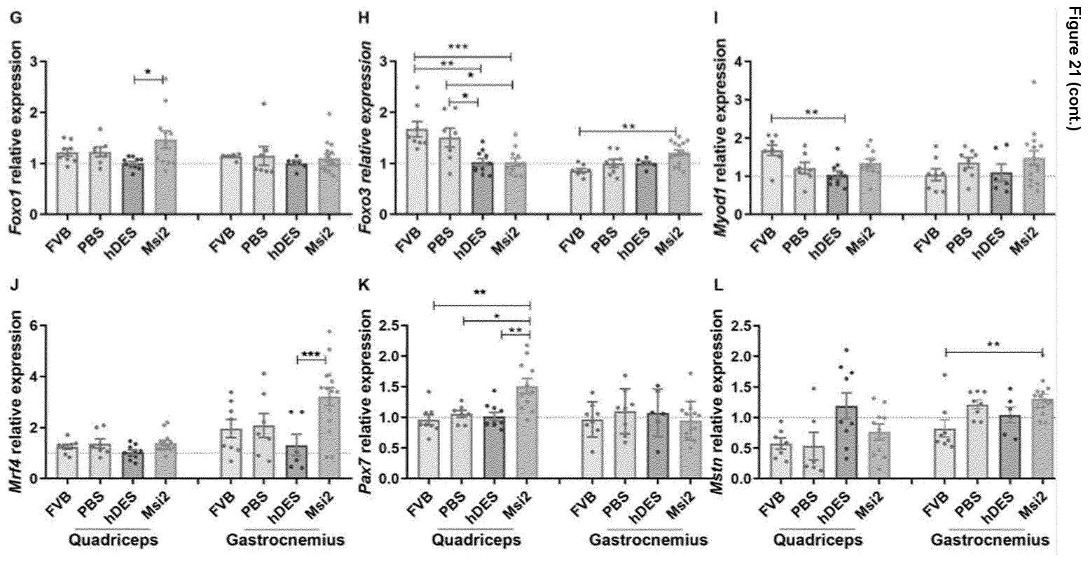

This example was carried out to confirm further that MSI2 overexpression is directly related to muscle atrophy and MD1 phenotype. The methods used herein are similar as in Example 8, and the results showed that the MSI2 overexpression enhances DM1-like phenotype in HSALR mice (FIG. 17), that MSI2 is overexpressed in skeletal muscle upon AAV9 infection (FIG. 18), that MSI2 overexpressing promotes reduced area of muscular fibers (FIG. 19), that the distribution of the area of myofibers is impaired upon MSI2 overexpression (FIG. 20), and that alterations in muscle homeostasis-related genes are detected in MSI2-overexpressing mice (FIG. 21). Similar as reported in Example 8, these results corroborate that MSI2 overexpression is sufficient to trigger the activation of molecular mechanisms that contribute to muscle atrophy in MD1.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for treatment of myotonic dystrophy in a mammal, comprising administering to the mammal an inhibitor of RNA-binding protein Musashi homolog 2 (MSI2), wherein the inhibitor:
i) is an oligonucleotide that comprises at least 7 consecutive nucleotides in length, wherein said 7 consecutive nucleotides have at least 90% identity over the complementary sequence of the full length sequence of SEQ ID NO: 1, or
ii) is the molecule 2,3,4,10-Tetrahydro-7,10-dimethyl-2,4-dioxobenzo[g]pteridine-8-carboxaldehyde, or any analogue or derivatives thereof.

2. The method according to claim 1, wherein said inhibitor is an oligonucleotide according to i), and wherein the oligonucleotide is selected from the group consisting of small interfering RNAs, antisense oligonucleotides, gapmers, morpholino oligomers, FANA oligonucleotides, agomiRs, miRNA mimics, antagomiRs, blockmiRs, PNAs, locked nucleic acid antisense oligonucleotides (LNAs), splice-switching oligonucleotides (SSOs), LNA-based splice-switching oligonucleotides (LNA SSOs), LNA/DNA mixmers and miRNA sponges.

3. The method according to claim 1, wherein said oligonucleotide comprises at least 10-25 nucleotides in length, from which at least 7 consecutive nucleotides in length have at least 90% identity over the complementary sequence of the full length sequence of SEQ ID NO: 2.

4. The method according to claim 1, wherein said oligonucleotide comprises at least 10-25 nucleotides in length, from which at least 7 consecutive nucleotides in length have at least 90% identity over the complementary sequence of the full sequence of any of the oligonucleotides of SEQ ID NO: 3 to SEQ ID NO: 37.

5. The method according to claim 1, wherein said oligonucleotide comprises at least 10-25 nucleotides in length, from which at least 7 consecutive nucleotides in length have at least 95% identity over the full sequence of any of the oligonucleotides of SEQ ID NO: 38 to 43 or 76, 77, or 78.

6. The method according to claim 1, wherein the myotonic dystrophy is myotonic dystrophy type 1.

7. The method according to claim 1, wherein the mammal is a human being.

8. The method according to claim 1, wherein the inhibitor is comprised in a pharmaceutical composition comprising a carrier and/or one or more pharmaceutically acceptable excipients.

9. An oligonucleotide that is between 10-50 nucleotides in length, of which at least 5 consecutive nucleotides have at least 90% identity over the complementary full length sequence of any of SEQ ID NO: 1 to SEQ ID NO: 37.

10. The oligonucleotide according to claim 9, wherein the oligonucleotide consists of a sequence that is identical to the complementary sequence of any of SEQ ID NO: 3 to SEQ ID NO: 37.

11. An oligonucleotide that is between 10-50 nucleotides in length, of which at least 5 consecutive nucleotides have at least 90% identity over the full length sequence of any of SEQ ID NO: 38 to 43 or 76, 77, or 78.

12. The oligonucleotide according to claim 11, wherein the oligonucleotide is selected from the group consisting of any of SEQ ID NO: 38 to 43 or 76, 77, or 78.

13. An expression vector comprising a sequence encoding a short hairpin RNA molecule (shRNA), wherein the shRNA comprises:
(i) a first sequence comprising an oligonucleotide according to claim 9 and;
(ii) a second sequence that is adjacent to the first sequence, wherein said second sequence is at least 95% complementary to the first sequence, wherein the first and the second sequences form a hairpin by hybridization, and
wherein said sequence encoding an shRNA molecule is operably linked to an RNA polymerase promoter.

14. The expression vector according to claim 13, wherein the vector is an adeno-associated vector.

15. A pharmaceutical composition comprising the oligonucleotide according to claim 9, and a carrier and/or one or more pharmaceutically acceptable excipients.

16-18. (canceled)

19. The oligonucleotide of claim 9, wherein the oligonucleotide is between 15-25 nucleotides in length, of which at least 5 consecutive nucleotides have at least 90% identity over the complementary full length sequence of any of SEQ ID NO: 1 to SEQ ID NO: 37.

20. The oligonucleotide of claim 11, wherein the oligonucleotide is between 15-25 nucleotides in length, of which at least 5 consecutive nucleotides have at least 90% identity over the full length sequence of any of SEQ ID NO: 38 to 43 or 76, 77, or 78.

21. An expression vector comprising a sequence encoding a short hairpin RNA molecule (shRNA), wherein the shRNA comprises:
(i) a first sequence comprising an oligonucleotide according to claim 11; and
(ii) a second sequence that is adjacent to the first sequence, wherein said second sequence is at least 95% complementary to the first sequence, wherein the first and the second sequences form a hairpin by hybridization, and
wherein said sequence encoding an shRNA molecule is operably linked to an RNA polymerase promoter.

22. The expression vector according to claim 21, wherein the vector is an adeno-associated vector.

23. A pharmaceutical composition comprising the oligonucleotide according to claim 11, and a carrier and/or one or more pharmaceutically acceptable excipients.


SEQUENCE LISTING (138 sequences)

SEQ ID NO: 1 - DNA, 6379 bp, Homo sapiens
gtgcgaggcagcggggctgagctaagccgagcccacgtgtgacggctctcgccgctgccccggctccgccgctcgcagagagattcggaggagcccgggcgggggggaggaggagggggaggagggagcggagatctcggggctcggagccggccgccgctccgctccgatcgctgtggggcttggttttttgggggtgggggggcgggggggctcagatatggaggcaaatgggagccaaggcacctcgggcagcgccaacgactcccagcacgaccccggtaaaatgtttatcggtggactgagctggcagacctcaccagatagccttagagactattttagcaaatttggagaaattagagaatgtatggtcatgagagatcccactacgaaacgctccagaggcttcggtttcgtcacgttcgcagacccagcaagtgtagataaagtattaggtcagccccaccatgagttagattccaagacgattgaccccaaagttgcatttcctcgtcgagcgcaacccaagatggtcacaagaacaaagaaaatatttgtaggcgggttatctgcgaacacagtagtggaagatgtaaagcaatatttcgagcagtttggcaaggtggaagatgcaatgctgatgtttgataaaactaccaacaggcacagagggtttggctttgtcacttttgagaatgaagatgttgtggagaaagtctgtgagattcatttccatgaaatcaataataaaatggtagaatgtaagaaagctcagccgaaagaagtcatgttcccacctgggacaagaggccgggcccggggactgccttacaccatggacgcgttcatgcttggcatggggatgctgggatatcccaacttcgtggcgacctatggccgtggctaccccggatttgctccaagctatggctatcagttcccaggcttcccagcagcggcttatggaccagtggcagcagcggcggtggcggcagcaagaggatcaggctccaacccggcgcggcccggaggcttcccgggggccaacagcccaggacctgtcgccgatctctacggccctgccagccaggactccggagtggggaattacataagtgcggccagcccacagccgggctcgggcttcggccacggcatagctggacctttgattgcaacggcctttacaaatggataccattgagcaggtgctttcgttgccatctcactctgagagcatacctggatgtccaggcaagactgggcgaagtttctgagtggccctttgtttaggtgatgtcctcagacctggacccccaccagcctcactccccatcccaaccagagatggctcacttcggatcgagggttgactacatctcatcatctcacgaatctgctgtaatataagacaacagcttttaaatgtgtatataacccatgatttcggttttgttttgttttgtttttcttgatggtttccctctccctccctctcttcccattctccttttaaatctctttgaatcacatttggtagtgattttgacttagtccagtagtcacatagctttaatatctagttcaaagctaaccatagtataattgttatattaaggagttattttttcttaaaacatttttttttgcttgttttggttctgttctcacttttaaaggatgctgagatggtaatatgactctccatattttggtaccaattctgagactgtatgaattttcaggtggaactttagcacacactgaagcaaagttgtgaagtgcagggcgggaggtgggcgtgagctttctattttgcgttgtagaagaagtgagatgtagtaagctaattaacagactttctagcagttctttttgtgatgtctctttgttaatctgagtctatctattttcggcaataaggtaaggacgacagtgttttgagtgtcctccttttctataagtgctttttttctgttgaaagaggtgatattataaggttttttgaaattgtgaattctaaaaaagaaatgttgtaaatacaattccattaactacatagaaactattaagaaagagagaatcaaaaatatttttgtgagggagtcggtcccaggcagtttgatgctctgtggaaggaggcgggaagggaacgttggccaagtcagttactgagatgaagatcgcccagctgccaggaccaccccaggacaagttagagcactgtttagctcctttgtctgtgtgatagacctaagaactgtattagtgttgtaccagcctattaacctcttgtctgtgcacagcttcaaatgttaccgtctagttagatttttatttaaaatatgaaaaactgcttttcccaagatgttttttaaaaacaaagctacaattttaatatttaacatatttaaagtttcaaagcacacctgtttggcttgggtgggggtggggtgggggggacattctttttcagtcttaatttttaaatatttgatcattttctattgtccaatcatttcagcacctccaaaggtccctaggacactttgcctctcttctccccctgccccccaccctgctcccacatctgggggcccatgggccaggagtggataagcctgcattaatacaacctttctccattcactttctatttacaaattaggaaagcaaccttttggtttatatatattttttttaatacctcagtgctgcaagtatcaccagagaggctatggaagaatttttttttaatttattgtagatgtaaacagaattttaaaaataaaaagtataaacatcactgcactgtgactggtgggaaaaactgacagtttcctctttgcacatgtttaacatttggctgttataatatatggtcctcggttggggaaagatacttatgatgaaggatattttttaatttaacttttttttaaatattggtaataggtcggcaacagcaactatagaagtacaactcaatagatggcattaaaacatattgtagtgtggatatatattttttcttttttaaaatgtgatattgacgttttattaatattttttaaattgttacgtttataaatttggtacttaaggcacagccagtatgagacactgaatgcgacatttattataaagagctgctgcactcctatttttataaattttactaacaaagtagactaatgtagacattcacagacatggtagggcaaaagcatcttcaaactaaagactccaaaatgctaactcagaaagaaagaaaaaacccgttttcaattctaatgaaacagcaacaacattttttttaattaaaaaaaaaatcatgttctttgtttttctaataaaatgttaggttgtttggtgaggtttttttgttgtttttttcctttctttgtttcttttcttttcccccaaacaaccagattaaatgctgagcgcttttaaaatatcaaaacttgttcaagtcataaaacaacaaaacataagttttatttaaaaaaaaaaaaagaaagaaaaaatagtaaattcccagaaattcagtgtttagacgagggaatttaattcctattttgtccatgttggtgatgtactgtacttcccttccttttctctgcatcccccatcacctcatagaagactctttgttgatcattgtatgttaataatgtataaaatggctatcttgtaagcgtgctgtcctggtactagtgtagcgactttttttctcctctttcttctagtacatattgataggtataacataattaaggtttaaaaaaaattagacatagttattcagatttaggaccagtaaggatagaactttctcttatttatgaaaaaaaatgctaataattttggggcagttttttcctttaattatttttttcaatttcaagtttaattttattttagctgatctgatgtggtttcaactaacccaaggtctcaccatgttaaaatgccggcggactctacggcgttttgtagatccccccccccccacccactgtgaaggggtgccatactaccttaaatgctaatgctagatatgcaaaactggatttttttaatttattttttaaaagagggaggcatggtatattaaaatgattttactaagagaaaaaatatttttttaagaatgctcagaagaaattgataatctgtgtgaatatgttttagatgtttatataccttttgaagagacccagtagcccatagcacaaatcttgtggaaatccgatatgttttaatgtggctacctaggtttaaggttcacgttagtccccccattccatctagaagtccattttgaaagatttttgtaaattcttttaacactgatgtttcagcctcgtctttgtttcagttaagctcaatggcgaacatgggaaccacctttcgccttccctgggggagaaaccctcttggctgatggcttttccccggaattatcaaacagccaccgggtgactttctggcttccagatccatctgcctgagacccccgaactcctctcctcccaagcagaggcgagtgagtggcattagctcccggacccattcccggtcctagctgggcatggggctgacggagatgaccaagccttggtctgctctctagcagcttccacagacttggctcgtggccttccttatatccactgggaacaaacagcctcgcgctctataccaaacagcctcgcgctctatcccagtcgcccattagcttgattcaaacaaagccccagcaggcctttgcgtttttatccttcataaccttcatcttaatttgaacttgtagcttggactttaaggtagcatggctctattgctgtcaatttactgtttcactgcacagcaatcacagccagtgaatgttacacacatcttgctagactagtataaaaatcattgggtaattgttggttctaatgacctgaaaggtgttcagtttttgttcttggttttgttttgtttttgatttcttgggggtgggttttgcttgttgttccttttcatttgggggtttttggggaaaaaatttatttttggttccaaatagaaaaacaaaacctattttgatctttagtgcaaacgagggctagggacttagcctcctccaccacctccacactgcttcattctgccattcactcactgcagcatattcaagaataaagcaatatcgtttactacattttttattgaaggtcagccatgctttctgtattatattgcatatgaaattgtttacaaaagaaacactaactcatacttctctttattggttgcaagtggcacgcaggaacagagggagagtggggggctggtgggggaggggagatttttttttttttttcttgaatgtgcttcgcaagccaggctatcttccaaggaaggcagacagtgggagagcagagggactgactgcaggcaagcacattgaagaaagacactggcgggtttccccaccctcaccccaaagcagaaaactagcagacgtcagctcagccccgtcctgggcacagacactacacaaggagacgctggaagttaagcaatactttaatactgtaatatgtttgttttctttttctttcttttttttctaccaaaaaaaagtaagtaaactaaaacacaaaaacatataaataaaatccatccctcttgtcggggacctgcaggggggcaaccttaatccaaacacctggctatcaaataatcagaatgtattgtctcagacaggatttcagttccgggaggcaggggcatgatgggggagggggccggaggctgagagacaaaagttccagagcctccctcgaaggttctctactactgtattctgtacataatgtaccatcccatgtggaatctgtgagtgtcctcttaagtagcgtgggctagccaatctgccgttcatggtgtattgtaaactccgaattccatatgtaataggatgcaagtctaagcgtttcatgtggacataaatgtatctaaataaaactttccctagcactgtggctgacctcacccttacttttatactttagtatgaaactgatgagaactttggtagtgagtattttttttatatatatacatatatatgtactatctatatatatatctcaagcatctttcaggtctttgtgtgtggctttcttaaagccctgttgtaaaaaattactatgtggatggcagtctctcacatcacagatgtggaaagtataattttatatttgtattttcaaataaataagtttgtgaaaggtttccatcctctactgtggtccagaaatcaatgtgtttgtctgacaaaaaaaaaaataaaataaaataaactgttttgaacaga

SEQ ID NO: 2 - DNA, 1292 bp, Artificial Sequence
  Partial MSI2 transcript (nucleotides 327-1618)
gccttagagactattttagcaaatttggagaaattagagaatgtatggtcatgagagatcccactacgaaacgctccagaggcttcggtttcgtcacgttcgcagacccagcaagtgtagataaagtattaggtcagccccaccatgagttagattccaagacgattgaccccaaagttgcatttcctcgtcgagcgcaacccaagatggtcacaagaacaaagaaaatatttgtaggcgggttatctgcgaacacagtagtggaagatgtaaagcaatatttcgagcagtttggcaaggtggaagatgcaatgctgatgtttgataaaactaccaacaggcacagagggtttggctttgtcacttttgagaatgaagatgttgtggagaaagtctgtgagattcatttccatgaaatcaataataaaatggtagaatgtaagaaagctcagccgaaagaagtcatgttcccacctgggacaagaggccgggcccggggactgccttacaccatggacgcgttcatgcttggcatggggatgctgggatatcccaacttcgtggcgacctatggccgtggctaccccggatttgctccaagctatggctatcagttcccaggcttcccagcagcggcttatggaccagtggcagcagcggcggtggcggcagcaagaggatcaggctccaacccggcgcggcccggaggcttcccgggggccaacagcccaggacctgtcgccgatctctacggccctgccagccaggactccggagtggggaattacataagtgcggccagcccacagccgggctcgggcttcggccacggcatagctggacctttgattgcaacggcctttacaaatggataccattgagcaggtgctttcgttgccatctcactctgagagcatacctggatgtccaggcaagactgggcgaagtttctgagtggccctttgtttaggtgatgtcctcagacctggacccccaccagcctcactccccatcccaaccagagatggctcacttcggatcgagggttgactacatctcatcatctcacgaatctgctgtaatataagacaacagcttttaaatgtgtatataacccatgatttcggttttgttttgttttgtttttcttgatggtttccctctccctccctctcttcccattctccttttaaatctctttgaatcacatttggtagtgattttgacttagtccagtagtcacatagctttaatatctagttcaaagctaaccatagtataattgttatatt

SEQ ID NO: 3 - DNA, 23 bp, Artificial Sequence
  potential target region
gccttagagactattttagcaaa

SEQ ID NO: 4 - DNA, 23 bp, Artificial Sequence
  potential target region
tagagactattttagcaaatttg

SEQ ID NO: 5 - DNA, 23 bp, Artificial Sequence
  potential target nucleotide
gactattttagcaaatttggaga

SEQ ID NO: 6 - DNA, 23 bp, Artificial Sequence
  potential target region
agcaaatttggagaaattagaga

SEQ ID NO: 7 - DNA, 23 bp, Artificial Sequence
  potential target region
agcaagtgtagataaagtattag

SEQ ID NO: 8 - DNA, 23 bp, Artificial Sequence
  potential target region
cacaagaacaaagaaaatatttg

SEQ ID NO: 9 - DNA, 23 bp, Artificial Sequence
  potential target region
aagaacaaagaaaatatttgtag

SEQ ID NO: 10 - DNA, 23 bp, Artificial Sequence
  potential target region
gtggaagatgtaaagcaatattt

SEQ ID NO: 11 - DNA, 23 bp, Artificial Sequence
  potential target region
tggaagatgtaaagcaatatttc

SEQ ID NO: 12 - DNA, 23 bp, Artificial Sequence
  potential target region
tgctgatgtttgataaaactacc

SEQ ID NO: 13 - DNA, 23 bp, Artificial Sequence
  potential target region
tggctttgtcacttttgagaatg

SEQ ID NO: 14 - DNA, 23 bp, Artificial Sequence
  potential target region
ggctttgtcacttttgagaatga

SEQ ID NO: 15 - DNA, 23 bp, Artificial Sequence
  potential target region
cacttttgagaatgaagatgttg

SEQ ID NO: 16 - DNA, 23 bp, Artificial Sequence
  potential target region
aagatgttgtggagaaagtctgt

SEQ ID NO: 17 - DNA, 23 bp, Artificial Sequence
  potential target region
gagaaagtctgtgagattcattt

SEQ ID NO: 18 - DNA, 23 bp, Artificial Sequence
  potential target region
ttcatttccatgaaatcaataat

SEQ ID NO: 19 - DNA, 23 bp, Artificial Sequence
  potential target region
ttccatgaaatcaataataaaat

SEQ ID NO: 20 - DNA, 23 bp, Artificial Sequence
  potential target region
tccaagctatggctatcagttcc

SEQ ID NO: 21 - DNA, 23 bp, Artificial Sequence
  potential target region
ttgattgcaacggcctttacaaa

SEQ ID NO: 22 - DNA, 23 bp, Artificial Sequence
  potential target region
cacgaatctgctgtaatataaga

SEQ ID NO: 23 - DNA, 23 bp, Artificial Sequence
  potential target region
aacagcttttaaatgtgtatata

SEQ ID NO: 24 - DNA, 23 bp, Artificial Sequence
  potential target region
cagcttttaaatgtgtatataac

SEQ ID NO: 25 - DNA, 23 bp, Artificial Sequence
  potential target region
agcttttaaatgtgtatataacc

SEQ ID NO: 26 - DNA, 23 bp, Artificial Sequence
  potential target region
tcccattctccttttaaatctct

SEQ ID NO: 27 - DNA, 23 bp, Artificial Sequence
  potential target region
ctccttttaaatctctttgaatc

SEQ ID NO: 28 - DNA, 23 bp, Artificial Sequence
  potential target region
tccttttaaatctctttgaatca

SEQ ID NO: 29 - DNA, 23 bp, Artificial Sequence
  potential target region
ctctttgaatcacatttggtagt

SEQ ID NO: 30 - DNA, 23 bp, Artificial Sequence
  potential target region
atcacatttggtagtgattttga

SEQ ID NO: 31 - DNA, 23 bp, Artificial Sequence
  potential target region
tggtagtgattttgacttagtcc

SEQ ID NO: 32 - DNA, 23 bp, Artificial Sequence
  potential target region
cacatagctttaatatctagttc

SEQ ID NO: 33 - DNA, 23 bp, Artificial Sequence
  potential target region
tagctttaatatctagttcaaag

SEQ ID NO: 34 - DNA, 23 bp, Artificial Sequence
  potential target region
agctttaatatctagttcaaagc

SEQ ID NO: 35 - DNA, 23 bp, Artificial Sequence
  potential target region
ttcaaagctaaccatagtataat

SEQ ID NO: 36 - DNA, 23 bp, Artificial Sequence
  potential target region
agctaaccatagtataattgtta

SEQ ID NO: 37 - DNA, 23 bp, Artificial Sequence
  potential target region
aaccatagtataattgttatatt

SEQ ID NO: 38 - DNA, 16 bp, Artificial Sequence
  ASO 1
tgacttctttcggctg

SEQ ID NO: 39 - DNA, 16 bp, Artificial Sequence
  ASO 3
ttggattaaggttgcc

SEQ ID NO: 40 - DNA, 21 bp, Artificial Sequence
  siRNA 1
tgacttctttcggctgagctt

SEQ ID NO: 41 - RNA, 23 nt, Artificial Sequence
  Sense AgomiR-7 (mimic of miR-107)
agcagcauuguacagggcuauca

SEQ ID NO: 42 - RNA, 23 nt, Artificial Sequence
  Antisense AgomiR-7 (mimic of miR-107)
ugauagcccuguacaaugcugcu

SEQ ID NO: 43 - DNA, 21 bp, Artificial Sequence
  siRNA 2
agttttatcaaacatcagcat

SEQ ID NO: 44 - DNA, 16 bp, Artificial Sequence
  ASOS SCRAMBLE (control)
ttccctgaaggttcct

SEQ ID NO: 45 - DNA, 20 bp, Artificial Sequence
  GAPDH fwd
catcttccaggagcgagatc

SEQ ID NO: 46 - DNA, 21 bp, Artificial Sequence
  GAPDH rev
gttcacacccatgacgaacat

SEQ ID NO: 47 - DNA, 20 bp, Artificial Sequence
  GPI fwd
cagggcatcatctgggacat

SEQ ID NO: 48 - DNA, 20 bp, Artificial Sequence
  GPI rev
tcttagccagctgctttccc

SEQ ID NO: 49 - DNA, 21 bp, Artificial Sequence
  HPRT1 fwd
tgacactggcaaaacaatgca

SEQ ID NO: 50 - DNA, 21 bp, Artificial Sequence
  HPRT1 REV
ggtccttttcaccagcaagct

SEQ ID NO: 51 - DNA, 23 bp, Artificial Sequence
  IGF1 fwd
ctcttcagttcgtgtgtggagac

SEQ ID NO: 52 - DNA, 22 bp, Artificial Sequence
  IGF1 rev
cagcctccttagatcacagctc

SEQ ID NO: 53 - DNA, 22 bp, Artificial Sequence
  MSI2 fwd
gcagacctcaccagatagcctt

SEQ ID NO: 54 - DNA, 22 bp, Artificial Sequence
  MSI2 rev
aagcctctggagcgtttcgtag

SEQ ID NO: 55 - DNA, 24 bp, Artificial Sequence
  MSTN fwd
tgagaatggtcatgatcttgctgt

SEQ ID NO: 56 - DNA, 24 bp, Artificial Sequence
  MSTN rev
tcatcacagtcaagaccaaaatcc

SEQ ID NO: 57 - DNA, 22 bp, Artificial Sequence
  mTOR fwd
agcatcggatgcttaggagtgg

SEQ ID NO: 58 - DNA, 22 bp, Artificial Sequence
  mTOR rev
cagccagtcatctttggagacc

SEQ ID NO: 59 - DNA, 24 bp, Artificial Sequence
  NFIX fwd
gagccctgttgatgacgtgttcta

SEQ ID NO: 60 - DNA, 24 bp, Artificial Sequence
  NFIX rev
ctgcacaaactccttcagtgagtc

SEQ ID NO: 61 - DNA, 22 bp, Artificial Sequence
  P21 fwd
aggtggacctggagactctcag

SEQ ID NO: 62 - DNA, 22 bp, Artificial Sequence
  P21 rev
tcctcttggagaagatcagccg

SEQ ID NO: 63 - DNA, 20 bp, Artificial Sequence
  PKM fwd
ctgaaggcagtgatgtcgcc

SEQ ID NO: 64 - DNA, 19 bp, Artificial Sequence
  PKM rev
acccggaggtccacgtctc

SEQ ID NO: 65 - DNA, 21 bp, Artificial Sequence
  ATP2A fwd
gatgatcttcaagctccgggc

SEQ ID NO: 66 - DNA, 20 bp, Artificial Sequence
  ATP2A rev
cagctctgcctgaagatgtg

SEQ ID NO: 67 - DNA, 22 bp, Artificial Sequence
  TGFBR1 fwd
gacaacgtcaggttctggctca

SEQ ID NO: 68 - DNA, 22 bp, Artificial Sequence
  TGFBR1 rev
ccgccactttcctctccaaact

SEQ ID NO: 69 - DNA, 2619 bp, Artificial Sequence
  AV9-hDES-mMSI2(V4)-V5-T2A-Nanoluc
gctagcgcggccgcacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccggttcgaacaggtaagcgcccctaaaatccctttggcacaatgtgtcctgaggggagaggcagcgacctgtagatgggacgggggcactaaccctcagggtttggggttctgaatgtgagtatcgccatctaagcccagtatttggccaatctcagaaagctcctggctccctggaggatggagagagaaaaacaaacagctcctggagcagggagagtgttggcctcttgctctccggctccctctgttgccctctggtttctccccaggttcgaaggatccatggaggcaaatgggagcccaggcacctcgggcagcgccaacgactcccagcacgaccccggtaaaatgtttatcggtggactgagctggcagacctcaccagatagccttagagactattttagcaaatttggagaaattagagaatgtatggtcatgagagatcccacaacgaaacgctccagaggcttcggtttcgtcaccttcgcagacccagcaagtgtagataaagtattaggtcagccccaccatgagttagattccaagacgattgacccaaaagttgcatttcctcgtcgagcgcaacctaagatggtcacaagaacaaagaaaatcttcgtaggaggattgtctgcaaacacagtagtggaagatgtaaagcagtatttcgagcagtttggcaaggtagaggatgcgatgctgatgttcgacaaaaccaccaacaggcacagagggtttggctttgtcacctttgagaatgaagacgttgtggagaaagtctgtgagattcatttccatgaaatcaataataaaatggtagaatgtaagaaagctcagccgaaagaagtcatgttcccacctgggacaagaggccgggcccgggggctgccatacaccatggatgcgttcatgcttggcatggggatgctgggctaccccaactttgtggcaacctatggcagaggctaccccggatttgctcctagctatggctaccagttcccaggcttcccggcggcagcttatggaccagtggcagcggcagctgtggcagcggctcgaggatcaggctccaacccggcgcggcccggaggcttcccgggggccaacagcccaggacctgtcgccgatctctacggccctgccagccaggactccggagtggggaattacataagcgcggccagcccacagccgggctccggcttcggccacggcatagctggacctttgattgcaacggcctttacaaatggataccactgaggtaagcctatccctaaccctctcctcggtctcgattctacggagggcagaggaagtctgctaacatgcggtgacgtcgaggagaatcctggcccaggatccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaagtatac

SEQ ID NO: 70 - DNA, 20 bp, Artificial Sequence
  Gapdh fwd M. musculus
atcaacgggaagcccatcac

SEQ ID NO: 71 - DNA, 21 bp, Artificial Sequence
  Gapdh rev M. musculus
cttccacaatgccaaagttgt

SEQ ID NO: 72 - DNA, 22 bp, Artificial Sequence
  Gtf2b fwd M. musculus
atggcggacagaatcaacctcc

SEQ ID NO: 73 - DNA, 22 bp, Artificial Sequence
  Gtf2b rev M. musculus
acaagcagaggctatcgcgtca

SEQ ID NO: 74 - DNA, 23 bp, Artificial Sequence
  Hprt1 fwd M. musculus
gttggatacaggccagactttgt

SEQ ID NO: 75 - DNA, 20 bp, Artificial Sequence
  Hprt1 Rev M. musculus
cacaggactagaacacctgc

SEQ ID NO: 76 - DNA, 16 bp, Artificial Sequence
  ASO1 with chemical modifications
  misc_feature 1,2,3,14,15,16: note=locked nucleic acid
  misc_feature 4,5,6,7,8,9,10,11,12,13: note=phosphorothioate linkages
tgacttctttcggctg

SEQ ID NO: 77 - DNA, 16 bp, Artificial Sequence
  ASO3 with chemical modifications
  misc_feature 1,2,3,14,15,16: note=locked nucleic acid
  misc_feature 4,5,6,7,8,9,10,11,12,13: note=phosphorothioate linkages
ttggattaaggttgcc

SEQ ID NO: 78 - RNA, 23 nt, Artificial Sequence
  AgomiR-107 antisense strand with chemical modifications and conjugated to cholesterol
  misc_feature 1,2,3,20,21,22,23: note=2primeO-methyl-modified phosphoramidites and phosphorothioate linkages
  misc_feature 4,5,6,7,8,9,10,11,12,13,14,15,16,17,18,19: note=2prime-O-methyl-modified phosphoramidites
  misc_feature 23: note=cholesterol conjugation
ugauagcccuguacaaugcugcu

SEQ ID NO: 79 - DNA, 23 bp, Artificial Sequence
  Msi2 fwd M. musculus
cagccgaaagaagtcatgttccc

SEQ ID NO: 80 - DNA, 22 bp, Artificial Sequence
  Msi2 rev M. musculus
cctctgccataggttgccacaa

SEQ ID NO: 81 - DNA, 23 bp, Artificial Sequence
  Mrf4 fwd M. musculus
atcagctacattgagcgtctaca

SEQ ID NO: 82 - DNA, 23 bp, Artificial Sequence
  Mrf4 rev M. musculus
cctggaatgatccgaaacacttg

SEQ ID NO: 83 - DNA, 20 bp, Artificial Sequence
  Mstn fwd M. musculus
ctcagacccgtcaagactcc

SEQ ID NO: 84 - DNA, 24 bp, Artificial Sequence
  Mstn rev M. musculus
ctctgccaaatcaataccagtgcc

SEQ ID NO: 85 - DNA, 22 bp, Artificial Sequence
  Atg4a fwd M. musculus
cagtctccacagcggatgagta

SEQ ID NO: 86 - DNA, 22 bp, Artificial Sequence
  Atg4a rev M. musculus
gtgtgatgggtgcttctgaacc

SEQ ID NO: 87 - DNA, 19 bp, Artificial Sequence
  Atg7 fwd M. musculus
gaccggtcttaccctgctc

SEQ ID NO: 88 - DNA, 20 bp, Artificial Sequence
  Atg7 rev M. musculus
tgtggttgcttgcttcagac

SEQ ID NO: 89 - DNA, 22 bp, Artificial Sequence
  P21 fwd M. musculus
tcgctgtcttgcactctggtgt

SEQ ID NO: 90 - DNA, 23 bp, Artificial Sequence
  P21 rev M. musculus
ccaatctgcgcttggagtgatag

SEQ ID NO: 91 - DNA, 20 bp, Artificial Sequence
  Pax7 fwd M. musculus
gagcactcggctaatcgaac

SEQ ID NO: 92 - DNA, 20 bp, Artificial Sequence
  Pax7 rev M. musculus
ccgtgtttctcatggttgtg

SEQ ID NO: 93 - DNA, 22 bp, Artificial Sequence
  Tgfbr1 fwd M. musculus
tgctccaaaccacagagtaggc

SEQ ID NO: 94 - DNA, 22 bp, Artificial Sequence
  Tgfbr1 rev M. musculus
cccagaacactaagcccattgc

SEQ ID NO: 95 - DNA, 20 bp, Artificial Sequence
  Atrogin1 fwd M. musculus
ctctgtaccatgccgttcct

SEQ ID NO: 96 - DNA, 20 bp, Artificial Sequence
  Atrogin1 rev M. musculus
ggctgctgaacagattctcc

SEQ ID NO: 97 - DNA, 20 bp, Artificial Sequence
  Trim63 fwd M. musculus
acctgctggtggaaaacatc

SEQ ID NO: 98 - DNA, 20 bp, Artificial Sequence
  Trim63 rev M. musculus
aggagcaagtaggcacctca

SEQ ID NO: 99 - DNA, 20 bp, Artificial Sequence
  Foxo1 fwd M. musculus
gagagctcagccgagaagag

SEQ ID NO: 100 - DNA, 20 bp, Artificial Sequence
  Foxo1 rev M. musculus
ctccctctggattgagcatc

SEQ ID NO: 101 - DNA, 24 bp, Artificial Sequence
  Foxo3a fwd M. musculus
caggctcctcactgtattcagcta

SEQ ID NO: 102 - DNA, 22 bp, Artificial Sequence
  Foxo3a rev M. musculus
cattgaacatgtccaggtccaa

SEQ ID NO: 103 - DNA, 20 bp, Artificial Sequence
  Myod1 fwd M. musculus
agcacgcacacttctctact

SEQ ID NO: 104 - DNA, 20 bp, Artificial Sequence
  Myod1 rev M musculus
ggcctcattcactttgctca

SEQ ID NO: 105 - DNA, 20 bp, Artificial Sequence
  Nanoluc Fwd M. musculus
gggaggtgtgtccagtttgt

SEQ ID NO: 106 - DNA, 20 bp, Artificial Sequence
  nanolucrev M. musculus
ttcaccgctcaggacaatcc

SEQ ID NO: 107 - DNA, 18 bp, Artificial Sequence
  HSA Fwd M. musculus
acgggtgcgtggtgtctc

SEQ ID NO: 108 - DNA, 21 bp, Artificial Sequence
  HSA rev M. musculus
ggtcaggatacctctcttgct

SEQ ID NO: 109 - DNA, 1635 bp, Artificial Sequence
  MSI2 transcript
caagtcagtcgatcctgtgtttgtgctcccgaaggacttggcgacttggctaacaaggagacatttgttatttcatttcctcatccattattcatgcagtgagagatggtcacaagaacaaagaaaatatttgtaggcgggttatctgcgaacacagtagtggaagatgtaaagcaatatttcgagcagtttggcaaggtggaagatgcaatgctgatgtttgataaaactaccaacaggcacagagggtttggctttgtcacttttgagaatgaagatgttgtggagaaagtctgtgagattcatttccatgaaatcaataataaaatggtagaatgtaagaaagctcagccgaaagaagtcatgttcccacctgggacaagaggccgggcccggggactgccttacaccatggacgcgttcatgcttggcatggggatgctgggatatcccaacttcgtggcgacctatggccgtggctaccccggatttgctccaagctatggctatcagttcccagactatttgccggtttcacaagacataatttttatcaactagctcttaagagaggcatagcaaagtgggggttgctaccatttctagagagagaggacacagtcccggcctgggctgcccccgctccagtcaatgctcactgaaagtctgtcttagctgcctgtttgaatgactgttctttttctcatttttaattcttggactcatgtcctcattgcttcactcaattaaaaaaaaattattctccagtcccctcccactttgcttcttgtatgcattgtgaccgaccccacttcctcagaatgtaacggggccagagggaaacttctcacaaacttcgtagagcctcctcaggggaagctaggaagaagacatcaaatgtttttaagtcatgaccaaacaggcttgttggggacatatcatggggtgagctttgaagtgctggtggtccagaggggttgcagatacgtgacttggagcacccgtgtcttacgatggacagtgataaaggtgaacacacagagacagactattctctaagaatgtgagaaacctgatctggaagaggagctatataaacactatctgactatctttgtcttttggggccagtggctgttggcataatcacaagcctgtctgtcttcgagaagggacagtggagtcatccaggtgctgccacatgacaggcacggtgggcaccgatccacagtgggccccgccttccccagctcgcctccctgcctgtgctggcctggccttgcctgctggcaccattggagtaggagggggtggaacacagggggcccatcctgatcaggccccatctcaaggttggcactcctgcccatcacccttagaaggatcttttcccatggcttgacttccttcatttccctaactgaaatacacccactctcttggaataatgacgtaccactcagttggaccctcaagagtcactgctttgtctgtgctggtagtttgtgagaagtgacccgcacgcttccatttgatgcatttgatgtgagtgaatccatacatttgaatgtcattgtccttgagaccctacatgtgcagtttggctcatctcattaaagatgcttgatgtaataa

SEQ ID NO: 110 - DNA, 2124 bp, Artificial Sequence
  MSI2 transcript
gccaaggcacctcgggcagcgccaacgactcccagcacgaccccggtaaaatgtttatcggtggactgagctggcagacctcaccagatagccttagagactattttagcaaatttggagaaattagagaatgtatggtcatgagagatcccactacgaaacgctccagaggcttcggtttcgtcacgttcgcagacccagcaagtgtagataaagtattaggtcagccccaccatgagttagattccaagacgattgaccccaaagttgcatttcctcgtcgagcgcaacccaagagttccttacaagacaatctgatttgatccgcaggagaatcctatggtcacaagaacaaagaaaatatttgtaggcgggttatctgcgaacacagtagtggaagatgtaaagcaatatttcgagcagtttggcaaggtggaagatgcaatgctgatgtttgataaaactaccaacaggcacagagggtttggctttgtcacttttgagaatgaagatgttgtggagaaagtctgtgagattcatttccatgaaatcaataataaaatggtagaatgtaagaaagctcagccgaaagaagtcatgttcccacctgggacaagaggccgggcccggggactgccttacaccatggacgcgttcatgcttggcatggggatgctgggatatcccaacttcgtggcgacctatggccgtggctaccccggatttgctccaagctatggctatcagttcccagactatttgccggtttcacaagacataatttttatcaactagctcttaagagaggcatagcaaagtgggggttgctaccatttctagagagagaggacacagtcccggcctgggctgcccccgctccagtcaatgctcactgaaagtctgtcttagctgcctgtttgaatgactgttctttttctcatttttaattcttggactcatgtcctcattgcttcactcaattaaaaaaaaattattctccagtcccctcccactttgcttcttgtatgcattgtgaccgaccccacttcctcagaatgtaacggggccagagggaaacttctcacaaacttcgtagagcctcctcaggggaagctaggaagaagacatcaaatgtttttaagtcatgaccaaacaggcttgttggggacatatcatggggtgagctttgaagtgctggtggtccagaggggttgcagatacgtgacttggagcacccgtgtcttacgatggacagtgataaaggtgaacacacagagacagactattctctaagaatgtgagaaacctgatctggaagaggagctatataaacactatctgactatctttgtcttttggggccagtggctgttggcataatcacaagcctgtctgtcttcgagaagggacagtggagtcatccaggtgctgccacatgacaggcacggtgggcaccgatccacagtgggccccgccttccccagctcgcctccctgcctgtgctggcctggccttgcctgctggcaccattggagtaggagggggtggaacacagggggcccatcctgatcaggccccatctcaaggttggcactcctgcccatcacccttagaaggatcttttcccatggcttgacttccttcatttccctaactgaaatacacccactctcttggaataatgacgtaccactcagttggaccctcaagagtcactgctttgtctgtgctggtagtttgtgagaagtgacccgcacgcttccatttgatgcatttgatgtgagtgaatccatacatttgaatgtcattgtccttgagaccctacatgtgcagtttggctcatctcattaaagatgcttgatgtaataattggttagtttccttttattttcctgcaggcttttccatgagtattatttttttcaaagaacaaatctgtatggcttttccccatctccatattttgttttgctatgaattgctttgctttggtgaacttgtcctagtatgcttgcctcacaaacgttttagccattgtgaattttcttcatctctgtaaatagttcatctgtgcttctccctgatgacgttttattttttttcccctgtaagcaaccgaggtag

SEQ ID NO: 111 - DNA, 500 bp, Artificial Sequence
  MSI2 transcript
tttccaattcagtgggctgctacaatccggcctttatcacgggctgctctggttcccatgtcccttctgggccagtgcctgagtttgtggtttgacatgagggctttaaccccttcctgtgccagcccctcactgtggtggccgggaagaaaaggccttccaggaaccggcgtggaaagttacagctcttgaggtctcgtcaaagtgggagatcgctgtggaaaccagggtttggctttgtcacttttgagaatgaagatgttgtggagaaagtctgtgagattcatttccatgaaatcaataataaaatggtagaatgtaagaaagctcagccgaaagaagtcatgttcccacctgggacaagaggccgggcccggggactgccttacaccatggacgcgttcatgcttggcatggggatgctgggatatcccaacttcgtggcgacctatggccgtggctaccccggatttgctccaagctatggctatcagttccca

SEQ ID NO: 112 - DNA, 5296 bp, Artificial Sequence
  MSI2 transcript
gctccaacccggcgcggcccggaggcttcccgggggccaacagcccaggacctgtcgccgatctctacggccctgccagccaggactccggagtggggaattacataagtgcggccagcccacagccgggctcgggcttcggccacggcatagctagcatacctggatgtccaggcaagactgggcgaagtttctgagtggccctttgtttaggtgatgtcctcagacctggacccccaccagcctcactccccatcccaaccagagatggctcacttcggatcgagggttgactacatctcatcatctcacgaatctgctgtaatataagacaacagcttttaaatgtgtatataacccatgatttcggttttgttttgttttgtttttcttgatggtttccctctccctccctctcttcccattctccttttaaatctctttgaatcacatttggtagtgattttgacttagtccagtagtcacatagctttaatatctagttcaaagctaaccatagtataattgttatattaaggagttattttttcttaaaacatttttttttgcttgttttggttctgttctcacttttaaaggatgctgagatggtaatatgactctccatattttggtaccaattctgagactgtatgaattttcaggtggaactttagcacacactgaagcaaagttgtgaagtgcagggcgggaggtgggcgtgagctttctattttgcgttgtagaagaagtgagatgtagtaagctaattaacagactttctagcagttctttttgtgatgtctctttgttaatctgagtctatctattttcggcaataaggtaaggacgacagtgttttgagtgtcctccttttctataagtgctttttttctgttgaaagaggtgatattataaggttttttgaaattgtgaattctaaaaaagaaatgttgtaaatacaattccattaactacatagaaactattaagaaagagagaatcaaaaatatttttgtgagggagtcggtcccaggcagtttgatgctctgtggaaggaggcgggaagggaacgttggccaagtcagttactgagatgaagatcgcccagctgccaggaccaccccaggacaagttagagcactgtttagctcctttgtctgtgtgatagacctaagaactgtattagtgttgtaccagcctattaacctcttgtctgtgcacagcttcaaatgttaccgtctagttagatttttatttaaaatatgaaaaactgcttttcccaagatgttttttaaaaacaaagctacaattttaatatttaacatatttaaagtttcaaagcacacctgtttggcttgggtgggggtggggtgggggggacattctttttcagtcttaatttttaaatatttgatcattttctattgtccaatcatttcagcacctccaaaggtccctaggacactttgcctctcttctccccctgccccccaccctgctcccacatctgggggcccatgggccaggagtggataagcctgcattaatacaacctttctccattcactttctatttacaaattaggaaagcaaccttttggtttatatatattttttttaatacctcagtgctgcaagtatcaccagagaggctatggaagaatttttttttaatttattgtagatgtaaacagaattttaaaaataaaaagtataaacatcactgcactgtgactggtgggaaaaactgacagtttcctctttgcacatgtttaacatttggctgttataatatatggtcctcggttggggaaagatacttatgatgaaggatattttttaatttaacttttttttaaatattggtaataggtcggcaacagcaactatagaagtacaactcaatagatggcattaaaacatattgtagtgtggatatatattttttcttttttaaaatgtgatattgacgttttattaatattttttaaattgttacgtttataaatttggtacttaaggcacagccagtatgagacactgaatgcgacatttattataaagagctgctgcactcctatttttataaattttactaacaaagtagactaatgtagacattcacagacatggtagggcaaaagcatcttcaaactaaagactccaaaatgctaactcagaaagaaagaaaaaacccgttttcaattctaatgaaacagcaacaacattttttttaattaaaaaaaaaatcatgttctttgtttttctaataaaatgttaggttgtttggtgaggtttttttgttgtttttttcctttctttgtttcttttcttttcccccaaacaaccagattaaatgctgagcgcttttaaaatatcaaaacttgttcaagtcataaaacaacaaaacataagttttatttaaaaaaaaaaaaagaaagaaaaaatagtaaattcccagaaattcagtgtttagacgagggaatttaattcctattttgtccatgttggtgatgtactgtacttcccttccttttctctgcatcccccatcacctcatagaagactctttgttgatcattgtatgttaataatgtataaaatggctatcttgtaagcgtgctgtcctggtactagtgtagcgactttttttctcctctttcttctagtacatattgataggtataacataattaaggtttaaaaaaaattagacatagttattcagatttaggaccagtaaggatagaactttctcttatttatgaaaaaaaatgctaataattttggggcagttttttcctttaattatttttttcaatttcaagtttaattttattttagctgatctgatgtggtttcaactaacccaaggtctcaccatgttaaaatgccggcggactctacggcgttttgtagatccccccccccccacccactgtgaaggggtgccatactaccttaaatgctaatgctagatatgcaaaactggatttttttaatttattttttaaaagagggaggcatggtatattaaaatgattttactaagagaaaaaatatttttttaagaatgctcagaagaaattgataatctgtgtgaatatgttttagatgtttatataccttttgaagagacccagtagcccatagcacaaatcttgtggaaatccgatatgttttaatgtggctacctaggtttaaggttcacgttagtccccccattccatctagaagtccattttgaaagatttttgtaaattcttttaacactgatgtttcagcctcgtctttgtttcagttaagctcaatggcgaacatgggaaccacctttcgccttccctgggggagaaaccctcttggctgatggcttttccccggaattatcaaacagccaccgggtgactttctggcttccagatccatctgcctgagacccccgaactcctctcctcccaagcagaggcgagtgagtggcattagctcccggacccattcccggtcctagctgggcatggggctgacggagatgaccaagccttggtctgctctctagcagcttccacagacttggctcgtggccttccttatatccactgggaacaaacagcctcgcgctctataccaaacagcctcgcgctctatcccagtcgcccattagcttgattcaaacaaagccccagcaggcctttgcgtttttatccttcataaccttcatcttaatttgaacttgtagcttggactttaaggtagcatggctctattgctgtcaatttactgtttcactgcacagcaatcacagccagtgaatgttacacacatcttgctagactagtataaaaatcattgggtaattgttggttctaatgacctgaaaggtgttcagtttttgttcttggttttgttttgtttttgatttcttgggggtgggttttgcttgttgttccttttcatttgggggtttttggggaaaaaatttatttttggttccaaatagaaaaacaaaacctattttgatctttagtgcaaacgagggctagggacttagcctcctccaccacctccacactgcttcattctgccattcactcactgcagcatattcaagaataaagcaatatcgtttactacattttttattgaaggtcagccatgctttctgtattatattgcatatgaaattgtttacaaaagaaacactaactcatacttctctttattggttgcaagtggcacgcaggaacagagggagagtggggggctggtgggggaggggagatttttttttttttttcttgaatgtgcttcgcaagccaggctatcttccaaggaaggcagacagtgggagagcagagggactgactgcaggcaagcacattgaagaaagacactggcgggtttccccaccctcaccccaaagcagaaaactagcagacgtcagctcagccccgtcctgggcacagacactacacaaggagacgctggaagttaagcaatactttaatactgtaatatgtttgttttctttttctttcttttttttctaccaaaaaaaagtaagtaaactaaaacacaaaaacatataaataaaatccatccctcttgtcggggacctgcaggggggcaaccttaatccaaacacctggctatcaaataatcagaatgtattgtctcagacaggatttcagttccgggaggcaggggcatgatgggggagggggccggaggctgagagacaaaagttccagagcctccctcgaaggttctctactactgtattctgtacataatgtaccatcccatgtggaatctgtgagtgtcctcttaagtagcgtgggctagccaatctgccgttcatggtgtattgtaaactccgaattccatatgtaataggatgcaagtctaagcgtttcatgtggacataaatgtatctaaataaaactttccctagcactgtggctgacctcacccttacttttatactttagtatgaaactgatgagaactttggtagtgagtattttttttatatatatacatatatatgtactatctatatatatatctcaagcatctttcaggtctttgtgtgtggctttcttaaagccctgttgtaaaaaattactatgtggatggcagtctctcacatcacagatgtggaaagtataattttatatttgtattttcaaataaataagtttgtgaaaggtttccatcctctactgtggtccagaaatcaatgtgtttgtctgacaaaaaaaaaaataaaataaaataaactgttttgaacaga

SEQ ID NO: 113 - DNA, 496 bp, Artificial Sequence
  MSI2 transcript
gcagcgccaacgactcccagcacgaccccggtaaaatgtttatcggtggactgagctggcagacctcaccagatagccttagagactattttagcaaatttggagaaattagagaatgtatggtcatgagagatcccactacgaaacgctccagaggcttcggtttcgtcacgttcgcagacccagcaagtgtagataaagtattaggtcagccccaccatgagttagattccaagacgattgaccccaaagttgcatttcctcgtcgagcgcaacccaagatggtcacaagaacaaagaaaatatttgtaggcgggttatctgcgaacacagtagtggaagatgtaaagcaatatttcgagcagtttggcaaggtggaagatgcaatgctgatgtttgataaaactaccaacaggcacagagggagagtagcgatttacgaagagcaaatggaagcgaaaaccccttttcttctttgggccggctgtgtattgct

SEQ ID NO: 114 - DNA, 2247 bp, Artificial Sequence
  MSI2 transcript
aacgactcccagcacgaccccggtaaaatgtttatcggtggactgagctggcagacctcaccagatagccttagagactattttagcaaatttggagaaattagagaatgtatggtcatgagagatcccactacgaaacgctccagaggcttcggtttcgtcacgttcgcagacccagcaagtgtagataaagtattaggtcagccccaccatgagttagattccaagacgattgaccccaaagttgcatttcctcgtcgagcgcaacccaagatggtcacaagaacaaagaaaatatttgtaggcgggttatctgcgaacacagtagtggaagatgtaaagcaatatttcgagcagtttggcaaggtggaagatgcaatgctgatgtttgataaaactaccaacaggcacagagggtttggctttgtcacttttgagaatgaagatgttgtggagaaagtctgtgagattcatttccatgaaatcaataataaaatggtagaatgtaagaaagctcagccgaaagaagtcatgttcccacctgggacaagaggccgggcccggggactgccttacaccatggacgcgttcatgcttggcatggggatgctgggatatcccaacttcgtggcgacctatggccgtggctaccccggatttgctccaagctatggctatcagttcccagccctattaccatatttaaatgcaagcttcccagcagcggcttatggaccagtggcagcagcggcggtggcggcagcaagaggatcagtcctgaatagctacagtgctcaaccgaattttggcgcgcccgcttccccggcaggctccaacccggcgcggcccggaggcttcccgggggccaacagcccaggacctgtcgccgatctctacggccctgccagccaggactccggagtggggaattacataagtgcggccagcccacagccgggctcgggcttcggccacggcatagctggacctttgattgcaacggcctttacaaatggataccattgagcaggtgctttcgttgccatctcactctgagagcatacctggatgtccaggcaagactgggcgaagtttctgagtggccctttgtttaggtgatgtcctcagacctggacccccaccagcctcactccccatcccaaccagagatggctcacttcggatcgagggttgactacatctcatcatctcacgaatctgctgtaatataagacaacagcttttaaatgtgtatataacccatgatttcggttttgttttgttttgtttttcttgatggtttccctctccctccctctcttcccattctccttttaaatctctttgaatcacatttggtagtgattttgacttagtccagtagtcacatagctttaatatctagttcaaagctaaccatagtataattgttatattaaggagttattttttcttaaaacatttttttttgcttgttttggttctgttctcacttttaaaggatgctgagatggtaatatgactctccatattttggtaccaattctgagactgtatgaattttcaggtggaactttagcacacactgaagcaaagttgtgaagtgcagggcgggaggtgggcgtgagctttctattttgcgttgtagaagaagtgagatgtagtaagctaattaacagactttctagcagttctttttgtgatgtctctttgttaatctgagtctatctattttcggcaataaggtaaggacgacagtgttttgagtgtcctccttttctataagtgctttttttctgttgaaagaggtgatattataaggttttttgaaattgtgaattctaaaaaagaaatgttgtaaatacaattccattaactacatagaaactattaagaaagagagaatcaaaaatatttttgtgagggagtcggtcccaggcagtttgatgctctgtggaaggaggcgggaagggaacgttggccaagtcagttactgagatgaagatcgcccagctgccaggaccaccccaggacaagttagagcactgtttagctcctttgtctgtgtgatagacctaagaactgtattagtgttgtaccagcctattaacctcttgtctgtgcacagcttcaaatgttaccgtctagttagatttttatttaaaatatgaaaaactgcttttcccaagatgttttttaaaaacaaagctacaattttaatatttaacatatttaaagtttcaaagc

SEQ ID NO: 115 - DNA, 331 bp, Artificial Sequence
  MSI2 transcript
attgaccccaaagttgcatttcctcgtcgagcgcaacccaaggcctgcctggacctgtgttctgcttctgtatcctggactgagaatcactgtgctgcctcctgaggagctgctgctggagtggaccttggggcagctagcacattgcctgtcttcctaaaggcatcggtgcacagggcacctgggaatgacagcgggcctgagagcagagctcagtggagatgctggacccctgaagatggtcacaagaacaaagaaaatatttgtaggcgggttatctgcgaacacagtagtggaagatgtaaagcaatatttcgagcagtttggcaag

SEQ ID NO: 116 - DNA, 5204 bp, Artificial Sequence
  MSI2 Transcript
gcttcccagcagcggcttatggaccagtggcagcagcggcggtggcggcagcaagaggatcagagcatacctggatgtccaggcaagactgggcgaagtttctgagtggccctttgtttaggtgatgtcctcagacctggacccccaccagcctcactccccatcccaaccagagatggctcacttcggatcgagggttgactacatctcatcatctcacgaatctgctgtaatataagacaacagcttttaaatgtgtatataacccatgatttcggttttgttttgttttgtttttcttgatggtttccctctccctccctctcttcccattctccttttaaatctctttgaatcacatttggtagtgattttgacttagtccagtagtcacatagctttaatatctagttcaaagctaaccatagtataattgttatattaaggagttattttttcttaaaacatttttttttgcttgttttggttctgttctcacttttaaaggatgctgagatggtaatatgactctccatattttggtaccaattctgagactgtatgaattttcaggtggaactttagcacacactgaagcaaagttgtgaagtgcagggcgggaggtgggcgtgagctttctattttgcgttgtagaagaagtgagatgtagtaagctaattaacagactttctagcagttctttttgtgatgtctctttgttaatctgagtctatctattttcggcaataaggtaaggacgacagtgttttgagtgtcctccttttctataagtgctttttttctgttgaaagaggtgatattataaggttttttgaaattgtgaattctaaaaaagaaatgttgtaaatacaattccattaactacatagaaactattaagaaagagagaatcaaaaatatttttgtgagggagtcggtcccaggcagtttgatgctctgtggaaggaggcgggaagggaacgttggccaagtcagttactgagatgaagatcgcccagctgccaggaccaccccaggacaagttagagcactgtttagctcctttgtctgtgtgatagacctaagaactgtattagtgttgtaccagcctattaacctcttgtctgtgcacagcttcaaatgttaccgtctagttagatttttatttaaaatatgaaaaactgcttttcccaagatgttttttaaaaacaaagctacaattttaatatttaacatatttaaagtttcaaagcacacctgtttggcttgggtgggggtggggtgggggggacattctttttcagtcttaatttttaaatatttgatcattttctattgtccaatcatttcagcacctccaaaggtccctaggacactttgcctctcttctccccctgccccccaccctgctcccacatctgggggcccatgggccaggagtggataagcctgcattaatacaacctttctccattcactttctatttacaaattaggaaagcaaccttttggtttatatatattttttttaatacctcagtgctgcaagtatcaccagagaggctatggaagaatttttttttaatttattgtagatgtaaacagaattttaaaaataaaaagtataaacatcactgcactgtgactggtgggaaaaactgacagtttcctctttgcacatgtttaacatttggctgttataatatatggtcctcggttggggaaagatacttatgatgaaggatattttttaatttaacttttttttaaatattggtaataggtcggcaacagcaactatagaagtacaactcaatagatggcattaaaacatattgtagtgtggatatatattttttcttttttaaaatgtgatattgacgttttattaatattttttaaattgttacgtttataaatttggtacttaaggcacagccagtatgagacactgaatgcgacatttattataaagagctgctgcactcctatttttataaattttactaacaaagtagactaatgtagacattcacagacatggtagggcaaaagcatcttcaaactaaagactccaaaatgctaactcagaaagaaagaaaaaacccgttttcaattctaatgaaacagcaacaacattttttttaattaaaaaaaaaatcatgttctttgtttttctaataaaatgttaggttgtttggtgaggtttttttgttgtttttttcctttctttgtttcttttcttttcccccaaacaaccagattaaatgctgagcgcttttaaaatatcaaaacttgttcaagtcataaaacaacaaaacataagttttatttaaaaaaaaaaaaagaaagaaaaaatagtaaattcccagaaattcagtgtttagacgagggaatttaattcctattttgtccatgttggtgatgtactgtacttcccttccttttctctgcatcccccatcacctcatagaagactctttgttgatcattgtatgttaataatgtataaaatggctatcttgtaagcgtgctgtcctggtactagtgtagcgactttttttctcctctttcttctagtacatattgataggtataacataattaaggtttaaaaaaaattagacatagttattcagatttaggaccagtaaggatagaactttctcttatttatgaaaaaaaatgctaataattttggggcagttttttcctttaattatttttttcaatttcaagtttaattttattttagctgatctgatgtggtttcaactaacccaaggtctcaccatgttaaaatgccggcggactctacggcgttttgtagatccccccccccccacccactgtgaaggggtgccatactaccttaaatgctaatgctagatatgcaaaactggatttttttaatttattttttaaaagagggaggcatggtatattaaaatgattttactaagagaaaaaatatttttttaagaatgctcagaagaaattgataatctgtgtgaatatgttttagatgtttatataccttttgaagagacccagtagcccatagcacaaatcttgtggaaatccgatatgttttaatgtggctacctaggtttaaggttcacgttagtccccccattccatctagaagtccattttgaaagatttttgtaaattcttttaacactgatgtttcagcctcgtctttgtttcagttaagctcaatggcgaacatgggaaccacctttcgccttccctgggggagaaaccctcttggctgatggcttttccccggaattatcaaacagccaccgggtgactttctggcttccagatccatctgcctgagacccccgaactcctctcctcccaagcagaggcgagtgagtggcattagctcccggacccattcccggtcctagctgggcatggggctgacggagatgaccaagccttggtctgctctctagcagcttccacagacttggctcgtggccttccttatatccactgggaacaaacagcctcgcgctctataccaaacagcctcgcgctctatcccagtcgcccattagcttgattcaaacaaagccccagcaggcctttgcgtttttatccttcataaccttcatcttaatttgaacttgtagcttggactttaaggtagcatggctctattgctgtcaatttactgtttcactgcacagcaatcacagccagtgaatgttacacacatcttgctagactagtataaaaatcattgggtaattgttggttctaatgacctgaaaggtgttcagtttttgttcttggttttgttttgtttttgatttcttgggggtgggttttgcttgttgttccttttcatttgggggtttttggggaaaaaatttatttttggttccaaatagaaaaacaaaacctattttgatctttagtgcaaacgagggctagggacttagcctcctccaccacctccacactgcttcattctgccattcactcactgcagcatattcaagaataaagcaatatcgtttactacattttttattgaaggtcagccatgctttctgtattatattgcatatgaaattgtttacaaaagaaacactaactcatacttctctttattggttgcaagtggcacgcaggaacagagggagagtggggggctggtgggggaggggagatttttttttttttttcttgaatgtgcttcgcaagccaggctatcttccaaggaaggcagacagtgggagagcagagggactgactgcaggcaagcacattgaagaaagacactggcgggtttccccaccctcaccccaaagcagaaaactagcagacgtcagctcagccccgtcctgggcacagacactacacaaggagacgctggaagttaagcaatactttaatactgtaatatgtttgttttctttttctttcttttttttctaccaaaaaaaagtaagtaaactaaaacacaaaaacatataaataaaatccatccctcttgtcggggacctgcaggggggcaaccttaatccaaacacctggctatcaaataatcagaatgtattgtctcagacaggatttcagttccgggaggcaggggcatgatgggggagggggccggaggctgagagacaaaagttccagagcctccctcgaaggttctctactactgtattctgtacataatgtaccatcccatgtggaatctgtgagtgtcctcttaagtagcgtgggctagccaatctgccgttcatggtgtattgtaaactccgaattccatatgtaataggatgcaagtctaagcgtttcatgtggacataaatgtatctaaataaaactttccctagcactgtggctgacctcacccttacttttatactttagtatgaaactgatgagaactttggtagtgagtattttttttatatatatacatatatatgtactatctatatatatatctcaagcatctttcaggtctttgtgtgtggctttcttaaagccctgttgtaaaaaattactatgtggatggcagtctctcacatcacagatgtggaaagtataattttatatttgtattttcaaataaataagtttgtgaaaggtttccatcctctactgtggtccagaaatcaatgtgtttgtctgacaaaaaaaaaaataaaataaaataaactgttttgaacaga

SEQ ID NO: 117 - DNA, 5141 bp, Artificial Sequence
  MSI2 Transcript
ctttattgactcctgcttttctattttttttcccagattgaccccaaagttgcatttcctcgtcgagcgcaacccaagagggcttcccagagttagcagtatttcctgggtttggaggaattgctgtttcttccataagtgtgaatcttcaaatttccaggttagtcatttgatcactcagagcttggatcagattacccaagagggaagctggaaaaatcccttacttggtaaaaccatgggtttgacctgtttttctctcccctttgttgtcccccttccttaactttgtttttaaatgagaaaagcaatatttcactttaatacactctgcagtgatgcattcaacttgaaaacgagacagaatgtttctcaaacttgtcaaagtgtgcattcctttcccaaccctcaactccacatctggaatgtttgtaggtgaattaacatgaaaccacttgggggagaacatctttccatgaattctagggatttaataataacaccttactgtgtatgacatacttcactttacggaggacttggctgttcattatctccatctgaattccatctcctggggcatttggctgttgtccctaaccttctctcagtctcagtgtgctcatctgtaaagtaggagtgatgacatagctctcccagaaggtttttgtgtagagtaaaagagcatggtctgcgacaaaggtcttcgtcattgtcattatttttcttgtcggttttctcatcattgttccggttgttgtttgcaagtgggacagcctgggttcggatgctggtggtgccactccttagctgtgtgaatttgaacaggtcgtttaacttccttgaaccttcgtttttccatctcatagagtttttctgcagattacgtaaattaataaaggtaaagggactgaaaaaaaaaagacgttgcatggtaatgccatagaagcgttaacctgctatcttatttatttatttatttatttttgagatgggatctcactgtgttgcccaggctggtcttaaactcctgggccaagctatcctcccacctctgccccccagagtgctgggattataggcgtgagccactgtgcctggccaacctgctatttttttctgccatttttattaagtcctcacatcaatcctgtgagaggttgagtaataataatgatactagttgaatttatatgccaggcactattgcagagactttattaatccatttaattctcatgcagccctatgaggagagtactagtatgagctcattttgtaaatgaggaaacggaggcacagatggatgtccaggagccatacccaggcaacctggctgtagaactgggcccttcaccactgtgccatgctgtgggggtacctcaggggatggcccctctgctatgcgacaggagatgggctcggagaggtcagcgggctgtgctgagggaaccatagccagaggcccagtactttgaagttctgcgttcctgggcctaggtgatttgccttgccccatcatgttataccagtgtgtgacactgtcttgtatcttttgatctgacccctcaggccatcttttcagatggttctggagttactcaaccccagtgaggcaacagagtaaatgccaaggttgtatggtggttgggaattgggacagggtggggagaaagtgtcttccctgtctacggttattttccaatagaatttgagcctggccaccgggatttcagactggagttagcgctgggggccaaaacccaggagcatagaactttaagtcgaccctgatttatttatcgattatctcccttcaataaaacttctgtgtttttgtgttcaggtcacagtggcagagggaaccctgcccctcctcgtgggatgggcgaggagcttgtaaatgatatgtcagtgtgtgtttcagttaaaatgagtttctctgctttctggctgtccctccttgctaacgctcgccagagaaatattccgtggaggtgttaaaacaagactagtggaaagaaatggtaacttaaaaagaaagaaaatccatggcagtgaagaacagatttcatattagtactgtcataatattctctaatgtatcatataaataaaaatctgtttagaccagaattttaataagtcaccaaaggaaggttttgaatcctacagtttatgtatccccctttgcgatgtaaatgttcacatctcaagtgaagtagagtggggagacgcttcggagactagaggctgttaattagtgaggtatattttgatcagggcaagaagactggttcattctatatcttcgtatttctctggatgattatttcattttgtaaaatatgtaagagttcccccccagccccttatctttttaaaaattaaaatggtcatgctcttctaaaaagatgacttaatttgaaggctttggagacatgaggatgatggggatccatcactccctgttctttgctccctttggactggagtgttggctgtctggaagagttttgcttgagctcgtgggttattttcttctatgtggaggaaccaagagactctttagcatctttcagcaagagcgaggtctgggtgtactcaccatctgtcttaattatccttgtgtataagaaaacattcccatcttttcccaattgccatttccttccttaacttttagaggccagaacctcatctgttgatgcagggagatctcacttctttaacctatttctagttgccttcagtcacagcggggtggccaggggcgagtcacttaagcctccccaaggctcagtttcctcctttgtaaaagagggagaattatagtactcactgcctgcagttagttgtttggaggataaatgcaaaattcttcactgggagtctggcataaaacagcactccgcaaatgtctgttgatcttattattatagaattattatttttattattattatttttaagacggagcctcactctgtcgcccaggctggagtgcagtggtgagatcttggctcactgcaacctccgcctcccgggttcaagcaattctcccgcctcagcctcccgactagctgggattacaggcacctgccatcatgcctggctaatttttgtatttttgtagaaatggggtttcaccatgttggccaggctggtcttgaactcctgacctcaggtgatccacctgccttggcctcccaaagtgctgggattacaggcatgagccaccgtgcccgaccagataattattttaatagaataaaaaaaagtacaatggtgaaatgactgaaggaaaatcttaattggaatgtaagctccctgagggcagggaactgttggtggctgctgtttggtaactaaatgaacgaactgagtccagcctgcttttttcaccgtggaggtagctgctacccagtaaagttacgttggttgcttcagatccacacttaggttgtagcagggctgagctagcagtcaggttttcctgttcctgtcttgtcctctgacgtggcttcagggccctggaaggttctctgctaagcacgcatccagatgggtgcaatggcagcccgagcgtgtacacgcacacctcctgttctgggggagtggtttcttggcagcttctcaagggcgaagggtgagttttcggcatctggccttcccttgctgctgtgggtcgggtcattctagcatcttgccatcttggatgatctgcagctgtcatctcggcagccaccatgaactggcctgccagtgggttttctcgttcccagcgaggatgtggtggtgtgtctgcagcccttttccacagcagcgaggacctgggaggattagtggcttagcttctttcttgtcggtgagcaccgctccttcctatgttccaagtcagtagcaggtgtcagcttaaggaggaggggcaccttgtcctacaaatgtcctttccctggattttgccgttcctcacatgctttcgtttcattgttgaggtcagtctcctaggactatgaagctcctgtgttctttgtcagatgatttttggcagacaggtgagcctcctctgctgcccgggaagggaggagaaggcagtctcggaggaaccagctgaccatctcaggtgccgcttggggcagcgtccattctttgtgtcctattttgtatttttaaacccggacaatcttcggccgaaaaccccgagaacacacaggggagatgtagttcatcatctctctcttcccgtttgatctagtgtgttcatttaacaagagctgggttaatccttgttcattgccaactgtatgcgtgcacccacttttggagggattcaggaaatactcggtgcttcctgatgtgcatggaacacagtctcgacgtcttagcttggtgcttaattaaggtccttcaaaatctgtaccagggtcttttccaccttatctctcaattcttctagtttttgctgccttgaaactgaatgctcacccttctttgagaaacctgtccttttgcagatttccctctctgcactgcccttcccttctctgtacacaacagaccctctttgaaagcccagcttcatcattatcttttccacgaagctgtccctgcccctttgtggaaacagtcatacttctttggatttccacagcacttaccttttctttggttaagccccttccaacaataataaattagcagaaacttgtggagcacatacatctgttgcatgttgtgctagacattcactcattcatttattcaccaaacaagcaccgaccgtttgccagatactgtgcaaaattcgggggatagtgtggagaatatgacaaagttcctgctgtcaccaaacttttgctctagtggggcaaataagcagtgagcaaacagataagtttatgacatgctctcaggtgtgatcaccaaagccagggaggagcgatggcgggagtggggtggaccatatctatggtacagtcagttgccactttcagtagttcagaaattatttaccaagtttctgctcagcaccggggattgcgttaggtactttgttctgagttttatggtatttaggttcatgccttatcttctccattggacacaagcatattttactaataattaaaaaaaaaaaa

SEQ ID NO: 118 - DNA, 274 bp, Artificial Sequence
  MSI2 Transcript
gtggaagatgcaatgctgatgtttgataaaactaccaacaggcacagaggtggattggctgaggcctgaaatggagaagggacttgtccaagatggttcagggcatggagtatggagcccactgttgggatcgccaagatgcccgataatagcgttcctcatccctgcctgaggagtgaacagactgagggggtttggctttgtcacttttgagaatgaagatgttgtggagaaagtctgtgagattcatttccatgaaatcaataataaaatg

SEQ ID NO: 119 - DNA, 472 bp, Artificial Sequence
  MSI2 Transcript
gtcaaaatggccgatctgacatcggtgctcacttctgttatgttttctccctctagtaaaatgtttatcggtggactgagctggcagacctcaccagatagccttagagactattttagcaaatttggagaaattagagaatgtatggtcatgagagatcccactacgaaacgctccagaggcttcggtttcgtcacgttcgcagacccagcaagtgtagataaagtattaggtcagccccaccatgagttagattccaagacgattgaccccaaagttgcatttcctcgtcgagcgcaacccaaggtgttctagctgagcgtttaagagcatggactctggaaccagactttgaatccttgctctgccactgcagctgtgtgaccttgagcaagctatctaaatattctgtgcctttgttttgtcatctgtacaatggagatgatgatggtatcaccttcatggggttgtc

SEQ ID NO: 120 - DNA, 1624 bp, Artificial Sequence
  MSI2 Transcript
tttctgttgtgtagttctgattttccgaacaggtgtttagatgatgaggggttatattaagaaagtactaaagaatacgtcttatcacgtgaatcagtatctttttattcaacatggatggagtgggagtggagaccaatctgttttatattcctgcaatctgatgaggatggtcacaagaacaaagaaaatatttgtaggcgggttatctgcgaacacagtagtggaagatgtaaagcaatatttcgagcagtttggcaaggtggaagatgcaatgctgatgtttgataaaactaccaacaggcacagagggtttggctttgtcacttttgagaatgaagatgttgtggagaaagtctgtgagattcatttccatgaaatcaataataaaatggtagaatgtaagaaagctcagccgaaagaagtcatgttcccacctgggacaagaggccgggcccggggactgccttacaccatggacgcgttcatgcttggcatggggatgctgggatatcccaacttcgtggcgacctatggccgtggctaccccggatttgctccaagctatggctatcagttcccaggcttcccagcagcggcttatggaccagtggcagcagcggcggtggcggcagcaagaggatcaggctccaacccggcgcggcccggaggcttcccgggggccaacagcccaggacctgtcgccgatctctacggccctgccagccaggactccggagtggggaattacataagtgcggccagcccacagccgggctcgggcttcggccacggcatagctggacctttgattgcaacggcctttacaaatggataccattgagcaggtgctttcgttgccatctcactctgagagcatacctggatgtccaggcaagactgggcgaagtttctgagtggccctttgtttaggtgatgtcctcagacctggacccccaccagcctcactccccatcccaaccagagatggctcacttcggatcgagggttgactacatctcatcatctcacgaatctgctgtaatataagacaacagcttttaaatgtgtatataacccatgatttcggttttgttttgttttgtttttcttgatggtttccctctccctccctctcttcccattctccttttaaatctctttgaatcacatttggtagtgattttgacttagtccagtagtcacatagctttaatatctagttcaaagctaaccatagtataattgttatattaaggagttattttttcttaaaacatttttttttgcttgttttggttctgttctcacttttaaaggatgctgagatggtaatatgactctccatattttggtaccaattctgagactgtatgaattttcaggtggaactttagcacacactgaagcaaagttgtgaagtgcagggcgggaggtgggcgtgagctttctattttgcgttgtagaagaagtgagatgtagtaagctaattaacagactttctagcagttctttttgtgatgtctctttgttaatctgagtctatctattttcggcaataaggtaaggacgacagtgttttgagtgtcctccttttctataagtgctttttttctgttgaaag

SEQ ID NO: 121 - DNA, 7612 bp, Artificial Sequence
  MSI2 transcript
gggatgtttttaaagggtttgtgtagaagggcagagacacccgtggaaagtgtcactgttgccctcaattgctggagagcctggaggctgtcataagtccactcatcaaagtaattgtttgccagtgttttgataacaagcaacagaaacaaccctggctgctgtaagcagcaaaggagattattagaaggagcttgggcagtgctggagaattgacaggatggctggagaacaggactcagaaaacaggcaggaactgggggagctgggtggtcagagccccagccctagccaaggggacaactcaggaatgacctggtcagggggccactgttgccaacactggatgccagattctgctactgacaccagaataaattcggagccagtccctcctcctctgccctaccagctccagaggcagatcctaaggagggcttccgtctgtcgagcccaggttcacaggcctgcctcccagcctccagccccaggaagagccctgaggccttctcagcttctgtgatggccgggggggtgggcctctctccaagacttacacacagggagaggctcaggggctgggcagccaaaaaatgaccacagacattcacctccaaagtcaaagcgaaagtcacgttctcggtggttgggtggccgtggtggcatcttaagagcaagaatgtactgaggttacatatacactgagtggagggaggcagagtgagcctgacccatacggccttcatttagcctgaatcaggcagggacttgaagaacgaagtggcctgagcagccacaatgactccctgagagctgaatgacacctcacatcactgggggttagtgacagatctgagatggtcacaccagcccctggacttgccagctgagtttcttgtgtgcccagtggccagcccagtggcagtttctacaagtgtctttcagtgaaccactcaccccctagcttgcctgctgagacaggggtctagaaacaatacgcttgtggtgaaacgcccatccagtcacacggcacggggctcctgagtcggagctacactttgtggtatgcatcttaacacatccagcctctcaccattctccaaagatcttggcacctccgtggagtgtcaaaactgcagtgtgggccatggctcatcaggcccagcgttttccagaggcccagggtgagccctgagtttggccgctaaactgtgactcctgagttcgcatgctctgtctggtgctggctctgttgtccactgccttgggaggatgcatcacgaggactttaatctcctactgtcaatagctttaaggtaaagtacatgcagggtaagctctccaaatgtcagagccaggcgcaggctttgtgtttggtgctgatgcatggcatttactctgaaggttcaggaaagtacatgggctttgcggggtctggctgcccaaatatgggttaattagcaacagctgacagcagcgcccccggccacaggagagaggtgacccagacccttaataaaaaaaccctcatgagagacaaattattctgtgaaggaaaataactcaggctttcctcattgccaccctccgtgagattttaccccagacctgaggcggctgtactaacaggactctgatctttctctttgtgttcaaggatatcccaacttcgtggcgacctatggccgtggctaccccggatttgctccaagctatggctatcagttcccaggtgagtggcttggtctcccagggctttggaagcacaagaggtgggctgcatttgcggggaggtgagaggacccctaaagagaatgcatttcttacatgcatccacttgaaaatgacctatacatgataaatttcaaatccactgaagttcaaggccaggatgcagctcagagtttttgattaactcaggtataactcactggtgccgggtatttgagaacggcagcttttaaagggaaagcagaacggaggcaggaggcctccctgtgctcacctcctttttctgaagcctcttccgggttttttctcactggggactgaactctaggcccagggctttctttcaccctctaccaccccttgcccgcctccccgctccctgtgtccacctgcccaatgtttgatgtctccccgcccttgcttcctttcctccacccctcctcctcctgctccgtcctgactgctgtggcttcggcgtccttctgggctgtgttgatgttgagctgctaggggttgggactgctgtccatcctgggtgagctgtttagattatcctttccctctgcgtgggtatcaagacttgaggcagagcccatggtgatgcccgccacatgccagggctgctccagctccaactccaggtagctggcaggcctgggacctcacctgggagaagcagcgaggggaaacatggtcttgtagggatggggtagggacaatgaaggaagcgatgttttacagggtatgcttttccccagcctttctcaaatctggagtacagataagaatatattggggtgtttgggggtctccatgtgcaaaggacagccgagattccagaggaggtgtagggacagagtcttgtcccctgtgggttttctgctgagggaggaggctccctggggagctgggctgcaggcggagtttcacccctgccttgtcgtcatgcctagacctggaggtctgggagtgtcagggtctgagatgaattaggaagcaaagggagtgaagacgcgtcgctggaagatggcgccctgacactgatgacaggagggcagagtgagggcttggcctggtctttgcctcagaccaagcattggggcgggaagggtgtagttggagagatgaggaggcatttgggggcacctagagccgtgctctatggcagacagatccttcaaccagcggtggaagccccaagctcttgtcctgtctccgataccacctggccaagtggccatgggcgggtcactcagcttctagggccttacttctctcttttggaaaagggggtctacgaggaatggtctttggggtctgtgcccctctccgattgtgtgggtcagagggtgcagggaaggcaggcacagaagcagaagtccaagatggaggagtttgagtgcaggtggctcccccaccagtgtcccctggggctgggatggttccccatctgggaggggcctcaacaagactcctcggcccgcactaaggaagccgtccccaaagatgccccccagcagcaggctgcttcaggatggccacaggctcccagacctgaagaaaagaagggcacatgcaagaaggaccaccacaagagaggccagcactcaaccagccactgtcattagactttcccatttccaggcagagacccccaccaggcacaggcacagaggtagaaactccttctgaaggggtcacagtacgcaaggggtaggacctgcatttggaccaggcggtctgggccttaactgccaccctccaccatctccctagtgtccatttttctggtacaataacactagccacaaagagctactaaaacttaggtttaaattaaagttggctgggtgcggtggctcacgcctgtaatcccaacactttgggaggccgaggtgggaggatcacttgaggtcaggagttcgagacccgcctggccaacatggtgaaaccccgtctctactaaaaatataaaaatgagccgggcgtggtgcccggcatctataatcccagctactcgggaggctgaggcaagagaatcacttgaacccaggaggcagaggttgcagtgagccgagatcacaccactatactccagcctggataacagagtgagactctgtctcaaaaataagaagaagaataagaagaagtaaaaagaaaatttcagttcctttagaaacactggccacatttcagtgactcagtaaccatgtgtggcaagtggctattgccagcacagaatgtagaacagtcaccgtggaaatttcttacgggctggtgctgttccagtgcctaaaggaagagggctgttactggggggtgcacggtccacacactcacacatgcgcaccactccttcctggagacacccatggggtcagtgtccctgtgcttcattctctgccagtcttgctcagcccagaagctggtggcttttcctctcacttcctcccctcgcttcccctccaggggatgacaggtgcaaaggctcacgcatctcggccctccaggtgcagcactgatctgtgcactcagcctgcacaacctgctcccctccgtaatcaggctgccattgcccctgagtgtgatgctagtcccaggaggacagagttgagggatcttaacatggaataattgggagaggacatcaatgtggaccctatgctaaggcccctgtctcatcagagggcctgccccggggcccccatgagctggctcaggagacatctagtagaactctgcctccatggcctgcatccctgggcctgccccaggcagacggacatgcagcagtgaccagattgaggctgggcagagtgagatagggtcccctgggtgcccagcccttcaccaaagccaagactttgggaactgggggggcgggggatggactgggctgcaggaaggaggatagagtggggttggagtgtttttgcccagggcccatccgtttgtcatggccacagtccctgcagcttaatacaatcaggtgggtttgaatgggacagaaatcaggcattacaatgtacagacagcagcttcctgacctttgggagttagctgccctgttctcaaccatcaaacttttatttttccatccttcccaccttcccactcactatggaagcctcttgattctcttaactaatggccagaaaggggccaccagtggccacaagagtaaggttgcctgctctcagggagggcaagagtggagtggccctgaactgggaagggtggccccatggctggggccgcccaccccactcatctgagaagccaggctagggtcagagtctattcccctcctgaccccctcccaaaggatatgctccttgtggagtgttgggtctgtgcttatcccagatgcctccccaagcagccaaatgtttccagacacaaagcctaggtaggtccatgtgtcattccagtcaagagcgcagggagtagctgagcccctctagaaccaaacttgatgactcctggagccacagcctcagggccagggctacagagaagcttctggatgccctgacttccatctgtcacctcatgccaataaggaaagtttcgggtggatctggccgaggcggctggggtacagaagggggacccagagatggggcagcccagccctctgccctctcctcatccccatcaggtagattctgcgaacccctgccatgccccggggatcctgtgacttcgtgacattacacagcagtcatgtcacctcccaactatcctgatgttttctttgtggtctttcttttcagtgtcctcactgtatgcccactttacccacttcccacctccggtccccctcatgctgtgcatggatcccgaacctctcccagcccctttctgagtccccctagcagggccatgcaagacagctagctgggagaaatgaaagcacgcccaggtcctagagagctccgagactcttcatcctggctgttctcactcataaaccaggtagcctgggtcccctttcaggaagatgcaatctgatcccctttatgaagggtcctcctttccacctggacttaccctttctatgaacatccatctcatctggttgggggtgcagatatcactcttagtagagcctcattctcaccatgtgacatggggctgcacggaccttcctgggccacacagctggtggcaagtggcacctggctgtgcccatcttcagaattctgtcagaataaatgttcctagagaaggctagacactggatagtttttagagtggggagtagttcttttctagaaaggctgggtggtgaccaaccactctgggaacagctccttcttcccacatccaggtgtccaccccagcctggaatttggatcaggatcccgtcttgaattattatgggttgtgatcagggatctcccctgcttcctctcttttccccactctggacttctcatttcagtttaatctgttaatttctgttcttatttcactttgcagactatttgccggtttcacaagacataatttttatcaactagctcttaagagaggcatagcaaagtgggggttgctaccatttctagagagagaggacacagtcccggcctgggctgcccccgctccagtcaatgctcactgaaagtctgtcttagctgcctgtttgaatgactgttctttttctcatttttaattcttggactcatgtcctcattgcttcactcaattaaaaaaaaattattctccagtcccctcccactttgcttcttgtatgcattgtgaccgaccccacttcctcagaatgtaacggggccagagggaaacttctcacaaacttcgtagagcctcctcaggggaagctaggaagaagacatcaaatgtttttaagtcatgaccaaacaggcttgttggggacatatcatggggtgagctttgaagtgctggtggtccagaggggttgcagatacgtgacttggagcacccgtgtcttacgatggacagtgataaaggtgaacacacagagacagactattctctaagaatgtgagaaacctgatctggaagaggagctatataaacactatctgactatctttgtcttttggggccagtggctgttggcataatcacaagcctgtctgtcttcgagaagggacagtggagtcatccaggtgctgccacatgacaggcacggtgggcaccgatccacagtgggccccgccttccccagctcgcctccctgcctgtgctggcctggccttgcctgctggcaccattggagtaggagggggtggaacacagggggcccatcctgatcaggccccatctcaaggttggcactcctgcccatcacccttagaaggatcttttcccatggcttgacttccttcatttccctaactgaaatacacccactctcttggaataatgacgtaccactcagttggaccctcaagagtcactgctttgtctgtgctggtagtttgtgagaagtgacccgcacgcttccatttgatgcatttgatgtgagtgaatccatacatttgaatgtcattgtccttgagaccctacatgtgcagtttggctcatctcattaaagatgcttgatgtaataattggttagtttccttttattttcctgcaggcttttccatgagtattatttttttcaaagaacaaatctgtatggcttttccccatctccatattttgttttgctatgaattgctttgctttggtgaacttgtcctagtatgcttgcctcacaaacgttttagccattgtgaattttcttcatctctgtaaatagttcatctgtgcttctccctgatgacgttttattttttttcccctgtaagcaaccgaggtagaaaaataaattgtttaccatgga

SEQ ID NO: 122 - DNA, 398 bp, Artificial Sequence
  MSI2 Transcript
ggcagaattgcttgaacccgggagacagatgttgcagtgagctgagatcgtgccactgcactccagactgggtgacagagcaagactccgtctcaaaagaaaaaaaaaaaagaacacagcacggagaagatgcccagagcccttcactgtgggcccatgagatgacacagatctttccactaggccatcctgttttgttgctggagaactttgtccctggaagggcagaaatgattattctggttgcccaaggcctggtggcccaggtggaagatgcaatgctgatgtttgataaaactaccaacaggcacagagggtttggctttgtcacttttgagaatgaagatgttgtggagaaagtctgtgagattcatttccatgaaatcaataataaaatg

SEQ ID NO: 123 - DNA, 6301 bp, Artificial Sequence
  MSI2 Transcript
gctaagccgagcccacgtgtgacggctctcgccgctgccccggctccgccgctcgcagagagattcggaggagcccgggcgggggggaggaggagggggaggagggagcggagatctcggggctcggagccggccgccgctccgctccgatcgctgtggggcttggttttttgggggtgggggggcgggggggctcagatatggaggcaaatgggagccaaggcacctcgggcagcgccaacgactcccagcacgaccccggtaaaatgtttatcggtggactgagctggcagacctcaccagatagccttagagactattttagcaaatttggagaaattagagaatgtatggtcatgagagatcccactacgaaacgctccagaggcttcggtttcgtcacgttcgcagacccagcaagtgtagataaagtattaggtcagccccaccatgagttagattccaagacgattgaccccaaagttgcatttcctcgtcgagcgcaacccaagatggtcacaagaacaaagaaaatatttgtaggcgggttatctgcgaacacagtagtggaagatgtaaagcaatatttcgagcagtttggcaaggtggaagatgcaatgctgatgtttgataaaactaccaacaggcacagagggtttggctttgtcacttttgagaatgaagatgttgtggagaaagtctgtgagattcatttccatgaaatcaataataaaatggtagaatgtaagaaagctcagccgaaagaagtcatgttcccacctgggacaagaggccgggcccggggactgccttacaccatggacgcgttcatgcttggcatggggatgctgggatatcccaacttcgtggcgacctatggccgtggctaccccggatttgctccaagctatggctatcagttcccaggcttcccagcagcggcttatggaccagtggcagcagcggcggtggcggcagcaagaggatcaggctccaacccggcgcggcccggaggcttcccgggggccaacagcccaggacctgtcgccgatctctacggccctgccagccaggactccggagtggggaattacataagtgcggccagcccacagccgggctcgggcttcggccacggcatagctggacctttgattgcaacggcctttacaaatggataccattgagcaggtgctttcgttgccatctcactctgaggtgatgtcctcagacctggacccccaccagcctcactccccatcccaaccagagatggctcacttcggatcgagggttgactacatctcatcatctcacgaatctgctgtaatataagacaacagcttttaaatgtgtatataacccatgatttcggttttgttttgttttgtttttcttgatggtttccctctccctccctctcttcccattctccttttaaatctctttgaatcacatttggtagtgattttgacttagtccagtagtcacatagctttaatatctagttcaaagctaaccatagtataattgttatattaaggagttattttttcttaaaacatttttttttgcttgttttggttctgttctcacttttaaaggatgctgagatggtaatatgactctccatattttggtaccaattctgagactgtatgaattttcaggtggaactttagcacacactgaagcaaagttgtgaagtgcagggcgggaggtgggcgtgagctttctattttgcgttgtagaagaagtgagatgtagtaagctaattaacagactttctagcagttctttttgtgatgtctctttgttaatctgagtctatctattttcggcaataaggtaaggacgacagtgttttgagtgtcctccttttctataagtgctttttttctgttgaaagaggtgatattataaggttttttgaaattgtgaattctaaaaaagaaatgttgtaaatacaattccattaactacatagaaactattaagaaagagagaatcaaaaatatttttgtgagggagtcggtcccaggcagtttgatgctctgtggaaggaggcgggaagggaacgttggccaagtcagttactgagatgaagatcgcccagctgccaggaccaccccaggacaagttagagcactgtttagctcctttgtctgtgtgatagacctaagaactgtattagtgttgtaccagcctattaacctcttgtctgtgcacagcttcaaatgttaccgtctagttagatttttatttaaaatatgaaaaactgcttttcccaagatgttttttaaaaacaaagctacaattttaatatttaacatatttaaagtttcaaagcacacctgtttggcttgggtgggggtggggtgggggggacattctttttcagtcttaatttttaaatatttgatcattttctattgtccaatcatttcagcacctccaaaggtccctaggacactttgcctctcttctccccctgccccccaccctgctcccacatctgggggcccatgggccaggagtggataagcctgcattaatacaacctttctccattcactttctatttacaaattaggaaagcaaccttttggtttatatatattttttttaatacctcagtgctgcaagtatcaccagagaggctatggaagaatttttttttaatttattgtagatgtaaacagaattttaaaaataaaaagtataaacatcactgcactgtgactggtgggaaaaactgacagtttcctctttgcacatgtttaacatttggctgttataatatatggtcctcggttggggaaagatacttatgatgaaggatattttttaatttaacttttttttaaatattggtaataggtcggcaacagcaactatagaagtacaactcaatagatggcattaaaacatattgtagtgtggatatatattttttcttttttaaaatgtgatattgacgttttattaatattttttaaattgttacgtttataaatttggtacttaaggcacagccagtatgagacactgaatgcgacatttattataaagagctgctgcactcctatttttataaattttactaacaaagtagactaatgtagacattcacagacatggtagggcaaaagcatcttcaaactaaagactccaaaatgctaactcagaaagaaagaaaaaacccgttttcaattctaatgaaacagcaacaacattttttttaattaaaaaaaaaatcatgttctttgtttttctaataaaatgttaggttgtttggtgaggtttttttgttgtttttttcctttctttgtttcttttcttttcccccaaacaaccagattaaatgctgagcgcttttaaaatatcaaaacttgttcaagtcataaaacaacaaaacataagttttatttaaaaaaaaaaaaagaaagaaaaaatagtaaattcccagaaattcagtgtttagacgagggaatttaattcctattttgtccatgttggtgatgtactgtacttcccttccttttctctgcatcccccatcacctcatagaagactctttgttgatcattgtatgttaataatgtataaaatggctatcttgtaagcgtgctgtcctggtactagtgtagcgactttttttctcctctttcttctagtacatattgataggtataacataattaaggtttaaaaaaaattagacatagttattcagatttaggaccagtaaggatagaactttctcttatttatgaaaaaaaatgctaataattttggggcagttttttcctttaattatttttttcaatttcaagtttaattttattttagctgatctgatgtggtttcaactaacccaaggtctcaccatgttaaaatgccggcggactctacggcgttttgtagatccccccccccccacccactgtgaaggggtgccatactaccttaaatgctaatgctagatatgcaaaactggatttttttaatttattttttaaaagagggaggcatggtatattaaaatgattttactaagagaaaaaatatttttttaagaatgctcagaagaaattgataatctgtgtgaatatgttttagatgtttatataccttttgaagagacccagtagcccatagcacaaatcttgtggaaatccgatatgttttaatgtggctacctaggtttaaggttcacgttagtccccccattccatctagaagtccattttgaaagatttttgtaaattcttttaacactgatgtttcagcctcgtctttgtttcagttaagctcaatggcgaacatgggaaccacctttcgccttccctgggggagaaaccctcttggctgatggcttttccccggaattatcaaacagccaccgggtgactttctggcttccagatccatctgcctgagacccccgaactcctctcctcccaagcagaggcgagtgagtggcattagctcccggacccattcccggtcctagctgggcatggggctgacggagatgaccaagccttggtctgctctctagcagcttccacagacttggctcgtggccttccttatatccactgggaacaaacagcctcgcgctctataccaaacagcctcgcgctctatcccagtcgcccattagcttgattcaaacaaagccccagcaggcctttgcgtttttatccttcataaccttcatcttaatttgaacttgtagcttggactttaaggtagcatggctctattgctgtcaatttactgtttcactgcacagcaatcacagccagtgaatgttacacacatcttgctagactagtataaaaatcattgggtaattgttggttctaatgacctgaaaggtgttcagtttttgttcttggttttgttttgtttttgatttcttgggggtgggttttgcttgttgttccttttcatttgggggtttttggggaaaaaatttatttttggttccaaatagaaaaacaaaacctattttgatctttagtgcaaacgagggctagggacttagcctcctccaccacctccacactgcttcattctgccattcactcactgcagcatattcaagaataaagcaatatcgtttactacattttttattgaaggtcagccatgctttctgtattatattgcatatgaaattgtttacaaaagaaacactaactcatacttctctttattggttgcaagtggcacgcaggaacagagggagagtggggggctggtgggggaggggagatttttttttttttttcttgaatgtgcttcgcaagccaggctatcttccaaggaaggcagacagtgggagagcagagggactgactgcaggcaagcacattgaagaaagacactggcgggtttccccaccctcaccccaaagcagaaaactagcagacgtcagctcagccccgtcctgggcacagacactacacaaggagacgctggaagttaagcaatactttaatactgtaatatgtttgttttctttttctttcttttttttctaccaaaaaaaagtaagtaaactaaaacacaaaaacatataaataaaatccatccctcttgtcggggacctgcaggggggcaaccttaatccaaacacctggctatcaaataatcagaatgtattgtctcagacaggatttcagttccgggaggcaggggcatgatgggggagggggccggaggctgagagacaaaagttccagagcctccctcgaaggttctctactactgtattctgtacataatgtaccatcccatgtggaatctgtgagtgtcctcttaagtagcgtgggctagccaatctgccgttcatggtgtattgtaaactccgaattccatatgtaataggatgcaagtctaagcgtttcatgtggacataaatgtatctaaataaaactttccctagcactgtggctgacctcacccttacttttatactttagtatgaaactgatgagaactttggtagtgagtattttttttatatatatacatatatatgtactatctatatatatatctcaagcatctttcaggtctttgtgtgtggctttcttaaagccctgttgtaaaaaattactatgtggatggcagtctctcacatcacagatgtggaaagtataattttatatttgtattttcaaataaataagtttgtgaaaggtttccatcctctactgtggtccagaaatcaatgtgtttgtctgacaaaaaaaaaaataaaataaaataaactgttttgaacaga

SEQ ID NO: 124 - DNA, 186 bp, Artificial Sequence
  MSI2 Transcript
gccccccaaaataagtctccgtgcctggggaaaggcaccgttcgcttagaaggatcactgagaattgactggagaatgacttccacagaaagcaccctcaggctccaacccggcgcggcccggaggcttcccgggggccaacagcccaggacctgtcgccgatctctacggccctgccagccagga

SEQ ID NO: 125 - DNA, 341 bp, Artificial Sequence
  MSI2 Transcript
gcttcccagcagcggcttatggaccagtggcagcagcggcggtggcggcagcaagaggatcaggatctgcacacctcctctcaaacactcttctgagcacgctcagcgggaaggctccaacccggcgcggcccggaggcttcccgggggccaacagcccaggacctgtcgccgatctctacggccctgccagccaggactccggagtggggaattacataagtgcggccagcccacagccgggctcgggcttcggccacggcatagctggacctttgattgcaacggcctttacaaatggataccattgagcaggtgctttcgttgccatctcactctgag

SEQ ID NO: 126 - DNA, 425 bp, Artificial Sequence
  MSI2 Transcript
taaaatgtttatcggtggactgagctggcagacctcaccagatagccttagagactattttagcaaatttggagaaattagagaatgtatggtcatgagagatcccactacgaaacgctccagaggcttcggtttcgtcacgttcgcagacccagcaagtgtagataaagtattaggtcagccccaccatgagttagattccaagacgattgaccccaaagttgcatttcctcgtcgagcgcaacccaagagggcttcccagagttagcagtatttcctgggtttggaggaattgctgtttcttccataagtgtgaatcttcaaatttccagatggtcacaagaacaaagaaaatatttgtaggcgggttatctgcgaacacagtagtggaagatgtaaagcaatatttcgagcagtttggcaag

SEQ ID NO: 127 - DNA, 1860 bp, Artificial Sequence
  MSI2 Transcript
gctttccttttagcttttgtaagttacacgtcaaaatggccgatctgacatcggtgctcacttctgttatgttttctccctctagtaaaatgtttatcggtggactgagctggcagacctcaccagatagccttagagactattttagcaaatttggagaaattagagaatgtatggtcatgagagatcccactacgaaacgctccagaggcttcggtttcgtcacgttcgcagacccagcaagtgtagataaagtattaggtcagccccaccatgagttagattccaagacgattgaccccaaagttgcatttcctcgtcgagcgcaacccaagatggtcacaagaacaaagaaaatatttgtaggcgggttatctgcgaacacagtagtggaagatgtaaagcaatatttcgagcagtttggcaaggtggaagatgcaatgctgatgtttgataaaactaccaacaggcacagagggtttggctttgtcacttttgagaatgaagatgttgtggagaaagtctgtgagattcatttccatgaaatcaataataaaatggtagaatgtaagaaagctcagccgaaagaagtcatgttcccacctgggacaagaggccgggcccggggactgccttacaccatggacgcgttcatgcttggcatggggatgctgggatatcccaacttcgtggcgacctatggccgtggctaccccggatttgctccaagctatggctatcagttcccagactatttgccggtttcacaagacataatttttatcaactagctcttaagagaggcatagcaaagtgggggttgctaccatttctagagagagaggacacagtcccggcctgggctgcccccgctccagtcaatgctcactgaaagtctgtcttagctgcctgtttgaatgactgttctttttctcatttttaattcttggactcatgtcctcattgcttcactcaattaaaaaaaaattattctccagtcccctcccactttgcttcttgtatgcattgtgaccgaccccacttcctcagaatgtaacggggccagagggaaacttctcacaaacttcgtagagcctcctcaggggaagctaggaagaagacatcaaatgtttttaagtcatgaccaaacaggcttgttggggacatatcatggggtgagctttgaagtgctggtggtccagaggggttgcagatacgtgacttggagcacccgtgtcttacgatggacagtgataaaggtgaacacacagagacagactattctctaagaatgtgagaaacctgatctggaagaggagctatataaacactatctgactatctttgtcttttggggccagtggctgttggcataatcacaagcctgtctgtcttcgagaagggacagtggagtcatccaggtgctgccacatgacaggcacggtgggcaccgatccacagtgggccccgccttccccagctcgcctccctgcctgtgctggcctggccttgcctgctggcaccattggagtaggagggggtggaacacagggggcccatcctgatcaggccccatctcaaggttggcactcctgcccatcacccttagaaggatcttttcccatggcttgacttccttcatttccctaactgaaatacacccactctcttggaataatgacgtaccactcagttggaccctcaagagtcactgctttgtctgtgctggtagtttgtgagaagtgacccgcacgcttccatttgatgcatttgatgtgagtgaatccatacatttgaatgtcattgtccttgagaccctacatgtgcagtttggctcatctcattaaagatgcttgatgt

SEQ ID NO: 128 - DNA, 465 bp, Artificial Sequence
  MSI2 Transcript
ggcagacctcaccagatagccttagagactattttagcaaatttggagaaattagagaatgtatggtcatgagagatcccactacgaaacgctccagaggcttcggtttcgtcacgttcgcagacccagcaagtgtagataaagtattaggtcagccccaccatgagttagattccaagacgattgaccccaaagttgcatttcctcgtcgagcgcaacccaaggtaagtaggagaataaacagtaggattttagcactcagagatgattgccaagaatttcaaatttcaaacagcattggccatgaactgttgaagcctggtattcactgttccttcggggtatcaggacaggctgggcaagtagtcctgtgagataaccatgcgtctctaagttagccatcccaaggtggcttctgaacatccacctgggggcagggacaagtctgatgcttagtgggaggac

SEQ ID NO: 129 - DNA, 1714 bp, Artificial Sequence
  MSI2 Transcript
agagccagagagaacttccagcgcaaaaggaaaataaaacttgtggctggtgtttgtgcaggagggtctccgccatcctgaagccccccgatcctggggcgtctcgggggccgccaaaggagcgccagggttaaaatgtttatcggtggactgagctggcagacctcaccagatagccttagagactattttagcaaatttggagaaattagagaatgtatggtcatgagagatcccactacgaaacgctccagaggcttcggtttcgtcacgttcgcagacccagcaagtgtagataaagtattaggtcagccccaccatgagttagattccaagacgattgaccccaaagttgcatttcctcgtcgagcgcaacccaagatggtcacaagaacaaagaaaatatttgtaggcgggttatctgcgaacacagtagtggaagatgtaaagcaatatttcgagcagtttggcaaggtggaagatgcaatgctgatgtttgataaaactaccaacaggcacagagggtttggctttgtcacttttgagaatgaagatgttgtggagaaagtctgtgagattcatttccatgaaatcaataataaaatggtagaatgtaagaaagctcagccgaaagaagtcatgttcccacctgggacaagaggccgggcccggggactgccttacaccatggacgcgttcatgcttggcatggggatgctgggatatcccaacttcgtggcgacctatggccgtggctaccccggatttgctccaagctatggctatcagttcccaggcttcccagcagcggcttatggaccagtggcagcagcggcggtggcggcagcaagaggatcagtcctgaatagctacagtgctcaaccgaattttggcgcgcccgcttccccggcaggctccaacccggcgcggcccggaggcttcccgggggccaacagcccaggacctgtcgccgatctctacggccctgccagccaggactccggagtggggaattacataagtgcggccagcccacagccgggctcgggcttcggccacggcatagctggacctttgattgcaacggcctttacaaatggataccattgagcaggtgctttcgttgccatctcactctgagagcatacctggatgtccaggcaagactgggcgaagtttctgagtggccctttgtttaggtgatgtcctcagacctggacccccaccagcctcactccccatcccaaccagagatggctcacttcggatcgagggttgactacatctcatcatctcacgaatctgctgtaatataagacaacagcttttaaatgtgtatataacccatgatttcggttttgttttgttttgtttttcttgatggtttccctctccctccctctcttcccattctccttttaaatctctttgaatcacatttggtagtgattttgacttagtccagtagtcacatagctttaatatctagttcaaagctaaccatagtataattgttatattaaggagttattttttcttaaaacatttttttttgcttgttttggttctgttctcacttttaaaggatgctgagatggtaatatgactctccatattttggtaccaattctgagactgtatgaattttcaggtggaactttagcacacactgaagcaaagttgtgaagtgcagggcgggaggtgggcgtgagct

SEQ ID NO: 130 - DNA, 534 bp, Artificial Sequence
  MSI2 Transcript
atggaggcaaatgggagccaaggcacctcgggcagcgccaacgactcccagcacgaccccggtaaaatgtttatcggtggactgagctggcagacctcaccagatagccttagagactattttagcaaatttggagaaattagagaatgtatggtcatgagagatcccactacgaaacgctccagaggcttcggtttcgtcacgttcgcagacccagcaagtgtagataaagtattaggtcagccccaccatgagttagattccaagacgattgaccccaaagttgcatttcctcgtcgagcgcaacccaaggtgttctagctgagcgtttaagagcatggactctggaaccagactttgaatccttgctctgccactgcagctgtgtgaccttgagcaagctatctaaatattctgtgcctttgttttgtcatctgtacaatggagatgatgatggtatcaccttcatggggttgtcataaggattatgggacttaacataataaagtgtttagaatggtgtctgcaacataa

SEQ ID NO: 131 - DNA, 577 bp, Artificial Sequence
  MSI2 Transcript
atttatgtgcccctgtgcatgggtgtggatgtgatttcctggcgtgcagggtggaagatgcaatgctgatgtttgataaaactaccaacaggcacagagggtttggctttgtcacttttgagaatgaagatgttgtggagaaagtctgtgagattcatttccatgaaatcaataataaaatggtagaatgtaagaaagctcagccgaaagaagtcatgttcccacctgggacaagaggccgggcccggggactgccttacaccatggacgcgttcatgcttggcatggggatgctgggatatcccaacttcgtggcgacctatggccgtggctaccccggatttgctccaagctatggctatcagttcccaggcttcccagcagcggcttatggaccagtggcagcagcggcggtggcggcagcaagaggatcagtcctgaatagctacagtgctcaaccgaattttggcgcgcccgcttccccggcaggctccaacccggcgcggcccggaggcttcccgggggccaacagcccaggacctgtcgccgatctctacggccctgccagccaggactc

SEQ ID NO: 132 - DNA, 493 bp, Artificial Sequence
  MSI2 Transcript
gatatggaggcaaatgggagccaaggcacctcgggcagcgccaacgactcccagcacgaccccggtaaaatgtttatcggtggactgagctggcagacctcaccagatagccttagagactattttagcaaatttggagaaattagagaatgtatggtcatgagagatcccactacgaaacgctccagaggcttcggtttcgtcacgttcgcagacccagcaagtgtagataaagtattaggtcagccccaccatgagttagattccaagacgtgcaggaggcagaagcggttggtttattgacatggaaacgaggatcttgtttactgttaccttaaaagctttgtttagattgaccccaaagttgcatttcctcgtcgagcgcaacccaagcattgctgactttcttcctccatggcacaggatttttgcccaaaacaaaagtctcaatttatttgttctttaaaaaaataaaaaccacactgaacaaaaa

SEQ ID NO: 133 - DNA, 246 bp, Artificial Sequence
  MSI2 Transcript
taaaatgtttatcggtggactgagctggcagacctcaccagccttagagactattttagcaaatttggagaaattagagaatgtatggtcatgagagatcccactacgaaacgctccagaggcttcggtttcgtcacgttcgcagacccagcaagtgtagataaagtattaggtcagccccaccatgagttagattccaagacgattgaccccaaagttgcatttcctcgtcgagcgcaacccaag

SEQ ID NO: 134 - DNA, 1921 bp, Artificial Sequence
  MSI2 transcript
acatttcagcaggcagaggcaggcaatatggccctgagcaagggaagtacagaagtgggaagtgcaggggacttcagatcacggggtgagggaggaggctcactgagagagaagaccagtactgtgctctgtggggccagctcatgagaccgcaaagatagtagcagctttgatgccaggctgggaattctgcctgcgctgttgggagccagtcacgtgttccacacaggtggaagatgcaatgctgatgtttgataaaactaccaacaggcacagagggtttggctttgtcacttttgagaatgaagatgttgtggagaaagtctgtgagattcatttccatgaaatcaataataaaatggtagaatgtaagaaagctcagccgaaagaagtcatgttcccacctgggacaagaggccgggcccggggactgccttacaccatggacgcgttcatgcttggcatggggatgctgggatatcccaacttcgtggcgacctatggccgtggctaccccggatttgctccaagctatggctatcagttcccagactatttgccggtttcacaagacataatttttatcaactagctcttaagagaggcatagcaaagtgggggttgctaccatttctagagagagaggacacagtcccggcctgggctgcccccgctccagtcaatgctcactgaaagtctgtcttagctgcctgtttgaatgactgttctttttctcatttttaattcttggactcatgtcctcattgcttcactcaattaaaaaaaaattattctccagtcccctcccactttgcttcttgtatgcattgtgaccgaccccacttcctcagaatgtaacggggccagagggaaacttctcacaaacttcgtagagcctcctcaggggaagctaggaagaagacatcaaatgtttttaagtcatgaccaaacaggcttgttggggacatatcatggggtgagctttgaagtgctggtggtccagaggggttgcagatacgtgacttggagcacccgtgtcttacgatggacagtgataaaggtgaacacacagagacagactattctctaagaatgtgagaaacctgatctggaagaggagctatataaacactatctgactatctttgtcttttggggccagtggctgttggcataatcacaagcctgtctgtcttcgagaagggacagtggagtcatccaggtgctgccacatgacaggcacggtgggcaccgatccacagtgggccccgccttccccagctcgcctccctgcctgtgctggcctggccttgcctgctggcaccattggagtaggagggggtggaacacagggggcccatcctgatcaggccccatctcaaggttggcactcctgcccatcacccttagaaggatcttttcccatggcttgacttccttcatttccctaactgaaatacacccactctcttggaataatgacgtaccactcagttggaccctcaagagtcactgctttgtctgtgctggtagtttgtgagaagtgacccgcacgcttccatttgatgcatttgatgtgagtgaatccatacatttgaatgtcattgtccttgagaccctacatgtgcagtttggctcatctcattaaagatgcttgatgtaataattggttagtttccttttattttcctgcaggcttttccatgagtattatttttttcaaagaacaaatctgtatggcttttccccatctccatattttgttttgctatgaattgctttgctttggtgaacttgtcctagtatgcttgcctcacaaacgttttagccattgtgaattttcttcatctctgtaaatagttcatctgtgcttctccctgatgacgttttattttttttcccctgtaagcaaccgaggtag

SEQ ID NO: 135 - DNA, 548 bp, Artificial Sequence
  MSI2 transcript
aaatgtcagagccaggcgcaggctttgtgtttggtgctgatgcatggcatttactctgaaggttcaggaaagtacatgggctttgcggggtctggctgcccaaatatgggttaattagcaacagctgacagcagcgcccccggccacaggagagaggtgacccagacccttaataaaaaaaccctcatgagagacaaattattctgtgaaggaaaataactcaggctttcctcattgccaccctccgtgagattttaccccagacctgaggcggctgtactaacaggactctgatctttctctttgtgttcaaggatatcccaacttcgtggcgacctatggccgtggctaccccggatttgctccaagctatggctatcagttcccagactatttgccggtttcacaagacataatttttatcaactagctcttaagagaggcatagcaaagtgggggttgctaccatttctagagagagaggacacagtcccggcctgggctgcccccgctccagtcaatgctcactgaaagtctgtcttagctgc

SEQ ID NO: 136 - DNA, 452 bp, Artificial Sequence
  MSI2 transcript
ccttttagcttttgtaagttacacgtcaaaatggccgatctgacatcggtgctcacttctgttatgttttctccctctagtaaaatgtttatcggtggactgagctggcagacctcaccagatagccttagagactattttagcaaatttggagaaattagagaatgtatggtcatgagagatcccactacgaaacgctccagaggcttcggtttcgtcacgttcgcagacccagcaagtgtagataaagtattaggtcagccccaccatgagttagattccaagacgattgaccccaaagttgcatttcctcgtcgagcgcaacccaagatggtcacaagaacaaagaaaatatttgtaggcgggttatctgcgaacacagtagtggaagatgtaaagcaatatttcgagcagtttggcaaggtttccgccggcagcccaggcctctccct

SEQ ID NO: 137 - DNA, 119 bp, Artificial Sequence
  MSI2 transcript
gagaaggaccgcaaactcacagaggaacgggtgtgagaccattcatatcatcagtccaccatagacctgtgtggaagatgcaatgctgatgtttgataaaactaccaacaggcacagag

SEQ ID NO: 138 - DNA, 710 bp, Artificial Sequence
  MSI2 transcript
gtggagaaagtctgtgagattcatttccatgaaatcaataataaaatggtagaatgtaagaaagctcagccgaaagaagtcatgttcccacctgggacaagaggccgggcccggggactgccttacaccatggacgcgttcatgcttggcatggggatgctgggtgagtctggacaggaccgcaggtcaccatggactgggagggctatggaggcctctactcccaactgggtcacctaccagtggggcaaactgcttcacctttctaagcctcagtttccttgtctgtagatgaggatgataattccccgttccaagacagttgtgatgattaagtgtgggtgtgtgtgtgtgcatgcatgtgtgtgtgtgtgtgtgtgtgtttgtatttataatattgccccatgcctggcttataggatatgttagactattttctctcttttccatctccttcctcaaaagaaggaaaagtccccctctatctgcctcagccctctcatctgagtgggagttcttaagatgtaaggactcctggctgacttgacttgtgtgggctaaggctacgttttctaaaacttgggagaggagggaagtggtaagggtgggcgataatcctgtctatttaaatgattaacatttttctcttgggatatcaaaatttgcatttaaatggatgttttaaatagcctgttttactctttatttgc